(19) United States
(12) Patent Application Publication
(10) Pub. No.: US 2019/0134195 A1
(43) Pub. Date: May 9, 2019

(54) COMPOSITIONS AND METHODS FOR THE TREATMENT OF HUMAN PAPILLOMAVIRUS (HPV)-ASSOCIATED DISEASES

(71) Applicant: ETUBICS CORPORATION, Seattle, WA (US)

(72) Inventors: Frank R. JONES, Seattle, WA (US); Joseph BALINT, Seattle, WA (US); Yvette LATCHMAN, Seattle, WA (US); Adrian RICE, Seattle, WA (US); Elizabeth GABITZSCH, Seattle, WA (US)

(21) Appl. No.: 16/306,076

(22) PCT Filed: Jun. 2, 2017
(86) PCT No.: PCT/US17/35841

§ 371 Date: Nov. 30, 2018

Related U.S. Application Data
(60) Provisional application No. 62/345,592, filed on Jun. 3, 2016.

(51) Int. Cl. A61K 39/395; A61P 31/20; A61P 35/00; A61K 39/235; A61K 47/00; C12N 15/113; A61K 39/39

(52) U.S. Cl. CPC A61K 39/39541; A61P 31/20; A61P 35/00; A61K 2039/53; A61K 47/00; C12N 15/1132; A61K 39/39; A61K 39/235

(57) ABSTRACT

Methods and compositions for constructing and producing recombinant adenovirus-based vector vaccines are provided. In particular aspects, there are be provided compositions and methods involving adenovirus vectors comprising genes for target antigens, such as novel antigens of HPV E6 and/or HPV E7 for use in treatment methods that generate highly reactive anti-HPV and anti-tumor immune responses in subjects with preexisting immunity to adenovirus.

### CROSS REFERENCE

This application claims the benefit of U.S. Provisional Patent Application No. 62/345,592 filed Jun. 3, 2016, the disclosure of which is herein incorporated by reference in its entirety.

### STATEMENT AS TO FEDERALLY SPONSORED RESEARCH

The invention was made with government support under SBIR Grant Numbers 1R43DE021973-01, 2R44DE021973-02, and 3R44DE021973-03S1 awarded by the National Institute of Dental and Craniofacial Research (NIDCR). The government has certain rights in the invention.

### BACKGROUND

Vaccines help the body fight diseases by training the immune system to recognize and destroy harmful substances and diseased cells. Vaccines can be largely grouped into two types, preventive and treatment vaccines. Prevention vaccines are given to healthy people to prevent the development of specific diseases, while treatment vaccines, also referred to as immunotherapies, are given to a person who has been diagnosed with disease to help stop the disease from growing and spreading or as a preventive measure.

Viral vaccines are currently being developed to vaccinate against infectious diseases and treat infectious disease-induced cancers by immunotherapy. These viral vaccines work by inducing expression of a small fraction of genes associated with a disease within the host's cells, which in turn, enhance the host's immune system to identify and destroy diseased cells containing infectious agents. As such, clinical response of a viral vaccine can depend on the ability of the vaccine to obtain a high-level immunogenicity and have sustained long-term expression.

Therefore, there remains a need to discover novel compositions and methods for enhanced therapeutic response to complex diseases such as cancer, such as human papillomavirus (HPV)-associated diseases or HPV-induced cancers.

### SUMMARY

In various aspects, the present disclosure provides a composition comprising a replication-defective virus vector comprising a nucleic acid sequence comprising one or more of: a) a nucleic acid sequence encoding an amino acid sequence at least 80%, at least 85%, at least 90%, at least 92%, at least 95%, at least 97%, or at least 99% identical to SEQ ID NO: 8, SEQ ID NO: 9, or SEQ ID NO: 10; b) a nucleic acid sequence encoding an amino acid sequence at least 80%, at least 85%, at least 90%, at least 92%, at least 95%, at least 97%, or at least 99% identical to SEQ ID NO: 12; c) a nucleic acid sequence having a region at least 80%, at least 85%, at least 90%, at least 92%, at least 95%, at least 97%, or at least 99% identical to SEQ ID NO: 2, SEQ ID NO: 3, or SEQ ID NO: 4; d) a nucleic acid sequence having a region at least 80%, at least 85%, at least 90%, at least 92%, at least 95%, at least 97%, or at least 99% identical to SEQ ID NO: 5, SEQ ID NO: 18, SEQ ID NO: 6, SEQ ID NO: 19, or SEQ ID NO: 7, SEQ ID NO: 20; and e) a nucleic acid sequence having a region at least 80%, at least 85%, at least 90%, at least 92%, at least 95%, at least 97%, or at least 99% identical to SEQ ID NO: 11, or SEQ ID NO: 21.

In some aspects, the vector comprises a nucleic acid sequence encoding an amino acid sequence at least 80%, at least 85%, at least 90%, at least 92%, at least 95%, at least 97%, or at least 99% identical to SEQ ID NO: 8. In other aspects, the vector comprises a nucleic acid sequence encoding an amino acid sequence at least 80%, at least 85%, at least 90%, at least 92%, at least 95%, at least 97%, or at least 99% identical to SEQ ID NO: 9. In still other aspects, the vector comprises a nucleic acid sequence encoding an amino acid sequence at least 80%, at least 85%, at least 90%, at least 92%, at least 95%, at least 97%, or at least 99% identical to SEQ ID NO: 10.

In other aspects, the vector comprises a nucleic acid sequence encoding an amino acid sequence at least 80%, at least 85%, at least 90%, at least 92%, at least 95%, at least 97%, or at least 99% identical to SEQ ID NO: 12. In other aspects, the vector comprises a nucleic acid sequence comprising a region at least 80%, at least 85%, at least 90%, at least 92%, at least 95%, at least 97%, or at least 99% identical to SEQ ID NO: 2. In still other aspects, the vector comprises a nucleic acid sequence comprising a region at least 80%, at least 85%, at least 90%, at least 92%, at least 95%, at least 97%, or at least 99% identical to SEQ ID NO: 3.

In still other aspects, the vector comprises a nucleic acid sequence comprising a region at least 80%, at least 85%, at least 90%, at least 92%, at least 95%, at least 97%, or at least 99% identical to SEQ ID NO: 4. In some aspects, the vector comprises a nucleic acid sequence comprising a region at least 80%, at least 85%, at least 90%, at least 92%, at least 95%, at least 97%, or at least 99% identical to SEQ ID NO: 5. In some aspects, the vector comprises a nucleic acid sequence comprising a region at least 80%, at least 85%, at least 90%, at least 92%, at least 95%, at least 97%, or at least 99% identical to SEQ ID NO: 18. In some aspects, the vector comprises a nucleic acid sequence comprising a region at least 80%, at least 85%, at least 90%, at least 92%, at least 95%, at least 97%, or at least 99% identical to SEQ ID NO: 6. In other aspects, the vector comprises a nucleic acid sequence comprising a region at least 80%, at least 85%, at least 90%, at least 92%, at least 95%, at least 97%, or at least 99% identical to SEQ ID NO: 19.

In some aspects, the vector comprises a nucleic acid sequence comprising a region at least 80%, at least 85%, at least 90%, at least 92%, at least 95%, at least 97%, or at least 99% identical to SEQ ID NO: 7. In other aspects, the vector comprises a nucleic acid sequence comprising a region at least 80%, at least 85%, at least 90%, at least 92%, at least 95%, at least 97%, or at least 99% identical to SEQ ID NO: 20. In some aspects, the vector comprises a nucleic acid sequence comprising a region at least 80%, at least 85%, at least 90%, at least 92%, at least 95%, at least 97%, or at least 99% identical to SEQ ID NO: 11. In some aspects, the vector comprises a nucleic acid sequence comprising a region at least 80%, at least 85%, at least 90%, at least 92%, at least 95%, at least 97%, or at least 99% identical to SEQ ID NO: 21.

In some aspects, the vector is an adenovirus vector. In further aspects, the vector comprises a deletion in an E1 region, an E2b region, an E3 region, an E4 region, or a combination thereof. In further aspects, the vector comprises a deletion in an E2b region. In still further aspects, the vector comprises a deletion in an E1 region, an E2b region, and an E3 region.

In some aspects, the composition or the vector further comprises a nucleic acid sequences encoding a costimulatory molecule. In some aspects, the costimulatory molecule comprises B7, ICAM-1, LFA-3, or a combination thereof. In further aspects, the costimulatory molecule comprises a combination of B7, ICAM-1, and LFA-3. In some aspects, the composition further comprises a plurality of nucleic acid sequences encoding a plurality of costimulatory molecules positioned in the same replication-defective virus vector. In some aspects, the composition further comprises a plurality of nucleic acid sequences encoding a plurality of costimulatory molecules positioned in separate replication-defective virus vectors. In some aspects, the composition comprises at least 5×10¹¹ replication-defective virus vectors.

In some aspects, the composition comprises a nucleotide sequence encoding a fusion protein comprising HPV E6 and HPV E7. In some aspects, the composition comprises: a first replication defective adenovirus vector comprising: a deletion in the E2b region, and a nucleic acid sequence encoding HPV E6; and a second replication defective adenovirus vector comprising: a deletion in the E2b region, and a nucleic acid sequence encoding HPV E7. In some aspects, the replication-defective virus vector further comprises a nucleic acid sequence encoding a selectable marker. In further aspects, the selectable marker is a lacZ protein, thymidine kinase, gpt, GUS, or a vaccinia K1L host range protein, or a combination thereof. In some aspects, the modified HPV antigen is a combination of the modified HPV E6 antigen and the modified HPV E7 antigen.

In further aspects, the modified HPV antigen is a non-oncogenic HPV antigen. In still further aspects, the modified HPV antigen binds to HLA-A2, HLA-A3, HLA-A24, or a combination thereof. In some aspects, the nucleic acid sequence has a region at least 80%, at least 85%, at least 90%, at least 92%, at least 95%, at least 97%, or at least 99% identical to positions 23-496 and 502-795 of SEQ ID NO: 2, SEQ ID NO: 3, SEQ ID NO: 4, or a combination thereof. In some aspects, the nucleic acid sequence has at least 80%, at least 85%, at least 90%, at least 92%, at least 95%, at least 97%, or at least 99% identity to SEQ ID NO: 5, SEQ ID NO: 18, SEQ ID NO: 6, SEQ ID NO: 19, SEQ ID NO: 7, or SEQ ID NO: 20. In some aspects, the nucleic acid sequence has at least 80%, at least 85%, at least 90%, at least 92%, at least 95%, at least 97%, or at least 99% identity to SEQ ID NO: 11 or SEQ ID NO: 21.

In some aspects, the replication-defective virus further comprises a nucleic acid sequence encoding one or more additional target antigens or immunological epitopes thereof. In further aspects, the one or more additional target antigens is a tumor neo-antigen, tumor neo-epitope, tumor-specific antigen, tumor-associated antigen, tissue-specific antigen, bacterial antigen, viral antigen, yeast antigen, fungal antigen, protozoan antigen, parasite antigen, mitogen, or a combination thereof. In some aspects, the one or more additional target antigens is CEA, folate receptor alpha, WT1, HPV E6, HPV E7, p53, MAGE-A1, MAGE-A2, MAGE-A3, MAGE-A4, MAGE-A6, MAGE-AO1, MAGE-A12, BAGE, DAM-6, -10, GAGE-1, -2, -8, GAGE-3, -4, -5, -6, -7B, NA88-A, NY-ESO-1, MART-1, MC1R, Gp100, PSCA, PSMA, PAP, Tyrosinase, TRP-1, TRP-2, ART-4, CAMEL, Cyp-B, Her2/neu, BRCA1, BRACHYURY, BRACHYIJRY (TIVS7-2, polymorphism), BRACHYURY (IVS7 T/C polymorphism), T BRACHYURY, T, hTERT, hTRT, iCE, MUC1, MUC1 (VNTR polymorphism), MUC1c, MUCin, MUC2, PRAME, P15, RU1, RU2, SART-1, SART-3, WT1, AFP, β-catenin/m, Caspase-8/m, CDK-4/m, Her2/neu, Her3, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist, or a combination thereof.

In some aspects, the one or more additional target antigens is CEA, Brachyury, and MUC1. In further aspects CEA comprises a sequence at least 80%, at least 85%, at least 90%, at least 92%, at least 95%, at least 97%, or at least 99% identical to SEQ ID NO: 22, SEQ ID NO: 24, or positions 1057-3165 of SEQ ID NO: 25. In some aspects, MUC1-c comprises a sequence at least 80%, at least 85%, at least 90%, at least 92%, at least 95%, at least 97%, or at least 99% identical to SEQ ID NO: 26 or SEQ ID NO: 27. In some aspects, Brachyury comprises a sequence at least 80%, at least 85%, at least 90%, at least 92%, at least 95%, at least 97%, or at least 99% identical to SEQ ID NO: 28.

In some aspects, the composition comprises from at least 1×10⁹ virus particles to at least 5×10¹² virus particles. In some aspects, the composition comprises at least 1×10¹¹ virus particles. In other aspects, the composition comprises at least 5×10¹¹ virus particles. In some aspects, the replication-defective virus vector further comprises a nucleic acid sequence encoding an immunological fusion partner.

In various aspects, the present disclosure provides a pharmaceutical composition comprising any one of the above described compositions and a pharmaceutically acceptable carrier.

In various aspects, the present disclosure provides a host cell comprising any one of the above described compositions.

In various aspects, the present disclosure provides a method of preparing a tumor vaccine, comprising preparing any pharmaceutical composition described above or preparing any composition described above.

In various aspects, the present disclosure provides a method of enhancing an HPV-specific immune response in a subject in need thereof, the method comprising administering a therapeutically effective amount of any composition described above or any pharmaceutical composition described above to the subject.

In various aspects, the present disclosure provides a method of preventing or treating a HPV-induced cancer in a subject in need thereof, the method comprising administering a therapeutically effective amount of any composition described above or any pharmaceutical composition described above to the subject. In some aspects, the administering eliminates HPV E6- or HPV E7-expressing cells in the subject. In some aspects, the method is a method of preventing a HPV-induced cancer in a subject determined to be HPV positive prior to the administering. In some aspects, the subject is positive for expression of HPV type 16 or HPV type 18 oncogenes.

In further aspects, the method further comprises administering an adjuvant, wherein the adjuvant comprises Freund's incomplete adjuvant, Freund's complete adjuvant, Merck adjuvant 65, AS-2, aluminum hydroxide gel (alum), aluminum phosphate, salts of calcium, iron or zinc, acylated tyrosine, acylated sugars, cationically or anionically derivatized polysaccharides, polyphosphazenes, biodegradable microspheres, monophosphoryl lipid A, quil A, GM-CSF, IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, or IL-32. In some aspects, the subject is HPV positive or expresses HPV E6 or HPV E7. In some aspects, the method further comprises administering to the subject an immune checkpoint inhibitor. In some aspects, the immune checkpoint inhibitor targets PD-1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA, or CD244.

In further aspects, the immune checkpoint inhibitor targets PD-1 or PDL1. In some aspects, the immune checkpoint inhibitor is an anti-PD-1 or anti-PDL1 antibody. In some aspects, the immune checkpoint inhibitor is an anti-PDL1 antibody. In further aspects, the immune checkpoint inhibitor is avelumab. In some aspects, the method is further comprises treating an HPV infection, an HPV-induced cancer, or an HPV-associated disease in a subject in need thereof. In some aspects, the subject has an HPV infection, an HPV-induced cancer, or an HPV-associated disease. In some aspects, the HPV-induced cancer is HPV-induced head and neck squamous cell carcinoma (HNSCC), oropharyngeal and tonsillar cancer, vaginal cancer, penis cancer, vulva cancer, anal cancer, or cervical cancer. In some aspects, the subject has HPV-positive squamous cell carcinoma of the cervix, vagina, vulva, head/neck, anus, or penis.

In some aspects, the subject has pre-existing immunity to Ad5. In some aspects, the administering the therapeutically effective amount of the composition is repeated at every three weeks. In some aspects, the pharmaceutical composition comprises at least 5×10¹¹ adenovirus vectors. In further aspects, the method further comprises administering to the subject a chemotherapy, radiation, or a combination thereof. In some aspects, a route of administration is intravenous, subcutaneous, intralymphatic, intratumoral, intradermal, intramuscular, intraperitoneal, intrarectal, intravaginal, intranasal, oral, via bladder instillation, or via scarification. In some aspects, the subject has enhanced immune response that is a cell-mediated or humoral response after the administering. In some aspects, the subject has enhanced immune response that is an enhancement of B-cell proliferation, CD4+ T cell proliferation, CD8+ T cell proliferation, or a combination thereof.

In some aspects, the subject has enhanced immune response that is an enhancement of IL-2 production, IFN-γ production or combination thereof. In further aspects, the subject has enhanced immune response that is an enhancement of antigen presenting cell proliferation, function or combination thereof. In some aspects, the subject has been previously administered an adenovirus vector. In some aspects, the subject is determined to have pre-existing immunity to adenovirus vectors.

In further aspects, the method further comprises administering to the subject a pharmaceutical composition comprising a population of engineered nature killer (NK) cells. In some aspects, the engineered NK cells comprise one or more NK cells that have been modified as essentially lacking the expression of KIR (killer inhibitory receptors), one or more NK cells that have been modified to express a high affinity CD16 variant, and one or more NK cells that have been modified to express one or more CARs (chimeric antigen receptors), or any combinations thereof. In some aspects, the engineered NK cells comprise one or more NK cells that have been modified as essentially lacking the expression KIR. In other aspects, the engineered NK cells comprise one or more NK cells that have been modified to express a high affinity CD16 variant. In still other aspects, the engineered NK cells comprise one or more NK cells that have been modified to express one or more CARs.

In some aspects, the CAR is a CAR for a tumor neo-antigen, tumor neo-epitope, WT1, HPV E6, HPV E7, p53, MAGE-A1, MAGE-A2, MAGE-A3, MAGE-A4, MAGE-A6, MAGE-AO1, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, PSA, PSM, PSMA, Tyrosinase, TRP-1, TRP-2, ART-4, CAMEL, CEA, Cyp-B, Her1, Her2/neu, Her3, Her4, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, PSCA, PSMA, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPl/mbcr-abl, ETV6/AML, LDLR/FUT, Pml/RARα, TEL/AML1, or any combination thereof.

In some aspects, the adenovirus vector is replication-defective. In some aspects, the replication-defective adenovirus vector is comprised in a cell. In further aspects, the cell is a dendritic cells (DC). In some aspects, the method further comprises administering a pharmaceutical composition comprising a therapeutically effective amount of IL-15 or a replication-defective vector comprising a nucleic acid sequence encoding IL-15. In some aspects, the method further comprises administering a pharmaceutical composition comprising a therapeutically effective amount of an IL-15 superagonist or a replication-defective vector comprising a nucleic acid sequence encoding for an IL-15 superagonist. In further aspects, the IL-15 superagonist is ALT-803.

In various aspects, the present disclosure provides a method of reducing HPV-expressing cells in a subject in need thereof, the method comprising administering an effective amount of a composition comprising a replication-defective virus vector comprising a nucleic acid sequence encoding a modified HPV E6, a modified HPV E7 antigen, or a combination thereof. In some aspects, the nucleic acid sequence encodes a modified HPV E6 and a modified HPV E7. In some aspects, the replication-defective virus vector comprises a) a nucleic acid sequence encoding an amino acid sequence at least 80%, at least 85%, at least 90%, at least 92%, at least 95%, at least 97%, or at least 99% identical to SEQ ID NO: 8, SEQ ID NO: 9, or SEQ ID NO: 10; b) a nucleic acid sequence encoding an amino acid sequence at least 80%, at least 85%, at least 90%, at least 92%, at least 95%, at least 97%, or at least 99% identical to SEQ ID NO: 12; c) a nucleic acid sequence having a region at least 80%, at least 85%, at least 90%, at least 92%, at least 95%, at least 97%, or at least 99% identical to SEQ ID NO: 2, SEQ ID NO: 3, or SEQ ID NO: 4; d) a nucleic acid sequence having a region at least 80%, at least 85%, at least 90%, at least 92%, at least 95%, at least 97%, or at least 99% identical to SEQ ID NO: 5, SEQ ID NO: 18, SEQ ID NO: 6, SEQ ID NO: 19, SEQ ID NO: 7, SEQ ID NO: 20; e) a nucleic acid sequence having a region at least 80%, at least 85%, at least 90%, at least 92%, at least 95%, at least 97%, or at least 99% identical to SEQ ID NO: 11 or SEQ ID NO: 21; f) a nucleic acid sequence encoding an amino acid sequence at least 80%, at least 85%, at least 90%, at least 92%, at least 95%, at least 97%, or at least 99% identical to SEQ ID NO: 13; g) a nucleic acid sequence encoding an amino acid sequence at least 80%, at least 85%, at least 90%, at least 92%, at least 95%, at least 97%, or at least 99% identical to SEQ ID NO: 14; or h) a nucleic acid sequence comprising a region at least 80%, at least 85%, at least 90%, at least 92%, at least 95%, at least 97%, or at least 99% identical to SEQ ID NO: 15.

In some aspects, the administering eliminates HPV E6 or HPV E7-expressing cells in the subject. In some aspects, the method further comprises preventing a HPV-induced cancer in a subject determined to be HPV positive prior to the administering. In some aspects, the vector is an adenovirus vector. In further aspects, the vector comprises a deletion in an E1 region, an E2b region, an E3 region, an E4 region, or a combination thereof. In still further aspects, the vector comprises a deletion in an E2b region. In still further aspects, the vector comprises a deletion in an E1 region, an E2b region, and an E3 region.

In some aspects, the composition or the vector further comprises a nucleic acid sequences encoding a costimulatory molecule. In some aspects, the costimulatory molecule comprises B7, ICAM-1, LFA-3, or a combination thereof. In further aspects, the costimulatory molecule comprises a combination of B7, ICAM-1, and LFA-3. In still further aspects, the composition further comprises a plurality of nucleic acid sequences encoding a plurality of costimulatory molecules positioned in the same replication-defective virus vector. In some aspects, the composition further comprises a plurality of nucleic acid sequences encoding a plurality of costimulatory molecules positioned in separate replication-defective virus vectors. In some aspects, the composition comprises at least 5×10¹¹ replication-defective virus vectors.

In some aspects, the composition comprises a nucleotide sequence encoding a fusion protein comprising HPV E6 and HPV E7. In some aspects, the composition comprises: a first replication defective adenovirus vector comprising: a deletion in the E2b region, and a nucleic acid sequence encoding HPV E6; and a second replication defective adenovirus vector comprising: a deletion in the E2b region, and a nucleic acid sequence encoding HPV E7. In some aspects, the replication-defective virus vector further comprises a nucleic acid sequence encoding a selectable marker.

In further aspects, the selectable marker is a lacZ protein, thymidine kinase, gpt, GUS, or a vaccinia K1L host range protein, or a combination thereof. In some aspects, the modified HPV E6 or HPV E7 antigen is a non-oncogenic HPV antigen. In some aspects, the modified HPV E6 or HPV E7 antigen binds to HLA-A2, HLA-A3, HLA-A24, or a combination thereof. In further aspects, the nucleic acid sequence comprises a region at least 80%, at least 85%, at least 90%, at least 92%, at least 95%, at least 97%, or at least 99% identical to positions 23-496 and 502-795 of SEQ ID NO: 2, SEQ ID NO: 3, SEQ ID NO: 4, or a combination thereof.

In other aspects, the nucleic acid sequence comprises at least 80%, at least 85%, at least 90%, at least 92%, at least 95%, at least 97%, or at least 99% identity to SEQ ID NO: 5, SEQ ID NO: 18, SEQ ID NO: 6, SEQ ID NO: 19, SEQ ID NO: 7, or SEQ ID NO: 20. In other aspects, the nucleic acid sequence comprises at least 80%, at least 85%, at least 90%, at least 92%, at least 95%, at least 97%, or at least 99% identity to SEQ ID NO: 11 or SEQ ID NO: 21. In some aspects, the subject is positive for expression of HPV type 16 or HPV type 18 oncogenes. In some aspects, the subject is determined to be HPV positive or expresses HPV E6 or HPV E7. In some aspects, the subject has an HPV infection.

In some aspects, the subject has been determined to have an HPV infection by oral wash or pap smear. In some aspects, the subject has pre-existing immunity to Ad5. In some aspects, the administering is repeated at every three weeks. In some aspects, the composition comprises at least 5×10¹¹ adenovirus vectors. In some aspects, a route of administration is intravenous, subcutaneous, intralymphatic, intratumoral, intradermal, intramuscular, intraperitoneal, intrarectal, intravaginal, intranasal, oral, via bladder instillation, or via scarification.

In some aspects, the route of administration is subcutaneous administration. In some aspects, the subject has been previously administered an adenovirus vector. In some aspects, the subject is determined to have pre-existing immunity to adenovirus vectors. In some aspects, the administering the therapeutically effective amount of the composition comprises 1×10⁹ to 5×10¹² virus particles per dose. In further aspects, the administering the therapeutically effective amount of the composition comprises at least 1×10¹¹ virus particles per dose. In still further aspects, the administering the therapeutically effective amount of the composition comprises at least 5×10¹¹ virus particles per dose.

In some aspects, the administering the therapeutically effective amount of the composition is followed by one or more booster immunizations comprising the same composition or pharmaceutical composition. In further aspects, the booster immunization is administered every one, two, or three months. In some aspects, the booster immunization is repeated three or more times. In some aspects, the administering the therapeutically effective amount is a primary immunization repeated every one, two, or three weeks for three times followed by a booster immunization repeated every one, two, or three months for three or more times.

### BRIEF DESCRIPTION OF THE DRAWINGS

FIG. 1A exemplifies changes in tumor size from immunotherapy of C57BL/6 mice (n=5/group) implanted on day 0 with 2×10⁵ non-palpable HPV E6/E7 TC-1 tumor cells and administered 1×10¹⁰ Ad5 [E1-, E2b-]-null virus particles (VPs) or 1×10¹⁰ Ad5 [E1-, E2b-]-E6/E7 VPs on days 1, 8, and 15. Tumor size was determined and volumes calculated according to the formula V=(tumor width²×tumor length)/2. Analysis of significance was performed between experimental and vector control groups using unpaired t-tests and significance is denoted by * (p<0.05) and ** (p<0.01).

FIG. 1B exemplifies a survival curve of the mice as described in FIG. 1A that was plotted and compared using the Mantel-Cox test. Significance is denoted by ** (p<0.01).

FIG. 2A exemplifies changes in tumor size from immunotherapy of C57BL/6 mice (n=4/group) implanted on day 0 with 2×10⁵ small palpable HPV E6/E7 TC-1 tumor cells and administered 1×10¹⁰ Ad5 [E1-, E2b-]-null VPs or 1×10¹⁰ Ad5 [E1-, E2b-]-E6/E7 VPs on days 6, 13, and 20. Tumor size was determined and volumes calculated according to the formula V=(tumor width²×tumor length)/2. Analysis of significance was performed between experimental and vector control groups using unpaired t-tests and significance is denoted by ** (p<0.01).

FIG. 2B exemplifies a survival curve of the mice as described in FIG. 2A that was plotted and compared using the Mantel-Cox test. Significance is denoted by ** (p<0.01).

FIG. 3A exemplifies changes in tumor size from immunotherapy of C57BL/6 mice (n=4/group) implanted on day 0 with 2×10⁵ large established HPV E6/E7 TC-1 tumor cells and administered 1×10¹⁰ Ad5 [E1-, E2b-]-null VPs or 1×10¹⁰ Ad5 [E1-, E2b-]-E6/E7 VPs on days 13, 20, and 27. Tumor size was determined and volumes calculated according to the formula V=(tumor width²×tumor length)/2. Analysis of significance was performed between experimental and vector control groups using unpaired t-tests and significance is denoted by ** (p<0.01).

FIG. 3B exemplifies a survival curve of the mice as described in FIG. 3A that was plotted and compared using the Mantel-Cox test. Significance is denoted by ** (p<0.01).

FIG. 4A exemplifies changes in tumor size from C57BL/6 mice (n=7/group) inoculated on day 0 with 2×10⁵ TC-1 tumor cells and administered treatments on days 10, 17, and 24 with 1×10¹⁰ Ad5 [E1-, E2b-]-null VPs plus 100 μg of isotype control rat IgG antibody. Tumor size was determined and volumes calculated according to the formula V=(tumor width²×length)/2. Tumor growth kinetics represents individual mice in each group.

FIG. 4B exemplifies changes in tumor size from C57BL/6 mice (n=7/group) inoculated on day 0 with 2×10⁵ TC-1 tumor cells and administered treatments on days 10, 17, and 24 with 1×10¹⁰ Ad5 [E1-, E2b-]-null VPs plus 100 μg anti-PD-1 antibody.

FIG. 4C exemplifies changes in tumor size from C57BL/6 mice (n=7/group) inoculated on day 0 with 2×10⁵ TC-1 tumor cells and administered treatments on days 10, 17, and 24 with 1×10¹⁰ Ad5 [E1-, E2b-]-E6/E7 VPs plus 100 μg isotype control rat IgG antibody.

FIG. 4D exemplifies changes in tumor size from C57BL/6 mice (n=7/group) inoculated on day 0 with 2×10⁵ TC-1 tumor cells and administered treatments on days 10, 17, and 24 with 1×10¹⁰ Ad5 [E1-, E2b-]-E6/E7 VPs plus 100 μg anti-PD-1 antibody.

FIG. 5 exemplifies a survival curve for C57BL/6 mice (n=7/group) treated as those in FIGS. 4A-D. The experiment was terminated on day 52 following tumor implantation. Mice treated with Ad5 [E1-, E2b-]-E6/E7 and control antibody exhibited significantly (p<0.008) longer survival compared to both groups of control mice (Ad5 [E1-, E2b-]-null and control antibody or Ad5 [E1-, E2b-]-null and anti-PD-1 antibody). 2 of 7 (29%) Ad5 [E1-, E2b-]-E6/E7 and control antibody treated mice remained alive at day 52. Mice treated with Ad5 [E1-, E2b-]-E6/E7 plus anti PD-1 antibody exhibited significantly (p<0.0006) longer survival as compared to both groups of controls. 4 of 7 (57%) Ad5 [E1-, E2b-]-E6/E7 plus anti-PD-1 antibody treated mice remained alive at day 52.

FIG. 6A exemplifies that Ad5 [E1-, E2b-]-E6/E7 promotes the recruitment of CD8+ tumor-infiltrating lymphocytes (TILs) into TC-1 tumors. C57BL/6 mice (n=5/group) were implanted with 2×10⁵ TC-1 tumor cells. Twelve days after implantation mice began treatment with Ad5 [E1-, E2b-]-null empty vector plus control IgG, Ad5 [E1-, E2b-]-null plus anti-PD-1 antibody, Ad5 [E1-, E2b-]-E6/E7 plus control IgG, or Ad5 [E1-, E2b-]-E6/E7 plus anti-PD-1 antibody. Vaccine was administered subcutaneously weekly and anti-PD-1 antibodies were administered via intraparietal injection every 3-4 days and tumors were analyzed on day 27. Ad5 [E1-, E2b-]-E6/E7 treatment significantly decreases the ratio of Treg/CD8+ TILs. Analysis of significance was performed using unpaired t-tests and significance is denoted by ns (p>0.05), * (p<0.05), ** (p<0.01), *** (p<0.001), or **** (p<0.0001).

FIG. 6B exemplifies that the reduction in the ratio of Treg/CD8+ TILs of FIG. 6A reduction is not driven by a reduction in the number of Tregs.

FIG. 6C exemplifies that the reduction in the ratio of Treg/CD8+ TILs of FIG. 6A is driven through an increase in the number of CD8+ TILs.

FIG. 7A exemplifies that Ad5 [E1-, E2b-]-E6/E7 plus anti-PD-1 antibody combination therapy promotes a pro-inflammatory tumor microenvironment. C57BL/6 mice (n=5/group) were tumor implanted, treated, and tumors were analyzed as in FIGS. 6A-C. The frequency of PD-1l CD4+ and CD8+ TILs is increased in tumors from mice treated with Ad5 [E1-, E2b-]-E6/E7. Tumors from mice treated with a combination of Ad5 [E1-, E2b-]-E6/E7 and anti-PD-1 antibody have a significantly lower frequency of PD-1+CD4⁺ and CD8 TILs (A), LAG-3⁺ CD8+ TILs (B), and (C). Analysis of significance was performed using unpaired t-tests and significance is denoted by ns (p>0.05), * (p<0.05), ** (p<0.01), or *** (p<0.001).

FIG. 7B exemplifies that tumors from mice treated with a combination of Ad5 [E1-, E2b-]-E6/E7 and anti-PD-1 antibody as in FIG. 7A have a significantly lower frequency of LAG-3⁺ CD8⁺ TILs bringing these levels more in line with tumors from control mice.

FIG. 7C exemplifies that tumors from mice treated with a combination of Ad5 [E1-, E2b-]-E6/E7 and anti-PD-1 antibody as in FIG. 7A have a significantly reduced expression level of PDL1.

FIG. 8 exemplifies cell mediated immune (CMI) dose responses as measured by ELISpot of splenocytes from C57BL/6 mice (n=5/group) immunized three times at 14-day intervals with doses of 1×10⁸, 1×10⁹, or 1×10¹⁰ Ad5 [E1-, E2b-]-E6/E7 VPs and assessed 14 days after the final immunization. The greatest induction of CMI was achieved with the 1×10¹⁰ VP dose. Positive control splenocytes were exposed to Con A.

FIG. 9A exemplifies activation of CD8-α⁺/IFN-γ⁺ splenocytes after immunization of C57BL/6 mice (n=5/group) immunized three times at two week intervals with 1×10¹⁰ VP Ad5 [E1-, E2b-]-E6/E7 VPs. Controls received 1×10¹⁰ Ad5 [E1-, E2b-]-null VPs. Splenocytes collected 14 days after the final immunization were assessed by flow cytometry. For positive controls, splenocytes were exposed to PMA/ionomycin.

FIG. 9B exemplifies activation of CD8-α⁺/IFN-γ+/TNF-α⁺ splenocytes after immunization of mice as described in FIG. 9A.

FIG. 10 exemplifies the effects of HPV immunotherapy in C57Bl/6 mice (n=7/group) implanted with HPV-E6/E7-Expressing TC-1 tumor cells (day 0) and treated by immunotherapy on days 10, 17, and 24 with 1×10¹⁰ Ad5-null VPs plus 100 μg control IgG antibody (intraperitoneal), 1×10¹⁰ Ad5-null VPs plus 100 μg anti-PD-1 antibody, 1×10¹⁰ Ad5 [E1-, E2b-]-E6/E7 VPs plus 100 μg mouse IgG antibody, or 1×10¹⁰ Ad5 [E1-, E2b-]-E6/E7 VPs plus 100 μg anti-PD-1 antibody. Immunotherapy with or without anti-PD-1 resulted in significant inhibition of tumor growth by day 23 (p<0.05). All control mice were terminated by day 23 due to tumor mass.

FIG. 11 exemplifies CMI response as assessed by flow cytometry. C57BL/6 mice were immunized three times with 10¹⁰ VP Ad5[E1-,E2b-]-null or 10¹⁰ VP Ad5[E1-,E2b-]-E6/E7 at two week intervals. Two weeks after the final immunization CD8α⁺ splenocytes were assayed for intracellular expression of IFNγ after 6 hour stimulation with antigen-specific peptide pools. Mean+/−standard deviation is plotted.

FIG. 12 exemplifies a result of immunotherapy of small established HPV E6/E7-Expressing tumors with Ad5 [E1-, E2b-]-E6/E7. C57BL/6 mice were implanted on day 0 with 2×10⁵ TC-1 tumor cells and administered 10¹⁰ VP Ad5 [E1-, E2b-]-null (vector control) or 10¹⁰ VP Ad5 [E1-, E2b-]-E6/E7 on days 6, 13, and 20 as indicated by arrows. (A) Tumor size was determined and volumes calculated according to the formula V=(tumor width²×tumor length)/2. On day 23, mice were euthanized from the vector control group. No analyses of significance could be performed after this 23 day time point and this is denoted by a dashed line. Analysis of significance was performed between experimental and vector control groups using unpaired t-tests and significance is denoted by ** (p<0.01). Error bars represent the standard error of the means.

FIG. 13 illustrates immunotherapy combined with chemotherapy/radiation treatment (CRT) of established HPV16-E6^{Δ}/E7^{Δ} expressing tumors. Established HPV16-E6^{Δ}/E7^{Δ} expressing tumors were treated with Ad5 [E1-, E2b-]-HPV16-E6^{Δ}/E7^{Δ} on days 7, 14, and 21 combined with cisplatin/radiation treatment on days 13, 20, and 27. Control tumor bearing mice were treated by injections with Ad-null combined with cisplatin/radiation treatment.

FIG. 14 illustrates the effect of CRT on CMI response. Non-tumor bearing mice were treated as described in FIG. 4 above. Two weeks after the last treatment, mice were assessed for CMI activity as determined by ELISpot assays for IFN-γ secreting splenocytes. Note the increased CMI responses in mice treated with combination therapy (Ad5 [E1-, E2b-]-HPV16-E6^{Δ}/E7^{Δ} plus CRT).

FIG. 15 exemplifies the treatment schema of a phase I/Ib trial of Ad5 [E1-, E2b-]-HPV16-E6^{Δ}/E7^{Δ} in healthy individuals that are HPV-16 positive by oral rinse or pap smear samples.

FIG. 16 exemplifies the study design and treatment schema of a phase I trial of Ad5 [E1-, E2b-]-HPV16-E6^{Δ}/E7Δ in individuals that have HPV-16 positive squamous cell carcinoma.

FIG. 17 exemplifies the treatment and correlative biomarker schema of a phase I trial of Ad5 [E1-, E2b-]-HPV16-E6^{Δ}/E7^{Δ} in individuals that have HPV-16 positive squamous cell carcinoma.

### DETAILED DESCRIPTION

The following passages describe different aspects of certain embodiments in greater detail. Each aspect may be combined with any other aspect or aspects unless clearly indicated to the contrary. In particular, any feature indicated as being preferred or advantageous may be combined with any other feature of features indicated as being preferred or advantageous.

Unless otherwise indicated, any embodiment can be combined with any other embodiment. A variety of aspects can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges include the range endpoints.

As used herein, unless otherwise indicated, the article “a” means one or more unless explicitly otherwise provided for. As used herein, unless otherwise indicated, terms such as “contain,” “containing,” “include,” “including,” and the like mean “comprising.” As used herein, unless otherwise indicated, the term “or” can be conjunctive or disjunctive. As used herein, unless otherwise indicated, any embodiment can be combined with any other embodiment.

### I. Adenovirus Vector Constructs

An “adenovirus” (Ad) refers to non-enveloped DNA viruses from the family Adenoviridae. These viruses can be found in, but are not limited to, human, avian, bovine, porcine and canine species. Some embodiments contemplate the use of any Ad from any of the four genera of the family Adenoviridae (e.g., Aviadenovirus, Mastadenovirus, Atadenovirus and Siadenovirus) as the basis of an E2b-deleted virus vector, or vector containing other deletions as described herein. In addition, several serotypes are found in each species. Ad also pertains to genetic derivatives of any of these viral serotypes, including but not limited to, genetic mutations, deletions or transpositions.

A “first generation adenovirus” refers to an Ad that has the early region 1 (E1) deleted. In additional cases, the early region 3 (E3) may also be deleted.

A “second generation adenovirus” refers to an Ad that has all or parts of the E1, E2, E3, and, in certain embodiments, E4 DNA gene sequences deleted (removed) from the virus.

“E2b-deleted” refers to a DNA sequence mutated in such a way so as to prevent expression and/or function of at least one E2b gene product. Thus, in certain embodiments, “E2b-deleted” is used in relation to a specific DNA sequence that is deleted (removed) from an Ad genome. E2b-deleted or “containing a deletion within an E2b region” refers to a deletion of at least one base pair within an E2b region of an Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, a deletion is of more than 150, 160, 170, 180, 190, 200, 250, or 300 base pairs within an E2b region of an Ad genome. An E2b deletion may be a deletion that prevents expression and/or function of at least one E2b gene product and therefore, encompasses deletions within exons of encoding portions of E2b-specific proteins as well as deletions within promoter and leader sequences. In certain embodiments, an E2b deletion is a deletion that prevents expression and/or function of one or both a DNA polymerase and a preterminal protein of an E2b region. In a further embodiment, “E2b-deleted” refers to one or more point mutations in a DNA sequence of this region of an Ad genome such that one or more encoded proteins is non-functional. Such mutations include residues that are replaced with a different residue leading to a change in an amino acid sequence that result in a nonfunctional protein.

“E1-deleted” refers to a DNA sequence that is mutated in such a way so as to prevent expression and/or function of at least one E1 gene product. Thus, in certain embodiments, “E1 deleted” is used in relation to a specific DNA sequence that is deleted (removed) from the Ad genome. E1 deleted or “containing a deletion within the E1 region” refers to a deletion of at least one base pair within the E1 region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E1 region of the Ad genome. An E1 deletion may be a deletion that prevents expression and/or function of at least one E1 gene product and therefore, encompasses deletions within exons of encoding portions of E1-specific proteins as well as deletions within promoter and leader sequences. In certain embodiments, an E1 deletion is a deletion that prevents expression and/or function of one or both of a trans-acting transcriptional regulatory factor of the E1 region. In a further embodiment, “E1 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

Compared to first generation adenovirus vectors, certain embodiments provide Second Generation E2b-deleted adenovirus vectors that contain deletions in the DNA polymerase gene (pol) and deletions of the pre-terminal protein (pTP). E2b-deleted vectors have up to a 13 kb gene-carrying capacity as compared to the 5 to 6 kb capacity of First Generation adenovirus vectors, easily providing space for nucleic acid sequences encoding any of a variety of target antigens. The E2b-deleted adenovirus vectors also have reduced adverse reactions as compared to first generation adenovirus vectors.

A “target antigen” or “target protein” refers to a molecule, such as a protein, against which an immune response is to be directed.

The innate immune response to wild type Ad can be complex, and it appears that Ad proteins expressed from adenovirus vectors play an important role. Specifically, the deletions of pre-terminal protein and DNA polymerase in the E2b-deleted vectors appear to reduce inflammation during the first 24 to 72 h following injection, whereas First Generation adenovirus vectors stimulate inflammation during this period. In addition, it has been reported that the additional replication block created by E2b deletion also leads to a 10,000 fold reduction in expression of Ad late genes, well beyond that afforded by E1, E3 deletions alone. The decreased levels of Ad proteins produced by E2b-deleted adenovirus vectors effectively reduce the potential for competitive, undesired, immune responses to Ad antigens, responses that prevent repeated use of the platform in Ad immunized or exposed subjects. The reduced induction of inflammatory response by second generation E2b-deleted vectors results in increased potential for the vectors to express desired vaccine antigens during the infection of antigen presenting cells (i.e., dendritic cells), decreasing the potential for antigenic competition, resulting in greater immunization of the vaccine to the desired antigen relative to identical attempts with First Generation adenovirus vectors. E2b-deleted adenovirus vectors provide an improved Ad-based vaccine candidate that is safer, more effective, and more versatile than previously described vaccine candidates using First Generation adenovirus vectors.

Thus, first generation, E1-deleted Adenovirus subtype 5 (Ad5)-based vectors, although promising platforms for use as cancer vaccines, are impeded in activity by naturally occurring or induced Ad-specific neutralizing antibodies. Without being bound by theory, Ad5-based vectors with deletions of the E1 and the E2b regions (Ad5 [E1-, E2b-]), the latter encoding the DNA polymerase and the pre-terminal protein, for example by virtue of diminished late phase viral protein expression, may avoid immunological clearance and induce more potent immune responses against the encoded tumor antigen transgene in Ad-immune hosts.

Some embodiments relate to methods and compositions (e.g., viral vectors) for generating immune responses against target antigens, in particular, those associated or related to infectious disease or proliferative cell disease such as cancer. Some embodiments relate to methods and compositions for generating immune responses in a subject against target antigens, in particular, those related to cell proliferation diseases such as cancer. In some embodiments, compositions and methods described herein relate to generating an immune response in a subject against cells expressing and/or presenting a target antigen or a target antigen signature comprising at least one target antigen. Some embodiments provide compositions and methods for immunotherapy against human papilloma virus (HPV) using a viral gene delivery platform to immunize against HPV gene E6, HPV gene E7, or a combination thereof combined with PD-1 checkpoint blockade. In certain embodiments, these compositions and methods utilize an Ad5 [E1-, E2b-]-HPV E6/E7 vaccine combined with an immune pathway checkpoint modulator. Ad5 [E1-, E2b-]-E6 can refer to Ad5 [E1-, E2b-]-HPV E6, or vice versa. Ad5 [E1-, E2b-]-E7 can refer to Ad5 [E1-, E2b-]-HPV E7, or vice versa. Ad5 [E1-, E2b-]-E6/E7 can refer to Ad5 [E1-, E2b-]-HPV E6/E7, or vice versa.

In general, adenoviruses are attractive for clinical use because they can have a broad tropism, they can infect a variety of dividing and non-dividing cell types and they can be used systemically as well as through more selective mucosal surfaces in a mammalian body. In addition, their relative thermostability further facilitates their clinical use. Adenoviruses are a family of DNA viruses characterized by an icosahedral, non-enveloped capsid containing a linear double-stranded genome. Generally, adenoviruses are found as non-enveloped viruses comprising double-stranded DNA genome, approximated ˜30-35 kilobases in size. Of the human Ads, none are associated with any neoplastic disease, and only cause relatively mild, self-limiting illness in immunocompetent subjects. In some embodiments, upon infection, the Ad genome or the genes in the adenoviral vectors described herein is not incorporated into the host gene and is processed extrachromasomal.

The first genes expressed by the virus are the E1 genes, which act to initiate high-level gene expression from the other Ad5 gene promoters present in the wild type genome. Viral DNA replication and assembly of progeny virions occur within the nucleus of infected cells, and the entire life cycle takes about 36 hr with an output of approximately 10⁴ virions per cell. The wild type Ad5 genome is approximately 36 kb, and encodes genes that are divided into early and late viral functions, depending on whether they are expressed before or after DNA replication. The early/late delineation is nearly absolute, since it has been demonstrated that super-infection of cells previously infected with an Ad5 results in lack of late gene expression from the super-infecting virus until after it has replicated its own genome. Without being bound by theory, this is likely due to a replication dependent cis-activation of the Ad5 major late promoter (MLP), preventing late gene expression (primarily the Ad5 capsid proteins) until replicated genomes are present to be encapsulated. The composition and methods as described herein, in some embodiments, take advantage of feature in the development of advanced generation Ad vectors/vaccines. The linear genome of the adenovirus is generally flanked by two origins for DNA replication (ITRs) and has eight units for RNA polymerase II-mediated transcription. The genome carries five early units E1A, E1B, E2, E3, E4, and E5, two units that are expressed with a delay after initiation of viral replication (IX and IVa2), and one late unit (L) that is subdivided into L1-L5. Some adenoviruses can further encode one or two species of RNA called virus-associated (VA) RNA.

Adenoviruses that induce innate and adaptive immune responses in human subjects are provided. By deletion or insertion of crucial regions of the viral genome, recombinant vectors are provided that have been engineered to increase their predictability and reduce unwanted side effects. In some aspects, there is provided an adenovirus vector comprising the genome deletion or insertion selected from the group consisting of: E1A, E1B, E2, E3, E4, E5, IX, IVa2, L1, L2, L3, L4, and L5, and any combination thereof.

Certain embodiments provide recombinant adenovirus vectors comprising an altered capsid. Generally, the capsid of an adenovirus is primarily comprises 20 triangular facets of an icosahedron each icosahedron contains 12 copies of hexon trimers. In addition there are also other several additional minor capsid proteins, IIa, VI, VIII, and IX.

Certain embodiments provide recombinant adenovirus vectors comprising one or more altered fiber proteins. In general the fiber proteins, which also form trimers, are inserted at the 12 vertices into the pentameric penton bases. The fiber can comprise of a thin N-terminal tail, a shaft, and a knob domain. The shaft can comprise a variable numbers of β-strand repeats. The knob can comprise one or more loops A, B, C, D, E, F, G, H, I, or J. The fiber knob loops can bind to cellular receptors. Certain embodiments provide adenovirus vectors to be used in vaccine systems for the treatment of cancers and infectious diseases.

Suitable adenoviruses that can be used with the present methods and compositions of the disclosure include but are not limited to species-specific adenovirus including human subgroups A, B1, B2, C, D, E, and F, or their crucial genomic regions as provided herein, which subgroups can further classified into immunologically distinct serotypes. Further, suitable adenoviruses that can be used with the present methods and compositions of the disclosure include, but are not limited to, species-specific adenovirus or their crucial genomic regions identified from primates, bovines, fowls, reptiles, or frogs.

Some adenoviruses serotypes preferentially target distinct organs. Serotypes such as AdHu1, AdHu2, and AdHu5 (subgenus C), generally effect the infect upper respiratory, while subgenera A and F effect gastrointestinal organs. Certain embodiments provide recombinant adenovirus vectors to be used in preferentially target distinct organs for the treatment of organ-specific cancers or organ-specific infectious diseases. In some applications the recombinant adenovirus vector is altered to reduce tropism to a specific organ in a mammal. In some applications the recombinant adenovirus vector is altered to increase tropism to a specific organ in a mammal.

The tropism of an adenovirus can be determined by their ability to attach to host cell receptors. In some instances the process of host cell attachment can involve the initial binding of the distal knob domain of the fiber to a host cell surface molecule followed by binding of the RGD motif within the penton base with αV integrins. Certain embodiments provide recombinant adenovirus vectors with altered tropism such that they can be genetic engineered to infect specific cell types of a host. Certain embodiments provide recombinant adenovirus vectors with altered tropism for the treatment of cell-specific cancers or cell-specific infectious diseases. Certain embodiments provide recombinant adenovirus vectors with altered fiber knob from one or more adenoviruses of subgroups A, B, C, D, or F, or a combination thereof or the insertion of RGD sequences. In some applications the recombinant adenovirus vectors comprising an altered fiber knob results in a vector with reduced tropism for one or more particular cell types. In some applications the recombinant adenovirus vectors comprising an altered fiber knob results in a vector with enhanced tropism for one or more particular cell types. In some applications the recombinant adenovirus vectors comprising an altered fiber knob results in a vector with reduced product-specific B or T-cell responses. In some applications the recombinant adenovirus vectors comprising an altered fiber knob results in a vector with enhanced product-specific B or T-cell responses.

Certain embodiments provide recombinant adenovirus vectors that are coated with other molecules to circumvent the effects of virus-neutralizing antibodies or improve transduction in to a host cell. Certain embodiments provide recombinant adenovirus vectors that are coated with an adaptor molecule that aids in the attachment of the vector to a host cell receptor. By way of example an adenovirus vector can be coated with adaptor molecule that connects coxsackie Ad receptor with CD40L resulting in increased transduction of dendritic cells, thereby enhancing immune responses in a subject. Other adenovirus vectors similarly engineered for enhancing the attachment to other target cell types are also contemplated.

First generation, or E1-deleted adenovirus vectors Ad5 [E1-] are constructed such that a transgene replaces only the E1 region of genes. Typically, about 90% of the wild-type Ad5 genome is retained in the vector. Ad5 [E1-] vectors have a decreased ability to replicate and cannot produce infectious virus after infection of cells that do not express the Ad5 E1 genes. The recombinant Ad5 [E1-] vectors are propagated in human cells (e.g., HEK 293 cells) allowing for Ad5 [E1-] vector replication and packaging. Ad5 [E1-] vectors have a number of positive attributes; one of the most important is their relative ease for scale up and cGMP production. Currently, well over 220 human clinical trials utilize Ad5 [E1-] vectors, with more than two thousand subjects given the virus sc, im, or iv. Additionally, Ad5 vectors do not integrate; their genomes remain episomal. Generally, for vectors that do not integrate into the host genome, the risk for insertional mutagenesis and/or germ-line transmission is extremely low if at all. Conventional Ad5 [E1-] vectors have a carrying capacity that approaches 7 kb.

Ad5-based vectors with deletions of the E1 and the E2b regions (Ad5 [E1-, E2b-]), the latter encoding the DNA polymerase and the pre-terminal protein, by virtue of diminished late phase viral protein expression, provide an opportunity to avoid immunological clearance and induce more potent immune responses against the encoded tumor antigen transgene in Ad-immune hosts. The new Ad5 platform has additional deletions in the E2b region, removing the DNA polymerase and the preterminal protein genes. The Ad5 [E1-, E2b-] platform has an expanded cloning capacity that is sufficient to allow inclusion of many possible genes. Ad5 [E1-, E2b-] vectors have up to about 12 kb gene-carrying capacity as compared to the 7 kb capacity of Ad5 [E1-] vectors, providing space for multiple genes if needed. In some embodiments, an insert of more than 1, 2, 3, 4, 5, 6, 7, 8, 9, 10, or 11 kb is introduced into an Ad5 vector, such as the Ad5 [E1-, E2b-] vector. Deletion of the E2b region confers advantageous immune properties on the Ad5 vectors, often eliciting potent immune responses to target transgene antigens while minimizing the immune responses to Ad viral proteins.

In some embodiments, the replication defective adenovirus vector comprises a modified sequence encoding a polypeptide with at least 50%, 60%, 65%, 70%, 75%, 80%, 85%, 90%, 95%, 98%, 99%, 99.5%, 99.9%, or 100% identity to a wild-type immunogenic polypeptide or a fragment thereof. In some embodiments, the replication defective adenovirus vector comprises a modified sequence encoding a subunit of a wild-type polypeptide. The compositions and methods, in some embodiments, relate to an adenovirus-derived vector comprising at least 60% sequence identity to SEQ ID NO: 17.

In some embodiments, an adenovirus-derived vector, optionally relating to a replication defective adenovirus, comprises a sequence with at least 75%, 80%, 85%, 90%, 95%, 98%, 99%, 99.5%, 99.8%, or 99.9% identity to SEQ ID NO: 17 or a sequence generated from SEQ ID NO: 17 by alternative codon replacements. In various embodiments, the adenovirus-derived vectors described herein have a deletion in the E2b region, and optionally, in the E1 region, the deletion conferring a variety of advantages to the use of the vectors in immunotherapy as described herein.

Certain regions within the adenovirus genome serve essential functions and may need to be substantially conserved when constructing the replication defective adenovirus vectors. These regions are further described in Lauer et al., J. Gen. Virol., 85, 2615-25 (2004), Leza et al., J. Virol., p. 3003-13 (1988), and Miralles et al., J. Bio Chem., Vol. 264, No. 18, p. 10763-72 (1983), which are incorporated by reference in their entirety. Recombinant nucleic acid vectors comprising a sequence with identity values of at least 50%, 60%, 65%, 70%, 75%, 80%, 85%, 90%, 95%, 98%, 99%, 99.5%, 99.8%, 99.9%, or 100% to a portion of SEQ ID NO: 17, such as a portion comprising at least about 100, 250, 500, 1000, or more bases of SEQ ID NO: 17 are used in some embodiments.

Certain embodiments contemplate the use of E2b-deleted adenovirus vectors, such as those described in U.S. Pat. Nos. 6,063,622; 6,451,596; 6,057,158; 6,083,750; and 8,298,549, which are each incorporated herein by reference in their entirety. The vectors with deletions in the E2b regions in many cases cripple viral protein expression and/or decrease the frequency of generating replication competent Ad (RCA). Propagation of these E2b-deleted adenovirus vectors can be done utilizing cell lines that express the deleted E2b gene products. Such packaging cell lines are provided herein; e.g., E.C7 (formally called C-7), derived from the HEK-2p3 cell line.

Further, the E2b gene products, DNA polymerase and preterminal protein, can be constitutively expressed in E.C7, or similar cells along with the E1 gene products. Transfer of gene segments from the Ad genome to the production cell line has immediate benefits: (1) increased carrying capacity; and, (2) a decreased potential of RCA generation, typically requiring two or more independent recombination events to generate RCA. The E1, Ad DNA polymerase and/or preterminal protein expressing cell lines used in some embodiments can enable the propagation of adenovirus vectors with a carrying capacity approaching 13 kb, without the need for a contaminating helper virus. In addition, when genes critical to the viral life cycle are deleted (e.g., the E2b genes), a further crippling of Ad to replicate or express other viral gene proteins occurs. This can decrease immune recognition of infected cells, and extend durations of foreign transgene expression.

E1, DNA polymerase, and preterminal protein deleted vectors are typically unable to express the respective proteins from the E1 and E2b regions. Further, they may show a lack of expression of most of the viral structural proteins. For example, the major late promoter (MLP) of Ad is responsible for transcription of the late structural proteins L1 through L5. Though the MLP is minimally active prior to Ad genome replication, the highly toxic Ad late genes are primarily transcribed and translated from the MLP only after viral genome replication has occurred. This cis-dependent activation of late gene transcription is a feature of DNA viruses in general, such as in the growth of polyoma and SV-40. The DNA polymerase and preterminal proteins are important for Ad replication (unlike the E4 or protein IX proteins). Their deletion can be extremely detrimental to adenovirus vector late gene expression, and the toxic effects of that expression in cells such as APCs.

The adenovirus vectors can include a deletion in the E2b region of the Ad genome and, optionally, the E1 region. In some cases, such vectors do not have any other regions of the Ad genome deleted. The adenovirus vectors can include a deletion in the E2b region of the Ad genome and deletions in the E1 and E3 regions. In some cases, such vectors have no other regions deleted. The adenovirus vectors can include a deletion in the E2b region of the Ad genome and deletions in the E1, E3 and partial or complete removal of the E4 regions. In some cases, such vectors have no other deletions. The adenovirus vectors can include a deletion in the E2b region of the Ad genome and deletions in the E1 and/or E4 regions. In some cases, such vectors contain no other deletions. The adenovirus vectors can include a deletion in the E2a, E2b, and/or E4 regions of the Ad genome. In some cases, such vectors have no other deletions. The adenovirus vectors can have the E1 and/or DNA polymerase functions of the E2b region deleted. In some cases, such vectors have no other deletions. The adenovirus vectors can have the E1 and/or the preterminal protein functions of the E2b region deleted. In some cases, such vectors have no other deletions. The adenovirus vectors can have the E1, DNA polymerase and/or the preterminal protein functions deleted. In some cases, such vectors have no other deletions. The adenovirus vectors can have at least a portion of the E2b region and/or the E1 region. In some cases, such vectors are not gutted adenovirus vectors. In this regard, the vectors may be deleted for both the DNA polymerase and the preterminal protein functions of the E2b region. The adenovirus vectors can have a deletion in the E1, E2b, and/or 100K regions of the adenovirus genome. The adenovirus vectors can comprise vectors having the E1, E2b and/or protease functions deleted. In some cases, such vectors have no other deletions. The adenovirus vectors can have the E1 and/or the E2b regions deleted, while the fiber genes have been modified by mutation or other alterations (for example to alter Ad tropism). Removal of genes from the E3 or E4 regions may be added to any of the adenovirus vectors mentioned. In certain embodiments, the adenovirus vector may be a gutted adenovirus vector.

“Gutted” or “gutless” refers to an Ad vector that has been deleted of all viral coding regions.

A “helper adenovirus” or “helper virus” refers to an Ad that can supply viral functions that a particular host cell cannot (the host may provide Ad gene products such as E1 proteins). This virus is used to supply, in trans, functions (e.g., proteins) that are lacking in a second virus, or helper dependent virus (e.g., a gutted or gutless virus, or a virus deleted for a particular region such as E2b or other region as described herein); the first replication-incompetent virus is said to “help” the second, helper dependent virus thereby permitting the production of the second viral genome in a cell.

Other regions of the Ad genome can be deleted. A “deletion” in a particular region of the Ad genome refers to a specific DNA sequence that is mutated or removed in such a way so as to prevent expression and/or function of at least one gene product encoded by that region (e.g., E2b functions of DNA polymerase or preterminal protein function). Deletions encompass deletions within exons encoding portions of proteins as well as deletions within promoter and leader sequences. A deletion within a particular region refers to a deletion of at least one base pair within that region of the Ad genome. More than one base pair can be deleted. For example, at least 20, 30, 40, 50, 60, 70, 80, 90, 100, 110, 120, 130, 140, or 150 base pairs can be deleted from a particular region. The deletion can be more than 150, 160, 170, 180, 190, 200, 250, or 300 base pairs within a particular region of the Ad genome. These deletions can prevent expression and/or function of the gene product encoded by the region. For example, a particular region of the Ad genome can include one or more point mutations such that one or more encoded proteins is non-functional. Such mutations include residues that are replaced with a different residue leading to a change in the amino acid sequence that result in a nonfunctional protein. Exemplary deletions or mutations in the Ad genome include one or more of E1a, E1b, E2a, E2b, E3, E4, L1, L2, L3, L4, L5, TP, POL, IV, and VA regions. Deleted adenovirus vectors can be made, for example, using recombinant techniques.

Ad vectors in certain embodiments can be successfully grown to high titers using an appropriate packaging cell line that constitutively expresses E2b gene products and products of any of the necessary genes that may have been deleted. HEK-293-derived cells that not only constitutively express the E1 and DNA polymerase proteins, but also the Ad-preterminal protein, can be used. E.C7 cells can be used, for example, to grow high titer stocks of the adenovirus vectors.

To delete critical genes from self-propagating adenovirus vectors, proteins encoded by the targeted genes can first be coexpressed in HEK-293 cells, or similar, along with E1 proteins. For example, those proteins which are non-toxic when coexpressed constitutively (or toxic proteins inducibly-expressed) can be selectively utilized. Coexpression in HEK-293 cells of the E1 and E4 genes is possible (for example utilizing inducible, not constitutive, promoters). The E1 and protein IX genes, a virion structural protein, can be coexpressed. Further coexpression of the E1, E4, and protein IX genes is also possible. E1 and 100K genes can be expressed in trans-complementing cell lines, as can E1 and protease genes.

Cell lines coexpressing E1 and E2b gene products for use in growing high titers of E2b-deleted Ad particles can be used. Useful cell lines constitutively express the approximately 140 kDa Ad-DNA polymerase and/or the approximately 90 kDa preterminal protein. Cell lines that possess high-level, constitutive coexpression of E1, DNA polymerase, and preterminal proteins, without toxicity (e.g., E.C7), are desirable for use in propagating replication-defective adenovirus vectors. These cell lines permit the propagation of adenovirus vectors deleted for the E1, DNA polymerase, and preterminal proteins.

The recombinant Ad can be propagated using, for example, tissue culture plates containing E.C7 cells infected with Ad vector virus stocks at an appropriate MOI (e.g., 5) and incubated at 37° C. for 40-96 h. The infected cells can be harvested, resuspended in 10 mM Tris-C1 (pH 8.0), and sonicated, and the virus can be purified by two rounds of cesium chloride density centrifugation. The virus containing band can be desalted over a column, sucrose or glycerol can be added, and aliquots can be stored at −80° C. Virus can be placed in a solution designed to enhance its stability, such as A195. The titer of the stock can be measured (e.g., by measurement of the optical density at 260 nm of an aliquot of the virus after lysis). Plasmid DNA, either linear or circular, encompassing the entire recombinant E2b-deleted adenovirus vector can be transfected into E.C7, or similar cells, and incubated at 37° C. until evidence of viral production is present (e.g., cytopathic effect). Conditioned media from cells can be used to infect more cells to expand the amount of virus produced before purification. Purification can be accomplished, for example, by two rounds of cesium chloride density centrifugation or selective filtration. Virus may be purified by chromatography using commercially available products or custom chromatographic columns.

The compositions as described herein can comprise enough virus to ensure that cells to be infected are confronted with a certain number of viruses. Thus, some embodiments provide a stock of recombinant Ad, such as an RCA-free stock of recombinant Ad. Viral stocks can vary considerably in titer, depending largely on viral genotype and the protocol and cell lines used to prepare them. Viral stocks can have a titer of at least about 10⁶, 10⁷, or 10⁸ virus particles (VPs)/mL, or higher, such as at least about 10⁹, 10¹⁰, 10¹¹, or 10¹² VPs/mL.

An “adenovirus 5 null (Ad5-null)” refers to a non-replicating Ad that does not contain any heterologous nucleic acid sequences for expression.

“Transfection” refers to the introduction of foreign nucleic acid into eukaryotic cells. Exemplary means of transfection include calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

“Stable transfection” or “stably transfected” refers to the introduction and integration of foreign nucleic acid, DNA or RNA, into the genome of the transfected cell. The term “stable transfectant” refers to a cell which has stably integrated foreign DNA into the genomic DNA.

A “reporter gene” indicates a nucleotide sequence that encodes a reporter molecule (e.g., an enzyme). A “reporter molecule” is detectable in any of a variety of detection systems, including, but not limited to, enzyme-based detection assays (e.g., ELISA, histochemical assays), fluorescent, radioactive, and luminescent systems. The E. coli β-galactosidase gene, green fluorescent protein (GFP), the human placental alkaline phosphatase gene, the chloramphenicol acetyltransferase (CAT) gene; and other reporter genes may be employed.

A “heterologous sequence” refers to a nucleotide sequence that is ligated to, or is manipulated to become ligated to, a nucleic acid sequence to which it is not ligated in nature, or to which it is ligated at a different location in nature. Heterologous nucleic acid may include a naturally occurring nucleotide sequence or some modification relative to the naturally occurring sequence.

A “transgene” refers to any gene coding region, either natural or heterologous nucleic acid sequences or fused homologous or heterologous nucleic acid sequences, introduced into cells or a genome of subject. Transgenes may be carried on any viral vector used to introduce transgenes to the cells of the subject.

“Generating an immune response” or “inducing an immune response” refers to a statistically significant change, e.g., increase or decrease, in the number of one or more immune cells (T-cells, B-cells, antigen-presenting cells, dendritic cells, neutrophils, and the like) or in the activity of one or more of these immune cells (CTL activity, HTL activity, cytokine secretion, change in profile of cytokine secretion, etc.).

### II. Viral Vectors for Immunotherapies and Vaccines

Recombinant viral vectors can be used to express protein coding genes or antigens (e.g., TAAs (tumor-associated antigens) and/or IDAAs (infectious-disease associated antigens)). The advantages of recombinant viral vector based vaccines and immunotherapy include high efficiency gene transduction, highly specific delivery of genes to target cells, induction of robust immune responses, and increased cellular immunity. Certain embodiments provide for recombinant adenovirus vectors comprising deletions or insertions of crucial regions of the viral genome. The viral vectors of provided herein can comprise heterologous nucleic acid sequences that encode one or more target antigens of interest, or variants, fragments or fusions thereof, against which it is desired to generate an immune response.

Human papillomavirus (HPV) vectors can be used to express antigens. For example, by modifying oncogenes in the genome, such as by deletion or insertion of crucial regions of the HPV viral genome, a recombinant vector can be engineered to increase predictability of infection and reduce unwanted side effects. An exemplary HPV vector is a fusion vector with an adenovirus vector. An exemplary HPV vector is Ad5 [E1-, E2b-]-HPV antigen viral vector comprising a modified non-oncogenic HPV E6 and/or HPV E7.

Studies in humans and animals have demonstrated that pre-existing immunity against Ad5 can be an inhibitory factor to commercial use of Ad-based vaccines. The preponderance of humans have antibody against Ad5, the most widely used subtype for human vaccines, with two-thirds of humans studied having lympho-proliferative responses against Ad5. This pre-existing immunity can inhibit immunization or re-immunization using typical Ad5 vaccines and may preclude the immunization of a vaccine against a second antigen, using an Ad5 vector, at a later time. Overcoming the problem of pre-existing anti-vector immunity has been a subject of intense investigation. Investigations using alternative human (non-Ad5 based) Ad5 subtypes or even non-human forms of Ad5 have been examined. Even if these approaches succeed in an initial immunization, subsequent vaccinations may be problematic due to immune responses to the novel Ad5 subtype. To avoid the Ad5 immunization barrier, and improve upon the limited efficacy of first generation Ad5 [E1-] vectors to induce optimal immune responses, some embodiments relate to a next generation Ad5 vector based vaccine platform.

In various embodiments, Ad5 [E1-, E2b-] vectors induce a potent cellular mediated immune (CMI), as well as antibodies against the vector expressed vaccine antigens even in the presence of Ad immunity. Ad5 [E1-, E2b-] vectors also have reduced adverse reactions as compared to Ad5 [E1-] vectors, in particular the appearance of hepatotoxicity and tissue damage. A key aspect of these Ad5 vectors is that expression of Ad late genes is greatly reduced. For example, production of the capsid fiber proteins could be detected in vivo for Ad5 [E1-] vectors, while fiber expression was ablated from Ad5 [E1-, E2b-] vector vaccines. The innate immune response to wild type Ad is complex. Proteins deleted from the Ad5 [E1-, E2b-] vectors generally play an important role. Specifically, Ad5 [E1-, E2b-] vectors with deletions of preterminal protein or DNA polymerase display reduced inflammation during the first 24 to 72 h following injection compared to Ad5 [E1-] vectors. In various embodiments, the lack of Ad5 gene expression renders infected cells invisible to anti-Ad activity and permits infected cells to express the transgene for extended periods of time, which develops immunity to the target.

It has been discovered that Ad5 [E1-, E2b-] vectors are not only are safer than, but appear to be superior to Ad5 [E1-] vectors in regard to induction of antigen-specific immune responses, making them much better suitable as a platform to deliver HPV E6 and/or HPV E7 vaccines that can result in a clinical response. In other cases, immune induction may take months.

Some embodiments contemplate increasing the capability for the Ad5 [E1-, E2b-] vectors to transduce dendritic cells, improving antigen-specific immune responses in the vaccine by taking advantage of the reduced inflammatory response against Ad5 [E1-, E2b-] vector viral proteins and the resulting evasion of pre-existing Ad immunity.

Attempts to overcome anti-Ad immunity have included use of alternative Ad serotypes and/or alternations in the Ad5 viral capsid protein each with limited success and the potential for significantly altering biodistribution of the resultant vaccines. Therefore, a completely novel approach was attempted by further reducing the expression of viral proteins from the E1 deleted Ad5 vectors, proteins known to be targets of pre-existing Ad immunity. Specifically, a novel recombinant Ad5 platform has been described with deletions in the early 1 (E1) gene region and additional deletions in the early 2b (E2b) gene region (Ad5 [E1-, E2b-]). Deletion of the E2b region (that encodes DNA polymerase and the pre-terminal protein) results in decreased viral DNA replication and late phase viral protein expression. This vector platform can be used to induce CMI responses in animal models of cancer and infectious disease and more importantly, this recombinant Ad5 gene delivery platform overcomes the barrier of Ad5 immunity and can be used in the setting of pre-existing and/or vector-induced Ad immunity thus enabling multiple homologous administrations of the vaccine. In particular embodiments, some embodiments relate to a replication defective adenovirus vector of serotype 5 comprising a sequence encoding an immunogenic polypeptide. The immunogenic polypeptide may be a mutant, natural variant, or a fragment thereof.

### III. Polynucleotides and Variants Encoding Antigen Targets

The terms “nucleic acid” and “polynucleotide” are used essentially interchangeably herein. Polynucleotides may be single-stranded (coding or antisense) or double-stranded, and may be DNA (e.g., genomic, cDNA, or synthetic) or RNA molecules. RNA molecules may include hnRNA molecules, which contain introns and correspond to a DNA molecule in a one-to-one manner, and mRNA molecules, which do not contain introns. Additional coding or non-coding sequences may, but need not, be present within a polynucleotide as described herein, and a polynucleotide may, but need not, be linked to other molecules and/or support materials. An isolated polynucleotide, as used herein, means that a polynucleotide is substantially away from other coding sequences. For example, an isolated DNA molecule as used herein does not contain large portions of unrelated coding DNA, such as large chromosomal fragments or other functional genes or polypeptide coding regions. This refers to the DNA molecule as originally isolated, and does not exclude genes or coding regions later added to the segment recombinantly in the laboratory.

As will be understood by those skilled in the art, the polynucleotides can include genomic sequences, extra-genomic and plasmid-encoded sequences and smaller engineered gene segments that express, or may be adapted to express target antigens as described herein, fragments of antigens, peptides and the like. Such segments may be naturally isolated, or modified synthetically by the hand of man.

Typically, polynucleotide variants will contain one or more substitutions, additions, deletions and/or insertions, preferably such that the immunogenicity of the epitope of the polypeptide encoded by the variant polynucleotide or such that the immunogenicity of the heterologous target protein is not substantially diminished relative to a polypeptide encoded by the native polynucleotide sequence. In some cases, the one or more substitutions, additions, deletions and/or insertions may result in an increased immunogenicity of the epitope of the polypeptide encoded by the variant polynucleotide. As described elsewhere herein, the polynucleotide variants can encode a variant of the target antigen, or a fragment (e.g., an epitope) thereof wherein the propensity of the variant polypeptide or fragment (e.g., epitope) thereof to react with antigen-specific antisera and/or T-cell lines or clones is not substantially diminished relative to the native polypeptide. The polynucleotide variants can encode a variant of the target antigen, or a fragment thereof wherein the propensity of the variant polypeptide or fragment thereof to react with antigen-specific antisera and/or T-cell lines or clones is substantially increased relative to the native polypeptide.

The term “variants” should also be understood to encompass homologous genes of xenogenic origin. In particular embodiments, variants or fragments of target antigens are modified such that they have one or more reduced biological activities. For example, an oncogenic protein target antigen may be modified to reduce or eliminate the oncogenic activity of the protein, or a viral protein may be modified to reduce or eliminate one or more activities or the viral protein. An example of a modified HPV E6 protein is an HPV E6 having a L26V mutation, resulting in a variant protein with increased immunogenicity.

When comparing polynucleotide sequences, two sequences are “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned. Optimal alignment of sequences for comparison may be conducted using the Megalign program in the Lasergene suite of bioinformatics software using default parameters. Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA), or by inspection. One example of algorithms that are suitable for determining percent sequence identity and sequence similarity are the BLAST and BLAST 2.0 algorithms. BLAST and BLAST 2.0 can be used, for example with the parameters described herein, to determine percent sequence identity for the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program uses as defaults a word length (W) of 11, and expectation (E) of 10, and the BLOSUM62 scoring matrix alignments, (B) of 50, expectation (E) of 10, M=5, N=−4 and a comparison of both strands.

The “percentage of sequence identity” can be determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence and multiplying the results by 100 to yield the percentage of sequence identity.

It will be appreciated by those of ordinary skill in the art that, as a result of the degeneracy of the genetic code, there are many nucleotide sequences that encode a particular antigen of interest, or fragment thereof, as described herein. Some of these polynucleotides bear minimal homology to the nucleotide sequence of any native gene. Nonetheless, polynucleotides that vary due to differences in codon usage are specifically contemplated. Further, alleles of the genes comprising the polynucleotide sequences provided herein are within the scope of some embodiments. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Certain embodiments provide nucleic acid sequences, also referred to herein as polynucleotides that encode one or more target antigens of interest, or fragments or variants thereof. As such, some embodiments provide polynucleotides that encode target antigens from any source as described further herein, vectors comprising such polynucleotides and host cells transformed or transfected with such expression vectors. In order to express a desired target antigen polypeptide, nucleotide sequences encoding the polypeptide, or functional equivalents, can be inserted into an appropriate Ad vector (e.g., using recombinant techniques). The appropriate adenovirus vector may contain the necessary elements for the transcription and translation of the inserted coding sequence and any desired linkers. Methods which are well known to those skilled in the art may be used to construct these adenovirus vectors containing sequences encoding a polypeptide of interest and appropriate transcriptional and translational control elements. These methods include in vitro recombinant DNA techniques, synthetic techniques, and in vivo genetic recombination.

Polynucleotides may comprise a native sequence (i.e., an endogenous sequence that encodes a target antigen polypeptide/protein/epitope or a portion thereof) or may comprise a sequence that encodes a variant, fragment, or derivative of such a sequence. Polynucleotide sequences can encode target antigen proteins. In some embodiments, polynucleotides represent a novel gene sequence optimized for expression in specific cell types that may substantially vary from the native nucleotide sequence or variant but encode a similar protein antigen.

In other related embodiments, polynucleotide variants have substantial identity to native sequences encoding proteins (e.g., target antigens of interest), for example those comprising at least 70% sequence identity, preferably at least 75%, 80%, 85%, 90%, 95%, 96%, 97%, 98%, or 99% or higher, sequence identity compared to a native polynucleotide sequence encoding the polypeptides (e.g., BLAST analysis using standard parameters). These values can be appropriately adjusted to determine corresponding identity of proteins encoded by two nucleotide sequences by taking into account codon degeneracy, amino acid similarity, reading frame positioning and the like. Polynucleotides can encode a protein comprising for example at least 70% sequence identity, preferably at least 75%, 80%, 85%, 90%, 95%, 96%, 97%, 98%, or 99% or higher, sequence identity compared to a protein sequence encoded by a native polynucleotide sequence.

Polynucleotides can comprise at least about 5, 10, 15, 20, 25, 30, 35, 40, 45, 50, 55, 60, 65, 70, 75, 80, 85, 90, 95, 100, 11, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 or more contiguous nucleotides encoding a polypeptide (e.g., target protein antigens), and all intermediate lengths there between. “Intermediate lengths”, in this context, refers to any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence may be extended at one or both ends by additional nucleotides not found in the native sequence encoding a polypeptide, such as an epitope or heterologous target protein. This additional sequence may consist of 1, 2, 3, 4, 5, 6, 7, 8, 9, 10, 11, 12, 13, 14, 15, 16, 17, 18, 19, or 20 nucleotides or more, at either end of the disclosed sequence or at both ends of the disclosed sequence.

The polynucleotides, regardless of the length of the coding sequence itself, may be combined with other DNA sequences, such as promoters, expression control sequences, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. Illustrative polynucleotide segments with total lengths of about 1000, 2000, 3000, 4000, 5000, 6000, 7000, 8000, 9000, 10,000, about 500, about 200, about 100, about 50 base pairs in length, and the like, (including all intermediate lengths) are contemplated to be useful in many embodiments.

A mutagenesis approach, such as site-specific mutagenesis, can be employed to prepare target antigen sequences. Specific modifications in a polypeptide sequence can be made through mutagenesis of the underlying polynucleotides that encode them. Site-specific mutagenesis can be used to make mutants through the use of oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. For example, a primer comprising about 14 to about 25 nucleotides or so in length can be employed, with about 5 to about 10 residues on both sides of the junction of the sequence being altered. Mutations may be made in a selected polynucleotide sequence to improve, alter, decrease, modify, or otherwise change the properties of the polynucleotide, and/or alter the properties, activity, composition, stability, or primary sequence of the encoded polypeptide.

Mutagenesis of polynucleotide sequences can be used to alter one or more properties of the encoded polypeptide, such as the immunogenicity of an epitope comprised in a polypeptide or the oncogenicity of a target antigen. Assays to test the immunogenicity of a polypeptide include, but are not limited to, T-cell cytotoxicity assays (CTL/chromium release assays), T-cell proliferation assays, intracellular cytokine staining, ELISA, ELISpot, etc. Other ways to obtain sequence variants of peptides and the DNA sequences encoding them can be employed. For example, recombinant vectors encoding the desired peptide sequence may be treated with mutagenic agents, such as hydroxylamine, to obtain sequence variants.

Polynucleotide segments or fragments encoding the polypeptides as described herein may be readily prepared by, for example, directly synthesizing the fragment by chemical means. Fragments may be obtained by application of nucleic acid reproduction technology, such as PCR, by introducing selected sequences into recombinant vectors for recombinant production.

A variety of vector/host systems may be utilized to contain and produce polynucleotide sequences. Exemplary systems include microorganisms such as bacteria transformed with recombinant bacteriophage, plasmid, or cosmid DNA vectors; yeast transformed with yeast vectors; insect cell systems infected with virus vectors (e.g., baculovirus); plant cell systems transformed with virus vectors (e.g., cauliflower mosaic virus, CaMV; tobacco mosaic virus, TMV) or with bacterial vectors (e.g., Ti or pBR322 plasmids); or animal cell systems.

Control elements or regulatory sequences present in an Ad vector may include those non-translated regions of the vector-enhancers, promoters, and 5′ and 3′ untranslated regions. Such elements may vary in their strength and specificity. Depending on the vector system and host utilized, any number of suitable transcription and translation elements, including constitutive and inducible promoters, may be used. For example, sequences encoding a polypeptide of interest may be ligated into an Ad transcription/translation complex consisting of the late promoter and tripartite leader sequence. Insertion in a non-essential E1 or E3 region of the viral genome may be used to obtain a viable virus which is capable of expressing the polypeptide in infected host cells. In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest (e.g., ATG initiation codon and adjacent sequences). Exogenous translational elements and initiation codons may be of various origins, both natural and synthetic. The efficiency of expression may be enhanced by the inclusion of enhancers which are appropriate for the particular cell system which is used. Specific termination sequences, either for transcription or translation, may also be incorporated in order to achieve efficient translation of the sequence encoding the polypeptide of choice.

A variety of protocols for detecting and measuring the expression of polynucleotide-encoded products (e.g., target antigens), can be used (e.g., using polyclonal or monoclonal antibodies specific for the product). Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed.

The Ad vectors can comprise a product that can be detected or selected for, such as a reporter gene whose product can be detected, such as by fluorescence, enzyme activity on a chromogenic or fluorescent substrate, and the like, or selected for by growth conditions. Exemplary reporter genes include green fluorescent protein (GFP), β-galactosidase, chloramphenicol acetyltransferase (CAT), luciferase, neomycin phosphotransferase, secreted alkaline phosphatase (SEAP), and human growth hormone (HGH). Exemplary selectable markers include drug resistances, such as neomycin (G418), hygromycin, and the like.

The Ad vectors can also comprise a promoter or expression control sequence. The choice of the promoter will depend in part upon the targeted cell type and the degree or type of control desired. Promoters that are suitable include, without limitation, constitutive, inducible, tissue specific, cell type specific, temporal specific, or event-specific. Examples of constitutive or nonspecific promoters include the SV40 early promoter, the SV40 late promoter, CMV early gene promoter, bovine papilloma virus promoter, and adenovirus promoter. In addition to viral promoters, cellular promoters are also amenable and useful in some embodiments. In particular, cellular promoters for the so-called housekeeping genes are useful (e.g., β-actin). Viral promoters are generally stronger promoters than cellular promoters. Inducible promoters may also be used. These promoters include MMTV LTR, inducible by dexamethasone, metallothionein, inducible by heavy metals, and promoters with cAMP response elements, inducible by cAMP, heat shock promoter. By using an inducible promoter, the nucleic acid may be delivered to a cell and will remain quiescent until the addition of the inducer. This allows further control on the timing of production of the protein of interest. Event-type specific promoters (e.g., HIV LTR) can be used, which are active or upregulated only upon the occurrence of an event, such as tumorigenicity or viral infection, for example. The HIV LTR promoter is inactive unless the tat gene product is present, which occurs upon viral infection. Some event-type promoters are also tissue-specific. Preferred event-type specific promoters include promoters activated upon viral infection.

Examples of promoters include promoters for α-fetoprotein, α-actin, myo D, carcinoembryonic antigen, VEGF-receptor; FGF receptor; TEK or tie 2; tie; urokinase receptor; E- and P-selectins; VCAM-1; endoglin; endosialin; αV-β3 integrin; endothelin-1; ICAM-3; E9 antigen; von Willebrand factor; CD44; CD40; vascular-endothelial cadherin; notch 4, high molecular weight melanoma-associated antigen; prostate specific antigen-1, probasin, FGF receptor, VEGF receptor, erb B2; erb B3; erb B4; MUC-1; HSP-27; int-1; int-2, CEA, HBEGF receptor; EGF receptor; tyrosinase, MAGE, IL-2 receptor; prostatic acid phosphatase, probasin, prostate specific membrane antigen, α-crystallin, PDGF receptor, integrin receptor, α-actin, SM1 and SM2 myosin heavy chains, calponin-h1, SM22 α-angiotensin receptor, IL-1, IL-2, IL-3, IL-4, IL-5, IL-6, IL-7, IL-8, IL-9, IL-10, IL-11, IL-12, IL-13, IL-14, IL-15, immunoglobulin heavy chain, immunoglobulin light chain, and CD4.

Repressor sequences, negative regulators, or tissue-specific silencers may be inserted to reduce non-specific expression of the polynucleotide. Multiple repressor elements may be inserted in the promoter region. Repression of transcription is independent of the orientation of repressor elements or distance from the promoter. One type of repressor sequence is an insulator sequence. Such sequences inhibit transcription and can silence background transcription. Negative regulatory elements can be located in the promoter regions of a number of different genes. The repressor element can function as a repressor of transcription in the absence of factors, such as steroids, as does the NSE in the promoter region of the ovalbumin gene. These negative regulatory elements can bind specific protein complexes from oviduct, none of which are sensitive to steroids. Three different elements are located in the promoter of the ovalbumin gene. Oligonucleotides corresponding to portions of these elements can repress viral transcription of the TK reporter. One of the silencer elements shares sequence identity with silencers in other genes (TCTCTCCNA (SEQ ID NO: 1)).

Elements that increase the expression of the desired target antigen can be incorporated into the nucleic acid sequence of the Ad vectors described herein. Exemplary elements include internal ribosome binding sites (IRESs). IRESs can increase translation efficiency. As well, other sequences may enhance expression. For some genes, sequences especially at the 5′ end may inhibit transcription and/or translation. These sequences are usually palindromes that can form hairpin structures. In some cases, such sequences in the nucleic acid to be delivered are deleted. Expression levels of the transcript or translated product can be assayed to confirm or ascertain which sequences affect expression. Transcript levels may be assayed by any known method, including Northern blot hybridization, RNase probe protection and the like. Protein levels may be assayed by any known method, including ELISA.

### IV. Antigen-Specific Immunotherapies and Vaccines

Certain embodiments provide single antigen or combination antigen immunization against HPV E6, HPV E7, or a combination thereof, utilizing such vectors and other vectors as provided herein. Certain embodiments provide therapeutic vaccines against HPV E6 and/or HPV E7 in subjects having HPV-induced or HPV-associated cancers. Other embodiments provide vaccines against HPV E6 and/or HPV E7 in subjects that are HPV positive without cancer but are at high risk for developing HPV induced cancers. Further, in various embodiments, the composition and methods provided herein can lead to clinical responses, such as altered disease progression or life expectancy.

Ad5 vector capsid interactions with dendritic cells (DCs) may trigger several beneficial responses, which may enhance the propensity of DCs to present antigens encoded by Ad5 vectors. For example, immature DCs, though specialized in antigen uptake, are relatively inefficient effectors of T-cell activation. DC maturation coincides with the enhanced ability of DCs to drive T-cell immunity. In some instances, the compositions and methods take advantage of an Ad5 infection resulting in direct induction of DC maturation. In some instances, Ad vector infection of immature bone marrow derived DCs from mice may upregulate cell surface markers normally associated with DC maturation (MHC I and II, CD40, CD80, CD86, and ICAM-1) as well as down-regulation of CD11c, an integrin down regulated upon myeloid DC maturation. In some instances, Ad vector infection triggers IL-12 production by DCs, a marker of DC maturation. Without being bound by theory, these events may possibly be due to Ad5 triggered activation of NF-κB pathways. Mature DCs can be efficiently transduced by Ad vectors, and do not lose their functional potential to stimulate the proliferation of naive T-cells at lower multiplicity of infection (MOI), as demonstrated by mature CD83+ human DC (derived from peripheral blood monocytes). However, mature DCs may also be less infectable than immature ones. Modification of capsid proteins can be used as a strategy to optimize infection of DC by Ad vectors, as well as enhancing functional maturation, for example using the CD40L receptor as a viral vector receptor, rather than using the normal CAR receptor infection mechanisms.

In some embodiments, the compositions and methods comprising an Ad5 [E1-, E2b-] vector(s) HPV E6 and/or HPV E7 antigen vaccine have effects of increased overall survival (OS) within the bounds of technical safety.

In some embodiments, the antigen targets are associated with benign tumors. In some embodiments, the antigens targeted are associated with pre-cancerous tumors.

As noted above, the adenovirus vectors comprise nucleic acid sequences that encode one or more target proteins or antigens of interest. In this regard, the vectors may contain nucleic acid encoding 1, 2, 3, 4, 5, 6, 7, 8, 9, 10, 11, 12, 13, 14, 15, 16, 17, 18, 19, 20, or more different target antigens of interest. The target antigens may be a full length protein or may be a fragment (e.g., an epitope) thereof. The adenovirus vectors may contain nucleic acid sequences encoding multiple fragments or epitopes from one target protein of interest or may contain one or more fragments or epitopes from numerous different target proteins of interest. A target antigen may comprise any substance against which it is desirable to generate an immune response but generally, the target antigen is a protein. A target antigen may comprise a full length protein, a subunit of a protein, an isoform of a protein, or a fragment thereof that induces an immune response (i.e., an immunogenic fragment). A target antigen or fragment thereof may be modified, e.g., to reduce one or more biological activities of the target antigen or to enhance its immunogenicity. The target antigen or target protein can be HPV E6, HPV E7, or both.

An “immunogenic fragment” refers to a fragment of a polypeptide that is specifically recognized (i.e., specifically bound) by a B-cell and/or T-cell surface antigen receptor resulting in a generation of an immune response specifically against a fragment.

In certain embodiments, immunogenic fragments bind to an MHC class I or class II molecule. An immunogenic fragment may “bind to” an MHC class I or class II molecule if such binding is detectable using any assay known in the art. For example, the ability of a polypeptide to bind to MHC class I may be evaluated indirectly by monitoring the ability to promote incorporation of ¹²⁵I labeled β-2-microglobulin (β-2m) into MHC class I/β2m/peptide heterotrimeric complexes. Alternatively, functional peptide competition assays that are known in the art may be employed. Immunogenic fragments of polypeptides may generally be identified using well known techniques. Representative techniques for identifying immunogenic fragments include screening polypeptides for the ability to react with antigen-specific antisera and/or T-cell lines or clones. An immunogenic fragment of a particular target polypeptide is a fragment that reacts with such antisera and/or T-cells at a level that is not substantially less than the reactivity of the full length target polypeptide (e.g., in an ELISA and/or T-cell reactivity assay). In other words, an immunogenic fragment may react within such assays at a level that is similar to or greater than the reactivity of the full length polypeptide. Such screens may be performed using methods known in the art.

In some embodiments, the viral vectors comprise heterologous nucleic acid sequences that encode one or more proteins, variants thereof, fusions thereof, or fragments thereof, that can modulate the immune response. In some embodiments the Second Generation E2b-deleted adenovirus vectors comprise a heterologous nucleic acid sequence. In some embodiments, the heterologous nucleic acid sequence is HPV E6 and HPV E7, a variant, a portion, or any combination thereof.

### V. HPV Antigen Targets

Target antigens may also include proteins, or variants or fragments thereof, associated with human papillomavirus (HPV), such as oncoproteins E6 and/or E7. In certain embodiments, the oncoprotein is modified to produce a non-oncogenic variant or a variant having reduced oncogenicity relative to the wild type protein. For example, the portion of the peptide that is responsible for binding a tumor suppressor protein (e.g., p53 and pRb) may be deleted or modified so that it no longer interacts with the tumor suppressor protein. In certain embodiments, HPV E6 and HPV E7 may be further modified to include an agonist epitope that binds to selected MHC molecules, e.g., HLA-A2, HLA-A3, and HLA-A24. For example, HPV E6 and/or HPV E7 may be modified to contain one or more agonist epitopes. In some instances, two or more target antigens may be used during immunization. For example, the E6 and/or E7 antigens can be expressed from the same vector, or separate vectors containing heterologous nucleotides encoding E6 and E7 target antigens used in combination. For example, an Ad5-E6 vector can be administered with an Ad5-E7 vector. In this example, the Ad5-E6 vector and Ad5-E7 vector may be administered simultaneously or they may be administered sequentially.

High-risk human papillomavirus (HPV) such as HPV type-16 (HPV-16) is associated with the etiology of cervical and more than 90% of HPV-related head and neck squamous cell carcinomas. Preventive vaccines such as HPV bivalent [Types 16 and 18] vaccine and recombinant and HPV quadrivalent [Types 6, 11, 16, and 18] vaccine can be a primary defense against HPV-associated cancers by preventing infection with the virus but reports indicate that they are not effective for active immunotherapy of established disease. The HPV early 6 (E6) and early 7 (E7) genes are expressed at high levels in HPV-induced cancers and are involved in the immortalization of primary human epidermal cells. Thus, these are ideal targets for tumor-specific immunotherapy because unlike many other tumor-associated antigens these viral antigens are “non-self” and thus do not have the potential to induce autoimmunity.

In certain embodiments, disclosed herein is a vaccine against human papilloma virus (HPV) that can be used to reduce, destroy, or eliminate HPV E6/E7-expressing cells in HPV positive subjects without cancer but with higher risk of developing HPV-induced or HPV-associated cancer.

In certain embodiments, disclosed is a vaccine or an immunotherapy in HPV positive subjects for treating HPV-induced or HPV-associated diseases, such as cancer.

The HPV vaccine of the disclosure uses a viral gene delivery platform to immunize against HPV-16 genes E6 and E7 (Ad5 [E1-, E2b-]-E6/E7). In some embodiments, the Ad5 [E1-, E2b-]-E6/E7 vaccine can be combined with a programmed death-ligand 1 (PD-1) blockade. Also disclosed herein is a vaccine comprised of a gene delivery vehicle (Ad5 [E1-, E2b-]) carrying modified genes for HPV-16 E6 and/or E7. The HPV E6 and/or E7 genes can be modified to render them non-oncogenic while retaining the antigenicity necessary to produce an immune response against HPV and HPV induced tumors. Also disclosed herein, HPV E6 and/or HPV E7 may be further modified to include an agonist epitope that binds to selected MHC molecules, e.g., HLA-A2, HLA-A3, and HLA-A24. For example, HPV E6 and/or HPV E7 may be modified to contain one or more agonist epitopes. The modified genes can be incorporated into a vaccine (Ad5 [E1-, E2b-]-E6; Ad5 [E1-, E2b-]-E7; or Ad5 [E1-, E2b-]-E6/E7). The Ad5 [E1-, E2b-]-E6 vaccine, Ad5 [E1-, E2b-]-E7 vaccine, or Ad5 [E1-, E2b-]-E6/E7 vaccine can retain the ability to induce an HPV-specific cell-mediated immune (CMI) response. In some embodiments, the Ad5 [E1-, E2b-]-E6/E7 vaccine can synergize with standard clinical therapy, enhancing immune-mediated clearance of an HPV E6/E7-expressing tumor. In some embodiments, the Ad5 [E1-, E2b-]-E6 vaccine can synergize with standard clinical therapy, enhancing immune-mediated clearance of an HPV E6-expressing tumor. In some embodiments, the Ad5 [E1-, E2b-]-E7 vaccine can synergize with standard clinical therapy, enhancing immune-mediated clearance of an HPV E7-expressing tumor.

Certain embodiments use the new Ad5 [E1-, E2b-] vector system to deliver a long sought-after need for developing a therapeutic vaccine against HPV E6 and/or HPV E7, overcome barriers found with other Ad5 systems and permit the immunization of people who have previously been exposed to Ad5.

To address the low immunogenicity of self-tumor antigens, a variety of advanced, multi-component vaccination strategies including co-administration of adjuvants and immune stimulating cytokines are provided. Some embodiments relate to recombinant viral vectors that provide innate pro-inflammatory signals, while simultaneously engineered to express the antigen of interest. Of particular interest are adenovirus serotype-5 (Ad5)-based immunotherapeutics that have been repeatedly used in humans to induce robust T-cell-mediated immune responses, all while maintaining an extensive safety profile.

A balance between activation and inhibitory signals regulates the interaction between T lymphocytes and tumor cells, wherein T cell responses are initiated through antigen recognition by T-cell receptors (TCRs). In some cases, when combined with chemotherapy/radiation treatment in HPV E6/E7-expressing tumor bearing mice, immunotherapy treatment with Ad5 [E1-, E2b-]-E6/E7 can result in significant improvement in overall survival as compared to subjects that receive chemotherapy/radiation alone.

In particular embodiments, the HPV antigen is modified to be a non-oncogenic HPV antigen or a modified HPV antigen with reduced oncogenicity as compared with a wild-type HPV. In certain embodiments, the modified HPV antigen is further modified to contain one or more agonist epitopes. For example, the antigen used herein is a modified HPV E6 antigen having an amino acid sequence set forth in or at least 50%, 60%, 65%, 70%, 75%, 80%, 85%, 90%, 95%, 98%, 99%, 99.5%, 99.9%, or 100% identical to SEQ ID NO: 8 (HPV16 E6 with E6A1 epitope), SEQ ID NO: 9 (HPV16 E6 with E6A3 epitope), SEQ ID NO: 10 (HPV16 E6 with E6A1+E6A3 epitopes), SEQ ID NO: 13, a modified HPV E7 antigen having an amino acid sequence set forth in or at least 50%, 60%, 65%, 70%, 75%, 80%, 85%, 90%, 95%, 98%, 99%, 99.5%, 99.9%, or 100% identical to SEQ ID NO: 12 (HPV16 E7 with E73 epitope), SEQ ID NO: 14, or a combination thereof. In particular embodiments, the nucleotide sequence of the antigen has a region at least 50%, 60%, 65%, 70%, 75%, 80%, 85%, 90%, 95%, 98%, 99%, 99.5%, 99.9%, or 100% identical to positions 23-496 and 502-795 of SEQ ID NO: 2 (HPV16 E6 with E6A1 epitope and E7 with E7A3 epitope), SEQ ID NO: 3 (HPV16 E6 with E6A3 epitope and E7 with E7A3 epitope), or SEQ ID NO: 4 (HPV16 E6 with E6A1 and E6A3 epitopes and E7 with E7A3 epitope), or a combination thereof. For example, the nucleic acid sequence has at least 80% identity to SEQ ID NO: 2, SEQ ID NO: 3, or SEQ ID NO: 4 (nucleotide sequences encoding both HPV E6 and E7 proteins). In further embodiments, the nucleic acid sequence has at least 50%, 60%, 65%, 70%, 75%, 80%, 85%, 90%, 95%, 98%, 99%, 99.5%, 99.9%, or 100% identity to any portion of or full-length to SEQ ID NO: 16 (the predicted sequence of an adenovirus vector expressing HPV E6 and E7), such as positions 1033 to 1845 of SEQ ID NO: 16. In certain embodiments, the nucleic acid sequence encodes fusion protein comprising a modified HPV E6 and a modified E7 antigen, such as a nucleic acid sequence at least 50%, 60%, 65%, 70%, 75%, 80%, 85%, 90%, 95%, 98%, 99%, 99.5%, 99.9%, or 100% identical to SEQ ID NO: 15.

In some embodiments, the HPV antigen comprises a modification that comprises a substitution of amino acids at positions 26, 98, 106 (e.g., SEQ ID NO: 8, SEQ ID NO: 9, or SEQ ID NO: 10), or a combination thereof, of HPV E6. In some embodiments, the HPV antigen comprises a modification that comprises a substitution of amino acids at position 86 (e.g., SEQ ID NO: 12) of HPV E7.

In one aspect, a composition is provided comprising a recombinant replication defective viral vector comprising a sequence encoding an HPV E6 antigen, wherein the sequence encoding the HPV E6 antigen has at least 80% sequence identity to SEQ ID NO: 5 (HPV16 E6 with E6A1 epitope), SEQ ID NO: 18 (HPV16 E6 with E6A1 epitope), SEQ ID NO: 6 (HPV16 E6 with E6A3 epitope), SEQ ID NO: 19 (HPV16 E6 with E6A3 epitope), SEQ ID NO: 7 (HPV16 E6 with E6A1 and E6A3 epitopes), SEQ ID NO: 20 (HPV16 E6 with E6A1 and E6A3 epitope), or at least 80% sequence identity to positions 23-496 of SEQ ID NO: 2, SEQ ID NO: 3, SEQ ID NO: 4. In some embodiments, the HPV E6 antigen comprises a sequence with at least 80% sequence identity to SEQ ID NO: 8, SEQ ID NO: 9, or SEQ ID NO: 10.

In one aspect, a composition is provided comprising a recombinant replication defective viral vector comprising a sequence encoding an HPV E7 antigen, wherein the sequence encoding the HPV E7 antigen has at least 80% sequence identity to SEQ ID NO: 11 (HPV16 E7 with E7A3 epitope) or SEQ ID NO: 21 (HPV16 E7 with E7A3 epitope), or at least 80% sequence identity to positions 502-795 of SEQ ID NO: 2. In some embodiments, the HPV E7 antigen comprises a sequence with at least 80% sequence identity to SEQ ID NO: 12.

In one aspect, a composition is provided comprising a recombinant replication defective viral vector comprising a sequence encoding an HPV E6/E7, wherein the sequence encoding the HPV E6 and HPV E7 antigens has at least 80% sequence identity to SEQ ID NO: 2, SEQ ID NO: 3, or SEQ ID NO: 4. In some embodiments, the HPV E6 and HPV E7 antigens comprise a sequence with at least 80% sequence identity to SEQ ID NO: 8, SEQ ID NO: 9, SEQ ID NO: 10, or SEQ ID NO: 12.

Additional non-limiting examples of target antigens include human epidermal growth factor receptor 2 (HER2/neu), carcinoembryonic antigen (CEA), a tumor neo-antigens or tumor neo-epitope, folate receptor alpha, WT1, brachyury (TIVS7-2, polymorphism), brachyury (IVS7 T/C polymorphism), T brachyury, T, hTERT, hTRT, iCE, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, EGFR, HER2/neu, MUC1, MUC1 (VNTR polymorphism), MUC1-c, MUC1-n, MUC2, PRAME, P15, RU1, RU2, SART-1, SART-3, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, human epidermal growth factor receptor 3 (HER3), alpha-actinin-4, ARTC1, CAR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyltransferase fusion protein, HLA-A2d, HLA-A1 1d, hsp70-2, KIAAO205, MART2, ME1, Myosin class I, NFYC, OGT, OS-9, pml-RARalpha fusion protein, PRDX5, PTPRK, K-ras, N-ras, RBAF600, SIRT2, SNRPDI, SYT-SSX1- or -SSX2 fusion protein, TGF-betaRII, triosephosphate isomerase, BAGE-1, GAGE-1, 2, 8, Gage 3, 4, 5, 6, 7, GnTVf, HERV-K-MEL, KK-LC-1, KM-HN-1, LAGE-1, MAGE-A1, MAGE-A2, MAGE-A3, MAGE-A4, MAGE-A6, MAGE-A9, MAGE-A10, MAGE-A12, MAGE-C2, mucin, NA-88, NY-ESO-1/LAGE-2, SAGE, Sp17, SSX-2, SSX-4, TAG-1, TAG-2, TRAG-3, TRP2-INT2g, XAGE-1b, gp100/Pmel17, mammaglobin-A, Melan-A/MART-1, NY-BR-1, OA1, RAB38/NY-MEL-1, TRP-1/gp75, adipophilin, AIM-2, ALDH1A1, BCLX (L), BCMA, BING-4, CPSF, cyclin Di, DKK1, ENAH (hMena), EP-CAM, EphA3, EZH2, FGF5, G250/MN/CAIX, IL13Ralpha2, intestinal carboxyl esterase, alpha fetoprotein, M-CSFI, MCSP, mdm-2, MMP-2, p53, PBF, PRAME, RAGE-1, RGS5, RNF43, RU2AS, secernin 1, SOX10, survivin, Telomerase, VEGF, or any combination thereof.

In some aspects, tumor neo-epitopes as used herein are tumor-specific epitopes, such as EQVWGMAVR (SEQ ID NO: 100) or CQGPEQVWGMAVREL (SEQ ID NO: 101) (R346W mutation of FLRT2), GETVTMPCP (SEQ ID NO: 102) or NVGETVTMPCPKVFS (SEQ ID NO: 103) (V73M mutation of VIPR2), GLGAQCSEA (SEQ ID NO: 104) or NNGLGAQCSEAVTLN (SEQ ID NO: 105) (R286C mutation of FCRL1), RKLTTELTI (SEQ ID NO: 106), LGPERRKLTTELTII (SEQ ID NO: 107), or PERRKLTTE (SEQ ID NO: 108) (S1613L mutation of FAT4), MDWVWMDTT (SEQ ID NO: 109), AVMDWVWMDTTLSLS (SEQ ID NO: 110), or VWMDTTLSL (SEQ ID NO: 111) (T2356M mutation of PIEZO2), GKTLNPSQT (SEQ ID NO: 112), SWFREGKTLNPSQTS (SEQ ID NO: 113), or REGKTLNPS (SEQ ID NO: 114) (A292T mutation of SIGLEC14), VRNATSYRC (SEQ ID NO: 115), LPNVTVRNATSYRCG (SEQ ID NO: 116), or NVTVRNATS (SEQ ID NO: 117) (D1143N mutation of SIGLEC1), FAMAQIPSL (SEQ ID NO: 118), PFAMAQIPSLSLRAV (SEQ ID NO: 119), or AQIPSLSLR (SEQ ID NO: 120) (Q678P mutation of SLC4A11).

Tumor-associated antigens may be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens may be, for example, proteins or protein fragments, complex carbohydrates, gangliosides, haptens, nucleic acids, other biological molecules or any combinations thereof.

### VI. CEA Antigen Targets

Disclosed herein include compositions comprising replication-defective vectors comprising one or more nucleic acid sequences encoding HPV E6 and/or E7antigen, and/or one or more nucleic acid sequences encoding mucin family antigen such as CEA, and/or one or more nucleic acid sequences encoding Brachyury, and/or one or more nucleic acid sequences encoding MUC1-c in same or separate replication-defective vectors.

CEA represents an attractive target antigen for immunotherapy since it is over-expressed in nearly all colorectal cancers and pancreatic cancers, and is also expressed by some lung and breast cancers, and uncommon tumors such as medullary thyroid cancer, but is not expressed in other cells of the body except for low-level expression in gastrointestinal epithelium. CEA contains epitopes that may be recognized in an MHC restricted fashion by T-cells.

It was discovered that multiple homologous immunizations with Ad5 [E1-, E2b-]-CEA(6D), encoding the tumor antigen CEA, induced CEA-specific cell-mediated immune (CMI) responses with antitumor activity in mice despite the presence of pre-existing or induced Ad5-neutralizing antibody. In the present phase I/II study, cohorts of patients with advanced colorectal cancer were immunized with escalating doses of Ad5 [E1-, E2b-]-CEA(6D). CEA-specific CMI responses were observed despite the presence of pre-existing Ad5 immunity in a majority (61.3%) of patients. Importantly, there was minimal toxicity, and overall patient survival (48% at 12 months) was similar regardless of pre-existing Ad5 neutralizing antibody titers. The results demonstrate that, in cancer patients, the novel Ad5 [E1-, E2b-] gene delivery platform generates significant CMI responses to the tumor antigen CEA in the setting of both naturally acquired and immunization-induced Ad5 specific immunity.

CEA antigen specific CMI can be, for example, greater than 10, 20, 30, 40, 50, 100, 200, 300, 400, 500, 600, 700, 800, 900, 1000, 5000, 10000, or more IFN-γ spot forming cells (SFC) per 10⁶ peripheral blood mononuclear cells (PBMC). In some embodiments, the immune response is raised in a human subject with a preexisting inverse Ad5 neutralizing antibody titer of greater than 50, 100, 150, 200, 300, 400, 500, 600, 700, 800, 900, 1000, 1500, 2000, 2500, 3000, 3500, 4000, 4500, 5000, 6000, 7000, 8000, 9000, 1000, 12000, 15000, or higher. The immune response may comprise a cell-mediated immunity and/or a humoral immunity as described herein. The immune response may be measured by one or more of intracellular cytokine staining (ICS), ELISpot, proliferation assays, cytotoxic T-cell assays including chromium release or equivalent assays, and gene expression analysis using any number of polymerase chain reaction (PCR) or RT-PCR based assays, as described herein and to the extent they are available to a person skilled in the art, as well as any other suitable assays known in the art for measuring immune response.

In some embodiments, the replication defective adenovirus vector comprises a modified sequence encoding a subunit with at least 75%, 80%, 85%, 90%, 95%, 98%, 99%, 99.5%, or 99.9% identity to a wild-type subunit of the polypeptide.

The immunogenic polypeptide may be a mutant CEA or a fragment thereof. In some embodiments, the immunogenic polypeptide comprises a mutant CEA with an Asn->Asp substitution at position 610. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the sequence encoding the immunogenic polypeptide comprises the sequence of SEQ ID NO: 22 (nucleic acid sequence for CEA-CAP1(6D)) or SEQ ID NO: 24 (amino acid sequence for the mutated CAP1(6D) epitope).

In some embodiments, the sequence encoding the immunogenic polypeptide comprises a sequence with at least 70% 75%, 80%, 85%, 90%, 95%, 98%, 99%, 99.5%, or 99.9% identity to SEQ ID NO: 22 or SEQ ID NO: 24 or a sequence generated from SEQ ID NO: 22 or SEQ ID NO: 24 by alternative codon replacements. In some embodiments, the immunogenic polypeptide encoded by the adenovirus vectors comprise up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human CEA sequence.

In some embodiments, the immunogenic polypeptide comprises a sequence from SEQ ID NO: 22 or SEQ ID NO: 24 or a modified version, e.g., comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, of SEQ ID NO: 22 or SEQ ID NO: 24.

Members of the CEA gene family are subdivided into three subgroups based on sequence similarity, developmental expression patterns and their biological functions: the CEA-related Cell Adhesion Molecule (CEACAM) subgroup containing twelve genes (CEACAM1, CEACAM3-CEACAM8, CEACAM16 and CEACAM18-CEACAM21), the Pregnancy Specific Glycoprotein (PSG) subgroup containing eleven closely related genes (PSG1-PSG11) and a subgroup of eleven pseudogenes (CEACAMP1-CEACAMP11). Most members of the CEACAM subgroup have similar structures that consist of an extracellular Ig-like domains composed of a single N-terminal V-set domain, with structural homology to the immunoglobulin variable domains, followed by varying numbers of C2-set domains of A or B subtypes, a transmembrane domain and a cytoplasmic domain. There are two members of CEACAM subgroup (CEACAM16 and CEACAM20) that show a few exceptions in the organization of their structures. CEACAM16 contains two Ig-like V-type domains at its N and C termini and CEACAM20 contains a truncated Ig-like V-type 1 domain. The CEACAM molecules can be anchored to the cell surface via their transmembrane domains (CEACAM5 thought CEACAM8) or directly linked to glycophosphatidylinositol (GPI) lipid moiety (CEACAM5, CEACAM18 thought CEACAM21).

CEA family members are expressed in different cell types and have a wide range of biological functions. CEACAMs are found prominently on most epithelial cells and are present on different leucocytes. In humans, CEACAM1, the ancestor member of CEA family, is expressed on the apical side of epithelial and endothelial cells as well as on lymphoid and myeloid cells. CEACAM1 mediates cell-cell adhesion through hemophilic (CEACAM1 to CEACAM1) as well as heterothallic (e.g., CEACAM1 to CEACAM5) interactions. In addition, CEACAM1 is involved in many other biological processes, such as angiogenesis, cell migration, and immune functions. CEACAM3 and CEACAM4 expression is largely restricted to granulocytes, and they are able to convey uptake and destruction of several bacterial pathogens including Neisseria, Moraxella, and Haemophilus species.

Thus, in various embodiments, compositions and methods relate to raising an immune response against a CEA, selected from the group consisting of CEACAM1, CEACAM3, CEACAM4, CEACAM5, CEACAM6, CEACAM7, CEACAM8, CEACAM16, CEACAM18, CEACAM19, CEACAM20, CEACAM21, PSG1, PSG2, PSG3, PSG4, PSG5, PSG6, PSG7, PSG8, PSG9, and PSG11. An immune response may be raised against cells, e.g., cancer cells, expressing or overexpressing one or more of the CEAs, using the methods and compositions. In some embodiments, the overexpression of the one or more CEAs in such cancer cells is over 5, 10, 20, 30, 40, 50, 60, 70, 80, 90, 100 fold, or more compared to non-cancer cells.

In certain embodiments, the CEA antigen used herein is a wild-type CEA antigen or a modified CEA antigen having a least a mutation in YLSGANLNL (SEQ ID NO: 23), a CAP1 epitope of CEA. The mutation can be conservative or non-conservative, substitution, addition, or deletion. In certain embodiments, the CEA antigen used herein has an amino acid sequence set forth in YLSGADLNL (SEQ ID NO: 24), a mutated CAP1 epitope. In further embodiments, the first replication-defective vector or a replication-defective vectors that express CEA has a nucleotide sequence at least 50%, 60%, 65%, 70%, 75%, 80%, 85%, 90%, 95%, 98%, 99%, 99.5%, 99.9%, or 100% identical to any portion of SEQ ID NO: 25 (the predicted sequence of an adenovirus vector expressing a modified CEA antigen), such as positions 1057 to 3165 of SEQ ID NO: 25 or full-length SEQ ID NO: 25.

### VII. Mucin Family Antigen Targets

Disclosed herein include compositions comprising replication-defective vectors comprising one or more nucleic acid sequences encoding HPV E6 and/or E7 antigen, and/or one or more nucleic acid sequences encoding mucin family antigen such as MUC1, and/or one or more nucleic acid sequences encoding Brachyury, and/or one or more nucleic acid sequences encoding CEA in same or separate replication-defective vectors.

The human mucin family (MUC1 to MUC21) includes secreted and transmembrane mucins that play a role in forming protective mucous barriers on epithelial surfaces in the body. These proteins function in to protecting the epithelia lining the respiratory, gastrointestinal tracts, and lining ducts in important organs such as, for example the mammary gland, liver, stomach, pancreas, and kidneys.

MUC1 (CD227) is a TAA that is over-expressed on a majority of human carcinomas and several hematologic malignancies. MUC1 (GenBank: X80761.1, NCBI: NM_001204285.1) and activates many important cellular pathways known to be involved in human disease. MUC1 is a heterodimeric protein formed by two subunits that is commonly overexpressed in several human cancers. MUC1 undergoes autoproteolysis to generate two subunits MUC1n and MUC1c that, in turn, form a stable noncovalent heterodimer.

The MUC1 C-terminal subunit (MUC1c) can comprise a 58 aa extracellular domain (ED), a 28 aa transmembrane domain (TM) and a 72 aa cytoplasmic domain (CD). The MUC1c also can contains a “CQC” motif that can allow for dimerization of MUC1 and it can also impart oncogenic function to a cell. In some cases, MUC1 can in part oncogenic function through inducing cellular signaling via MUC1c. MUC1c can interact with EGFR, ErbB2 and other receptor tyrosine kinases and contributing to the activation of the PI3K→AKT and MEK-ERK cellular pathways. In the nucleus, MUC1c activates the Wnt/β-catenin, STAT, and NF-κB RelA cellular pathways. In some cases MUC1 can impart oncogenic function through inducing cellular signaling via MUC1n. The MUC1 N-terminal subunit (MUC1n) can comprise variable numbers of 20 amino acid tandem repeats that can be glycosylated. MUC1 is normally expressed at the surface of glandular epithelial cells and is over-expressed and aberrantly glycosylated in carcinomas. MUC1 is a TAA that can be utilized as a target for tumor immunotherapy. Several clinical trials have been and are being performed to evaluate the use of MUC1 in immunotherapeutic vaccines. Importantly, these trials indicate that immunotherapy with MUC1 targeting is safe and may provide survival benefit.

However, clinical trials have also shown that MUC1 is a relatively poor immunogen. To overcome this, the inventors have identified a T lymphocyte immune enhancer peptide sequence in the C terminus region of the MUC1 oncoprotein (MUC1-C or MUC1c). Compared with the native peptide sequence, the agonist in their modified MUC1-C (a) bound HLA-A2 at lower peptide concentrations, (b) demonstrated a higher avidity for HLA-A2, (c) when used with antigen-presenting cells, induced the production of more IFN-γ by T-cells than with the use of the native peptide, and (d) was capable of more efficiently generating MUC1-specific human T-cell lines from cancer patients. Importantly, T-cell lines generated using the agonist epitope were more efficient than those generated with the native epitope for the lysis of targets pulsed with the native epitope and in the lysis of HLA-A2 human tumor cells expressing MUC1. Additionally, the inventors have identified additional CD8+ cytotoxic T lymphocyte immune enhancer agonist sequence epitopes of MUC1-C.

In certain aspects, there is provided a potent MUC1-C modified for immune enhancer capability (mMUC1-C or MUC1-C or MUC1c). The present disclosure provides a potent MUC1-C modified for immune enhancer capability incorporated it into a recombinant Ad5 [E1-, E2b-] platform to produce a new and more potent immunotherapeutic vaccine. For example, the immunotherapeutic vaccine can be Ad5 [E1-, E2b-]-mMUC1-C for treating MUC1 expressing cancers or infectious diseases.

Post-translational modifications play an important role in controlling protein function in the body and in human disease. For example, in addition to proteolytic cleavage discussed above, MUC1 can have several post-translational modifications such as glycosylation, sialylation, palmitoylation, or a combination thereof at specific amino acid residues. Provided herein are immunotherapies targeting glycosylation, sialylation, phosphorylation, or palmitoylation modifications of MUC1.

MUC1 can be highly glycosylated (N- and O-linked carbohydrates and sialic acid at varying degrees on serine and threonine residues within each tandem repeat, ranging from mono- to penta-glycosylation). Differentially O-glycosylated in breast carcinomas with 3,4-linked GlcNAc. N-glycosylation consists of high-mannose, acidic complex-type and hybrid glycans in the secreted form MUC1/SEC, and neutral complex-type in the transmembrane form, MUC1/TM.4. The present disclosure provides for immunotherapies targeting differentially O-glycosylated forms of MUC1.

Further, MUC1 can be sialylated. Membrane-shed glycoproteins from kidney and breast cancer cells have preferentially sialyated core 1 structures, while secreted forms from the same tissues display mainly core 2 structures. The O-glycosylated content is overlapping in both these tissues with terminal fucose and galactose, 2- and 3-linked galactose, 3- and 3,6-linked GalNAc-ol and 4-linked GlcNAc predominating. The present disclosure provides for immunotherapies targeting various sialylation forms of MUC1. Dual palmitoylation on cysteine residues in the CQC motif is required for recycling from endosomes back to the plasma membrane. The present disclosure provides for immunotherapies targeting various palmitoylation forms of MUC1.

Phosphorylation can affect MUC1's ability to induce specific cell signaling responses that are important for human health. The present disclosure provides for immunotherapies targeting various phosphorylated forms of MUC1. For example, MUC1 can be phosphorylated on tyrosine and serine residues in the C-terminal domain. Phosphorylation on tyrosines in the C-terminal domain can increase nuclear location of MUC1 and β-catenin. Phosphorylation by PKC delta can induce binding of MUC1 to β-catenin/CTNNB1 and decrease formation of β-catenin/E-cadherin complexes. Src-mediated phosphorylation of MUC1 can inhibit interaction with GSK3B. Src- and EGFR-mediated phosphorylation of MUC1 on Tyr-1229 can increase binding to β-catenin/CTNNB1. GSK3B-mediated phosphorylation of MUC1 on Ser-1227 can decrease this interaction, but restores the formation of the P3-cadherin/E-cadherin complex. PDGFR-mediated phosphorylation of MUC1 can increase nuclear colocalization of MUC1CT and CTNNB1. The present disclosure provides for immunotherapies targeting different phosphorylated forms of MUC1, MUC1c, and MUC1n known to regulate its cell signaling abilities.

The disclosure provides for immunotherapies that modulate MUC1c cytoplasmic domain and its functions in the cell. The disclosure provides for immunotherapies that comprise modulating a CQC motif in MUC1c. The disclosure provides for immunotherapies that comprise modulating the extracellular domain (ED), the transmembrane domain (TM), the cytoplasmic domain (CD) of MUC1c, or a combination thereof. The disclosure provides for immunotherapies that comprise modulating MUC1c's ability to induce cellular signaling through EGFR, ErbB2, or other receptor tyrosine kinases. The disclosure provides for immunotherapies that comprise modulating MUC1c's ability to induce PI3K→AKT, MEK→ERK, Wnt/β-catenin, STAT, NF-κB RelA cellular pathways, or combination thereof.

In some embodiments, the MUC1c immunotherapy can further comprise HPV E6 and/or E7, CEA, or Brachyury immunotherapy in the same replication-defective virus vectors or separate replication-defective virus vectors.

The disclosure also provides for immunotherapies that modulate MUC1n and its cellular functions. The disclosure also provides for immunotherapies comprising tandem repeats of MUC1n, the glycosylation sites on the tandem repeats of MUC1n, or a combination thereof. In some embodiments, the MUC1n immunotherapy further comprises HPV E6 and/or E7, CEA, or Brachyury immunotherapy in the same replication-defective virus vectors or separate replication-defective virus vectors.

The disclosure also provides vaccines comprising MUC1n, MUC1c, HPV E6 and/or E7, brachyury, CEA, or a combination thereof. The disclosure provides vaccines comprising MUC1c and HPV E6 and/or E7, brachyury, CEA, or a combination thereof. The disclosure also provides vaccines targeting MUC1n and HPV E6 and/or E7, Brachyury, CEA, or a combination thereof. In some embodiments, the antigen combination is contained in one vector as provided herein. In some embodiments, the antigen combination is contained in a separate vector as provided herein.

The present invention relates to a replication defective adenovirus vector of serotype 5 comprising a sequence encoding an immunogenic polypeptide. The immunogenic polypeptide may be an isoform of MUC1 or a subunit or a fragment thereof. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human MUC1 sequence.

In some embodiments, a MUC1-c antigen of this disclosure can be a modified MUC1 and can have a nucleotide sequence that is at least 80%, at least 85%, at least 90%, at least 92%, at least 95%, at least 97%, or at least 99% identical to SEQ ID NO: 26. In certain embodiments, a MUC1-c antigen of this disclosure can have a nucleotide sequence as set forth in SEQ ID NO: 26.

In some embodiments, a MUC1-c antigen of this disclosure can be a modified MUC1 and can have an amino sequence that is at least 80%, at least 85%, at least 90%, at least 92%, at least 95%, at least 97%, or at least 99% identical to SEQ ID NO: 27. In certain embodiments, a MUC1-c antigen of this disclosure can have an amino acid sequence as set forth in SEQ ID NO: 27.

### VIII. Brachyury Antigen Targets

Disclosed herein include compositions comprising replication-defective vectors comprising one or more nucleic acid sequences encoding HPV E6 and/or E7antigen, and/or one or more nucleic acid sequences encoding mucin family antigen such as MUC1, and/or one or more nucleic acid sequences encoding Brachyury, and/or one or more nucleic acid sequences encoding CEA in same or separate replication-defective vectors.

The disclosure provides for immunotherapies that comprise one or more antigens to Brachyury. Brachyury (also known as the “T” protein in humans) is a member of the T-box family of transcription factors that play key roles during early development, mostly in the formation and differentiation of normal mesoderm and is characterized by a highly conserved DNA-binding domain designated as T-domain. The epithelial to mesenchymal transition (EMT) is a key step during the progression of primary tumors into a metastatic state in which Brachyury plays a crucial role. The expression of Brachyury in human carcinoma cells induces changes characteristic of EMT, including up-regulation of mesenchymal markers, down-regulation of epithelial markers, and an increase in cell migration and invasion. Conversely, inhibition of Brachyury resulted in down-regulation of mesenchymal markers and loss of cell migration and invasion and diminished the ability of human tumor cells to form metastases. Brachyury can function to mediate epithelial-mesenchymal transition and promotes invasion.

The disclosure also provides for immunotherapies that modulate Brachyury effect on epithelial-mesenchymal transition function in cell proliferation diseases, such as cancer. The disclosure also provides immunotherapies that modulate Brachyury's ability to promote invasion in cell proliferation diseases, such as cancer. The disclosure also provides for immunotherapies that modulate the DNA binding function of T-box domain of Brachyury. In some embodiments, the Brachyury immunotherapy can further comprise one or more antigens to HPV E6 and/or E7, CEA, or MUC1, MUC1c or MUC1n.

Brachyury expression is nearly undetectable in most normal human tissues and is highly restricted to human tumors and often overexpressed making it an attractive target antigen for immunotherapy. In humans, Brachyury is encoded by the T gene (GenBank: AJ001699.1, NCBI: NM_003181.3). There are at least two different isoforms produced by alternative splicing found in humans. Each isoform has a number of natural variants.

Brachyury is immunogenic and Brachyury-specific CD8+ T-cells expanded in vitro can lyse Brachyury expressing tumor cells. These features of Brachyury make it an attractive tumor associated antigen (TAA) for immunotherapy. The Brachyury protein is a T-box transcription factor. It can bind to a specific DNA element, a near palindromic sequence “TCACACCT” through a region in its N-terminus, called the T-box to activate gene transcription when bound to such a site.

The disclosure also provides vaccines comprising Brachyury, HPV E6 and/or E7, MUC1, CEA, or a combination thereof. In some embodiments, the antigen combination is contained in one vector as provided herein. In some embodiments, the antigen combination is contained in a separate vector as provided herein.

In particular embodiments, the present invention relates to a replication defective adenovirus vector of serotype 5 comprising a sequence encoding an immunogenic polypeptide. The immunogenic polypeptide may be an isoform of Brachyury or a subunit or a fragment thereof. In some embodiments, the replication defective adenovirus vector comprises a sequence encoding a polypeptide with at least 70%, 75%, 80%, 85%, 90%, 95%, 98%, 99%, 99.5%, or 99.9% identity to the immunogenic polypeptide. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human Brachyury sequence.

In some embodiments, a Brachyury antigen of this disclosure can have an amino sequence that is at least 80%, at least 85%, at least 90%, at least 92%, at least 95%, at least 97%, or at least 99% identical to SEQ ID NO: 28. In certain embodiments, a Brachyury antigen of this disclosure can have an amino acid sequence as set forth in SEQ ID NO: 28.

### IX. Combination Therapies

Certain embodiments provide a combination immunotherapy and vaccine compositions for the treatment and prevention of cancer and infectious diseases. Some embodiments provide combination multi-targeted vaccines, immunotherapies and methods for enhanced therapeutic response to complex diseases such as infectious diseases and cancers. Each component of the combination therapy can be independently included in a vaccine composition for prevention of HPV infection or immunotherapy of an HPV-associated disease.

“Treatment” can refer to administration of a therapeutically effective dose of the vaccines of this disclosure to a subject. The treatment can be administered in a pharmaceutical composition to a subject. The subject can be suffering from a disease condition at the time of treatment and, in this case, the treatment can be referred to as therapeutic vaccination. The subject can also be healthy and disease free at the time of treatment and, in this case, the treatment can be referred to as a preventative vaccination.

A “subject” refers to any animal, including, but not limited to, humans, non-human primates (e.g., rhesus or other types of macaques), mice, pigs, horses, donkeys, cows, sheep, rats and fowls. A “subject” can be used herein interchangeably with “individual” or “patient.”

In some embodiments, any vaccine described herein (e.g., Ad5[E1-, E2b-]-E6; Ad5[E1-, E2b-]-E7; or Ad5[E1-, E2b-]-E6/E7) can be combined with low dose chemotherapy or low dose radiation. For example, in some embodiment, any vaccine described herein (e.g., Ad5[E1-, E2b-]-E6; Ad5[E1-, E2b-]-E7; or Ad5[E1-, E2b-]-E6/E7) can be combined with chemotherapy, such that the dose of chemotherapy administered is lower than the clinical standard of care. In some embodiments, the chemotherapy can be cyclophosphamide. The cyclophosphamide can administered at a dose that is lower than the clinical standard of care dosing. For example, the chemotherapy can be administered at 50 mg twice a day (BID) on days 1-5 and 8-12 every 2 weeks for a total of 8 weeks. In some embodiments, any vaccine described herein (e.g., Ad5[E1-, E2b-]-E6; Ad5[E1-, E2b-]-E7; or Ad5[E1-, E2b-]-E6/E7) can be combined with radiation, such that the dose of radiation administered is lower than the clinical standard of care. For example, in some embodiments, concurrent sterotactic body radiotherapy (SBRT) at 8 Gy can be given on day 8, 22, 36, 50 (every 2 weeks for 4 doses). Radiation can be administered to all feasible tumour sites using SBRT.

In some aspects, combination immunotherapies and vaccines provided herein can comprise a multi-targeted immunotherapeutic approach against antigens associated with the development of cancer such as tumor associated antigen, (TAA) or antigens know to be involved in a particular infectious disease, such as infectious disease associated antigen (IDAA). In some aspects, combination immunotherapies and vaccines provided herein can comprise a multi-targeted antigen signature immunotherapeutic approach against antigens associated with the development of cancer or infectious disease. The compositions and methods, in various embodiments, provide viral based vectors expressing a variant of HPV E6 and/or HPV E7 for immunization of a disease, as provided herein. These vectors can raise an immune response against HPV E6 and/or HPV E7.

In some aspects, the vector comprises at least one antigen. In some aspects, the vector comprises at least two antigens. In some aspects, the vaccine formulation comprises 1:1 ratio of vector to antigen. In some aspects, the vaccine comprises 1:2 ratio of vector to antigen. In some aspects, the vaccine comprises 1:3 ratio of vector to antigen. In some aspects, the vaccine comprises 1:4 ratio of vector to antigen. In some aspects, the vaccine comprises 1:5 ratio of vector to antigen. In some aspects, the vaccine comprises 1:6 ratio of vector to antigen. In some aspects, the vaccine comprises 1:7 ratio of vector to antigen. In some aspects, the vaccine comprises 1:8 ratio of vector to antigen. In some aspects, the vaccine comprises 1:9 ratio of vector to antigen. In some aspects, the vaccine comprises 1:10 ratio of vector to antigen.

In some aspects, the vaccine is a combination vaccine, wherein the vaccine comprises at least two vectors each containing at least a single antigen.

When a mixture of different antigens are simultaneously administered or expressed from a same or different vector in a subject, they may compete with one another. As a result the formulations comprising different concentration and ratios of expressed antigens in a combination immunotherapy or vaccine must be evaluated and tailored to the subject or group of subjects to ensure that effective and sustained immune responses occur after administration.

Composition that comprises multiple antigens can be present at various ratios. For example, formulations with more than vector can have various ratios. For example, immunotherapies or vaccines can have two different vectors in a stoichiometry of 1:1, 1:2, 1:3, 1:4, 1:5, 1:6, 1:7, 1:8, 1:9, 1:10, 1:15, 1:20, 1:30, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:5, 4:6, 4:7, 4:8, 5:1, 5:3, 5:4, 5:6, 5:7, 5:8, 6:1, 6:3, 6:4, 6:5, 6:7, 6:8, 7:1, 7:3, 7:4, 7:5, 7:6, 7:8, 8:1, 8:3, 8:4, 8:5, 8:6, or 8:7.

Certain embodiments provide combination immunotherapies comprising multi-targeted immunotherapeutic directed TAAs. Certain embodiments provide combination immunotherapies comprising multi-targeted immunotherapeutic directed to IDAAs.

In some embodiments, at least one of the recombinant nucleic acid vectors is a replication defective adenovirus vector that comprises a replication defective adenovirus 5 vector comprising a first identity value. In some embodiments, the replication defective adenovirus vector comprises a deletion in the E2b region. In some embodiments, the replication defective adenovirus vector further comprises a deletion in the E1 region. In some embodiments, the first identity value is at least 90%. In some embodiments, the first identity value is at least 95%. In some embodiments, the first identity value is at least 99%. In some embodiments, the first identity value is 100%. In some embodiments, the first identity value is at least 90%. In some embodiments, the first identity value is at least 95%. In some embodiments, the first identity value is at least 99%. In some embodiments, the first identity value is 100%. In some embodiments, the first identity value is at least 90%. In some embodiments, the first identity value is at least 95%. In some embodiments, the first identity value is at least 99%. In some embodiments, the first identity value is 100%.

In certain embodiments, there is provided a method of treating a HPV-expression cancer in an subject in need thereof, the method comprising: administering to the subject a pharmaceutical composition comprising a replication-defective vector comprising a nucleic acid sequence encoding a HPV antigen or any suitable antigen; and administering to the subject an immune checkpoint inhibitor. The method may further comprise administering to the subject a radiation therapy, a chemotherapy, or a combination thereof.

A. Immune Pathway Checkpoint Modulators

In some embodiments, combination therapy includes compositions that are administered with one or more immune checkpoint modulator, such as immune checkpoint inhibitors. In some embodiments, the composition comprises a replication-defective vector comprising a nucleotide sequence encoding a target antigen, such as HPV E6, HPV E7, or a combination thereof.

A balance between activation and inhibitory signals regulates the interaction between T lymphocytes and disease cells, wherein T-cell responses are initiated through antigen recognition by the T-cell receptor (TCR). The inhibitory pathways and signals are referred to as immune checkpoints. In normal circumstances, immune checkpoints play a critical role in control and prevention of autoimmunity and also protect from tissue damage in response to pathogenic infection.

In certain aspect, there are provided combination immunotherapies comprising viral vector based vaccines and compositions for modulating immune checkpoint inhibitory pathways for the treatment of cancer and infectious diseases. In some embodiments, modulating is increasing expression or activity of a gene or protein. In some embodiments, modulating is decreasing expression or activity of a gene or protein. In some embodiments, modulating affects a family of genes or proteins.

In general, the immune inhibitory pathways are initiated by ligand-receptor interactions. It is now clear that in diseases, the disease can co-opt immune-checkpoint pathways as mechanism for inducing immune resistance in a subject.

The induction of immune resistance or immune inhibitory pathways in a subject by a given disease can be blocked by molecular compositions such as siRNAs, antisense, small molecules, mimic, a recombinant form of ligand, receptor or protein, or antibodies (which can be an Ig fusion protein) that are known to modulate one or more of the Immune Inhibitory Pathways or a combination thereof. For example, preliminary clinical findings with blockers of immune-checkpoint proteins, such as cytotoxic T-lymphocyte-associated antigen 4 (CTLA4) and programmed cell death protein 1 (PD-1) have shown promise for enhancing antitumor immunity.

Because diseased cells can express multiple inhibitory ligands, and disease-infiltrating lymphocytes express multiple inhibitory receptors, dual or triple blockade of immune checkpoints proteins may enhance anti-disease immunity. Combination immunotherapies as provide herein can comprise one or more molecular compositions of the following immune-checkpoint proteins or fragments thereof: PD-1, PDL1, PDL2, CD28, CD80, CD86, CTLA4, B7RP1, ICOS, B7RPI, B7-H3 (also known as CD276), B7-H4 (also known as B7-S1, B7x and VCTN1), BTLA (also known as CD272), HVEM, KIR, TCR, LAG3 (also known as CD223), CD137, CD137L, OX40, OX40L, CD27, CD70, CD40, CD40L, TIM3 (also known as HAVcr2), GAL9, A2aR, ADORA CD276, VTCN1, IDO1, KIR3DL1, HAVCR2, VISTA, and CD244.

In some embodiments, the immune pathway checkpoint modulator activates or potentiates an immune response. In some embodiments, the immune pathway checkpoint modulator inhibits an immune response inhibitor. In some embodiments, the immune pathway checkpoint inhibits an immune response.

In some embodiments, the molecular composition comprises siRNAs. In some embodiments, the molecular composition comprises a small molecule. In some embodiments, the molecular composition comprises a recombinant form of a ligand. In some embodiments, the molecular composition comprises a recombinant form of a receptor. In some embodiments, the molecular composition comprises an antibody. In some embodiments, the combination therapy comprises more than one molecular composition and/or more than one type of molecular composition. As it will be appreciated by those in the art, future discovered proteins of the immune checkpoint inhibitory pathways are also envisioned to be encompassed in certain aspects.

In some embodiments, combination immunotherapies comprise molecular compositions for the modulation of CTLA4. In some embodiments, combination immunotherapies comprise molecular compositions for the modulation PD-1. In some embodiments, combination immunotherapies comprise molecular compositions for the modulation PDL1. In some embodiments, combination immunotherapies comprise molecular compositions for the modulation LAG3. In some embodiments, combination immunotherapies comprise molecular compositions for the modulation B7-H3. In some embodiments, combination immunotherapies comprise molecular compositions for the modulation B7-H4. In some embodiments, combination immunotherapies comprise molecular compositions for the modulation TIM3. In some embodiment, the immune pathway checkpoint modulator is a monoclonal or polyclonal antibody directed to PD-1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3 (i.e., HAVcr2), GAL9, and A2aR. In some embodiments, modulation is an increase or enhancement of expression. In other embodiments, modulation is the decrease of absence of expression.

Two exemplary immune checkpoint inhibitors include the cytotoxic T lymphocyte associated antigen-4 (CTLA-4) and the programmed cell death protein-1 (PD-1). CTLA-4 can be expressed exclusively on T-cells where it regulates early stages of T-cell activation. CTLA-4 interacts with the co-stimulatory T-cell receptor CD28 which can result in signaling that inhibits T-cell activity. Once TCR antigen recognition occurs, CD28 signaling may enhances TCR signaling, in some cases leading to activated T-cells and CTLA-4 inhibits the signaling activity of CD28. Certain embodiments provide immunotherapies as provided herein in combination with anti-CTLA-4 monoclonal antibody for the treatment of proliferative disease and cancer. Certain embodiments provide immunotherapies as provided herein in combination with CTLA-4 molecular compositions for the treatment of proliferative disease and cancer.

Programmed death cell protein ligand-1 (PDL1) is a member of the B7 family and is distributed in various tissues and cell types. PDL1 can interact with PD-1 inhibiting T-cell activation and CTL mediated lysis. Significant expression of PDL1 has been demonstrated on various human tumors and PDL1 expression is one of the key mechanisms in which tumors evade host antitumor immune responses. Programmed death-ligand 1 (PDL1) and programmed cell death protein-1 (PD-1) interact as immune checkpoints. This interaction can be a major tolerance mechanism which results in the blunting of anti-tumor immune responses and subsequent tumor progression. PD-1 is present on activated T cells and PDL1, the primary ligand of PD-1, is often expressed on tumor cells and antigen-presenting cells (APCs) as well as other cells, including B cells. Significant expression of PDL1 has been demonstrated on various human tumors including HPV-associated head and neck cancers. PDL1 interacts with PD-1 on T cells inhibiting T cell activation and cytotoxic T lymphocyte (CTL) mediated lysis. Certain embodiments provide immunotherapies as provided herein in combination with anti-PD-1 or anti-PDL1 monoclonal antibody for the treatment of proliferative disease and cancer. Certain embodiments provide immunotherapies as provided herein in combination with anti-PD-1 antibody or anti-PDL1 molecular compositions for the treatment of proliferative disease and cancer. Certain embodiments provide immunotherapies as provided herein in combination with anti-CTLA-4 monoclonal antibody and anti-PD-1 monoclonal antibody for the treatment of proliferative disease and cancer. Certain embodiments provide immunotherapies as provided herein in combination with anti-CTLA-4 monoclonal antibody and PDL1 monoclonal antibody for the treatment of proliferative disease and cancer. Certain embodiments provide immunotherapies as provided herein in combination with anti-CTLA-4 monoclonal antibody, anti-PD-1 monoclonal antibody, or anti-PDL1 monoclonal antibody, or a combination thereof, for the treatment of proliferative disease and cancer.

Certain embodiments provide immunotherapies as provided herein in combination with several antibodies directed against the PDL1/PD-1 pathway that are in clinical development for cancer treatment. In certain embodiments, anti-PDL1 antibodies may be used. Compared with anti-PD-1 antibodies that target T-cells, anti-PDL1 antibodies that target tumor cells are expected to have less side effects, including a lower risk of autoimmune-related safety issues, as blockade of PDL1 leaves the PDL2/PD-1 pathway intact to promote peripheral self-tolerance.

To this end, avelumab, a fully human IgG1 anti-PDL1 antibody (drug code MSB0010718C) has been produced. Avelumab selectively binds to PDL1 and competitively blocks its interaction with PD-1.

Avelumab is also cross-reactive with murine PDL1, thus allowing in vivo pharmacology studies to be conducted in normal laboratory mice. However, due to immunogenicity directed against the fully human avelumab molecule, the dosing regimen was limited to three doses given within a week.

The key preclinical pharmacology findings for avelumab are summarized below. Avelumab showed functional enhancement of primary T cell activation in vitro in response to antigen-specific and antigen non-specific stimuli; and significant inhibition of in vivo tumor growth (PDL1 expressing MC38 colon carcinoma) as a monotherapy. The in vivo efficacy of avelumab is driven by CD8+ T cells, as evidenced by complete abrogation of anti-tumor activity when this cell type was systemically depleted. Its combination with localized, fractionated radiotherapy resulted in complete regression of established tumors with generation of anti-tumor immune memory. Its antibody-dependent cell-mediated cytotoxicity (ADCC) was demonstrated against human tumor cells in vitro; furthermore, studies in ADCC deficient settings in vivo support a contribution of ADCC to anti-tumor efficacy. Additional findings of Avelumab include: no complement-dependent cytotoxicity was observed in vitro. Immunomonitoring assays with translational relevance for the clinic further support an immunological mechanism of action: consistent increases in CD8+PD-1+ T cells and CD8+ effector memory T cells as measured by fluorescence-activated cell sorter (FACS); enhanced tumor-antigen-specific CD8+ T cell responses as measured by pentamer staining and enzyme-linked immunosorbent spot (ELISPOT) assays.

Despite reports indicating that anti-tumor radiographic responses were unlikely using agents that interfere with PD-1-PDL1 binding in colorectal cancer, there have been reports of radiographic responses. Additionally, a correlation has been demonstrated in multiple clinical trials indicating that PDL1 expression levels on tumor tissue predict the likelihood of radiographic response. However, it has become clear that PDL1 expression, as it is currently measured, is not a definitive requirement for anti-tumor efficacy. It has been noted that colorectal tumors rarely express PDL1 compared with other tumors that are more likely to respond to PD-1-PDL1 blockade. However, it is known that a strong anti-tumor T cell response, producing IFN-γ, will induce PDL1 expression.

In some embodiments, without being bound by theory, it was contemplated that an underlying immune response is necessary for PD-1-PDL1 blockade to have an anti-tumor effect. Without being bound by theory, it was further contemplated that this combination of an immune checkpoint inhibitor with the standard therapy and an adenoviral vector composition such as Ad5-E6/E7 immunizations may be capable of induction of PDL1 expression and thereby increase the anti-tumor activity of PD-1-PDL1 blockade.

In some embodiments, other antibodies that selectively bind PDLlare employed, such as pembrolizumab, nivolumab, pidilizumab, atezolizumab, BMS-936559, MPDL3280A, and MEDI4736.

Some embodiments provide Ad5 [E1-, E2b-]-E6/E7 immunizations combined with PD-1 blockade that can increase an anti-tumor effect. A CMI response induced by the Ad5 [E1-, E2b-]-E6/E7 vaccine can be characterized to show kinetics of an anti-tumor response to evaluate the therapeutic potential of treating small versus large established tumors. Some embodiments provide Ad5 [E1-, E2b-]-E6 immunizations combined with PD-1 blockade that can increase an anti-tumor effect. A CMI response induced by the Ad5 [E1-, E2b-]-E6 vaccine can be characterized to show kinetics of an anti-tumor response to evaluate the therapeutic potential of treating small versus large established tumors. Some embodiments provide Ad5 [E1-, E2b-]-E7 immunizations combined with PD-1 blockade that can increase an anti-tumor effect. A CMI response induced by the Ad5 [E1-, E2b-]-E7 vaccine can be characterized to show kinetics of an anti-tumor response to evaluate the therapeutic potential of treating small versus large established tumors.

Immune checkpoint molecules can be expressed by T cells. Immune checkpoint molecules can effectively serve as “brakes” to down-modulate or inhibit an immune response. Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1), SIGLECIO (GeneBank accession number: AY358337.1), 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, ILIORA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3 which directly inhibit immune cells. For example, PD-1 can be combined with an adenoviral vaccine to treat a subject in need thereof. TABLE 1, without being exhaustive, shows exemplary immune checkpoint genes that can be inactivated to improve the efficiency of the adenoviral vaccine. Immune checkpoints gene can be selected from such genes listed in TABLE 1 and others involved in co-inhibitory receptor function, cell death, cytokine signaling, arginine tryptophan starvation, TCR signaling, Induced T-reg repression, transcription factors controlling exhaustion or anergy, and hypoxia mediated tolerance.

TABLE 1
Exemplary immune checkpoint genes

| Gene | NCBI # | | | Genome |
|---|---|---|---|---|
| Symbol | (GRCh38.p2) | Start | Stop | location |
| ADORA2A | 135 | 24423597 | 24442360 | 22q11.23 |
| CD276 | 80381 | 73684281 | 73714518 | 15q23-q24 |
| VTCN1 | 79679 | 117143587 | 117270368 | 1p13.1 |
| BTLA | 151888 | 112463966 | 112499702 | 3q13.2 |
| CTLA4 | 1493 | 203867788 | 203873960 | 2q33 |
| IDO1 | 3620 | 39913809 | 39928790 | 8p12-p11 |
| KIR3DL1 | 3811 | 54816438 | 54830778 | 19q13.4 |
| LAG3 | 3902 | 6772483 | 6778455 | 12p13.32 |
| PDCD1 | 5133 | 241849881 | 241858908 | 2q37.3 |
| HAVCR2 | 84868 | 157085832 | 157109237 | 5q33.3 |
| VISTA | 64115 | 71747556 | 71773580 | 10q22.1 |
| CD244 | 51744 | 160830158 | 160862902 | 1q23.3 |
| CISH | 1154 | 50606454 | 50611831 | 3p21.3 |

The combination of an adenoviral-based vaccine and an immune pathway checkpoint modulator may result in reduction in cancer recurrences in treated subjects, as compared to either agent alone. In yet another embodiment the combination of an adenoviral-based vaccine and an immune pathway checkpoint modulator may result in reduction in the presence or appearance of metastases or micro metastases in treated subjects, as compared to either agent alone. In another embodiment, the combination of an adenoviral-based vaccine and an immune pathway checkpoint modulator may result improved overall survival of treated subjects, as compared to either agent alone. In some cases, the combination of an adenoviral vaccine and an immune pathway checkpoint modulator may increase the frequency or intensity of tumor-specific T cell responses in subjects compared to either agent alone.

Some embodiments also disclose the use of immune checkpoint inhibition to improve performance of an adenoviral vector-based vaccine. The immune checkpoint inhibition may be administered at the time of the vaccine. The immune checkpoint inhibition may also be administered after a vaccine. Immune checkpoint inhibition may occur simultaneously to an adenoviral vaccine administration. Immune checkpoint inhibition may occur 1, 2, 3, 4, 5, 6, 7, 8, 9, 10, 15, 20, 30, 40, 50, or 60 minutes after vaccination. Immune checkpoint inhibition may also occur 1, 2, 3, 4, 5, 6, 7, 8, 9, 10, 11, 12, 13, 14, 15, 16, 17, 18, 19, 20, 21, 22, 23, or 24 hours post vaccination. In some cases, immune inhibition may occur 1, 2, 3, 4, 5, 6, or 7 days after vaccination. Immune checkpoint inhibition may occur at any time before or after vaccination.

In another aspect, there is provided a vaccine comprising an antigen and an immune pathway checkpoint modulator. Some embodiments pertain to a method for treating a subject having a condition that would benefit from downregulation of an immune checkpoint, PD-1 for example, and its natural binding partner(s) on cells of the subject.

An immune pathway checkpoint modulator may be combined with an adenoviral vaccine comprising nucleotide sequences encoding any antigen. For example, an antigen can be HPV E6 and/or HPV E7. An immune pathway checkpoint modulator may produce a synergistic effect when combined with a vaccine. An immune pathway checkpoint modulator may also produce an additive effect when combined with a vaccine.

In particular embodiments, a checkpoint immune inhibitor may be combined with a vector comprising nucleotide sequences encoding any antigen, optionally with chemotherapy or any other cancer care or therapy, such as VEGF inhibitors, angiogenesis inhibitors, radiation, other immune therapy, or any suitable cancer care or therapy.

B. Natural Killer (NK) Cells

In certain embodiments, native or engineered NK cells may be provided to be administered to a subject in need thereof, in combination with adenoviral vector-based compositions or immunotherapy as described herein.

The immune system is a tapestry of diverse families of immune cells each with its own distinct role in protecting from infections and diseases. Among these immune cells are the natural killer, or NK, cells as the body's first line of defense. NK cells have the innate ability to rapidly seek and destroy abnormal cells, such as cancer or virally-infected cells, without prior exposure or activation by other support molecules. In contrast to adaptive immune cells such as T cells, NK cells have been utilized as a cell-based “off-the-shelf” treatment in phase 1 clinical trials, and have demonstrated tumor killing abilities for cancer.

1. aNK Cells

In addition to native NK cells, there may be provided NK cells for administering to a subject that does not express Killer Inhibitory Receptors (KIR), which diseased cells often exploit to evade the killing function of NK cells. This unique activated NK cell, or aNK cell, lacks these inhibitory receptors while retaining the broad array of activating receptors which enable the selective targeting and killing of diseased cells. aNK cells also carry a larger pay load of granzyme and perforin containing granules, thereby enabling them to deliver a far greater payload of lethal enzymes to multiple targets.

2. taNK Cells

Chimeric antigen receptor (CAR) technology is among the most novel cancer therapy approaches currently in development. CARs are proteins that allow immune effector cells to target cancer cells displaying specific surface antigen (target-activated Natural Killer) is a platform in which aNK cells are engineered with one or more CARs to target proteins found on cancers and is then integrated with a wide spectrum of CARs. This strategy has multiple advantages over other CAR approaches using subject or donor sourced effector cells such as autologous T-cells, especially in terms of scalability, quality control and consistency.

Much of the cancer cell killing relies upon ADCC (antibody dependent cell-mediated cytotoxicity) whereupon effector immune cells attach to antibodies, which are in turn bound to the target cancer cell, thereby facilitating killing of the cancer by the effector cell. NK cells are the key effector cell in the body for ADCC and utilize a specialized receptor (CD16) to bind antibodies.

3. haNK Cells

Studies have shown that perhaps only 20% of the human population uniformly expresses the “high-affinity” variant of CD16 (haNK cells), which is strongly correlated with more favorable therapeutic outcomes compared to patients with the “low-affinity” CD16. Additionally, many cancer patients have severely weakened immune systems due to chemotherapy, the disease itself or other factors.

In certain aspects, NK cells are modified to express high-affinity CD16 (haNK cells). As such, haNK cells may potentiate the therapeutic efficacy of a broad spectrum of antibodies directed against cancer cells, and may be used in combination with immunotherapy or vectors described herein.

C. Costimulatory Molecules

In addition to the use of a recombinant adenovirus-based vector vaccine containing HPV antigens, co-stimulatory molecules can be incorporated into said vaccine that will increase immunogenicity.

Initiation of an immune response requires at least two signals for the activation of naive T cells by APCs (Damle, et al. J Immunol 148:1985-92 (1992); Guinan, et al. Blood 84:3261-82 (1994); Hellstrom, et al. Cancer Chemother Pharmacol 38:S40-44 (1996); Hodge, et al. Cancer Res 39:5800-07 (1999)). An antigen specific first signal is delivered through the T cell receptor (TCR) via the peptide/major histocompatability complex (MHC) and causes the T cell to enter the cell cycle. A second, or costimulatory, signal may be delivered for cytokine production and proliferation.

At least three distinct molecules normally found on the surface of professional antigen presenting cells (APCs) have been reported as capable of providing the second signal critical for T cell activation: B7-1 (CD80), ICAM-1 (CD54), and LFA-3 (human CD58) (Damle, et al. J Immunol 148:1985-92 (1992); Guinan, et al. Blood 84: 3261-82 (1994); Wingren, et al. Crit Rev Immunol 15: 235-53 (1995); Parra, et al. Scand. J Immunol 38: 508-14 (1993); Hellstrom, et al. Ann NY. Acad Sci 690: 225-30 (1993); Parra, et al. J Immunol 158: 637-42 (1997); Sperling, et al. J Immunol 157: 3909-17 (1996); Dubey, et al. J Immunol 155: 45-57 (1995); Cavallo, et al. Eur J Immunol 25: 1154-62 (1995)).

These costimulatory molecules have distinct T cell ligands. B7-1 interacts with the CD28 and CTLA-4 molecules, ICAM-1 interacts with the CD11a/CD18 (LFA-1/beta-2 integrin) complex, and LFA-3 interacts with the CD2 (LFA-2) molecules. Therefore, in a certain embodiment, it would be desirable to have a recombinant adenovirus vector that contains B7-1, ICAM-1, and LFA-3, respectively, that, when combined with a recombinant adenovirus-based vector vaccine containing one or more nucleic acids encoding target antigens such as HPV antigens, will further increase/enhance anti-tumor immune responses directed to specific target antigens.

### X. Immunological Fusion Partner Antigen Targets

The viral vectors or composition described herein may further comprise nucleic acid sequences that encode proteins, or an “immunological fusion partner,” that can increase the immunogenicity of the target antigen such as an HPV E6 and/or E7 antigen, or any target antigen disclosed herein. In this regard, the protein produced following immunization with the viral vector containing such a protein may be a fusion protein comprising the target antigen of interest fused to a protein that increases the immunogenicity of the target antigen of interest. Furthermore, combination therapy with Ad5[E1-, E2b-] vectors encoding for HPV E6 and/or E7 antigens and an immunological fusion partner can result in boosting the immune response, such that the combination of both therapeutic moieties acts to synergistically boost the immune response than either the Ad5[E1-, E2b-] vectors encoding for HPV E6 and/or E7 antigens alone, or the immunological fusion partner alone. For example, combination therapy with Ad5[E1-, E2b-] vectors encoding for HPV E6 and/or E7 antigens and an immunological fusion partner can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), antibody dependent cellular phagocytosis (ADCP) mechanisms, or any combination thereof. This synergistic boost can vastly improve survival outcomes after administration to a subject in need thereof. In certain embodiments, combination therapy with Ad5[E1-, E2b-] vectors encoding for HPV E6 and/or E7 antigens and an immunological fusion partner can result in generating an immune response comprises an increase in target antigen-specific CTL activity of about 1.5 to 20, or more fold in a subject administered the adenovirus vectors as compared to a control. In another embodiment, generating an immune response comprises an increase in target-specific CTL activity of about 1.5 to 20, or more fold in a subject administered the Ad5[E1-, E2b-] vectors encoding for HPV E6 and/or E7 antigens and an immunological fusion partner as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-αc), or other cytokines, of about 1.5 to 20, or more fold as compared to a control. In a further embodiment, generating an immune response comprises an increase in target-specific antibody production of between 1.5 and 5 fold in a subject administered the Ad5[E1-, E2b-] vectors encoding for HPV E6 and/or E7 antigens and an immunological fusion partner as described herein as compared to an appropriate control. In another embodiment, generating an immune response comprises an increase in target-specific antibody production of about 1.5 to 20, or more fold in a subject administered the adenovirus vector as compared to a control.

As an additional example, combination therapy with Ad5[E1-, E2b-] vectors encoding for target epitope antigens and an immunological fusion partner can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), antibody dependent cellular phagocytosis (ADCP) mechanisms, or any combination thereof. This synergistic boost can vastly improve survival outcomes after administration to a subject in need thereof. In certain embodiments, combination therapy with Ad5[E1-, E2b-] vectors encoding for target epitope antigens and an immunological fusion partner can result in generating an immune response comprises an increase in target antigen-specific CTL activity of about 1.5 to 20, or more fold in a subject administered the adenovirus vectors as compared to a control. In another embodiment, generating an immune response comprises an increase in target-specific CTL activity of about 1.5 to 20, or more fold in a subject administered the Ad5[E1-, E2b-] vectors encoding for target epitope antigens and an immunological fusion partner as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-α), or other cytokines, of about 1.5 to 20, or more fold as compared to a control. In a further embodiment, generating an immune response comprises an increase in target-specific antibody production of between 1.5 and 5 fold in a subject administered the adenovirus vectors as described herein as compared to an appropriate control. In another embodiment, generating an immune response comprises an increase in target-specific antibody production of about 1.5 to 20, or more fold in a subject administered the adenovirus vector as compared to a control.

In one embodiment, such an immunological fusion partner is derived from a Mycobacterium sp., such as a Mycobacterium tuberculosis-derived Ra12 fragment. The immunological fusion partner derived from Mycobacterium sp. can be any one of the sequences set forth in SEQ ID NO: 29-SEQ ID NO: 37. Ra12 compositions and methods for their use in enhancing the expression and/or immunogenicity of heterologous polynucleotide/polypeptide sequences are described in U.S. Pat. No. 7,009,042, which is herein incorporated by reference in its entirety. Briefly, Ra12 refers to a polynucleotide region that is a subsequence of a Mycobacterium tuberculosis MTB32A nucleic acid. MTB32A is a serine protease of 32 kDa encoded by a gene in virulent and avirulent strains of M. tuberculosis. The nucleotide sequence and amino acid sequence of MTB32A have been described (see, e.g., U.S. Pat. No. 7,009,042; Skeiky et al., Infection and Immun. 67:3998-4007 (1999), incorporated herein by reference in their entirety). C-terminal fragments of the MTB32A coding sequence can be expressed at high levels and remain as soluble polypeptides throughout the purification process. Moreover, Ra12 may enhance the immunogenicity of heterologous immunogenic polypeptides with which it is fused. A Ra12 fusion polypeptide can comprise a 14 kDa C-terminal fragment corresponding to amino acid residues 192 to 323 of MTB32A. Other Ra12 polynucleotides generally can comprise at least about 15, 30, 60, 100, 200, 300, or more nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants can have at least about 70%, 80%, or 90% identity, or more, to a polynucleotide sequence that encodes a native Ra12 polypeptide or a portion thereof.

In certain aspects, an immunological fusion partner can be derived from protein D, a surface protein of the gram-negative bacterium Haemophilus influenzae B. The immunological fusion partner derived from protein D can be the sequence set forth in SEQ ID NO: 38. In some cases, a protein D derivative comprises approximately the first third of the protein (e.g., the first N-terminal 100-110 amino acids). A protein D derivative may be lipidated. Within certain embodiments, the first 109 residues of a Lipoprotein D fusion partner is included on the N-terminus to provide the polypeptide with additional exogenous T-cell epitopes, which may increase the expression level in E. coli and may function as an expression enhancer. The lipid tail may ensure optimal presentation of the antigen to antigen presenting cells. Other fusion partners can include the non-structural protein from influenza virus, NS1 (hemagglutinin). Typically, the N-terminal 81 amino acids are used, although different fragments that include T-helper epitopes may be used.

In certain aspects, the immunological fusion partner can be the protein known as LYTA, or a portion thereof (particularly a C-terminal portion). The immunological fusion partner derived from LYTA can the sequence set forth in SEQ ID NO: 39. LYTA is derived from Streptococcus pneumoniae, which synthesizes an N-acetyl-L-alanine amidase known as amidase LYTA (encoded by the LytA gene). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus can be employed. Within another embodiment, a repeat portion of LYTA may be incorporated into a fusion polypeptide. A repeat portion can, for example, be found in the C-terminal region starting at residue 178. One particular repeat portion incorporates residues 188-305.

In some embodiments, the target antigen is fused to an immunological fusion partner, also referred to herein as an “immunogenic component,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Rα, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can produce a protein with substantial identity to one or more of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-10, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Rα, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can encode a nucleic acid encoding a protein with substantial identity to one or more of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-13, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Rα, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen fusion further comprises one or more immunological fusion partner, also referred to herein as an “immunogenic components,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Rα, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The sequence of IFN-γ can be, but is not limited to, a sequence as set forth in SEQ ID NO: 40. The sequence of TNFα can be, but is not limited to, a sequence as set forth in SEQ ID NO: 41. The sequence of IL-2 can be, but is not limited to, a sequence as set forth in SEQ ID NO: 42. The sequence of IL-8 can be, but is not limited to, a sequence as set forth in SEQ ID NO: 43. The sequence of IL-12 can be, but is not limited to, a sequence as set forth in SEQ ID NO: 44. The sequence of IL-18 can be, but is not limited to, a sequence as set forth in SEQ ID NO: 45. The sequence of IL-7 can be, but is not limited to, a sequence as set forth in SEQ ID NO: 46. The sequence of IL-3 can be, but is not limited to, a sequence as set forth in SEQ ID NO: 47. The sequence of IL-4 can be, but is not limited to, a sequence as set forth in SEQ ID NO: 48. The sequence of IL-5 can be, but is not limited to, a sequence as set forth in SEQ ID NO: 49. The sequence of IL-6 can be, but is not limited to, a sequence as set forth in SEQ ID NO: 50. The sequence of IL-9 can be, but is not limited to, a sequence as set forth in SEQ ID NO: 51. The sequence of IL-10 can be, but is not limited to, a sequence as set forth in SEQ ID NO: 52. The sequence of IL-13 can be, but is not limited to, a sequence as set forth in SEQ ID NO: 53. The sequence of IL-15 can be, but is not limited to, a sequence as set forth in SEQ ID NO: 54. The sequence of IL-16 can be, but is not limited to, a sequence as set forth in SEQ ID NO: 81. The sequence of IL-17 can be, but is not limited to, a sequence as set forth in SEQ ID NO: 82. The sequence of IL-23 can be, but is not limited to, a sequence as set forth in SEQ ID NO: 83. The sequence of IL-32 can be, but is not limited to, a sequences as set forth in SEQ ID NO: 84.

In some embodiments, the target antigen is fused or linked to an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, , IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Rα, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen is co-expressed in a cell with an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Rα, IL-37, TSLP, LIF, OSM, LT-α, LT-β3, CD40 ligand, Fas ligand, CD27 ligand, CD30 ligand, 4-1BBL, Trail, OPG-L, APRIL, LIGHT, TWEAK, BAFF, TGF-β1, and MIF.

In some embodiments, the target antigen is fused or linked to an immunological fusion partner, comprising CpG ODN (a non-limiting example sequence is shown in SEQ ID NO: 55), cholera toxin (a non-limiting example sequence is shown in SEQ ID NO: 56), a truncated A subunit coding region derived from a bacterial ADP-ribosylating exotoxin (a non-limiting example sequence is shown in (a non-limiting example sequence is shown in SEQ ID NO: 57), a truncated B subunit coding region derived from a bacterial ADP-ribosylating exotoxin (a non-limiting example sequence is shown in SEQ ID NO: 58), Hp91 (a non-limiting example sequence is shown in SEQ ID NO: 59), CCL20 (a non-limiting example sequence is shown in SEQ ID NO: 60), CCL3 (a non-limiting example sequence is shown in SEQ ID NO: 61), GM-CSF (a non-limiting example sequence is shown in SEQ ID NO: 62), G-CSF (a non-limiting example sequence is shown in SEQ ID NO: 63), LPS peptide mimic (non-limiting example sequences are shown in SEQ ID NO: 64-SEQ ID NO: 75), shiga toxin (a non-limiting example sequence is shown in SEQ ID NO: 76), diphtheria toxin (a non-limiting example sequence is shown in SEQ ID NO: 77), or CRM197 (a non-limiting example sequence is shown in SEQ ID NO: 80).

In some embodiments, the target antigen is fused or linked to an immunological fusion partner, comprising an IL-15 superagonist. Interleukin 15 (IL-15) is a naturally occurring inflammatory cytokine secreted after viral infections. Secreted IL-15 can carry out its function by signaling via the its cognate receptor on effector immune cells, and thus, can lead to overall enhancement of effector immune cell activity.

Based on IL-15's broad ability to stimulate and maintain cellular immune responses, it is believed to be a promising immunotherapeutic drug that could potentially cure certain cancers. However, major limitations in clinical development of IL-15 can include low production yields in standard mammalian cell expression systems and short serum half-life. Moreover, the IL-15:IL-15Rα complex, comprising proteins co-expressed by the same cell, rather than the free IL-15 cytokine, can be responsible for stimulating immune effector cells bearing IL-15 βγc receptor.

To contend with these shortcomings, a novel IL-15 superagonist mutant (IL-15N72D) was identified that has increased ability to bind IL-15Rβγc and enhanced biological activity. Addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC50) for supporting IL-15-dependent cell growth that was greater than 10-fold lower than that of free IL-15 cytokine.

In some embodiments, the IL-15 superagonist can be a novel IL-15 superagonist mutant (IL-15N72D). In certain embodiments, addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC50) for supporting IL-15-dependent cell growth that can be greater than 10-fold lower than that of free IL-15 cytokine

Thus, in some embodiments, the present disclosure provides a IL-15N72D:IL-15Rα/Fc super-agonist complex with an EC50 for supporting IL-15-dependent cell growth that is greater than 2-fold lower, greater than 3-fold lower, greater than 4-fold lower, greater than 5-fold lower, greater than 6-fold lower, greater than 7-fold lower, greater than 8-fold lower, greater than 9-fold lower, greater than 10-fold lower, greater than 15-fold lower, greater than 20-fold lower, greater than 25-fold lower, greater than 30-fold lower, greater than 35-fold lower, greater than 40-fold lower, greater than 45-fold lower, greater than 50-fold lower, greater than 55-fold lower, greater than 60-fold lower, greater than 65-fold lower, greater than 70-fold lower, greater than 75-fold lower, greater than 80-fold lower, greater than 85-fold lower, greater than 90-fold lower, greater than 95-fold lower, or greater than 100-fold lower than that of free IL-15 cytokine.

In some embodiments, the IL-15 super agonist is a biologically active protein complex of two IL-15N72D molecules and a dimer of soluble IL-15Rα/Fc fusion protein, also known as ALT-803. The composition of ALT-803 and methods of producing and using ALT-803 are described in U.S. Patent Application Publication 2015/0374790, which is herein incorporated by reference. It is known that a soluble IL-15Rα fragment, containing the so-called “sushi” domain at the N terminus (Su), can bear most of the structural elements responsible for high affinity cytokine binding. A soluble fusion protein can be generated by linking the human IL-15RαSu domain (amino acids 1-65 of the mature human IL-15Rα protein) with the human IgG1 CH2-CH3 region containing the Fc domain (232 amino acids). This IL-15RαSu/IgG1 Fc fusion protein can have the advantages of dimer formation through disulfide bonding via IgG1 domains and ease of purification using standard Protein A affinity chromatography methods.

In some embodiments, ALT-803 can have a soluble complex consisting of 2 protein subunits of a human IL-15 variant associated with high affinity to a dimeric IL-15Rα sushi domain/human IgG1 Fc fusion protein. The IL-15 variant is a 114 amino acid polypeptide comprising the mature human IL-15 cytokine sequence with an Asn to Asp substitution at position 72 of helix C N72D). The human IL-15R sushi domain/human IgG1 Fc fusion protein comprises the sushi domain of the IL-15R subunit (amino acids 1-65 of the mature human IL-15Rα protein) linked with the human IgG1 CH2-CH3 region containing the Fc domain (232 amino acids). Aside from the N72D substitution, all of the protein sequences are human. Based on the amino acid sequence of the subunits, the calculated molecular weight of the complex comprising two IL-15N72D polypeptides (an example IL-15N72D sequence is shown in SEQ ID NO: 78) and a disulfide linked homodimeric IL-15RαSu/IgG1 Fc protein (an example IL-15RαSu/Fc domain is shown in SEQ ID NO: 79) is 92.4 kDa. In some embodiments, a recombinant vector encoding for a target antigen and for ALT-803 can have any sequence described herein to encode for the target antigen and can have SEQ ID NO: 78, SEQ ID NO: 78, SEQ ID NO: 79, and SEQ ID NO: 79 in any order, to encode for ALT-803. In other embodiments, an IL-15 superagonist, such as ALT-803, can be administered as a separate pharmaceutical composition before or after immunization with a recombinant vector encoding for a target antigen. In further embodiments, an IL-15 superagonist, such as ALT-803, can be administered in a separate pharmaceutical composition as a protein complex or as a recombinant vector, which encodes for the protein complex.

Each IL-15N720 polypeptide has a calculated molecular weight of approximately 12.8 kDa and the IL-15RαSu/IgG 1 Fc fusion protein has a calculated molecular weight of approximately 33.4 kDa. Both the IL-15N72D and IL-15RαSu/IgG 1 Fc proteins can be glycosylated resulting in an apparent molecular weight of ALT- 803 of approximately 114 kDa by size exclusion chromatography. The isoelectric point (pI) determined for ALT-803 can range from approximately 5.6 to 6.5. Thus, the fusion protein can be negatively charged at pH 7.

Combination therapy with Ad5[E1-, E2b-] vectors encoding for HPV E6 and/or E7 and ALT-803 can result in boosting the immune response, such that the combination of both therapeutic moieties acts to synergistically boost the immune response than either therapy alone. For example, combination therapy with Ad5[E1-, E2b-] vectors encoding for HPV E6 and/or E7 antigens and ALT-803 can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), or antibody dependent cellular phagocytosis (ADCP) mechanisms. Combination therapy with Ad5[E1-, E2b-] vectors encoding for HPV E6 and/or E7 antigens and ALT-803 can synergistically boost any one of the above responses, or a combination of the above responses, to vastly improve survival outcomes after administration to a subject in need thereof.

Any of the immunogenicity enhancing agents described herein can be fused or linked to a target antigen by expressing the immunogenicity enhancing agents and the target antigen in the same recombinant vector, using any recombinant vector described herein.

Nucleic acid sequences that encode for such immunogenicity enhancing agents can be any one of SEQ ID NO: 29-SEQ ID NO: 84 and are summarized in TABLE 2.

TABLE 2
Sequences of Immunogenicity Enhancing Agents

| SEQ ID NO | Sequence |
|---|---|
| SEQ ID NO: 29 | TAASDNFQLSQGGQGFAIPIGQAMAIAGQIRSGGGSPTVHIGPTAF |
| | LGLGVVDNNGNGARVQRVVGSAPAASLGISTGDVITAVDGAPINS |
| | ATAMADALNGHHPGDVISVTWQTKSGGTRTGNVTLAEGPPA |
| SEQ ID NO: 30 | MHHHHHHTAASDNFQLSQGGQGFAIPIGQAMAIAGQIRSGGGSPT |
| | VHIGPTAFLGLGVVDNNGNGARVQRVVGSAPAASLGISTGDVITA |
| | VDGAPINSATAMADALNGHHPGDVISVTWQTKSGGTRTGNVTLA |
| | EGPPAEFDDDDKDPPDPHQPDMTKGYCPGGRWGFGDLAVCDGE |
| | KYPDGSFWHQWMQTWFTGPQFYFDCVSGGEPLPGPPPPGGCGGA |
| | IPSEQPNAP |
| SEQ ID NO: 31 | MHHHHHHTAASDNFQLSQGGQGFAIPIGQAMAIAGQIRSGGGSPT |
| | VHIGPTAFLGLGVVDNNGNGARVQRVVGSAPAASLGISTGDVITA |
| | VDGAPINSATAMADALNGHHPGDVISVTWQTKSGGTRTGNVTLA |
| | EGPPAEFPLVPRGSPMGSDVRDLNALLPAVPSLGGGGGCALPVSG |
| | AAQWAPVLDFAPPGASAYGSLGGPAPPPAPPPPPPPPPHSFIKQEPS |
| | WGGAEPHEEQCLSAFTVHFSGQFTGTAGACRYGPFGPPPPSQASS |
| | GQARMFPNAPYLPSCLESQPAIRNQGYSTVTFDGTPSYGHTPSHH |
| | AAQFPNHSFKHEDPMGQQGSLGEQQYSVPPPVYGCHTPTDSCTGS |
| | QALLLRTPYSSDNLYQMTSQLECMTWNQMNLGATLKGHSTGYES |
| | DNHTTPILCGAQYRIHTHGVFRGIQDVRRVPGVAPTLVRSASETSE |
| | KRPFMCAYSGCNKRYFKLSHLQMHSRKHTGEKPYQCDFKDCERR |
| | FFRSDQLKRHQRRHTGVKPFQCKTCQRKFSRSDHLKTHTRTHTGE |
| | KPFSCRWPSCQKKFARSDELVRHHNMHQRNMTKLQLAL |
| SEQ ID NO: 32 | MHHHHHHTAASDNFQLSQGGQGFAIPIGQAMAIAGQIRSGGGSPT |
| | VHIGPTAFLGLGVVDNNGNGARVQRVVGSAPAASLGISTGDVITA |
| | VDGAPINSATAMADALNGHHPGDVISVTWQTKSGGTRTGNVTLA |
| | EGPPAEFIEGRGSGCPLLENVISKTINPQVSKTEYKELLQEFIDDNA |
| | TTNAIDELKECFLNQTDETLSNVEVFMQLIYDSSLCDLF |
| SEQ ID NO: 33 | MHHHHHHTAASDNFQLSQGGQGFAIPIGQAMAIAGQIRSGGGSPT |
| | VHIGPTAFLGLGVVDNNGNGARVQRVVGSAPAASLGISTGDVITA |
| | VDGAPINSATAMADALNGHHPGDVISVTWQTKSGGTRTGNVTLA |
| | EGPPAEFMVDFGALPPEINSARMYAGPGSASLVAAAQMWDSVAS |
| | DLFSAASAFQSVVWGLTVGSWIGSSAGLMVAAASPYVAWMSVT |
| | AGQAELTAAQVRVAAAAYETAYGLTVPPPVIAENRAELMILIATN |
| | LLGQNTPAIAVNEAEYGEMWAQDAAAMFGYAAATATATATLLP |
| | FEEAPEMTSAGGLLEQAAAVEEASDTAAANQLMNNVPQALQQLA |
| | QPTQGTTPSSKLGGLWKTVSPHRSPISNMVSMANNHMSMTNSGV |
| | SMTNTLSSMLKGFAPAAAAQAVQTAAQNGVRAMSSLGSSLGSSG |
| | LGGGVAANLGRAASVGSLSVPQAWAAANQAVTPAARALPLTSLT |
| | SAAERGPGQMLGGLPVGQMGARAGGGLSGVLRVPPRPYVMPHSP |
| | AAGDIAPPALSQDRFADFPALPLDPSAMVAQVGPQVVNINTKLGY |
| | NNAVGAGTGIVIDPNGVVLTNNHVIAGATDINAFSVGSGQTYGVD |
| | VVGYDRTQDVAVLQLRGAGGLPSAAIGGGVAVGEPVVAMGNSG |
| | GQGGTPRAVPGRVVALGQTVQASDSLTGAEETLNGLIQFDAAIQP |
| | GDSGGPVVNGLGQVVGMNTAAS |
| SEQ ID NO: 34 | TAASDNFQLSQGGQGFAIPIGQAMAIAGQI |
| SEQ ID NO: 35 | TAASDNFQLSQGGQGFAIPIGQAMAIAGQIKLPTVHIGPTAFLGLG |
| | VVDNNGNGARVQRVVGSAPAASLGISTGDVITAVDGAPINSATA |
| | MADALNGHHPGDVISVTWQTKSGGTRTGNVTLAEGPPA |
| SEQ ID NO: 36 | TAASDNFQLSQGGQGFAIPIGQAMAIAGQIRSGGGSPTVHIGPTAF |
| | LGLGVVDNNGNGARVQRVVGSAPAASLGISTGDVITAVDGAPINS |
| | ATAMADALNGHHPGDVISVTWQTKSGGTRTGNVTLAE |
| SEQ ID NO: 37 | MSNSRRRSLRWSWLLSVLAAVGLGLATAPAQAAPPALSQDRFAD |
| | FPALPLDPSAMVAQVGPQVVNINTKLGYNNAVGAGTGIVIDPNGV |
| | VLTNNHVIAGATDINAFSVGSGQTYGVDVVGYDRTQDVAVLQLR |
| | GAGGLPSAAIGGGVAVGEPVVAMGNSGGQGGTPRAVPGRVVAL |
| | GQTVQASDSLTGAEETLNGLIQFDAAIQPGDSGGPVVNGLGQVVG |
| | MNTAASDNFQLSQGGQGFAIPIGQAMAIAGQIRSGGGSPTVHIGPT |
| | AFLGLGVVDNNGNGARVQRVVGSAPAASLGISTGDVITAVDGAPI |
| | NSATAMADALNGHHPGDVISVTWQTKSGGTRTGNVTLAEGPPA |
| SEQ ID NO: 38 | MKLKTLALSLLAAGVLAGCSSHSSNMANTQMKSDKIIIAHRGASG |
| | YLPEHTLESKALAFAQQADYLEQDLAMTKDGRLVVIHDHFLDGL |
| | TDVAKKFPHRHRKDGRYYVIDFTLKEIQSLEMTENFETKDGKQAQ |
| | VYPNRFPLWKSHFRIHTFEDEIEFIQGLEKSTGKKVGIYPEIKAPWF |
| | HHQNGKDIAAETLKVLKKYGYDKKTDMVYLQTFDFNELKRIKTE |
| | LLPQMGMDLKLVQLIAYTDWKETQEKDPKGYWVNYNYDWMFK |
| | PGAMAEVVKYADGVGPGWYMLVNKEESKPDNIVYTPLVKELAQ |
| | YNVEVHPYTVRKDALPAFFTDVNQMYDVLLNKSGATGVFTDFPD |
| | TGVEFLKGIK |
| SEQ ID NO: 39 | MEINVSKLRTDLPQVGVQPYRQVHAHSTGNPHSTVQNEADYHWR |
| | KDPELGFFSHIVGNGCIMQVGPVDNGAWDVGGGWNAETYAAVE |
| | LIESHSTKEEFMTDYRLYIELLRNLADEAGLPKTLDTGSLAGIKTH |
| | EYCTNNQPNNHSDHVDPYPYLAKWGISREQFKHDIENGLTIETGW |
| | QKNDTGYWYVHSDGSYPKDKFEKINGTWYYFDSSGYMLADRWR |
| | KHTDGNWYWFDNSGEMATGWKKIADKWYYFNEEGAMKTGWV |
| | KYKDTWYYLDAKEGAMVSNAFIQSADGTGWYYLKPDGTLADRP |
| | EFRMSQMA |
| SEQ ID NO: 40 | MKYTSYILAFQLCIVLGSLGCYCQDPYVKEAENLKKYFNAGHSDV |
| | ADNGTLFLGILKNWKEESDRKIMQSQIVSFYFKLFKNFKDDQSIQK |
| | SVETIKEDMNVKFFNSNKKKRDDFEKLTNYSVTDLNVQRKAIHEL |
| | IQVMAELSPAAKTGKRKRSQMLFRGRRASQ |
| SEQ ID NO: 41 | MSTESMIRDVELAEEALPKKTGGPQGSRRCLFLSLFSFLIVAGATT |
| | LFCLLHFGVIGPQREEFPRDLSLISPLAQAVRSSSRTPSDKPVAHVV |
| | ANPQAEGQLQWLNRRANALLANGVELRDNQLVVPSEGLYLIYSQ |
| | VLFKGQGCPSTHVLLTHTISRIAVSYQTKVNLLSAIKSPCQRETPEG |
| | AEAKPWYEPIYLGGVFQLEKGDRLSAEINRPDYLDFAESGQVYFGI |
| | IAL |
| SEQ ID NO: 42 | MYRMQLLSCIALSLALVTNSAPTSSSTKKTQLQLEHLLLDLQMILN |
| | GINNYKNPKLTRMLTFKFYMPKKATELKHLQCLEEELKPLEEVLN |
| | LAQSKNFHLRPRDLISNINVIVLELKGSETTFMCEYADETATIVEFL |
| | NRWITFCQSIISTLT |
| SEQ ID NO: 43 | MTSKLAVALLAAFLISAALCEGAVLPRSAKELRCQCIKTYSKPFHP |
| | KFIKELRVIESGPHCANTEIIVKLSDGRELCLDPKENWVQRVVEKF |
| | LKRAENS |
| SEQ ID NO: 44 | MEPLVTWVVPLLFLFLLSRQGAACRTSECCFQDPPYPDADSGSAS |
| | GPRDLRCYRISSDRYECSWQYEGPTAGVSHFLRCCLSSGRCCYFA |
| | AGSATRLQFSDQAGVSVLYTVTLWVESWARNQTEKSPEVTLQLY |
| | NSVKYEPPLGDIKVSKLAGQLRMEWETPDNQVGAEVQFRHRTPSS |
| | PWKLGDCGPQDDDTESCLCPLEMNVAQEFQLRRRQLGSQGSSWS |
| | KWSSPVCVPPENPPQPQVRFSVEQLGQDGRRRLTLKEQPTQLELPE |
| | GCQGLAPGTEVTYRLQLHMLSCPCKAKATRTLHLGKMPYLSGAA |
| | YNVAVISSNQFGPGLNQTWHIPADTHTEPVALNISVGTNGTTMYW |
| | PARAQSMTYCIEWQPVGQDGGLATCSLTAPQDPDPAGMATYSWS |
| | RESGAMGQEKCYYITIFASAHPEKLTLWSTVLSTYHFGGNASAAG |
| | TPHHVSVKNHSLDSVSVDWAPSLLSTCPGVLKEYVVRCRDEDSK |
| | QVSEHPVQPTETQVTLSGLRAGVAYTVQVRADTAWLRGVWSQP |
| | QRFSIEVQVSDWLIFFASLGSFLSILLVGVLGYLGLNRAARHLCPPL |
| | PTPCASSAIEFPGGKETWQWINPVDFQEEASLQEALVVEMSWDKG |
| | ERTEPLEKTELPEGAPELALDTELSLEDGDRCKAKM |
| SEQ ID NO: 45 | MAAEPVEDNCINFVAMKFIDNTLYFIAEDDENLESDYFGKLESKLS |
| | VIRNLNDQVLFIDQGNRPLFEDMTDSDCRDNAPRTIFIISMYKDSQ |
| | PRGMAVTISVKCEKISTLSCENKIISFKEMNPPDNIKDTKSDIIFFQR |
| | SVPGHDNKMQFESSSYEGYFLACEKERDLFKLILKKEDELGDRSI |
| | MFTVQNED |
| SEQ ID NO: 46 | MFHVSFRYIFGLPPLILVLLPVASSDCDIEGKDGKQYESVLMVSID |
| | QLLDSMKEIGSNCLNNEFNFFKRHICDANKEGMFLFRAARKLRQF |
| | LKMNSTGDFDLHLLKVSEGTTILLNCTGQVKGRKPAALGEAQPTK |
| | SLEENKSLKEQKKLNDLCFLKRLLQEIKTCWNKILMGTKEH |
| SEQ ID NO: 47 | MSRLPVLLLLQLLVRPGLQAPMTQTTSLKTSWVNCSNMIDEIITHL |
| | KQPPLPLLDFNNLNGEDQDILMENNLRRPNLEAFNRAVKSLQNAS |
| | AIESILKNLLPCLPLATAAPTRHPIHIKDGDWNEFRRKLTFYLKTLE |
| | NAQAQQTTLSLAIF |
| SEQ ID NO: 48 | MGLTSQLLPPLFFLLACAGNFVHGHKCDITLQEIIKTLNSLTEQKTL |
| | CTELTVTDIFAASKNTTEKETFCRAATVLRQFYSHHEKDTRCLGA |
| | TAQQFHRHKQLIRFLKRLDRNLWGLAGLNSCPVKEANQSTLENFL |
| | ERLKTIMREKYSKCSS |
| SEQ ID NO: 49 | MRMLLHLSLLALGAAYVYAIPTEIPTSALVKETLALLSTHRTLLIA |
| | NETLRIPVPVHKNHQLCTEEIFQGIGTLESQTVQGGTVERLFKNLSL |
| | IKKYIDGQKKKCGEERRRVNQFLDYLQEFLGVMNTEWIIES |
| SEQ ID NO: 50 | MNSFSTSAFGPVAFSLGLLLVLPAAFPAPVPPGEDSKDVAAPHRQP |
| | LTSSERIDKQIRYILDGISALRKETCNKSNMCESSKEALAENNLNLP |
| | KMAEKDGCFQSGFNEETCLVKIITGLLEFEVYLEYLQNRFESSEEQ |
| | ARAVQMSTKVLIQFLQKKAKNLDAITTPDPTTNASLLTKLQAQNQ |
| | WLQDMTTHLILRSFKEFLQSSLRALRQM |
| SEQ ID NO: 51 | MVLTSALLLCSVAGQGCPTLAGILDINFLINKMQEDPASKCHCSA |
| | NVTSCLCLGIPSDNCTRPCFSERLSQMTNTTMQTRYPLIFSRVKKS |
| | VEVLKNNKCPYFSCEQPCNQTTAGNALTFLKSLLEIFQKEKMRGM |
| | RGKI |
| SEQ ID NO: 52 | MHSSALLCCLVLLTGVRASPGQGTQSENSCTHFPGNLPNMLRDLR |
| | DAFSRVKTFFQMKDQLDNLLLKESLLEDFKGYLGCQALSEMIQFY |
| | LEEVMPQAENQDPDIKAHVNSLGENLKTLRLRLRRCHRFLPCENK |
| | SKAVEQVKNAFNKLQEKGIYKAMSEFDIFINYIEAYMTMKIRN |
| SEQ ID NO: 53 | MALLLTTVIALTCLGGFASPGPVPPSTALRELIEELVNITQNQKAPL |
| | CNGSMVWSINLTAGMYCAALESLINVSGCSAIEKTQRMLSGFCPH |
| | KVSAGQFSSLHVRDTKIEVAQFVKDLLLHLKKLFREGQFNRNFESI |
| | IICRDRT |
| SEQ ID NO: 54 | MDFQVQIFSFLLISASVIMSRANWVNVISDLKKIEDLIQSMHIDATL |
| | YTESDVHPSCKVTAMKCFLLELQVISLESGDASIHDTVENLIILANN |
| | SLSSNGNVTESGCKECEELEEKNIKEFLQSFVHIVQMFINTS |
| SEQ ID NO: 55 | MEGDGSDPEPPDAGEDSKSENGENAPIYCICRKPDINCFMIGCDNC |
| | NEWFHGDCIRITEKMAKAIREWYCRECREKDPKLEIRYRHKKSRE |
| | RDGNERDSSEPRDEGGGRKRPVPDPNLQRRAGSGTGVGAMLARG |
| | SASPHKSSPQPLVATPSQHHQQQQQQIKRSARMCGECEACRRTED |
| | CGHCDFCRDMKKFGGPNKIRQKCRLRQCQLRARESYKYFPSSLSP |
| | VTPSESLPRPRRPLPTQQQPQPSQKLGRIREDEGAVASSTVKEPPEA |
| | TATPEPLSDEDLPLDPDLYQDFCAGAFDDNGLPWMSDTEESPFLD |
| | PALRKRAVKVKHVKRREKKSEKKKEERYKRHRQKQKHKDKWK |
| | HPERADAKDPASLPQCLGPGCVRPAQPSSKYCSDDCGMKLAANRI |
| | YEILPQRIQQWQQSPCIAEEHGKKLLERIRREQQSARTRLQEMERR |
| | FHELEAIILRAKQQAVREDEESNEGDSDDTDLQIFCVSCGHPINPRV |
| | ALRHMERCYAKYESQTSFGSMYPTRIEGATRLFCDVYNPQSKTYC |
| | KRLQVLCPEHSRDPKVPADEVCGCPLVRDVFELTGDFCRLPKRQC |
| | NRHYCWEKLRRAEVDLERVRVWYKLDELFEQERNVRTAMTNRA |
| | GLLALMLHQTIQHDPLTTDLRSSADR |
| SEQ ID NO: 56 | MIKLKFGVFFTVLLSSAYAHGTPQNITDLCAEYHNTQIYTLNDKIF |
| | SYTESLAGKREMAIITFKNGAIFQVEVPGSQHIDSQKKAIERMKDT |
| | LRIAYLTEAKVEKLCVWNNKTPHAIAAISMAN |
| SEQ ID NO: 57 | MVKIIFVFFIFLSSFSYANDDKLYRADSRPPDEIKQSGGLMPRGQNE |
| | YFDRGTQMNINLYDHARGTQTGFVRHDDGYVSTSISLRSAHLVG |
| | QTILSGHSTYYIYVIATAPNMFNVNDVLGAYSPHPDEQEVSALGGI |
| | PYSQIYGWYRVHFGVLDEQLHRNRGYRDRYYSNLDIAPAADGYG |
| | LAGFPPEHRAWREEPWIHHAPPGCGNAPRSSMSNTCDEKTQSLGV |
| | KFLDEYQSKVKRQIFSGYQSDIDTHNRIKDEL |
| SEQ ID NO: 58 | MIKLKFGVFFTVLLSSAYAHGTPQNITDLCAEYHNTQIHTLNDKIL |
| | SYTESLAGNREMAIITFKNGATFQVEVPGSQHIDSQKKAIERMKDT |
| | LRIAYLTEAKVEKLCVWNNKTPHAIAAISMAN |
| SEQ ID NO: 59 | DPNAPKRPPSAFFLFCSE |
| SEQ ID NO: 60 | MCCTKSLLLAALMSVLLLHLCGESEAASNFDCCLGYTDRILHPKFI |
| | VGFTRQLANEGCDINAIIFHTKKKLSVCANPKQTWVKYIVRLLSK |
| | KVKNM |
| SEQ ID NO: 61 | MQVSTAALAVLLCTMALCNQFSASLAADTPTACCFSYTSRQIPQN |
| | FIADYFETSSQCSKPGVIFLTKRSRQVCADPSEEWVQKYVSDLELS |
| | A |
| SEQ ID NO: 62 | MWLQSLLLLGTVACSISAPARSPSPSTQPWEHVNAIQEARRLLNLS |
| | RDTAAEMNETVEVISEMFDLQEPTCLQTRLELYKQGLRGSLTKLK |
| | GPLTMMASHYKQHCPPTPETSCATQIITFESFKENLKDFLLVIPFDC |
| | WEPVQE |
| SEQ ID NO: 63 | MAGPATQSPMKLMALQLLLWHSALWTVQEATPLGPASSLPQSFL |
| | LKCLEQVRKIQGDGAALQEKLCATYKLCHPEELVLLGHSLGIPWA |
| | PLSSCPSQALQLAGCLSQLHSGLFLYQGLLQALEGISPELGPTLDTL |
| | QLDVADFATTIWQQMEELGMAPALQPTQGAMPAFASAFQRRAG |
| | GVLVASHLQSFLEVSYRVLRHLAQP |
| SEQ ID NO: 64 | QEINSSY |
| SEQ ID NO: 65 | SHPRLSA |
| SEQ ID NO: 66 | SMPNPMV |
| SEQ ID NO: 67 | GLQQVLL |
| SEQ ID NO: 68 | HELSVLL |
| SEQ ID NO: 69 | YAPQRLP |
| SEQ ID NO: 70 | TPRTLPT |
| SEQ ID NO: 71 | APVHSSI |
| SEQ ID NO: 72 | APPHALS |
| SEQ ID NO: 73 | TFSNRFI |
| SEQ ID NO: 74 | VVPTPPY |
| SEQ ID NO: 75 | ELAPDSP |
| SEQ ID NO: 76 | TPDCVTGKVEYTKYNDDDTFTVKVGDKELFTNRWNLQSLLLSAQ |
| | ITGMTVTIKQNACHNGGGFSEVIFR |
| SEQ ID NO: 77 | MSRKLFASILIGALLGIGAPPSAHAGADDVVDSSKSFVMENFSSYH |
| | GTKPGYVDSIQKGIQKPKSGTQGNYDDDWKGFYSTDNKYDAAG |
| | YSVDNENPLSGKAGGVVKVTYPGLTKVLALKVDNAETIKKELGL |
| | SLTEPLMEQVGTEEFIKRFGDGASRVVLSLPFAEGSSSVEYINNWE |
| | QAKALSVELEINFETRGKRGQDAMYEYMAQACAGNRVRRSVGSS |
| | LSCINLDWDVIRDKTKTKIESLKEHGPIKNKMSESPNKTVSEEKAK |
| | QYLEEFHQTALEHPELSELKTVTGTNPVFAGANYAAWAVNVAQV |
| | IDSETADNLEKTTAALSILPGIGSVMGIADGAVHHNTEEIVAQSIAL |
| | SSLMVAQAIPLVGELVDIGFAAYNFVESIINLFQVVHNSYNRPAYS |
| | PGHKTQPFLHDGYAVSWNTVEDSIIRTGFQGESGHDIKITAENTPL |
| | PIAGVLLPTIPGKLDVNKSKTHISVNGRKIRMRCRAIDGDVTFCRP |
| | KSPVYVGNGVHANLHVAFHRSSSEKIHSNEISSDSIGVLGYQKTVD |
| | HTKVNSKLSLFFEIKS |
| SEQ ID NO: 78 | NWVNVISDLKKIEDLIQSMHIDATLYTESDVHPSCKVTAMKCFLL |
| | ELQVISLESGDASIHDTVENLIILANDSLSSNGNVTESGCKECEELE |
| | EKNIKEFLQSFVHIVQMFINTS |
| SEQ ID NO: 79 | ITCPPPMSVEHADIWVKSYSLYSRERYICNSGFKRKAGTSSLTECV |
| | LNKATNVAHWTTPSLKCIREPKSCDKTHTCPPCPAPELLGGPSVFL |
| | FPPKPKDTLMISRTPEVTCVVVDVSHEDPEVKFNWYVDGVEVHN |
| | AKTICPREEQYNSTYRVVSVLTVLHQDWLNGKEYKCKVSNKALP |
| | APIEKTISKAKGQPREPQVYTLPPSRDELTKNQVSLTCLVKGFYPS |
| | DIAVEWESNGQPENNYKTTPPVLDSDGSFFLYSKLTVDKSRWQQG |
| | NVFSCSVMHEALHNHYTQKSLSLSPGK |
| SEQ ID NO: 80 | GADDVVDSSKSFVMENFSSYHGTKPGYVDSIQKGIQKPKSGTQGN |
| | YDDDWKEFYSTDNKYDAAGYSVDNENPLSGKAGGVVKVTYPGL |
| | TKVLALKVDNAETIKKELGLSLTEPLMEQVGTEEFIKRFGDGASR |
| | VVLSLPFAEGSSSVEYINNWEQAKALSVELEINFETRGKRGQDAM |
| | YEYMAQACAGNRVRRSVGSSLSCINLDWDVIRDKTKTKIESLKEH |
| | GPIKNKMSESPNKTVSEEKAKQYLEEFHQTALEHPELSELKTVTGT |
| | NPVFAGANYAAWAVNVAQVIDSETADNLEKTTAALSILPGIGSVM |
| | GIADGAVHHNTEEIVAQSIALSSLMVAQAIPLVGELVDIGFAAYNF |
| | VESIINLFQVVHNSYNRPAYSPGHKTQPFLHDGYAVSWNTVEDSII |
| | RTGFQGESGHDIKITAENTPLPIAGVLLPTIPGKLDVNKSKTHISVN |
| | GRKIRMRCRAIDGDVTFCRPKSPVYVGNGVHANLHVAFHRSSSEK |
| | IHSNEISSDSIGVLGYQKTVDHTKVNSKLSLFFEIKS |
| SEQ ID NO: 81 | MESHSRAGKSRKSAKFRSISRSLMLCNAKTSDDGSSPDEKYPDPFE |
| | ISLAQGKEGIFHSSVQLADTSEAGPSSVPDLALASEAAQLQAAGND |
| | RGKTCRRIFFMKESSTASSREKPGKLEAQSSNFLFPKACHQRARSN |
| | STSVNPYCTREIDFPMTKKSAAPTDRQPYSLCSNRKSLSQQLDCPA |
| | GKAAGTSRPTRSLSTAQLVQPSGGLQASVISNIVLMKGQAKGLGF |
| | SIVGGKDSIYGPIGIYVKTIFAGGAAAADGRLQEGDEILELNGESM |
| | AGLTHQDALQKFKQAKKGLLTLTVRTRLTAPPSLCSHLSPPLCRSL |
| | SSSTCITKDSSSFALESPSAPISTAKPNYRIMVEVSLQKEAGVGLGIG |
| | LCSVPYFQCISGIFVHTLSPGSVAHLDGRLRCGDEIVEISDSPVHCL |
| | TLNEVYTILSRCDPGPVPIIVSRHPDPQVSEQQLKEAVAQAVENTK |
| | FGKERHQWSLEGVKRLESSWHGRPTLEKEREKNSAPPHRRAQKV |
| | MIRSSSDSSYMSGSPGGSPGSGSAEKPSSDVDISTHSPSLPLAREPV |
| | VLSIASSRLPQESPPLPESRDSHPPLRLKKSFEILVRKPMSSKPKPPP |
| | RKYFKSDSDPQKSLEERENSSCSSGHTPPTCGQEARELLPLLLPQE |
| | DTAGRSPSASAGCPGPGIGPQTKSSTEGEPGWRRASPVTQTSPIKH |
| | PLLKRQARMDYSFDTTAEDPWVRISDCIKNLFSPIMSENHGHMPL |
| | QPNASLNEEEGTQGHPDGTPPKLDTANGTPKVYKSADSSTVKKGP |
| | PVAPKPAWFRQSLKGLRNRASDPRGLPDPALSTQPAPASREHLGS |
| | HIRASSSSSSIRQRISSFETFGSSQLPDKGAQRLSLQPSSGEAAKPLG |
| | KHEEGRFSGLLGRGAAPTLVPQQPEQVLSSGSPAASEARDPGVSES |
| | PPPGRQPNQKTLPPGPDPLLRLLSTQAEESQGPVLKMPSQRARSFP |
| | LTRSQSCETKLLDEKTSKLYSISSQVSSAVMKSLLCLPSSISCAQTP |
| | CIPKEGASPTSSSNEDSAANGSAETSALDTGFSLNLSELREYTEGLT |
| | EAKEDDDGDHSSLQSGQSVISLLSSEELKKLIEEVKVLDEATLKQL |
| | DGIHVTILHKEEGAGLGFSLAGGADLENKVITVHRVFPNGLASQE |
| | GTIQKGNEVLSINGKSLKGTTHHDALAILRQAREPRQAVIVTRKLT |
| | PEAMPDLNSSTDSAASASAASDVSVESTEATVCTVTLEKMSAGLG |
| | FSLEGGKGSLHGDKPLTINRIFKGAASEQSETVQPGDEILQLGGTA |
| | MQGLTRFEAWNIIKALPDGPVTIVIRRKSLQSKETTAAGDS |
| SEQ ID NO: 82 | MTPGKTSLVSLLLLLSLEAIVKAGITIPRNPGCPNSEDKNFPRTVMV |
| | NLNIHNRNTNTNPKRSSDYYNRSTSPWNLHRNEDPERYPSVIWEA |
| | KCRHLGCINADGNVDYHMNSVPIQQEILVLRREPPHCPNSFRLEKI |
| | LVSVGCTCVTPIVHHVA |
| SEQ ID NO: 83 | RAVPGGSSPAWTQCQQLSQKLCTLAWSAHPLVGHMDLREEGDEE |
| | TTNDVPHIQCGDGCDPQGLRDNSQFCLQRIHQGLIFYEKLLGSDIF |
| | TGEPSLLPDSPVGQLHASLLGLSQLLQPEGHHWETQQIPSLSPSQP |
| | WQRLLLRFKILRSLQAFVAVAARVFAHGAATLSPIWELKKDVYV |
| | VELDWYPDAPGEMVVLTCDTPEEDGITWTLDQSSEVLGSGKTLTI |
| | QVKEFGDAGQYTCHKGGEVLSHSLLLLHKKEDGIWSTDILKDQK |
| | EPKNKTFLRCEAKNYSGRFTCWWLTTISTDLTFSVKSSRGSSDPQG |
| | VTCGAATLSAERVRGDNKEYEYSVECQEDSACPAAEESLPIEVMV |
| | DAVHKLKYENYTSSFFIRDIIKPDPPKNLQLKPLKNSRQVEVSWEY |
| | PDTWSTPHSYFSLTFCVQVQGKSKREKKDRVFTDKTSATVICRKN |
| | ASISVRAQDRYYSSSWSEWASVPCS |
| SEQ ID NO: 84 | MCFPKVLSDDMKKLKARMVMLLPTSAQGLGAWVSACDTEDTVG |
| | HLGPWRDKDPALWCQLCLSSQHQAIERFYDKMQNAESGRGQVM |
| | SSLAELEDDFKEGYLETVAAYYEEQHPELTPLLEKERDGLRCRGN |
| | RSPVPDVEDPATEEPGESFCDKVMRWFQAMLQRLQTWWHGVLA |
| | WVKEKVVALVHAVQALWKQFQSFCCSLSELFMSSFQSYGAPRGD |
| | KEELTPQKCSEPQSSK |

In some embodiments, the nucleic acid sequences for the target antigen and the immunological fusion partner are not separated by any nucleic acids. In other embodiments, a nucleic acid sequence that encodes for a linker can be inserted between the nucleic acid sequence encoding for any target antigen described herein and the nucleic acid sequence encoding for any immunological fusion partner described herein. Thus, in certain embodiments, the protein produced following immunization with the viral vector containing a target antigen, a linker, and an immunological fusion partner can be a fusion protein comprising the target antigen of interest followed by the linker and ending with the immunological fusion partner, thus linking the target antigen to an immunological fusion partner that increases the immunogenicity of the target antigen of interest via a linker. In some embodiments, the sequence of linker nucleic acids can be from about 1 to about 150 nucleic acids long, from about 5 to about 100 nucleic acids along, or from about 10 to about 50 nucleic acids in length. In some embodiments, the nucleic acid sequences may encode one or more amino acid residues. In some embodiments, the amino acid sequence of the linker can be from about 1 to about 50, or about 5 to about 25 amino acid residues in length. In some embodiments, the sequence of the linker comprises less than 10 amino acids. In some embodiments, the linker can be a polyalanine linker, a polyglycine linker, or a linker with both alanines and glycines.

Nucleic acid sequences that encode for such linkers can be any one of SEQ ID NO: 85-SEQ ID NO: 99 and are summarized in TABLE 3.

TABLE 3
Sequences of Linkers

| SEQ ID NO | Sequence |
|---|---|
| SEQ ID NO: 85 | MAVPMQLSCSR |
| SEQ ID NO: 86 | RSTG |
| SEQ ID NO: 87 | TR |
| SEQ ID NO: 88 | RSQ |
| SEQ ID NO: 89 | RSAGE |
| SEQ ID NO: 90 | RS |
| SEQ ID NO: 91 | GG |
| SEQ ID NO: 92 | GSGGSGGSG |
| SEQ ID NO: 93 | GGSGGSGGSGG |
| SEQ ID NO: 94 | GGSGGSGGSGGSGG |
| SEQ ID NO: 95 | GGSGGSGGSGGSGGSGG |
| SEQ ID NO: 96 | GGSGGSGGSGGSGGSGGSGG |
| SEQ ID NO: 97 | GGSGGSGGSGGSGGSGGSGGSGG |
| SEQ ID NO: 98 | GGSGGSGGSGGSGGSG |
| SEQ ID NO: 99 | GSGGSGGSGGSGGSGG |

### XI. Formulations

Some embodiments provide pharmaceutical compositions comprising a vaccination regime that can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. The compositions described throughout can be formulated into a pharmaceutical medicament and be used to treat a human or mammal, in need thereof, diagnosed with a disease, e.g., cancer.

For administration, viral vector stock can be combined with an appropriate buffer, physiologically acceptable carrier, excipient or the like. In certain embodiments, an appropriate number of virus particles (VP) are administered in an appropriate buffer, such as, sterile PBS or saline. In certain embodiment, vector compositions disclosed herein are provided in specific formulations for subcutaneously, parenterally, intravenously, intramuscularly, or even intraperitdneally administration. In certain embodiments, formulations in a solution of the active compounds as free base or pharmacologically acceptable salts may be prepared in water suitably mixed with a surfactant, such as hydroxypropylcellulose. Dispersions may also be prepared in glycerol, liquid polyethylene glycols, squalene-based emulsion, Squalene-based oil-in-water emulsions, water-in-oil emulsions, oil-in-water emulsions, nonaqueous emulsions, water-in-paraffin oil emulsion, and mixtures thereof and in oils. In other embodiments, viral vectors may are provided in specific formulations for pill form administration by swallowing or by suppository.

Illustrative pharmaceutical forms suitable for injectable use include sterile aqueous solutions or dispersions and sterile powders for the extemporaneous preparation of sterile injectable solutions or dispersions (see, e.g., U.S. Pat. No. 5,466,468). Fluid forms to the extent that easy syringability exists may be preferred. Forms that are stable under the conditions of manufacture and storage are provided in some embodiments. In various embodiments, forms are preserved against the contaminating action of microorganisms, such as bacteria, molds and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, and thimerosal. It may be suitable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution can be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see, e.g., “Remington's Pharmaceutical Sciences” 15th Edition, pages 1035-1038 and 1570-1580). Some variation in dosage may occur depending on the condition of the subject being treated.

Carriers of formulation can comprise any and all solvents, dispersion media, vehicles, coatings, diluents, antibacterial and antifungal agents, isotonic and absorption delaying agents, buffers, carrier solutions, suspensions, colloids, and the like. Except insofar as any conventional media or agent is incompatible with the active ingredient, its use in the therapeutic compositions is contemplated. Supplementary active ingredients can also be incorporated into the compositions.

In certain embodiments, the viral vectors may be administered in conjunction with one or more immunostimulants, such as an adjuvant. An immunostimulant refers to essentially any substance that enhances or potentiates an immune response (antibody and/or cell-mediated) to an antigen. One type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. In some embodiments, the viral vectors may be administered in conjunction with any of the following commercially available adjuvants: Freund's Incomplete Adjuvant and Complete Adjuvant (Difco Laboratories); Merck Adjuvant 65 (Merck and Company, Inc.) AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. In some embodiments, the viral vectors may be administered in conjunction with cytokines as adjuvants, such as GM-CSF, IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, and/or IL-32, and others, like growth factors.

Within certain embodiments, the adjuvant composition can be one that induces an immune response predominantly of the Th1 type. High levels of Th1-type cytokines (e.g., IFN-γ, TNFα, IL-2 and IL-12) tend to favor the induction of cell-mediated immune responses to an administered antigen. In contrast, high levels of Th2-type cytokines (e.g., IL-4, IL-5, IL-6, and IL-10) tend to favor the induction of humoral immune responses. Following application of a vaccine as provided herein, a subject may support an immune response that includes Th1- and/or Th2-type responses. Within certain embodiments, in which a response is predominantly Th1-type, the level of Th1-type cytokines will increase to a greater extent than the level of Th2-type cytokines. The levels of these cytokines may be readily assessed using standard assays. Thus, various embodiments relate to therapies raising an immune response against a target antigen, for example HPV E6 and/or HPV E7, using cytokines, e.g., IFN-γ, TNFα, IL-2, IL-8, IL-12, IL-18, IL-7, IL-3, IL-4, IL-5, IL-6, IL-9, IL-10, IL-13, and/or IL-15 supplied concurrently with a replication defective viral vector treatment. In some embodiments, a cytokine or a nucleic acid encoding a cytokine, is administered together with a replication defective viral described herein. In some embodiments, cytokine administration is performed prior or subsequent to viral vector administration. In some embodiments, a replication defective viral vector capable of raising an immune response against a target antigen, for example, HPV E6 and/or HPV E7, further comprises a sequence encoding a cytokine.

Certain illustrative adjuvants for eliciting a predominantly Th1-type response include, for example, a combination of monophosphoryl lipid A, such as 3-de-O-acylated monophosphoryl lipid A, together with an aluminum salt. MPL® adjuvants are commercially available (see, e.g., U.S. Pat. Nos. 4,436,727; 4,877,611; 4,866,034 and 4,912,094). CpG-containing oligonucleotides (in which the CpG dinucleotide is unmethylated) also induce a predominantly Th1 response. (see, e.g., WO 96/02555, WO 99/33488 and U.S. Pat. Nos. 6,008,200 and 5,856,462). Immunostimulatory DNA sequences can also be used. Another adjuvant for use comprises a saponin, such as Quil A, or derivatives thereof, including QS21 and QS7 (Aquila Biopharmaceuticals Inc.), Escin; Digitonin; or Gypsophila or Chenopodium quinoa saponins. Other formulations may include more than one saponin in the adjuvant combinations, e.g., combinations of at least two of the following group comprising QS21, QS7, Quil A, β-escin, or digitonin.

In some embodiments, the compositions may be delivered by intranasal sprays, inhalation, and/or other aerosol delivery vehicles. The delivery of drugs using intranasal microparticle resins and lysophosphatidyl-glycerol compounds can be employed (see, e.g., U.S. Pat. No. 5,725,871). Likewise, illustrative transmucosal drug delivery in the form of a polytetrafluoroetheylene support matrix can be employed (see, e.g., U.S. Pat. No. 5,780,045).

Liposomes, nanocapsules, microparticles, lipid particles, vesicles, and the like, can be used for the introduction of the compositions into suitable hot cells/organisms. Compositions as described herein may be formulated for delivery either encapsulated in a lipid particle, a liposome, a vesicle, a nanosphere, or a nanoparticle or the like. Alternatively, compositions as described herein can be bound, either covalently or non-covalently, to the surface of such carrier vehicles. Liposomes can be used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery. In some embodiments, liposomes are formed from phospholipids dispersed in an aqueous medium and spontaneously form multilamellar concentric bilayer vesicles (i.e., multilamellar vesicles (MLVs)).

In some embodiments, pharmaceutically-acceptable nanocapsule formulations of the compositions are provided. Nanocapsules can generally entrap compounds in a stable and reproducible way. To avoid side effects due to intracellular polymeric overloading, such ultrafine particles (sized around 0.1 μm) may be designed using polymers able to be degraded in vivo.

The compositions in some embodiments comprise or are administered with a chemotherapeutic agent (e.g., a chemical compound useful in the treatment of cancer). Chemotherapeutic cancer agents that can be used in combination with the disclosed T cell include, but are not limited to, mitotic inhibitors (vinca alkaloids), such as vincristine, vinblastine, vindesine and Navelbine™ (vinorelbine,5′-noranhydroblastine); topoisomerase I inhibitors, such as camptothecin compounds (e.g., Camptosar™ (irinotecan HCL), Hycamtin™ (topotecan HCL) and other compounds derived from camptothecin and its analogues); podophyllotoxin derivatives, such as etoposide, teniposide and mitopodozide; alkylating agents such as cisplatin or carboplatin, cyclophosphamide, nitrogen mustard, trimethylene thiophosphoramide, carmustine, busulfan, chlorambucil, belustine, uracil mustard, chlomaphazin, and dacarbazine; antimetabolites such as cytosine arabinoside, fluorouracil, methotrexate, mercaptopurine, azathioprime, and procarbazine; antibiotics, such as doxorubicin, bleomycin, dactinomycin, daunorubicin, mithramycin, mitomycin, mytomycin C, and daunomycin; anti-tumor antibodies; dacarbazine; azacytidine; amsacrine; melphalan; ifosfamide; and mitoxantrone.

Compositions disclosed herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cisplatin, carboplatin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In certain aspects, a pharmaceutical composition comprising IL-15 may be administered to an subject in need thereof, in combination with one or more therapy provided herein, particularly one or more adenoviral vectors comprising nucleic acid sequences encoding one or more target antigens such as HPV antigens described herein.

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

IL-15 can enhance the anti-tumor immunity of CD8+ T cells in pre-clinical models. A phase I clinical trial to evaluate the safety, dosing, and anti-tumor efficacy of IL-15 in patients with metastatic melanoma and renal cell carcinoma (kidney cancer) has begun to enroll patients at the National Institutes of Health. IL-15 disclosed herein may also include mutants of IL-15 that are modified to maintain the function of its native form.

IL-15 is 14-15 kDa glycoprotein encoded by the 34 kb region 4q31 of chromosome 4, and by the central region of chromosome 8 in mice. The human IL-15 gene comprises nine exons (1-8 and 4A) and eight introns, four of which (exons 5 through 8) code for the mature protein. Two alternatively spliced transcript variants of this gene encoding the same protein have been reported. The originally identified isoform, with long signal peptide of 48 amino acids (IL-15 LSP) consisted of a 316 bp 5′-untranslated region (UTR), 486 bp coding sequence and the C-terminus 400 bp 3′-UTR region. The other isoform (IL-15 SSP) has a short signal peptide of 21 amino acids encoded by exons 4A and 5. Both isoforms shared 11 amino acids between signal sequences of the N-terminus. Although both isoforms produce the same mature protein, they differ in their cellular trafficking. IL-15 LSP isoform was identified in Golgi apparatus [GC], early endosomes and in the endoplasmic reticulum (ER). It exists in two forms, secreted and membrane-bound particularly on dendritic cells. On the other hand, IL-15 SSP isoform is not secreted and it appears to be restricted to the cytoplasm and nucleus where it plays an important role in the regulation of cell cycle.

It has been demonstrated that two isoforms of IL-15 mRNA are generated by alternatively splicing in mice. The isoform which had an alternative exon 5 containing another 3′ splicing site, exhibited a high translational efficiency, and the product lack hydrophobic domains in the signal sequence of the N-terminus. This suggests that the protein derived from this isoform is located intracellularly. The other isoform with normal exon 5, which is generated by integral splicing of the alternative exon 5, may be released extracellularly.

Although IL-15 mRNA can be found in many cells and tissues including mast cells, cancer cells or fibroblasts, this cytokine is produce as a mature protein mainly by dendritic cells, monocytes and macrophages. This discrepancy between the wide appearance of IL-15 mRNA and limited production of protein might be explained by the presence of the twelve in humans and five in mice upstream initiating codons, which can repress translation of IL-15 mRNA. Translational inactive mRNA is stored within the cell and can be induced upon specific signal. Expression of IL-15 can be stimulated by cytokine such as GM-CSF, double-strand mRNA, unmethylated CpG oligonucleotides, lipopolysaccharide (LPS) through Toll-like receptors (TLR), interferon gamma (IFN-γ) or after infection of monocytes herpes virus, Mycobacterium tuberculosis, and Candida albicans.

### XII. Methods of Preparation

In some embodiments, compositions and methods make use of human cytolytic T-cells (CTLs), such as those that recognize an HPV E6 and/or HPV E7 epitope which bind to selected MHC molecules, e.g., HLA-A2, HLA-A3, and HLA-A24. Subjects expressing MHC molecules of certain serotypes, e.g., HLA-A2, HLA-A3, and HLA-A24 may be selected for therapy using the methods and compositions as described herein. For example, subjects expressing MHC molecules of certain serotypes, e.g., HLA-A2, HLA-A3, and HLA-A24, may be selected for a therapy including raising an immune response against HPV E6 and/or HPV E7, using the methods and compositions described herein.

In various embodiments, these T-cells can be generated by in vitro cultures using antigen-presenting cells pulsed with the epitope of interest to stimulate peripheral blood mononuclear cells. In addition, T-cell lines can also be generated after stimulation with HPV E6 and/or HPV E7 latex beads, HPV E6 and/or HPV E7 protein-pulsed plastic adherent peripheral blood mononuclear cells, or DCs sensitized with HPV E6 and/or HPV E7 RNA. T-cells can also be generated from subjects immunized with a vaccine vector encoding HPV E6 and/or HPV E7 immunogen.

Some embodiments relate to an HLA-A2 restricted epitope of HPV E6 and/or HPV E7, with ability to stimulate CTLs from cancer patients immunized with vaccine HPV E6 and/or HPV E7. The sequences include a heteroclitic (nonanchor position) mutation, resulting in an amino acid change that enhances recognition by the T-cell receptor. Some embodiments incorporate amino acid changes at one or more positions (e.g., 26, 98, 106) of HPV E6, (e.g., 86) of HPV E7, or combinations thereof. Compared to the non-mutated antigen, incorporation of agonist epitopes can enhance the sensitization of CTLs by 100 to 1,000 times. Thus, HPV E6 and HPV E7 nucleic acid sequences encoding such variant antigens are provided in some embodiments.

### XIII. Methods of Treating HPV-Associated Diseases

In certain embodiments, there is provided a method of enhancing an immune response in an subject in need thereof, the method comprising: administering to the subject a pharmaceutical composition comprising a replication-defective adenovirus vector comprising a nucleic acid sequence encoding an HPV antigen; and administering to the subject an immune checkpoint inhibitor. In certain embodiments, the method may be further defined as treating an HPV infection or an HPV-associated disease, such as an HPV-associated cancer, including, but not limited to, head and neck squamous cell carcinoma (HNSCC), oropharyngeal and tonsillar cancer, cervical cancer, penis cancer, vulva cancer, or anal cancer.

A. Human Papilloma Virus (HPV)-Associated HNSCC

Evidence has demonstrated that infection with high-risk HPV16 is associated with the development and progression of HPV-associated HNSCC and, more specifically, the HPV early 6 (E6) and early 7 (E7) genes contribute to cancer development. The prevalence of head and neck cancers in the United States is estimated to be about 370,000 and from 25% to 38% of these are HPV-associated HNSCC. Thus, the prevalence of HPV-associated HNSCC is estimated to range from 92,750 to 140,000 cases. A recent study on HPV-associated HNSCC estimated an incidence of about 35,000 new cases in the United States, with an expected 7,600 cancer related deaths annually despite current therapy. Thus, there remains an unmet medical need to investigate new treatment methods for this patient population. Based upon the estimated prevalence of HPV-associated HNSCC, this population qualifies for orphan product drug development by the FDA and Etubics has received orphan product designation for the development of a new immunotherapeutic vaccine (Ad5 [E1-, E2b-]-E6/E7) to treat HPV-associated HNSCC.

B. HIV and IIPV-Associated Oropharyngeal and Tonsillar Cancer

Human papilloma virus (HPV) is responsible for as many as 100,000 cases of head and neck squamous cell carcinoma (HNSSC) worldwide per year. The majority of these are oropharyngeal and tonsillar cancers. In the United States, prevalence estimates of oropharynx HPV infection range from 9.2 to 18.6 percent. HPV type16 (HPV16) is the most prevalent HPV found in oral carcinomas and is involved in the etiology of these cancers. The incidence of tonsillar cancer in the United States has increased by 2-3% per year from 1973 to 1995. HIV-infected subjects have a 2 to 6-fold increase in risk of developing oropharyngeal and tonsillar cancers. Despite significant advances in the therapy of AIDS, this pandemic continues to be responsible for devastating morbidity and mortality throughout the world, especially in regions with limited access to antiretroviral medications. HPV infection and disease has not dramatically declined since the introduction of potent combination therapy to control HIV and highly active antiretroviral therapy appears to have little beneficial effect on HPV-associated oral disease. Thus, it remains imperative to investigate new vaccines that can be applied to HIV and HIV-associated malignancies.

Certain aspects provide a therapeutic strategy for HIV-associated malignancy based on the pathogenic role of HPV. The vaccine to be used is based upon a new recombinant adenovirus serotype 5 (Ad5) vector platform (Ad5 [E1-, E2b-]) described herein. This recombinant vector allows for the insertion of specific disease associated antigen genes that will be expressed after direct transfection of antigen presenting cells. Importantly, this new vaccine can be utilized in multiple homologous immunization regimens designed to stimulate potent cell-mediated immune (CMI) responses against specific target antigens and has the potential to become an important immunotherapeutic agent in the battle against HIV/HPV-associated oropharyngeal and tonsillar malignancies.

Patients with HPV-associated HNSCC are administered a multi-facetted treatment, and immunotherapy with the Ad5 [E1-, E2b-]-E6 vaccine, Ad5 [E1-, E2b-]-E7 vaccine, Ad5 [E1-, E2b-]-E6/E7 vaccine can play an important role in the armamentarium of treatments against this disease.

C. HPV-Associated Cervical Cancer

Cervical cancer is the second leading cause of cancer-related death in women. It is known that oncogenic human papillomavirus (HPV) plays a critical etiological role in anogenital cancers and at least 70% of cervical cancers are associated with type 16 (HPV-16) or 18 (HPV-18). HPV-16 and 18 are also the virus types with which the majority of vulval and vaginal pre-cancer are associated. Vulvar intraepithelial neoplasia is a chronic premalignant disorder of the vulvar skin that is caused by high-risk types of human papillomavirus (HPV); HPV-16 is involved in more than 75% of cases. The lifetime risk of a woman acquiring any HPV infection is more than 80%. Half of women acquire cervical infection within 3 years of initiating sexual activity. About 90% of HPV infections are cleared by the immune system within 6-24 months. The prevalence of HPV infection in sexually active women is 10-20% and even higher in young women. HPV-16/18 bivalent (Cervarix) and HPV-6/11/16/18 quadrivalent (Gardasil) vaccines are highly effective in preventing vaccine-type HPV-related genital pre-cancer in women who are HPV-negative at the time of vaccination. Although these vaccines are highly effective at preventing HPV infection, there is still a population of women who are not vaccinated and become HPV infected and thus are at high risk of developing neoplasia. In a recent meta-analysis study, there was no indication that the above HPV vaccines given to women with evidence of prior vaccine-type HPV exposure can prevent premalignant lesions related to these HPV types over a 3 to 4-year time frame. It is this population of women that are believed to benefit from vaccination with this new adenoviral vaccine (Ad5 [E1-, E2b-]-E6/E7 vaccine; Ad5 [E1-, E2b-]-E6 vaccine; Ad5 [E1-, E2b-]-E7 vaccine) designed to prevent development of HPV-associated cancer.

### XIV. Methods of Reducing HPV-Positive Cells in HPV-Positive Subjects

In certain embodiments, the present disclosure provides a method of reducing HPV infection or preventing the development of HPV-induced cancer in subjects who are HPV-positive or are at risk for developing HPV-induced cancer at the time of prophylaxis or prior to administering an Ad5 [E1-, E2b-]-E6/E7 vaccine, Ad5 [E1-, E2b-]-E6 vaccine, and/or Ad5 [E1-, E2b-]-E7 vaccine. In some embodiments, administration of a HPV-E6/E7 vaccine, HPV-E6 vaccine, and/or HPV E7 vaccine as described herein can destroy HPV-infected cells and thereby prevent the development of HPV induced cancer. In certain embodiments, the subjects do not have HPV-induced or HPV-associated cancer or are determined to not to have a HPV-induced or HPV-associated cancer prior to the administering the Ad5 [E1-, E2b-]-E6/E7 vaccine, Ad5 [E1-, E2b-]-E6 vaccine, and/or Ad5 [E1-, E2b-]-E7 vaccine.

Among sexually transmitted infections (STIs), HPV is the most frequently spread virus. Symptoms of HPV infection can go unnoticed, leading to transmission without knowledge of disease status. HPV infection can result in chronic diseases such as genital warts and cancer. Reducing the rates of HPV infection can be achieved through preventative vaccination. However, in some cases, before vaccination with existing vaccine, an HPV infection can occur and result in expression and propagation of HPV oncogenes that may lead to the development of cancer. For example, an HPV infection can be HPV type 16 or HPV type 18, or a combination thereof, which result in infection and expression of the early 6 (E6) and/or early 7 (E7) oncogenes. Vaccination against HPV can be used in preventing the propagation of HPV oncogenes, including E6 and E7. In certain embodiments, the Ad5 [E1-, E2b-]-E6/E7 immunotherapy, Ad5 [E1-, E2b-]-E6 immunotherapy, and/or Ad5 [E1-, E2b-]-E7 immunotherapy of the present disclosure can be administered prophylactically to vaccinate HPV positive subjects and reduce or eliminate HPV infection that may cause the development of HPV-induced or HPV-associated cancers. In certain aspects, the reduction in HPV-positive cells can be determined by any methods available in the art for protein or nucleic acid detection, such as PCR.

### XV. Dosages and Administration

Compositions and methods as described herein contemplate various dosage and administration regimens during vaccination for reduction of HPV infection by reducing, destroying, or eliminating HPV E6/E7-expressing cells to prevent HPV-associated cancers or treatment of HPV-associated cancers or diseases. Subjects can receive one or more replication defective adenovirus or adenovirus vector, for example Ad5 [E1-, E2B-]-HPV E6, Ad5 [E1-, E2b-]-HPV E7, and/or Ad5 [E1-, E2b-]-HPV E6/E7, that is capable of raising an immune response in an subject against a target antigen described herein. In various embodiments, the replication defective adenovirus is administered at a dose that suitable for effecting such immune response. In some embodiments, the replication defective adenovirus is administered at a dose from about 1×10⁸ virus particles to about 5×10¹³ virus particles per immunization. In some embodiments, the replication defective adenovirus is administered at a dose from about 1×10⁹ to about 5×10¹² virus particles per immunization. In some embodiments, the replication defective adenovirus is administered at a dose from about 1×10⁸ virus particles to about 5×10⁸ virus particles per immunization. In some embodiments, the replication defective adenovirus is administered at a dose from about 5×10⁸ virus particles to about 1×10⁹ virus particles per immunization. In some embodiments, the replication defective adenovirus is administered at a dose from about 1×10⁹ virus particles to about 5×10⁹ virus particles per immunization. In some embodiments, the replication defective adenovirus is administered at a dose from about 5×10⁹ virus particles to about 1×10¹⁰ virus particles per immunization. In some embodiments, the replication defective adenovirus is administered at a dose from about 1×10¹⁰ virus particles to about 5×10¹⁰ virus particles per immunization. In some embodiments, the replication defective adenovirus is administered at a dose from about 5×10¹⁰ virus particles to about 1×10¹¹ virus particles per immunization. In some embodiments, the replication defective adenovirus is administered at a dose from about 1×10¹¹ virus particles to about 5×10¹¹ virus particles per immunization. In some embodiments, the replication defective adenovirus is administered at a dose from about 5×10¹¹ virus particles to about 1×10¹² virus particles per immunization. In come embodiments, the replication defective adenovirus is administered at a dose from about 1×10¹² virus particles to about 5×10¹² virus particles per immunization. In some embodiments, the replication defective adenovirus is administered at a dose from about 5×10¹² virus particles to about 1×10¹³ virus particles per immunization. In some embodiments, the replication defective adenovirus is administered at a dose from about 1×10¹³ virus particles to about 5×10¹³ virus particles per immunization. In some embodiments, the replication defective adenovirus is administered at a dose from about 1×10⁸ virus particles to about 5×10¹⁰ virus particles per immunization. In some embodiments, the replication defective adenovirus is administered at a dose from about 1×10¹⁰ virus particles to about 5×10¹² virus particles per immunization. In some embodiments, the replication defective adenovirus is administered at a dose from about 1×10¹¹ virus particles to about 5×10¹³ virus particles per immunization. In some embodiments, the replication defective adenovirus is administered at a dose from about 1×10⁸ virus particles to about 1×10¹⁰ virus particles per immunization. In some embodiments, the replication defective adenovirus is administered at a dose from about 1×10¹⁰ virus particles to about 1×10¹² virus particles per immunization. In some embodiments, the replication defective adenovirus is administered at a dose from about 1×10¹¹ virus particles to about 5×10¹³ virus particles per immunization. In some cases, the replication defective adenovirus is administered at a dose that is greater than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×1010¹¹, 1×10¹², 1.5×10¹², 2×10¹², 3×10¹², or more virus particles (VP) per immunization. In some cases, the replication defective adenovirus is administered at a dose that is less than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹¹, 1×10¹², 1.5×10¹², 2×10¹², 3×10¹², or more virus particles per immunization. In some embodiments, the replication defective adenovirus can be formulated or administered at any of the doses described above in a single dose. In some embodiments, the replication defective adenovirus can be formulated and administered at a concentration of 1×10⁹-3×10¹², 1×10⁹-1×10¹¹, or 5×10⁹-5×10¹¹ virus particles (VPs) per single dose for immunization. In some cases, the replication defective adenovirus is administered at a dose of 10 μg, 20 μg, 30 μg, 40 μg, 50 μg, 60 μg, 70 μg, 80 μg, 90 μg, 100 μg, or more of virus particles per immunization. In various embodiments, a desired dose described herein is administered in a suitable volume of formulation buffer, for example a volume of about 0.1-10 mL, 0.2-8 mL, 0.3-7 mL, 0.4-6 mL, 0.5-5 mL, 0.6-4 mL, 0.7-3 mL, 0.8-2 mL, 0.9-1.5 mL, 0.95-1.2 mL, or 1.0-1.1 mL. Those of skill in the art appreciate that the volume may fall within any range bounded by any of these values (e.g., about 0.5 mL to about 1.1 mL). Administration of virus particles can be through a variety of suitable paths for delivery, for example it can be by injection (e.g., intradermally, intracutaneously, intramuscularly, intravenously or subcutaneously), intranasally (e.g., by aspiration), in pill form (e.g., swallowing, suppository for vaginal or rectal delivery. In some embodiments, a subcutaneous delivery may be preferred and can offer greater access to dendritic cells.

Administration of virus particles to a subject may be repeated. Repeated deliveries of virus particles may follow a schedule or alternatively, may be performed on an as needed basis. For example, an subject's immunity against a target antigen, for example HPV E6 and/or HPV E7 may be tested and replenished as necessary with additional deliveries. In some embodiments, schedules for delivery include administrations of virus particles at regular intervals. Joint delivery regimens may be designed comprising one or more of a period with a schedule and/or a period of need based administration assessed prior to administration. For example, a therapy regimen may include an administration, such as subcutaneous administration once every three, every four, every five, every six, every seven, every eight, every nine, every ten, every eleven, every twelve, every thirteen, every fourteen, every fifteen, every sixteen, every seventeen, every eighteen, every nineteen, or every twenty weeks then another immunotherapy treatment every three months until removed from therapy for any reason including death. Another example regimen comprises three administrations every three, every four, every five, every six, every seven, every eight, every nine, every ten, every eleven, every twelve, every thirteen, every fourteen, every fifteen, every sixteen, every seventeen, every eighteen, every nineteen, or every twenty weeks then another set of three immunotherapy treatments every three months. Another example regimen comprises a first period with a first number of administrations at a first frequency, a second period with a second number of administrations at a second frequency, a third period with a third number of administrations at a third frequency, etc., and optionally one or more periods with undetermined number of administrations on an as needed basis. The number of administrations in each period can be independently selected and can for example be 1, 2, 3, 4, 5, 6, 7, 8, 9, 10, 11, 12, 13, 14, 15, 16, 17, 18, 19, 20, or more. The frequency of the administration in each period can also be independently selected, can for example be about every day, every other day, every third day, twice a week, once a week, once every other week, every three weeks, every month, every six weeks, every other month, every third month, every fourth month, every fifth month, every sixth month, once a year etc. The immunization regimen can take a total period of up to 1, 2, 3, 4, 5, 6, 7, 8, 9, 10, 11, 12, 13, 14, 15, 16, 17, 18, 19, 20, 21, 22, 23, 24, 30, 36 months, or more. The scheduled interval between immunizations may be modified so that the interval between immunizations is revised by up to a fifth, a fourth, a third, or half of the interval. For example, for a 3-week interval schedule, an immunization may be repeated between 20 and 28 days (3 weeks−1 day to 3 weeks+7 days). For the first 3 immunizations, if the second and/or third immunization is delayed, the subsequent immunizations may be shifted allowing a minimum amount of buffer between immunizations. For example, for a three week interval schedule, if an immunization is delayed, the subsequent immunization may be scheduled to occur no earlier than 17, 18, 19, or 20 days after the previous immunization. In some embodiments, a booster immunization can be administered after any of the above described primary vaccine immunizations. In some embodiments, the administering the therapeutically effective amount is followed by one or more booster immunizations comprising the same composition or pharmaceutical composition as the primary immunization. In some aspects, the booster immunization is administered every one, two, three, four, five, six, seven, eight, nine, ten, eleven, or twelve months or more. In some aspects, the booster immunization is repeated three four, five, six, seven, eight, nine, ten, eleven, or twelve or more times. In some aspects, the administering the therapeutically effective amount is a primary immunization repeated every one, two, or three weeks for three four, five, six, seven, eight, nine, ten, eleven, or twelve or more times followed by a booster immunization repeated every one, two, three, four, five, six, seven, eight, nine, ten, eleven, or twelve or more months for three or more times.

Compositions, such as Ad5 [E1-, E2B-]-HPV E6, Ad5 [E1-, E2b-]-HPV E7, and Ad5 [E1-, E2b-]-HPV E6/E7 virus particles, can be provided in various states, for example, at room temperature, on ice, or frozen. Compositions may be provided in a container of a suitable size, for example a vial of 2 mL vial. In one embodiment, a 2-ml vial with 1.0 mL of extractable vaccine contains 5×10¹¹ total virus particles/mL. Storage conditions including temperature and humidity may vary. For example, compositions for use in therapy may be stored at room temperature, 4° C., −20° C., or lower.

In one aspect, a method of selecting a human for administration of the compositions is provided comprising: determining a HLA subtype of the human; and administering the composition to the human, if the HLA subtype is determined to be one of a preselected subgroup of HLA subtypes. In some embodiments, the preselected subgroup of HLA subtypes comprises one or more of HLA-A2, HLA-A3, and HLA-A24.

In one aspect, a method of treating a human for cancer or an infectious disease is provided comprising administering the recombinant viral vector to the human.

In one aspect, a method of generating an immune response in a human to HPV E6, HPV E7, or a combination thereof, is provided comprising administering to the human the composition. In some embodiments, the administering step is repeated at least once. In some embodiments, the administering step is repeated after about 2, 3, 4, 5, 6, 7, 8, 9, 10, 11, 12, 13, 14, 15, 16, 17, 18, 19, or 20 weeks following a previous administering step. In some embodiments, the administering step is repeated after about 2, 3, 4, 5, 6, 7, 8, 9, 10, 11, 12, 13, 14, 15, 16, 17, 18, 19, 20, 21, 22, 23, or 24 months following a previous administering step. In some embodiments, the administering step is repeated twice.

In various embodiments, general evaluations are performed on the subjects receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

General evaluations may include one or more of medical history, ECOG Performance Score, Karnofsky performance status, and complete physical examination with weight by the attending physician. Any other treatments, medications, biologics, or blood products that the subject is receiving or has received since the last visit may be recorded. Subjects may be followed at the clinic for a suitable period, for example approximately 30 minutes, following receipt of vaccine to monitor for any adverse reactions. Local and systemic reactogenicity after each dose of vaccine is assessed daily for a selected time, for example for 3 days (on the day of immunization and 2 days thereafter). Diary cards may be used to report symptoms and a ruler may be used to measure local reactogenicity. Immunization injection sites may be assessed. CT scans or MRI of the chest, abdomen, and pelvis may be performed.

In various embodiments, hematological and biochemical evaluations are performed on the subjects receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization. Hematological and biochemical evaluations may include one or more of blood test for chemistry and hematology, CBC with differential, Na, K, Cl, CO₂, BUN, creatinine, Ca, total protein, albumin, total bilirubin, alkaline phosphatase, AST, ALT, glucose, and ANA.

In various embodiments, biological markers are evaluated on subjects receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Biological marker evaluations may include one or more of measuring antibodies to HPV E6 and/or HPV E7, or the Ad5 vector, from a serum sample of adequate volume, for example about 5 ml Biomarkers (e.g., CEA or CA15-3) may be reviewed if determined and available.

In various embodiments, an immunological assessment is performed on subjects receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Peripheral blood, for example about 90 mL may be drawn prior to each immunization and at a time after at least some of the immunizations, to determine whether there is an effect on the immune response at specific time points during the study and/or after a specific number of immunizations. Immunological assessment may include one or more of assaying peripheral blood mononuclear cells (PBMC) for T-cell responses to HPV E6 and/or HPV E7 using ELISpot, proliferation assays, multi-parameter flow cytometric analysis, and cytoxicity assays. Serum from each blood draw may be archived and sent and determined.

In various embodiments, in the case of therapeutic treatment of an HPV-associated disease, a tumor assessment is performed on subjects receiving treatment according to the methods and compositions as described herein. One or more of any tests may be performed as needed or in a scheduled basis, such as prior to treatment, on weeks 0, 3, 6, etc. A different set of tests may be performed concurrent with immunization vs. at time points without immunization. Tumor assessment may include one or more of CT or MRI scans of chest, abdomen, or pelvis performed prior to treatment, at a time after at least some of the immunizations and at approximately every three months following the completion of a selected number, for example 2, 3, or 4, of first treatments and for example until removal from treatment.

Immune responses against a target antigen described herein, such as an HPV antigen, may be evaluated from a sample, such as a peripheral blood sample of a subject using one or more suitable tests for immune response, such as ELISpot, cytokine flow cytometry, or antibody response. A positive immune response can be determined by measuring a T-cell response. A T-cell response can be considered positive if the mean number of spots adjusted for background in six wells with antigen exceeds the number of spots in six control wells by 10 and the difference between single values of the six wells containing antigen and the six control wells is statistically significant at a level of p≤0.05 using the Student's t-test. Immunogenicity assays may occur prior to each immunization and at scheduled time points during the period of the treatment. For example, a time point for an immunogenicity assay at around week 1, 2, 3, 4, 5, 6, 7, 8, 9, 10, 11, 12, 13, 14, 15, 18, 20, 24, 30, 36, or 48 of a treatment may be scheduled even without a scheduled immunization at this time. In some cases, a subject may be considered evaluable for immune response if they receive at least a minimum number of immunizations, for example 1, 2, 3, 4, 5, 6, 7, 8, 9, or more immunizations.

In some embodiments, the immune response comprises generation of an antibody to the antigen. In some embodiments, the immune response comprises cell-mediated immunity (CMI). In some embodiments, the sequence encoding the HPV E6 antigen has at least 80% sequence identity to SEQ ID NO: 8, SEQ ID NO: 9, or SEQ ID NO: 10. In some embodiments, the sequence encoding the HPV E7 antigen has at least 80% sequence identity to SEQ ID NO: 12. In some embodiments, the antigen comprises a modification of 25, 15, 10, 5, or less amino acids. In some embodiments, the recombinant viral vector comprises a replication defective adenovirus vector. In some embodiments, the recombinant viral vector comprises a replication defective adenovirus 5 vector. In some embodiments, the replication defective adenovirus vector comprises a deletion in an E2b gene region. In some embodiments, the replication defective adenovirus vector comprises a deletion in an E1 gene region. In some embodiments, the replication defective adenovirus vector comprises a deletion in an E3 gene region. In some embodiments, the replication defective adenovirus vector comprises a deletion in an E4 gene region. In some embodiments, the recombinant viral vector effects overexpression of the antigen in transfected cells. In some embodiments, the recombinant viral induces a specific immune response against cells expressing the antigen in a human that is at least 2, 3, 4, 5, 6, 7, 8, 9, 10, 15, 20, or 25 fold over basal. In some embodiments, the human has an inverse Ad5 neutralizing antibody titer of greater than 50, 75, 100, 125, 150, 160, 175, or 200. In some embodiments, the human has an inverse Ad5 neutralizing antibody titer of greater than 250, 500, 750, 1000, 1500, 2000, 2500, 3000, 3500, 4000, 4500, or 4767. In some embodiments, the immune response is measured as antigen-specific antibody response.

In some embodiments, the immune response is measured as antigen-specific cell-mediated immunity (CMI). In some embodiments, the immune response is measured as antigen-specific IFN-γ secretion. In some embodiments, the immune response is measured as antigen-specific IL-2 secretion. In some embodiments, the immune response against the antigen is measured by ELISpot assay. In some embodiments, the antigen-specific CMI is greater than 25, 50, 75, 100, 150, 200, 250, or 300 IFN-γ spot forming cells (SFC) per 10⁶ peripheral blood mononuclear cells (PBMC). In some embodiments, the immune response is measured by T-cell lysis of HPV E6 and/or HPV E7 antigen pulsed antigen-presenting cells, allogeneic antigen expressing cells from a tumor cell line or from an autologous tumor.

In some embodiments, in the case of therapeutic treatment of an HPV-associated disease, disease progression or clinical response determination is made according to the RECIST 1.1 criteria among subjects with measurable/evaluable disease. In some embodiments, therapies using the methods and compositions as described herein affect a Complete Response (CR; disappearance of all target lesions for target lesions or disappearance of all non-target lesions and normalization of tumor marker level for non-target lesions) in a subject receiving the therapy. In some embodiments, therapies using the methods and compositions affect a Partial Response (PR; at least a 30% decrease in the sum of the LD of target lesions, taking as reference the baseline sum LD for target lesions) in a subject receiving the therapy.

In some embodiments, therapies using the methods and compositions affect a Stable Disease (SD; neither sufficient shrinkage to qualify for PR nor sufficient increase to qualify for PD, taking as reference the smallest sum LD since the treatment started for target lesions) in a subject receiving the therapy. In some embodiments, therapies using the methods and compositions as described herein affect an Incomplete Response/Stable Disease (SD; persistence of one or more non-target lesion(s) or/and maintenance of tumor marker level above the normal limits for non-target lesions) in a subject receiving the therapy. In some embodiments, therapies using the methods and compositions as described herein affect a Progressive Disease (PD; at least a 20% increase in the sum of the LD of target lesions, taking as reference the smallest sum LD recorded since the treatment started or the appearance of one or more new lesions for target lesions or persistence of one or more non-target lesion(s) or/and maintenance of tumor marker level above the normal limits for non-target lesions) in an subject receiving the therapy.

### XVI. Kits

Certain embodiments provide compositions, methods and kits for generating an immune response in a subject to fight HPV infection and HPV-associated or HPV-induced cancer. Certain embodiments provide compositions, methods and kits for generating an immune response against a target antigen or cells expressing or presenting a target antigen or a target antigen signature comprising at least one target antigen. The compositions, immunotherapy, or vaccines may be supplied in the form of a kit. The kits may further comprise instructions regarding the dosage and or administration including treatment regimen information.

In some embodiments, kits comprise the compositions and methods for providing combination multi-targeted cancer immunotherapy. In some embodiments, kits comprise the compositions and methods for the combination multi-targeted treatment of an infectious disease. In some embodiment's kits may further comprise components useful in administering the kit components and instructions on how to prepare the components. In some embodiments, the kit can further comprise software for monitoring a subject before and after treatment with appropriate laboratory tests, or communicating results and subject data with medical staff.

The components comprising the kit may be in dry or liquid form. If they are in dry form, the kit may include a solution to solubilize the dried material. The kit may also include transfer factor in liquid or dry form. If the transfer factor is in dry form, the kit will include a solution to solubilize the transfer factor. The kit may also include containers for mixing and preparing the components. The kit may also include instrument for assisting with the administration such for example needles, tubing, applicator, inhalant, syringe, pipette, forceps, measured spoon, eye dropper or any such medically approved delivery vehicle. In some embodiments, the kits or drug delivery systems as described herein also include a means for containing compositions disclosed herein in close confinement for commercial sale and distribution.

In one aspect a kit for inducing an immune response in a human is provided comprising: a composition comprising a therapeutic solution of a volume in the range of 0.8-1.2 mL, the therapeutic solution comprising at least 1.0×10¹¹ virus particles; wherein the virus particles comprise a recombinant replication defective adenovirus vector; a composition comprising of a therapeutic solution of a molecular composition comprising an immune pathway checkpoint modulator and; instructions.

In some embodiments, the therapeutic solution comprises 1.0-5.5×10¹¹ virus particles. In some embodiments, adenovirus vector is capable of effecting overexpression of the modified HPV E6 and/or HPV E7 in transfected cells. In some embodiments, the adenovirus vector comprises a nucleic acid sequence encoding an antigen that induces a specific immune response against HPV E6 and/or HPV E7 expressing cells in a human. In some embodiments, the immune pathway checkpoint modulator targets an endogenous immune pathway checkpoint protein or fragment thereof selected from the group consisting of: PD-1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA, and CD244. In some embodiments, the molecular composition comprises siRNAs, antisense, small molecules, mimic, a recombinant form of a ligand, a recombinant form of a receptor, antibodies, or a combination thereof.

In some embodiments, the instructions are for the treatment of a proliferative disease or cancer. In some embodiments, the adenovirus vector comprises a replication defective adenovirus 5 vector. In some embodiments, the therapeutic solution comprises at least 1.0×10¹¹, 2.0×10¹¹, 3.0×10¹¹, 3.5×10¹¹, 4.0×10¹¹, 4.5×10¹¹, 4.8×10¹¹, 4.9×10¹¹, 4.95×10¹¹, or 4.99×10¹¹ virus particles comprising the recombinant nucleic acid vector. In some embodiments, the therapeutic solution comprises at most 7.0×10¹¹, 6.5×10¹¹, 6.0×10¹¹, 5.5×10¹¹, 5.2×10¹¹, 5.1×10¹¹, 5.05×10¹¹, or 5.01×10¹¹ virus particles. In some embodiments, the therapeutic solution comprises 1.0-7.0×10¹¹ or 1.0-5.5×10¹¹ virus particles. In some embodiments, the therapeutic solution comprises 4.5-5.5×10¹¹ virus particles. In some embodiments, the therapeutic solution comprises 4.8-5.2×10¹¹ virus particles. In some embodiments, the therapeutic solution comprises 4.9-5.1×10¹¹ virus particles. In some embodiments, the therapeutic solution comprises 4.95-5.05×10¹¹ virus particles. In some embodiments, the therapeutic solution comprises 4.99-5.01×10¹¹ virus particles In some embodiments, the kit further comprises an immunogenic component. In some embodiments, the immunogenic component comprises a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, and IL-32. In some embodiments, the immunogenic component is selected from the group consisting of IL-7, a nucleic acid encoding IL-7, a protein with substantial identity to IL-7, and a nucleic acid encoding a protein with substantial identity to IL-7.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Production and Evaluation of Ad5 Vectors Containing HPV E6 and/or HPV E7

This example describes production and evaluation of Ad5 [E1-, E2b-]-HPV E6/E7 vector. HPV E6 and HPV E7 are non-oncogenic variants of native E6 and E7 proteins.

### Viral Construction

Ad5 [E1-, E2b-]-E6/E7 was constructed and produced. Briefly, the transgenes were sub-cloned into the Ad5 [E1-, E2b-] vector using a homologous recombination-based approach and the replication deficient virus was propagated in the E.C7 packaging cell line, CsCl₂ purified, and infectious titer was determined as plaque forming units (PFU) on an E.C7 cell monolayer. The virus particle (VP) concentration was determined by sodium dodecyl sulfate (SDS) disruption and spectrophotometry at 260 nm and 280 nm. As a vector control, Ad5 [E1-, E2b-]-null was employed, which is the Ad5 platform backbone with no transgene insert.

### Immunization and Splenocyte Preparation

Female C57BL/6 mice (n=5/group) were injected subcutaneously (SQ) with varying doses of Ad5 [E1-, E2b-]-E6/E7 or Ad5 [E1-, E2b-]-null. Doses were administered in 25 μL injection buffer (20 mM HEPES with 3% sucrose) and mice were immunized three times at 14-day intervals. Fourteen days after the final injection, spleens and sera were collected. Serum from mice was frozen at −20° C. until evaluation. Suspensions of splenocytes were generated by disrupting the spleen capsule and gently pressing the contents through a 70 Rpm nylon cell strainer. Red blood cells were lysed by the addition of red cell lysis buffer and after lysis, the splenocytes were washed twice in R10 (RPMI 1640 supplemented with L-glutamine (2 mM), HEPES (20 mM) (Corning, Corning, N.Y.), penicillin (10 U U/ml) and streptomycin (100 μg/mL), and 10% fetal bovine serum. Splenocytes were assayed for cytokine production by ELISpot and flow cytometry.

### Enzyme-Linked Immunosorbent Spot (ELISpot) Assay

HPV E6 and HPV E7 specific interferon-γ (IFN-γ) secreting T cells were determined by ELISpot assays using freshly isolated mouse splenocytes prepared as described above. The ELISpot assay was performed. Pools of overlapping peptides spanning the entire coding sequences of HPV E6 and HPV E7 were synthesized as 15-mers with 11-amino acid overlaps (and lyophilized peptide pools were dissolved in DMSO). Splenocytes (2×10⁵ cells) were stimulated with 2 μg/mL/peptide of overlapping 15-mer peptides in pools derived from E6 or E7. Cells were stimulated with Concanavalin A (Con A) at a concentration of 0.06 μg/per well as a positive control. Overlapping 15-mer complete peptide pools derived from SIV-Nef (AIDS Research and Reference Reagent Program, Division of AIDS, NIAID, NIH) were used as irrelevant peptide controls. The numbers of Spot Forming Cells (SFC) were determined using an Immunospot ELISpot plate reader (and results reported as the number of SFC per 10⁶ splenocytes).

### Intracellular Cytokine Stimulation

Splenocytes were prepared as described for the ELISpot assay above. Stimulation assays were performed using 10⁶ live splenocytes per well in 96-well U-bottom plates. Splenocytes in R10 media were stimulated by the addition of HPV E6, HPV E7, or SIV-Nef peptide pools at 21 μg/mL/peptide for 6 h at 37° C. in 5% CO₂, with protein transport inhibitor (GolgiStop, BD) added two hours after initiation of incubation. Stimulated splenocytes were then stained for lymphocyte surface markers CD8a and CD4, fixed with paraformaldehyde, permeabilized, and stained for intracellular accumulation of IFN-γ and TNF-α. Fluorescent-conjugated antibodies against mouse CD8a (clone 53-6.7), CD4 (clone RM4-5), IFN-γ (clone XMG1.2), and TNF-α (clone MP6-XT22) were purchased from BD and staining was performed in the presence of anti-CD16/CD32 (clone 2.4G2). Flow cytometry was performed using an Accuri C6 Flow Cytometer (BD) and analyzed using BD Accuri C6 Software.

### Tumor Immunotherapy

For in vivo tumor immunotherapy studies, female C57BL/6 mice, 8-10 weeks old, were implanted with 2×10⁵ TC-1 HPV E6/E7-Expressing tumor cells SQ in the left flank. Mice were treated three times at 7-day intervals with SQ injections of 10¹⁰ VP Ad5 [E1-, E2b-]-E6/E7. Control mice were injected with 10¹⁰ VP Ad5 [E1-, E2b-]-null under the same protocol. In combinational studies, mice were given 100 μg of rat anti-PD-1 antibody (clone RMP1-14) or an isotype rat control antibody (clone 2A3) IP at the same time as immunization. Rat anti-PD-1 antibody and rat IgG2a isotype control antibodies were purchased from BioXcell. Tumor size was measured by two opposing dimensions (a, b) and volume was calculated according to the formula V=(tumor width²×tumor length)/2 where a was the shorter dimension. Animals were euthanized when tumors reached 1500 mm³ or when tumors became ulcerated.

### Analysis of Tumor-Infiltrating Cells (TILs) by Flow Cytometry

Four groups of 8-10 week old female C57BL/6 mice (n=5/group) were implanted with 2×10⁵ TC-1 tumor cells SQ in the left flank at day 0. Two of these groups were immunized SQ with 10¹⁰ VP Ad5 [E1-, E2b-]-null vector control and the other two groups SQ with 10¹⁰ VP Ad5 [E1-, E2b-]-E6/E7 vaccine. These immunizations were administered twice at 7-day intervals starting on day 12. In addition to immunizations, mice in one Ad5 [E1-, E2b-]-E6/E7 group and one Ad5 [E1-, E2b-]-null group were administered 100 μg rat anti-PD-1 antibody (clone RMP1-14) SQ at days 12 and 16, and 100 μg hamster anti-PD-1 antibody (clone J43) at days 19 and 23 to increase the effective dose of anti-PD-1 antibody. To control for treatment with these immune pathway checkpoint modulators, mice in the remaining Ad5 [E1-, E2b-]-E6/E7 and Ad5 [E1-, E2b-]-null groups were administered the relevant rat and hamster control IgG antibodies on the same days. Hamster anti-PD-1 antibody and isotype control were purchased from BioXcell. At day 27, tumors were measured, excised, and weighed. Tumors were minced and digested with a mixture of collagenase IV (1 mg/ml), hyaluronidase (100 g/ml), and DNase IV (200 U/ml) in Hank's Balanced Salt Solution (HBSS) at room temperature for 30 min and rotating at 80 rpm. Enzymes were purchased from Sigma-Aldrich. After digestion, the tumor suspension was placed through a 70 μm nylon cell strainer and centrifuged. Red cells were removed by the addition of red cell lysis buffer (Sigma-Aldrich) and after lysis, the tumor suspensions were washed twice in phosphate buffered saline (PBS) containing 1% (w/v) bovine serum albumin and resuspended in fluorescent activated cell sorting (FACS) buffer (PBS pH 7.2, 1% fetal bovine serum, and 2 mM EDTA) for staining. Fluorescent-conjugated antibodies against CD45 (30-F11), CD4 (RM4-5), and PDL1 (MIH5) were purchased from BD. Fluorescent-conjugated antibodies against CD83 (H35-17.2), CD25 (PC61.5), FoxP3 (FJK-16s), PD-1 (RMP1-30), LAG-3 (C9B7W), and CTLA4 (UC10-4B9) were all purchased from eBioscience. Surface staining was performed for 30 minutes at 4° C. in 100 μL FACS buffer containing anti-CD16/CD32 antibody (clone 2.4G2). Stained cells were washed in FACS buffer, fixed with paraformaldehyde, and (if needed) permeabilized in permeabilization buffer (eBioscience) before staining with fluorescent-conjugated anti-FoxP3 antibody or anti-CTLA4 antibody for 60 minutes at 4° C. in 100 μL permeabilization buffer containing anti-CD16/CD32 antibody (clone 2.4G2). Cells were washed with permeabilization buffer, washed back into FACS buffer, and a fixed volume of each sample was analyzed by flow cytometry using a BD Accuri C6 flow cytometer. Tumor cells were defined as CD45⁻ events in a scatter gate that includes small and large cells. CD4⁺ TILs were defined as CD45+/CD4⁺ events in a lymphocyte scatter gate. CD8⁺ TILs were defined as CD45+/CD8β⁺ events in a lymphocyte scatter gate. Regulatory T cells (Tregs) were defined as CD45+/CD4+/CD25+/FoxP3⁺ events in a lymphocyte scatter gate. Effector CD4+ T cells were defined as CD45+/CD4+/CD25-/FoxP3⁻ events in a lymphocyte scatter gate. Isotype-matched control antibodies were used to determine positive expression of FoxP3, PDL1, PD-1, LAG-3, and CTLA4. Flow cytometry was performed using an Accuri C6 Flow Cytometer (BD) and analyzed in BD Accuri C6 Software.

### HPV E6/E7 Specific Cell-Mediated Immune Responses Induced by Ad5 [E1-, E2b-]-E6/E7

A study was performed to determine the effect of increasing doses of Ad5 [E1-, E2b-]-E6/E7 immunizations on the induction of CMI responses in mice. Groups of C57BL/6 mice (n=5/group) were immunized SQ three times at 14-day intervals with 10⁸, 10⁹, or 10¹⁰ VP Ad5 [E1-, E2b-]-E6/E7. Control mice received 10⁸ VP, 10⁹ VP, or 10¹⁰ VP Ad5 [E1-, E2b-]-null (empty vector controls). Two weeks after the last immunization, splenocyte CMI responses were assessed by ELISpot analysis for IFN-γ secreting cells. A dose effect was observed and the highest CMI response level was obtained by immunizations with 10¹⁰ VP Ad5 [E1-, E2b-]-E6/E7. No responses were detected in control mice injected with Ad5 [E1-, E2b-]-null.

Intracellular accumulation of IFN-γ and TNF-α in both CD8α⁺ and CD4⁺ splenocytes populations were also determined in mice immunized with 10¹⁰ VP Ad5 [E1-, E2b-]-E6/E7. Intracellular cytokine staining (ICS) after stimulation with overlapping peptide pools revealed E6 and E7 antigen-specific IFN-γ accumulation in CD8α⁺ lymphocytes isolated from all mice immunized with Ad5 [E1-, E2b-]-E6/E7. Peptide-stimulated splenocytes were also stained for the intracellular accumulation of TNF-α, and a significant population of multifunctional (IFN-γ+/TNF-α⁺) CD8α⁺ splenocytes specific for both E6 and E7 were able to be detected.

### Treatment of HPV E6/E7-Expressing Tumors

The anti-tumor effect of immunotherapy treatment in mice bearing HPV E6/E7 TC-1 tumors was investigated. These tumor cells expressed PDL1 as assessed by flow cytometry analysis. When labeled with PE-conjugated anti-PDL1, the TC-1 cells had a median fluorescent intensity (MFI) of 537 whereas cells labeled with a PE-conjugated isotype control antibody had an MFI of 184, demonstrating the presence of the immune suppressive PDL1 on the surface of the TC-1 cells (data not shown). Two groups of C57BL/6 mice (n=5/group) were inoculated with 2×10⁵ TC-1 tumor cells SQ into the right subcostal area on day 0. On days 1, 8, and 14 mice were treated by SQ injections of 10¹⁰ VP Ad5 [E1-, E2b-]-null (vector control) or 10¹′ VP Ad5 [E1-, E2b-]-E6/E7. All mice were monitored for tumor size and tumor volumes were calculated. Mice immunized with Ad5 [E1-, E2b-]-E6/E7 had significantly smaller tumors than control mice beginning on day 12 (p<0.01) and remained significantly smaller for the remainder of the experiment (p<0.02), including 3 of 5 mice showing complete tumor regression. Tumors in mice from the vector control treated group began reaching the threshold for euthanasia starting on day 26 and all mice in this group were euthanized by day 33, whereas mice in the Ad5 [E1-, E2b-]-E6/E7 treated group were all alive with complete tumor regression of small tumors (<150 mm³) at the end of experiment on day 36.

To determine if immunotherapy with Ad5 [E1-, E2b-]-E6/E7 was effective against larger tumors, TC-1 tumor cells were implanted in two groups of C57BL/6 mice (n=4/group) and then delayed weekly treatment with Ad5 [E1-, E2b-]-E6/E7 for 6 days post tumor implantation, at a time when tumors were small but palpable. Mice beginning treatment on day 6 initially demonstrated tumor growth similar to the control group; however, beginning on day 16, tumor regression was observed. The tumors in mice that began treatment on day 6 were significantly smaller (p<0.05) than the control group beginning on day 20 and 3 of 4 mice had complete regression by day 27. Ad5 [E1-, E2b-]-E6/E7 administration beginning on day 6 also conferred a significant survival benefit (p<0.01).

Finally, to determine if immunotherapy with Ad5 [E1-, E2b-]-E6/E7 was effective against large established tumors, TC-1 tumor cells were implanted in two groups of C57BL/6 mice (n=4/group) then delayed weekly treatment with Ad5 [E1-, E2b-]-E6/E7 until 13 days post tumor implantation, when tumors were −100 mm³. In this treatment group, initial tumor growth was observed to be similar to the control group but some mice in the control group reached euthanasia criteria on day 23, preventing analysis of significance at further time points. However, tumor volumes in the Ad5 [E1-, E2b-]-E6/E7 treated group were below the euthanasia threshold through day 29, at which point tumors from all mice in the vector control group had exceeded 1500 mm³ and were euthanized. These results indicate that in the TC-1 tumor model the Ad5 [E1-, E2b-]-E6/E7 immunotherapeutic was a potent inhibitor of tumor growth and lead to significant overall survival benefit; however, complete clearance of tumors was only observed when treatment was initiated in smaller tumors. Furthermore, these results demonstrate that, despite the presence of immune suppressing PDL1 on tumor cells, immunotherapeutic treatment with Ad5 [E1-, E2b-]-E6/E7 resulted in significant inhibition of tumor growth.

### Example 2

### Induction of Immune Responses to HPV E6 and HPV E7

This example describes the use of Ad5 [E1-, E2b-]-E6/E7 products for inducing immune responses to HPV E6 and HPV E7 for the treatment of HPV E6/E7-expressing tumors.

### Treatment of HPV E6/E7-Expressing Tumors

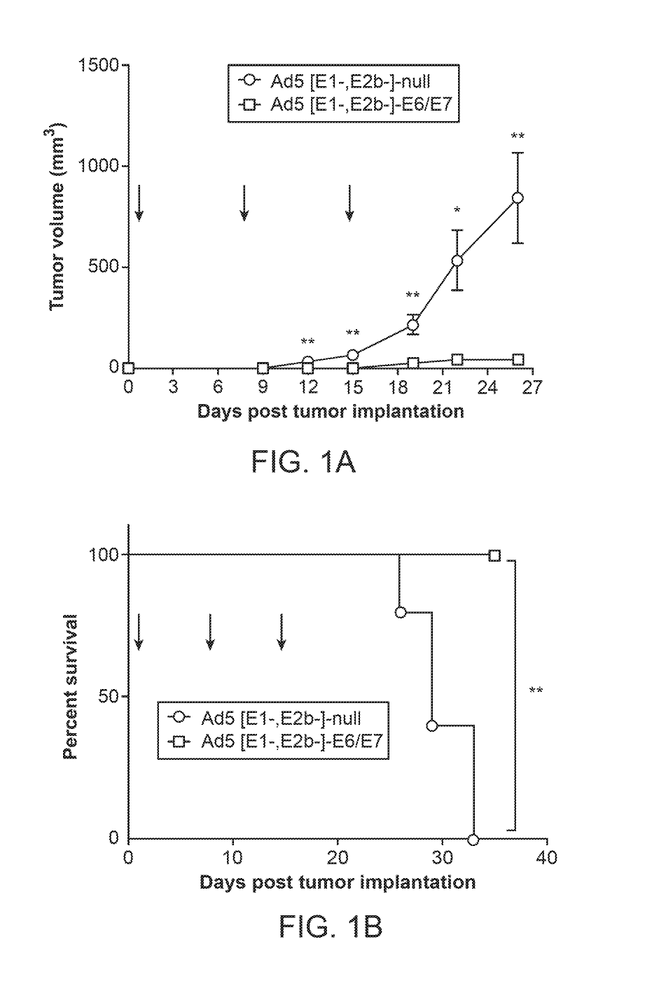

Previously, anti-tumor effect of immunotherapy treatment in mice bearing HPV E6/E7 TC-1 tumors was investigated. These tumor cells expressed PDL1 as assessed by flow cytometry analysis. When labeled with PE-conjugated anti-PDL1, the TC-1 cells had a median fluorescent intensity (MFI) of 537 whereas cells labeled with a PE-conjugated isotype control antibody had an MFI of 184, demonstrating the presence of the immune suppressive PDL1 on the surface of the TC-1 cells. Two groups of C57BL/6 mice (n=5/group) were inoculated with 2×10⁵ TC-1 tumor cells SQ into the right subcostal area on day 0. On days 1, 8, and 14 mice were treated by SQ injections of 10¹⁰ VP Ad5 [E1-, E2b-]-null (vector control) or 10¹⁰ VP Ad5 [E1-, E2b-]-E6/E7. All mice were monitored for tumor size and tumor volumes were calculated. Mice immunized with Ad5 [E1-, E2b-]-E6/E7 had significantly smaller tumors than control mice beginning on day 12 (p<0.01) and remained significantly smaller for the remainder of the experiment (p<0.02), including 3 of 5 mice showing complete tumor regression (FIG. 1A). Tumors in mice from the vector control treated group began reaching the threshold for euthanasia starting on day 26 and all mice in this group were euthanized by day 33, whereas mice in the Ad5 [E1-, E2b-]-E6/E7 treated group were all alive with complete tumor regression of small tumors (<150 mm³) at the end of experiment on day 36 (FIG. 1B).

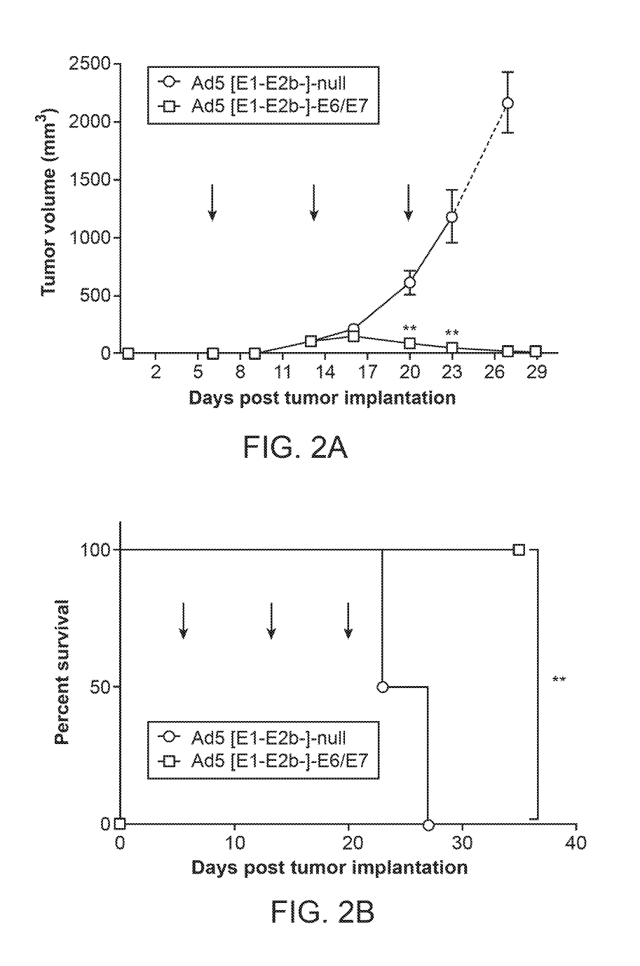

To determine if immunotherapy with Ad5 [E1-, E2b-]-E6/E7 was effective against larger tumors, TC-1 tumor cells were implanted in two groups of C57BL/6 mice (n=4/group) and then delayed weekly treatment with Ad5 [E1-, E2b-]-E6/E7 for 6 days post tumor implantation, at a time when tumors were small but palpable. Mice beginning treatment on day 6 initially demonstrated tumor growth similar to the control group; however, beginning on day 16, tumor regression was observed (FIG. 2A). The tumors in mice that began treatment on day 6 were significantly smaller (p<0.05) than the control group beginning on day 20, and 3 of 4 mice had complete regression by day 27. Ad5 [E1-, E2b-]-E6/E7 administration beginning on day 6 also conferred a significant survival benefit (p<0.01) (FIG. 2B).

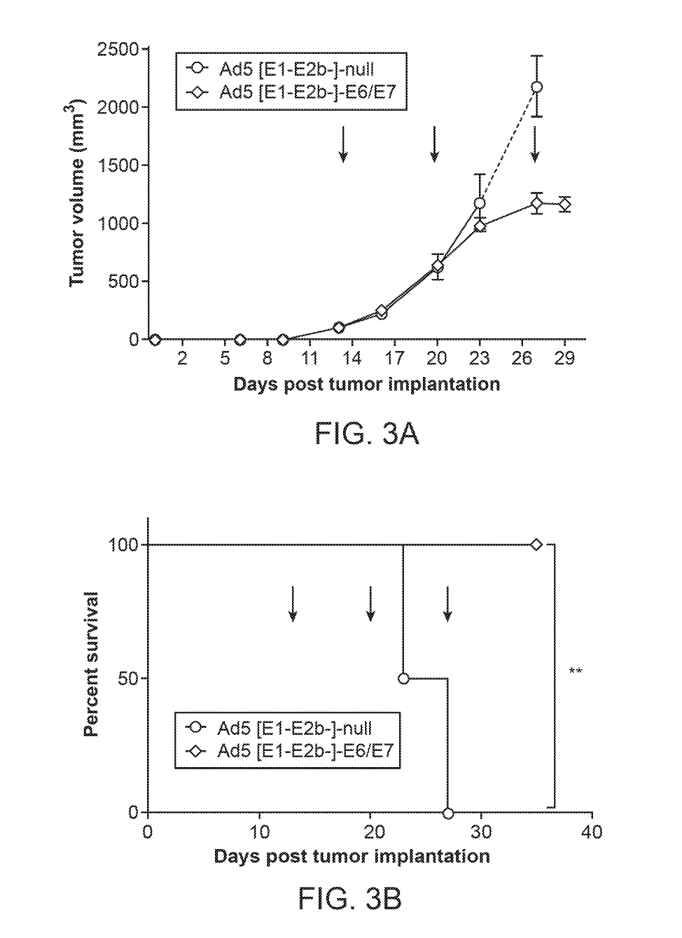

Finally, to determine if immunotherapy with Ad5 [E1-, E2b-]-E6/E7 was effective against large established tumors, TC-1 tumor cells were implanted in two groups of C57BL/6 mice (n=4/group) then delayed weekly treatment with Ad5 [E1-, E2b-]-E6/E7 until 13 days post tumor implantation, when tumors were −100 mm³. In this treatment group, initial tumor growth was observed to be similar to the control group but some mice in the control group reached euthanasia criteria on day 23, preventing analysis of significance at further time points (FIG. 3A). However, tumor volumes in the Ad5 [E1-, E2b-]-E6/E7 treated group were below the euthanasia threshold through day 29, at which point tumors from all mice in the vector control group had exceeded 1500 mm³ and were euthanized (FIG. 3B). These results indicate that in the TC-1 tumor model the Ad5 [E1-, E2b-]-E6/E7 immunotherapeutic was a potent inhibitor of tumor growth and lead to significant overall survival benefit; however complete clearance of tumors was only observed when treatment was initiated in smaller tumors. Furthermore, these results demonstrate that, despite the presence of immune suppressing PDL1 on tumor cells, immunotherapeutic treatment with Ad5 [E1-, E2b-]-E6/E7 resulted in significant inhibition of tumor growth.

Combination Immunotherapy with Immune Checkpoint Inhibition

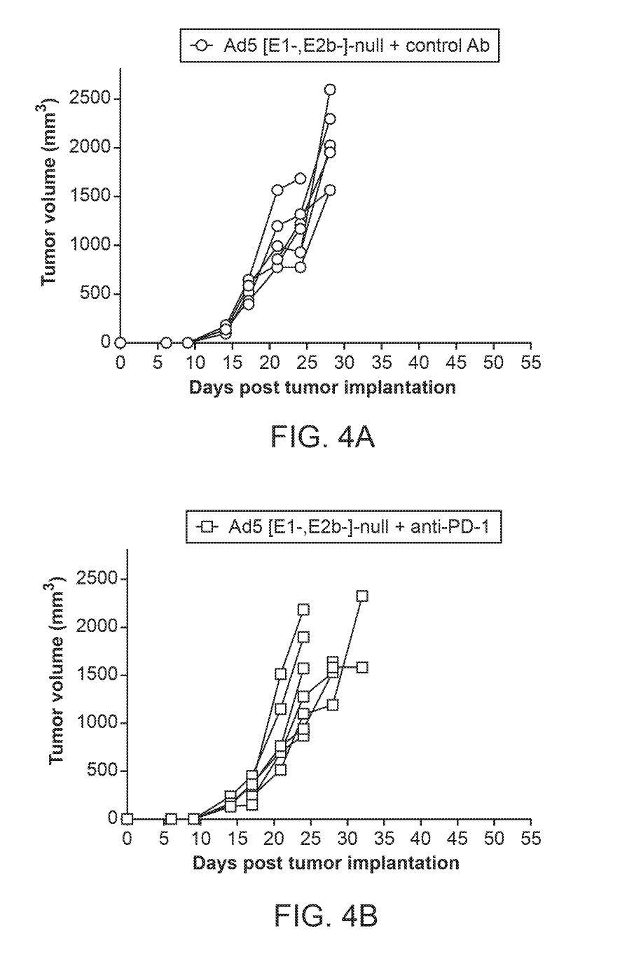

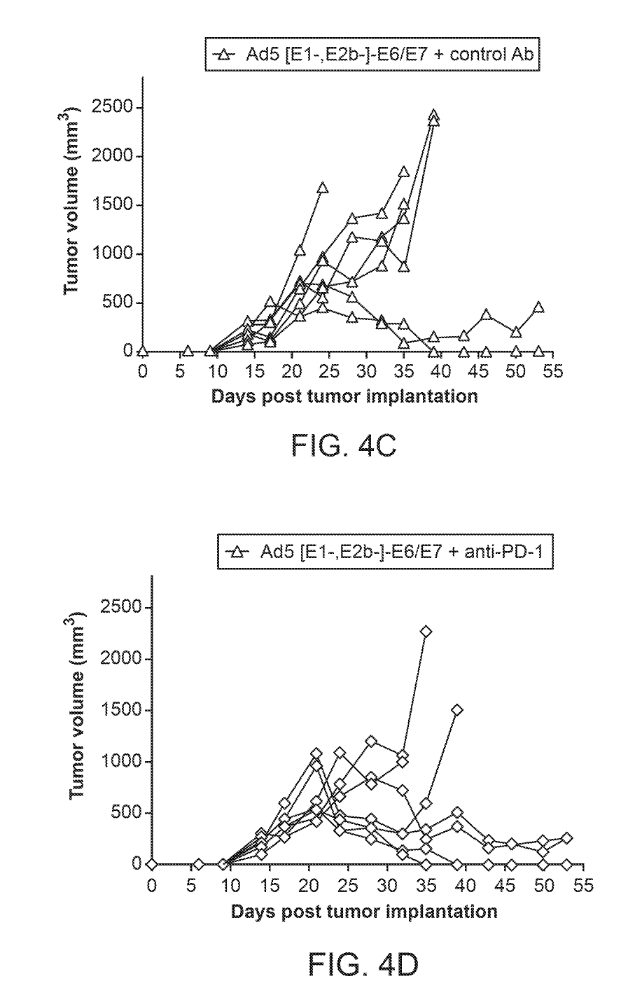

To determine if the therapeutic effect of Ad5 [E1-, E2b-]-E6/E7 could be improved in the setting of large tumors, anti-PD-1 antibody was co-administered. Four groups of mice (n=7/group) were implanted with 2×10⁵ TC-1 tumor cells on day 0 and beginning on day 10 the mice received weekly administrations of SQ 10¹⁰ VP Ad5 [E1-, E2b-]-E6/E7 plus IP 100 μg anti-PD-1 antibody, 10¹⁰ VP Ad5 [E1-, E2b-]-null plus 100 μg anti-PD-1 antibody, 10¹⁰ VP Ad5 [E1-, E2b-]-E6/E7 plus 100 μg rat IgG2a isotype control antibody, or 10¹⁰ VP Ad5 [E1-, E2b-]-null plus 100 μg rat IgG2a isotype control antibody. Tumor size was monitored over time and mice were euthanized when tumor size exceeded 1500 mm³ or when tumor ulceration was present. Control mice that received Ad5 [E1-, E2b-]-null plus 100 μg rat IgG2a isotype control antibody (FIG. 4A) and mice treated with Ad5 [E1-, E2b-]-null plus 100 μg anti-PD-1 antibody (FIG. 4B) exhibited a similar tumor growth pattern. No significant survival benefit was observed between these two groups. Mice that received Ad5 [E1-, E2b-]-E6/E7 plus rat IgG2a isotype control antibody had a delayed tumor growth pattern as compared to the controls and 2 of the mice had tumor regressions to near baseline level at day 52 post tumor implantation (FIG. 4C). Four of the 7 mice that received Ad5 [E1-, E2b-]-E6/E7 and anti-PD-1 antibody had tumor regression starting at day 25, and two of these resulted in tumor clearance through the end of experiment at day 53 (FIG. 4D).

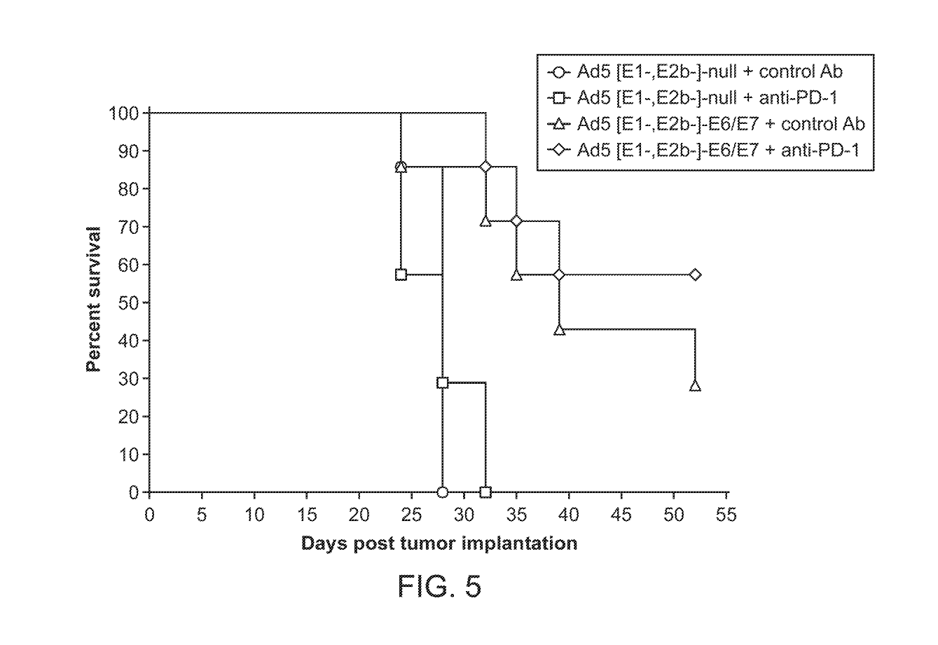

Mice treated with Ad5 [E1-, E2b-]-E6/E7 plus rat IgG2a isotype control antibody (FIG. 5) also experienced a survival benefit with 28.6% of the animals surviving at termination of the study whereas 100% of the control mice (Ad5 [E1-, E2b-]-null plus rat IgG2a isotype control antibody) and the Ad5 [E1-, E2b-]-null plus anti-PD-1 antibody treated mice had to be terminated by day 28 and 32, respectively (FIG. 5). Mice treated with both Ad5 [E1-, E2b-]-E6/E7 and anti-PD-1 antibody had the greatest treatment benefit (FIG. 5), demonstrating delayed tumor growth and a significant improvement (P≤0.0006) in survival as compared to the controls.

Mouse anti-rat IgG antibody responses were induced by the second injection (endpoint antibody titer 1:200 by ELISA, data not shown) with rat anti-PD-1 antibody, and these responses were dramatically increased by the third injection (endpoint antibody titer 1:4000 to 1:8000 by ELISA, data not shown). This anti-rat antibody response may explain why no anti-tumor activity was observed after injections with anti-PD-1 antibody alone. Also, it is likely that the first and possibly the second injections of anti-PD-1 antibody combined with Ad5 [E1-, E2b-]-E6/E7 immunotherapy were effective but the third injection with anti-PD-1 antibody was effectively neutralized by the induced mouse anti-rat IgG antibody response.

### Tumor Microenvironment Following Combination Immunotherapy

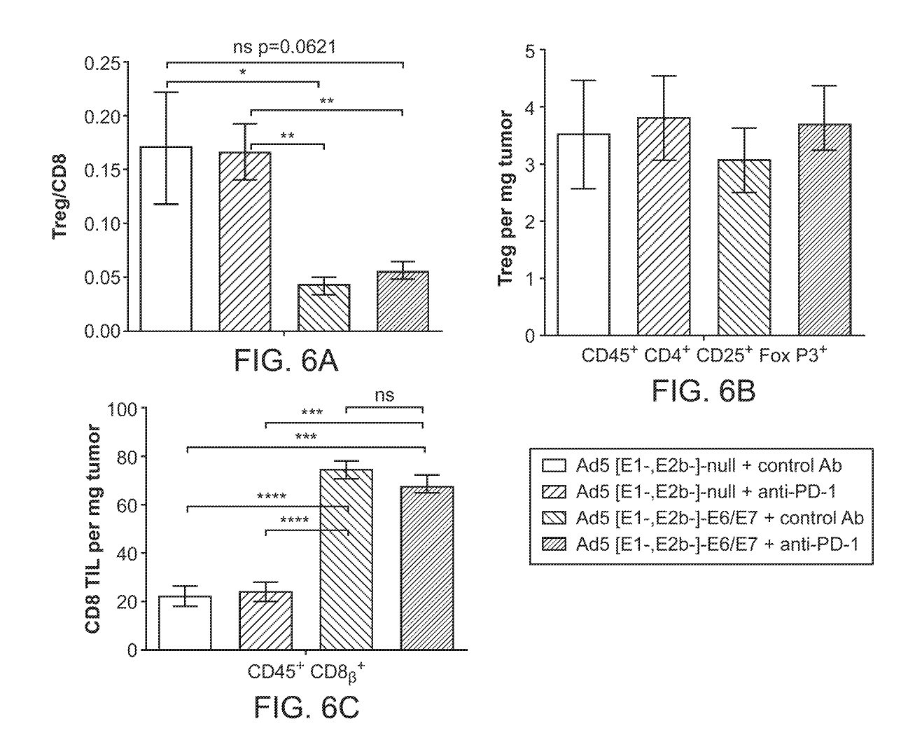

To analyze cell populations that contributed to delayed tumor growth and survival in Ad5 [E1-, E2b-]-E6/E7 treated mice, tumor-infiltrating lymphocytes (TILs) were by flow cytometry. Four groups of mice were implanted with 2×10⁵ TC-1 cells and began treatment 10 days later with two weekly immunizations of Ad5 [E1-, E2b-]-E6/E7 plus PD-1 antibody. On day 27 whole tumors were collected and processed as described in the materials and methods. The number of infiltrating CD8⁺ T cells per mg of tumor was significantly increased in the Ad5 [E1-, E2b-]-E6/E7 treated groups as compared to the groups that received Ad5 [E1-, E2b-]-null (FIG. 6C). Anti-PD-1 antibody treatment had little or no effect on the number of infiltrating CD8⁺ T cells (FIG. 6C). There was no difference between any of the four groups, in terms of the number of infiltrating Tregs (CD4⁺ CD25⁺Foxp3⁺) per mg of tumor (FIG. 6B). However, the increase in CD8⁺ T cells led to a decrease in the Treg:CD8⁺ T cell ratio in the tumor microenvironment when the mice were treated with the Ad5 [E1-, E2b-]-E6/E7 vaccine or Ad5 [E1-, E2b-]-E6/E7 vaccine plus anti-PD-1 antibody treatment (FIG. 6A).

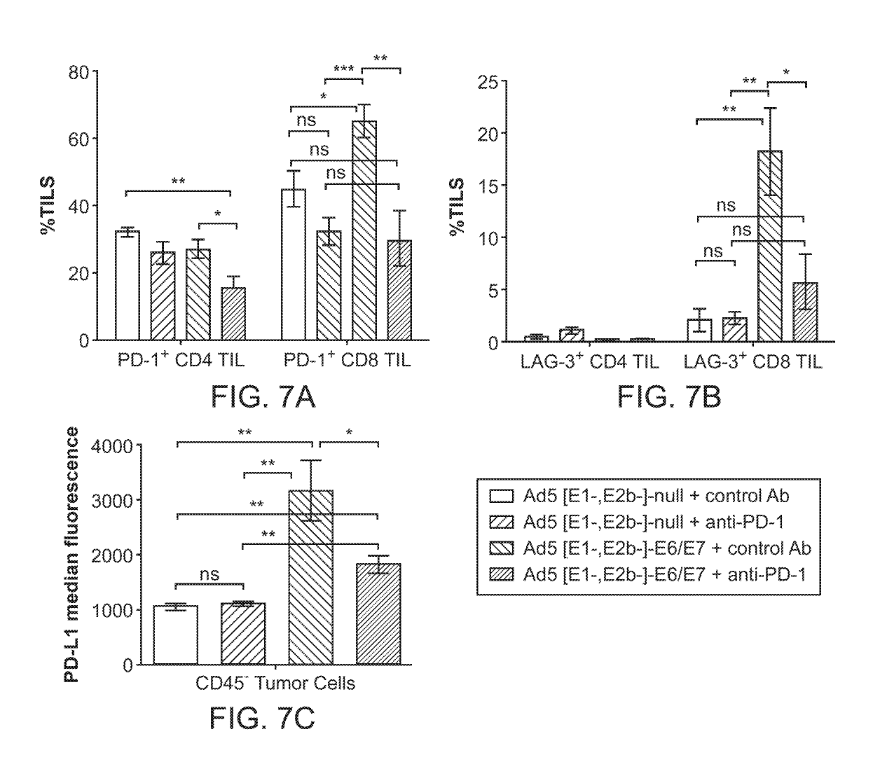

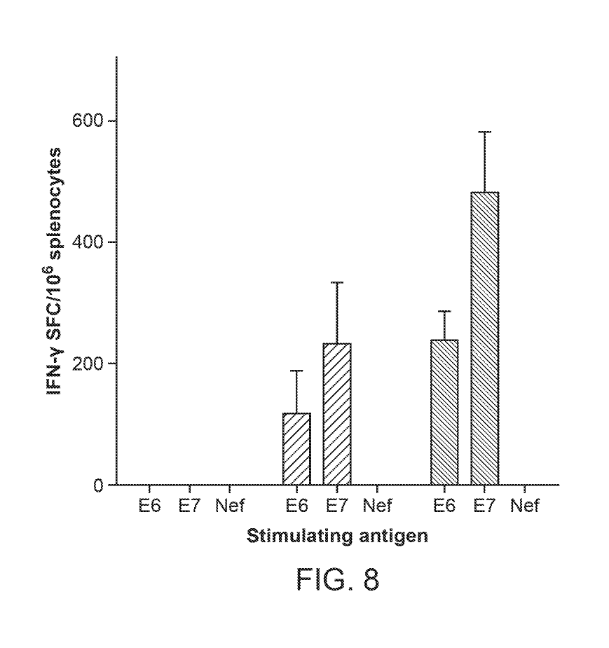

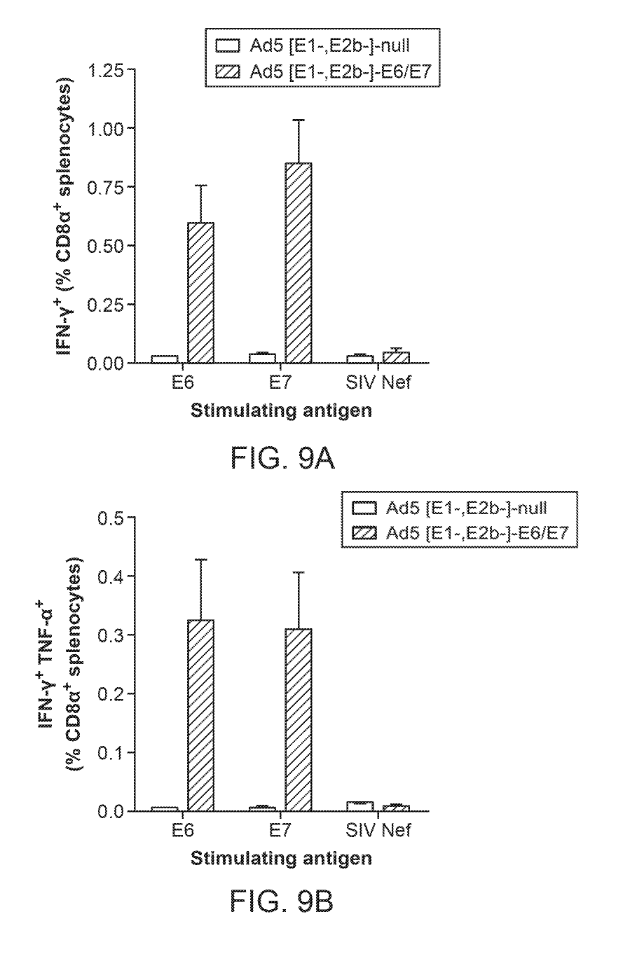

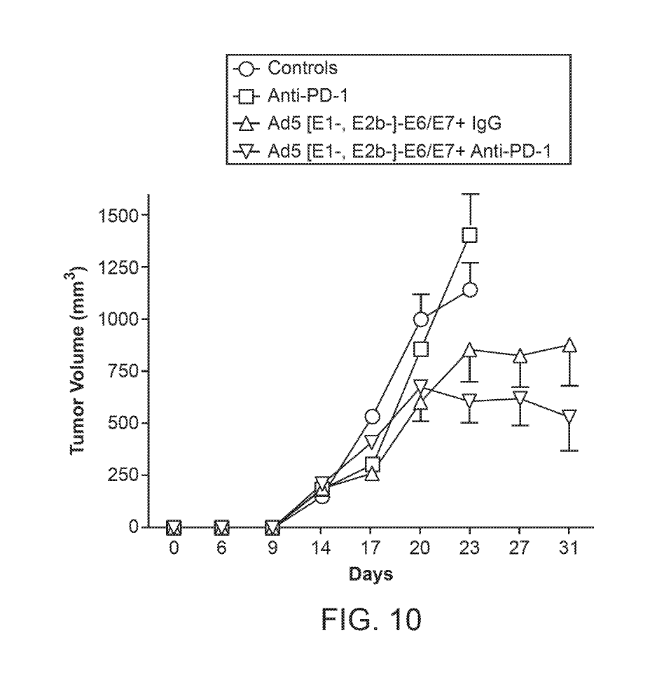

To further study the synergistic/additive effect of anti-PD-1 antibody to Ad5 [E1-, E2b-]-E6/E7 immunotherapy, the expression of PD-1, LAG-3, and CTLA-4 was examined on TILs. The expression of these co-inhibitory molecules on T cells within the tumor microenvironment has been shown to down regulate activation of antigen-specific T cells. Immunizations with Ad5 [E1-, E2b-]-E6/E7 plus control antibody treatment significantly increased the fraction of PD-1⁺ and LAG-3⁺ CD8⁺ TILs, whereas, expression of these co-inhibitory molecules on CD4⁺ TILs was unaffected by this treatment. The percentage of CD4⁺ and CD8⁺ TILs expressing CTLA-4 was not significantly affected by vaccine treatment (data not shown). Combining anti-PD-1 antibody injections with Ad5 [E1-, E2b-]-E6/E7 vaccine treatment resulted in a significant reduction in the fraction of PD-1⁺ CD8⁺ and CD4⁺ TILs, as compared with those found in tumors from mice treated with Ad5 [E1-, E2b-]-E6/E7 plus control antibody (p=0.0083 for CD8⁺ TILs and p=0.0016 for CD4⁺ TILs). Furthermore the fraction of PD-1⁺ CD8⁺ TILs was decreased to the level of expression observed in the Ad5 [E1-, E2b-]-null treated control groups, and the fraction of PD-1⁺ CD4⁺ TILs was significantly reduced to below that observed in the control groups (p=0.0016, FIG. 7A). In addition, the percentage of LAG-3⁺ CD8⁺ TILs was also observed to decrease when the Ad5 [E1-, E2b-]-E6/E7 immunization was combined with the anti-PD-1 antibody (p=0.0363, FIG. 7B). Since it has previously been shown that vaccine treatment can enhance PDL1 expression on tumor cells ex vivo, the expression of PDL1 was examined on tumor cells. There was an augmentation in the median fluorescence intensity of PDL1 on tumor cells after vaccine treatment. However, PDL1 expression was reduced in mice treated with the combination of Ad5 [E1-, E2b-]-E6/E7 and anti-PD-1 antibody, although this level was still significantly expressed above that observed in Ad5 [E1-, E2b-]-null treated control mice.

In summary, the data demonstrate that Ad5 [E1-, E2b-]-E6/E7 can induce HPV E6/E7 directed CMI responses in a dose dependent manner, which results in upregulation of PDL1 on tumor cells. Multiple homologous immunizations in tumor bearing mice with the highest dose of vaccine resulted in significant anti-tumor activity and increased survival, particularly in mice bearing small tumors. Importantly, a greater degree of anti-tumor activity was achieved when immunotherapy with Ad5 [E1-, E2b-]-E6/E7 was combined with anti-PD-1 antibody in mice with large tumors. Overall, immunizations with the Ad5 [E1-, E2b-]-E6/E7 vaccine combined with anti-PD-1 antibody results in an increase in CD8⁺ and CD4⁺ effector populations that have a less exhaustive/anergic phenotype and therefore favor the balance to a more pro-inflammatory state in the tumor microenvironment. The observation that the combined treatment was associated with reductions in large tumor mass indicates that immunotherapy with Ad5 [E1-, E2b-]-E6/E7 combined with anti-PD-1 antibody might increase clinical effectiveness during the immunotherapy of subjects with HPV-associated head and neck or cervical cancers. Furthermore, the data suggests that clinical trials with the Ad5 [E1-, E2b-]-E6/E7 vaccine should be combined with an immune pathway checkpoint modulator and remains a high priority.

### Example 3

### Clinical Trial of Ad5 [E1-, E2b-]-E6/E7 Vaccine

This example describes the evaluation of safety and immunogenicity of immunizations with the Ad5 [E1-, E2b-]-E6/E7 vaccine in subjects that are human papilloma virus type 16 (HPV-16) positive, in subjects with HPV-associated head and neck squamous cell carcinoma (HNSCC), and in subjects with HPV-associated cervical cancer.

Current interventions in HNSCC patients include therapy with cisplatin and radiation or cetuximab and radiation. However, many HNSCC patients that initially respond or do not respond ultimately relapse. The vaccine is designed to induce anti-tumor T cell-mediated immune responses directed against the early 6 (E6) and early 7 (E7) genes of HPV. One of the important features of the vaccine is that it can be combined with chemotherapy/radiation treatment.

The backbone of the vaccine is an adenovirus serotype 5 (Ad5) vector that has been modified by removal of the E1, E2b, and E3 genes and insertion of a modified fused non-oncogenic HPV E6/E7 gene. The resulting recombinant replication-defective vector can only be propagated in the newly engineered, proprietary human 293 based cell line (E.C7) that supplies the E1 and E2b gene functions in trans required for vector production.

No gene transfer insertion is proposed for this protocol; the product functions and remains episomal.

The vaccine product is used to induce HPV E6/E7 specific cell-mediated immune responses in a safe and effective manner in subjects. An open-label, dose-escalation clinical study is conducted to evaluate the safety and immunogenicity of Ad5 [E1-, E2b-]-E6/E7 vaccine injections. The dosage levels to be evaluated are 5×10¹⁰, 1×10¹¹, and 5×10¹¹ virus particles (VP) of Ad5 [E1-, E2b-]-E6/E7 vaccine. Subjects are enrolled into successive increasing dosage levels involving three (3) cohorts of subjects that are monitored for dose-limiting toxicity (DLT). Each subject is given Ad5 [E1-, E2b-]-E6/E7 vaccine by SQ injection every 3 weeks for 3 immunizations. Assessment of DLT for dose escalation is made after all subjects in a cohort have had a study visit at least 3 weeks after receiving their last dose of vaccine.

The Ad5 backbone expressing HPV E6/E7 is used for the immunization (vaccination) of subjects that are HPV-16+ and at high risk for developing HPV+ cancers or who have HPV+ cancers. The subjects are animals, such as humans, non-human primates (e.g., rhesus or other types of macaques), mice, pigs, horses, donkeys, cows, sheep, rats, or fowls.

Induction of CMI Responses after Ad5 [E1-, E2b-]-E6/E7 Vaccination as Assessed by Flow Cytometry

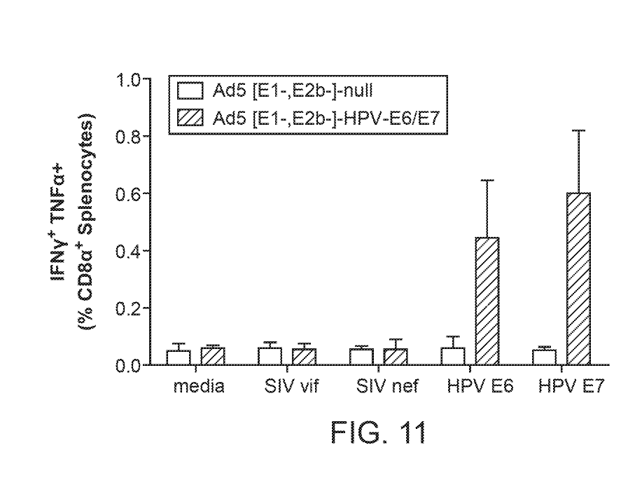

To assess CMI induction by flow cytometry following multiple homologous immunizations with Ad5 [E1-, E2b-]-E6/E7, groups of C57Bl/6 mice (n=5/group) were immunized three times SQ at 2-week intervals with 10¹⁰ VP of Ad5 [E1-, E2b-]-E6/E7. Two weeks following the last immunization, splenocytes were exposed to HPV E6/E7 peptides or irrelevant antigens and analyzed by flow cytometry for the number of IFN-γ and/or TNFα expressing T cells. As shown in FIG. 11, both IFN-γ and/or TNFα expressing T cells were induced as a result of multiple homologous immunizations with the highest dose of Ad5 [E1-, E2b-]-E6/E7. Specificity studies revealed that CMI responses were specific to HPV E6 and E7 and there were no responses against irrelevant antigens such as SIV-vif or SIV-nef. Toxicology

An extensive pre-clinical toxicology study is conducted to assess the toxicity of Ad5 [E1-, E2b-]-E6/E7 following SQ injections on in C57Bl/6 mice. Toxicity endpoints are assessed at various time points post-injection. The animals is administered up to 3 SQ injections on Days 1, 22, and 43, with either vehicle control or Ad5 [E1-, E2b-]-E6/E7 at a dose consistent with that to be used in clinical trials accounting for difference in body mass. Evaluations consist of effects on body weights, body weight gain, food consumption pathology, blood hematology analyses, blood chemistry analyses, and test on coagulation time.

Treatment of Established HPV E6/E7-Expressing Tumors with Vaccine Alone

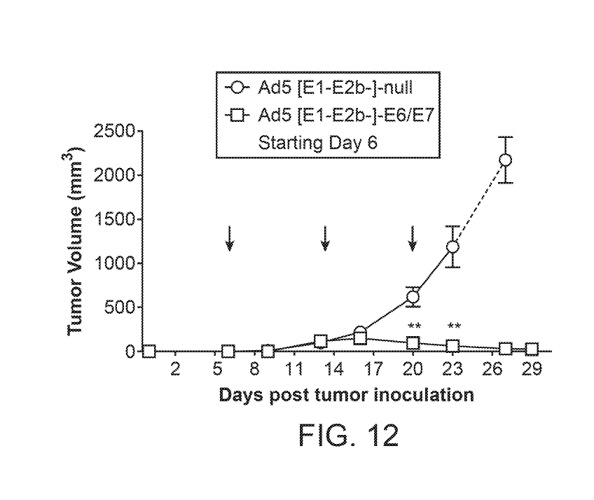

The effectiveness of treating established HPV E6/E7-expressing tumors in vivo with Ad5 [E1-, E2b-]-E6/E7 was evaluated. C57Bl/6 mice were implanted SQ into the right subcostal with 10⁶ HPV E6/E7-expressing tumor cells on day 0. Tumors were palpable by days 4-6. On days 6, 13, and 20, mice were treated by SQ injections of 10¹⁰ VP of Ad5 [E1-, E2b-]-null (empty vector controls) or 10¹⁰ VP of Ad5 [E1-, E2b-]-E6/E7. All mice were monitored for tumor growth and tumor volumes were calculated. As shown in FIG. 12, mice immunized with Ad5 [E1-, E2b-]-E6/E7 had significantly smaller tumors than control mice (p<0.01). These results demonstrate that the Ad5 [E1-, E2b-]-E6/E7 vector platform has the potential to be utilized as an immunotherapeutic agent to treat HPV E6/E7-expressing tumors.

Treatment of Established HPV E6/E7 Expressing Tumors with Vaccine and Chemotherapy/Radiation Treatment

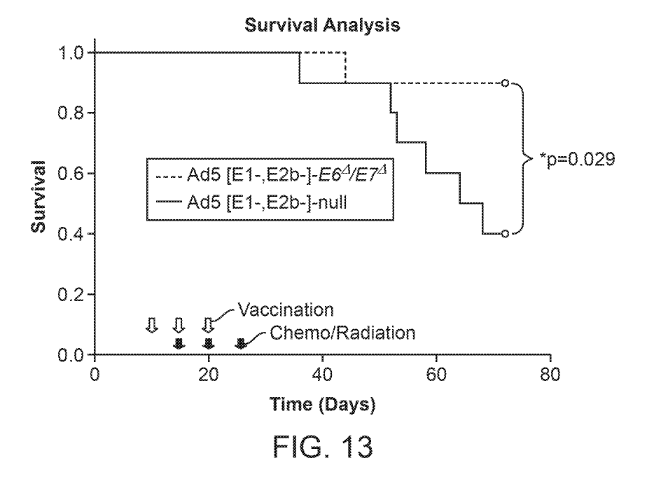

The effectiveness of treating HPV16 E6/E7 expressing tumors in vivo with Ad5 [E1-, E2b-]-HPV16-E6^{Δ}/E7^{Δ} combined with chemotherapy/radiation treatment was evaluated. C57Bl/6 mice were implanted SQ with 10⁶ HPV16 E6/E7 expressing tumor cells on day 0. Established HPV16-E6^{Δ}/E7^{Δ} expressing tumors were treated by immunotherapy on days 7, 14, and 21 combined with cisplatin/radiation treatment on days 13, 20, and 27. Control tumor bearing mice were treated by injections with Ad-null (empty vector control) combined with cisplatin/radiation treatment. As shown in FIG. 13, combination treatment using Ad5 [E1-, E2b-]-HPV16-E6^{Δ}/E7^{Δ} and chemotherapy/radiation resulted in significant extension of survival time as compared with control mice receiving treatment Ad5 [E1-, E2b-]-null and chemotherapy/radiation. These results showed that vaccine immunotherapy can be combined with chemotherapy/radiation treatment and that this combination results in a significantly greater extension of survival in a mouse model of HPV16 E6/E7 expressing cancer.

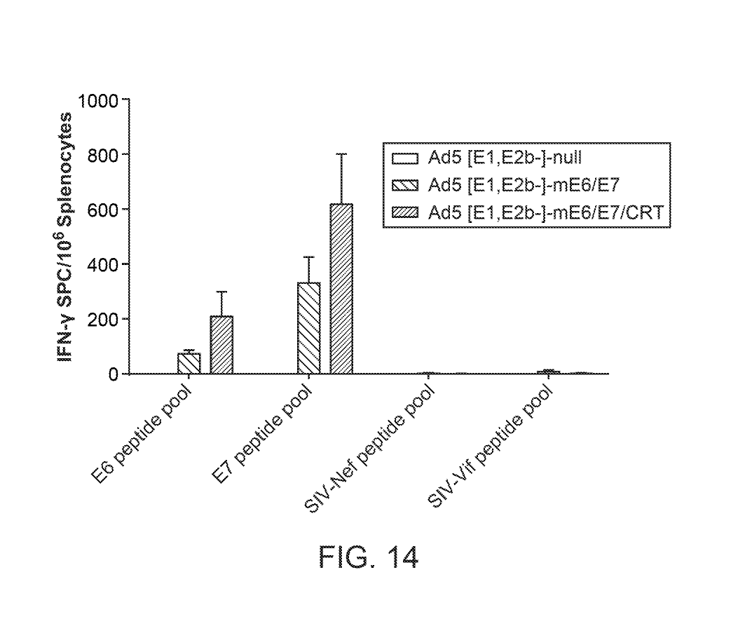

In light of these results, the effects on the immune response of combined immunizations with Ad5 [E1-, E2b-]-E6/E7 and cisplatin/radiation treatment versus cisplatin/radiation treatment alone were investigated in a murine model. The combination of Ad5 [E1-, E2b-]-HPV16-E6^{Δ}/E7^{Δ} immunizations plus cisplatin/radiation treatment resulted in the induction of greater CMI responses as compared to immunizations with Ad5 [E1-, E2b-]-HPV16-E6^{Δ}/E7^{Δ} alone (FIG. 14). These results indicate that immunotherapy can be combined with chemotherapy treatment in order to achieve greater anti-tumor CMI responses.

In summary, Ad5 [E1-, E2b-]-E6/E7 is a non-oncogenic vaccine targeting HPV E6 and HPV E7 that induces robust immune responses. Ad5 [E1-, E2b-]-E6/E7 induced potent CMI against HPV E6/E7 in mice assessed in ELISpot and flow cytometry studies. Ad5 [E1-, E2b-]-E6/E7 significantly inhibited progression of established tumors in a murine model of HPV E6/E7-expressing cancer. Immunotherapy with Ad5 [E1-, E2b-]-E6/E7 could be combined with chemotherapy/radiation treatment to significantly increase survival in tumor bearing mice. The goal is to further develop this novel Ad5 vector system that overcomes barriers found with other Ad5 systems and clinically tests this vaccine to determine that significant HPV E6/E7 directed immune responses are induced in immunized (vaccinated) subjects. The results of this clinical study establish the safety and immunogenicity of using this new Ad5 [E1-, E2b-]-E6/E7 vaccine.

### Example 4

### Production and Evaluation of Ad5 Vectors Containing HPV E6 and/or HPV E7 Agonist Epitope Variants

This example shows that the Ad5 [E1-, E2b-]-E6, Ad5 [E1-, E2b-]-E7, or Ad5 [E1-, E2b-]-E6/E7 products containing various agonist epitopes are constructed and evaluated in a similar fashion. These vectors are used in Examples 4-6.

### Viral Construction

Ad5 [E1-, E2b-]-E6, Ad5 [E1-, E2b-]-E7, or Ad5 [E1-, E2b-]-E6/E7 vaccine is an adenovirus serotype 5 (Ad5) vector that has been modified by removal of the E1, E2b, and E3 genes and insertion of modified HPV E6 and/or HPV E7 genes that have agonist epitope variants with coding sequences set forth in SEQ ID NO: 2, SEQ ID NO: 3, SEQ ID NO: 4, SEQ ID NO: 5, SEQ ID NO: 18, SEQ ID NO: 6, SEQ ID NO: 19, SEQ ID NO: 7, SEQ ID NO: 20, SEQ ID NO: 11, and SEQ ID NO: 21.

In addition, Ad5 [E1-, E2b-]-E6, Ad5 [E1-, E2b-]-E7, or Ad5 [E1-, E2b-]-E6/E7 vaccine is an adenovirus serotype 5 (Ad5) vector that has been modified by removal of the E1, E2b, and E3 genes and insertion of modified HPV E6 and/or HPV E7 genes encoding HPV antigens set forth in the following sequences: (1) SEQ ID NO: 8 (HPV16 E6 with E6A1 epitope) and SEQ ID NO: 12 (HPV16 E7 with E7A3 epitope), (2) SEQ ID NO: 9 (HPV16 E6 with E6A3 epitope) and SEQ ID NO: 12 (HPV16 E7 with E7A3 epitope), and (3) SEQ ID NO: 10 (HPV16 E6 with E6A1+E6A3 epitopes), and SEQ ID NO: 12 (HPV16 E7 with E7A3 epitope). Any one of the following sequences, which encodes for HPVE6 or HPV E7 antigens is used alone, or any HPV E6 sequence is combined with any HPV E7 sequence to obtain an E6/E7 vaccine: SEQ ID NO: 18 (HPV16 E6 with E6A1 epitope), SEQ ID NO: 19 (HPV16 E6 with E6A3 epitope), SEQ ID NO: 20 (HPV16 E6 with E6A1 and E6A3 epitope), SEQ ID NO: 21 (HPV16 E7 with E7A3 epitope), SEQ ID NO: 13 (HPV16 E6 with JL), SEQ ID NO: 8 (HPV16 E6 with NCI E6A1 epitope), SEQ ID NO: 9 (HPV16 E6 with NCI E6A3 epitope), SEQ ID NO: 10 (HPV16 E6 with E6A1 and E6A3 epitopes), SEQ ID NO: 14 (HPV16 E7 with JL), SEQ ID NO: 12 (HPV16 E7 with NCI E7A3 epitope), SEQ ID NO: 15 (HPV E6E7), SEQ ID NO: 2 (HPV16 E6E7 with E6A1 and E7A3 epitopes), SEQ ID NO: 3 (HPV16 E6E7 with E6A3 and E7A3 epitopes), SEQ ID NO: 4 (HPV16 E6E7 with E6A1, E6A3, and E7A3 epitopes).

Briefly, the transgenes are sub-cloned into the Ad5 [E1-, E2b-] vector using a homologous recombination-based approach and the replication deficient virus is propagated in the E.C7 packaging cell line, CsCl₂ purified, and infectious titer expressed as plaque forming units (PFU) is determined on an E.C7 cell monolayer. The virus particle (VP) concentration is determined by sodium dodecyl sulfate (SDS) disruption and spectrophotometry at 260 nm and 280 nm. As a vector control, Ad5 [E1-, E2b-]-null (e.g., SEQ ID NO: 14) is employed, which is the Ad5 platform backbone with no transgene insert.

### Immunization and Splenocyte Preparation

Female C57BL/6 mice (n=5/group) are injected subcutaneously (SQ) with varying doses of Ad5 [E1-, E2b-]-E6, Ad5 [E1-, E2b-]-E7, Ad5 [E1-, E2b-]-E6/E7, or Ad5 [E1-, E2b-]-null. Doses are administered in 25 μL injection buffer (20 mM HEPES with 3% sucrose) and mice are immunized three times at 14-day intervals. Fourteen days after the final injection, spleens and sera are collected. Serum from mice are frozen at −20° C. until evaluation. Suspensions of splenocytes are generated by disrupting the spleen capsule and gently pressing the contents through a 70 μm nylon cell strainer. Red blood cells are lysed by the addition of red cell lysis buffer and after lysis, the splenocytes are washed twice in R10 (RPMI 1640 supplemented with L-glutamine (2 mM), HEPES (20 mM) (Corning, Corning, N.Y.), penicillin (100 U/ml) and streptomycin (100 μg/mL), and 10% fetal bovine serum. Splenocytes are assayed for cytokine production by ELISpot and flow cytometry.

### Enzyme-Linked Immunosorbent Spot (ELISpot) Assay

HPV E6 and HPV E7 specific interferon-γ (IFN-γ) secreting T cells are determined by ELISpot assays using freshly isolated mouse splenocytes prepared as described above. The ELISpot assay is performed. Pools of overlapping peptides spanning the entire coding sequences of HPV E6 and HPV E7 are synthesized as 15-mers with 11-amino acid overlaps (and lyophilized peptide pools are dissolved in DMSO). Splenocytes (2×10⁵ cells) are stimulated with 2 μg/mL/peptide of overlapping 15-mer peptides in pools derived from E6 or E7. Cells are stimulated with Concanavalin A (Con A) at a concentration of 0.06 μg/per well as a positive control. Overlapping 15-mer complete peptide pools derived from SIV-Nef (AIDS Research and Reference Reagent Program, Division of AIDS, NIAID, NIH) are used as irrelevant peptide controls. The numbers of Spot Forming Cells (SFC) are determined using an Immunospot ELISpot plate reader, and results reported as the number of SFC per 10⁶ splenocytes.

### Intracellular Cytokine Stimulation

Splenocytes are prepared as described for the ELISpot assay above. Stimulation assays are performed using 10⁶ live splenocytes per well in 96-well U-bottom plates. Splenocytes in R10 media are stimulated by the addition of HPV E6, HPV E7, or SIV-Nef peptide pools at 2 μg/mL/peptide for 6 h at 37° C. in 5% CO₂, with protein transport inhibitor (GolgiStop, BD) added two hours after initiation of incubation. Stimulated splenocytes are stained for lymphocyte surface markers CD8a and CD4, fixed with paraformaldehyde, permeabilized, and stained for intracellular accumulation of IFN-γ and TNF-α. Fluorescent-conjugated antibodies against mouse CD8a (clone 53-6.7), CD4 (clone RM4-5), IFN-γ (clone XMG1.2), and TNF-α (clone MP6-XT22) are purchased from BD and staining is performed in the presence of anti-CD16/CD32 antibody (clone 2.4G2). Flow cytometry is performed using an Accuri C6 Flow Cytometer (BD) and analyzed using BD Accuri C6 Software.

### Tumor Immunotherapy

For in vivo tumor immunotherapy studies, female C57BL/6 mice, 8-10 weeks old, are implanted with 2×10⁵ TC-1 HPV E6/E7-expressing tumor cells SQ in the left flank. Mice are treated three times at 7-day intervals with SQ injections of 10¹⁰ VP Ad5 [E1-, E2b-]-E6, Ad5 [E1-, E2b-]-E7, or Ad5 [E1-, E2b-]-E6/E7. Control mice are injected with 10¹⁰ VP Ad5 [E1-, E2b-]-null under the same protocol. In combinational studies, mice are given 100 μg of rat anti-PD-1 antibody (clone RMP1-14) or an isotype rat control antibody (clone 2A3) IP at the same time as immunization. Rat anti-PD-1 antibody and rat IgG2a isotype control antibodies are purchased from BioXcell. Tumor size is measured by two opposing dimensions (a, b; e.g., a=tumor width and b=tumor length) and volume is calculated according to the formula V=(a²×b)/2 where a is the shorter dimension. Animals were euthanized when tumors reached 1500 mm³ or when tumors-became ulcerated.

### Analysis of Tumor-Infiltrating Cells (TILs) by Flow Cytometry

Four groups of 8-10 week old female C57BL/6 mice (n=5/group) are implanted with 2×10⁵ TC-1 tumor cells SQ in the left flank at day 0. Two of these groups are immunized SQ with 10¹⁰ VP Ad5 [E1-, E2b-]-null vector control and the other two groups SQ with 10¹⁰ VP of Ad5 [E1-, E2b-]-E6, Ad5 [E1-, E2b-]-E7, or Ad5 [E1-, E2b-]-E6/E7 vaccine. These immunizations are administered twice at 7-day intervals starting on day 12. In addition to immunizations, mice in one Ad5 [E1-, E2b-]-E6, Ad5 [E1-, E2b-]-E7, or Ad5 [E1-, E2b-]-E6/E7 group, and one Ad5 [E1-, E2b-]-null group are administered 100 μg rat anti-PD-1 antibody (clone RMP1-14) SQ at days 12 and 16 and 100 μg hamster anti-PD-1 antibody (clone J43) at days 19 and 23 to increase the effective dose of anti-PD-1 antibody. To control for treatment with these immune pathway checkpoint modulators, mice in the remaining Ad5 [E1-, E2b-]-E6, Ad5 [E1-, E2b-]-E7, or Ad5 [E1-, E2b-]-E6/E7 group, and Ad5 [E1-, E2b-]-null group are administered the relevant rat and hamster control IgG antibodies on the same days. Hamster anti-PD-1 antibody and isotype control are purchased from BioXcell. At day 27, tumors are measured, excised, and weighed. Tumors are minced and digested with a mixture of collagenase IV (1 mg/ml), hyaluronidase (100 μg/ml), and DNase IV (200 U/ml) in Hank's Balanced Salt Solution (HBSS) at room temperature for 30 min and rotating at 80 rpm. Enzymes are purchased from Sigma-Aldrich. After digestion, the tumor suspension is placed through a 70 μm nylon cell strainer and centrifuged. Red cells are removed by the addition of red cell lysis buffer (Sigma-Aldrich) and after lysis, the tumor suspensions are washed twice in phosphate buffered saline (PBS) containing 1% (w/v) bovine serum albumin and resuspended in fluorescent activated cell sorting (FACS) buffer (PBS pH 7.2, 1% fetal bovine serum, and 2 mM EDTA) for staining. Fluorescent-conjugated antibodies against CD45 (30-F111), CD4 (RM4-5), and PDL1 (MIH5) are purchased from BD. Fluorescent-conjugated antibodies against CD8(3 (H35-17.2), CD25 (PC61.5), FoxP3 (FJK-16s), PD-1 (RMP1-30), LAG-3 (C9B7W), and CTLA4 (UC10-4B9) were all purchased from eBioscience. Surface staining is performed for 30 minutes at 4° C. in 100 L FACS buffer containing anti-CD16/CD32 antibody (clone 2.4G2). Stained cells are washed in FACS buffer, fixed with paraformaldehyde, and (if needed) permeabilized in permeabilization buffer (eBioscience) before staining with fluorescent-conjugated anti-FoxP3 antibody or anti-CTLA4 antibody for 60 minutes at 4° C. in 100 μL permeabilization buffer containing anti-CD16/CD32 antibody (clone 2.4G2). Cells are washed with permeabilization buffer, washed back into FACS buffer, and a fixed volume of each sample is analyzed by flow cytometry using a BD Accuri C6 flow cytometer. Tumor cells are defined as CD45⁻ events in a scatter gate that includes small and large cells. CD4⁺ TILs are defined as CD45⁺/CD4⁺ events in a lymphocyte scatter gate. CD8⁺ TILs are defined as CD45⁺/CD8β⁺ events in a lymphocyte scatter gate. Regulatory T cells (Tregs) are defined as CD45⁺/CD4⁺/CD25⁺/FoxP3⁺ events in a lymphocyte scatter gate. Effector CD4⁺ T cells are defined as CD45+/CD4+/CD25-/FoxP3⁻ events in a lymphocyte scatter gate. Isotype-matched control antibodies are used to determine positive expression of FoxP3, PDL1, PD-1, LAG-3, and CTLA4. Flow cytometry is performed using an Accuri C6 Flow Cytometer (BD) and analyzed in BD Accuri C6 Software.

### HPV E6/E7 Specific Cell-Mediated Immune Responses Induced by Ad5 [E1-, E2b-]-E6/E7

A study is performed to determine the effect of increasing doses of Ad5 [E1-, E2b-]-E6, Ad5 [E1-, E2b-]-E7, or Ad5 [E1-, E2b-]-E6/E7 immunizations on the induction of CMI responses in mice. Groups of C57BL/6 mice (n=5/group) are immunized SQ three times at 14-day intervals with 10⁸, 10⁹, or 10¹⁰ VP Ad5 [E1-, E2b-]-E6/E7. Control mice receive 10⁸ VP, 10⁹ VP, or 10¹⁰ VP Ad5 [E1-, E2b-]-null (empty vector controls). Two weeks after the last immunization, splenocyte CMI responses are assessed by ELISpot analysis for IFN-γ secreting cells. Intracellular accumulation of IFN-γ and TNF-α in both CD8α⁺ and CD4⁺ splenocytes populations is also determined in mice immunized with 10¹⁰ VP of Ad5 [E1-, E2b-]-E6, Ad5 [E1-, E2b-]-E7 or Ad5 [E1-, E2b-]-E6/E7.

### Treatment of HPV E6/E7-Expressing Tumors

The anti-tumor effect of immunotherapy treatment in mice bearing HPV E6/E7 TC-1 tumors is studied. Two groups of C57BL/6 mice (n=5/group) are inoculated with 2×10⁵ TC-1 tumor cells SQ into the right subcostal area on day 0. On days 1, 8, and 14 mice are treated by SQ injections of 10¹⁰ VP Ad5 [E1-, E2b-]-null (vector control) or 10¹⁰ VP of Ad5 [E1-, E2b-]-E6, Ad5 [E1-, E2b-]-E7, or Ad5 [E1-, E2b-]-E6/E7. All mice are monitored for tumor size and tumor volumes calculated.

To determine if immunotherapy with Ad5 [E1-, E2b-]-E6, Ad5 [E1-, E2b-]-E7, or Ad5 [E1-, E2b-]-E6/E7 is effective against larger tumors, TC-1 tumor cells are implanted in two groups of C57BL/6 mice (n=4/group) and then delayed weekly treatment with Ad5 [E1-, E2b-]-E6, Ad5 [E1-, E2b-]-E7, or Ad5 [E1-, E2b-]-E6/E7 for 6 days post tumor implantation, at a time when tumors are expected to be small but palpable.

Finally, to determine if immunotherapy with Ad5 [E1-, E2b-]-E6, Ad5 [E1-, E2b-]-E7, or Ad5 [E1-, E2b-]-E6/E7 is effective against large established tumors, TC-1 tumor cells are implanted in two groups of C57BL/6 mice (n=4/group) then delayed weekly treatment with Ad5 [E1-, E2b-]-E6, Ad5 [E1-, E2b-]-E7, or Ad5 [E1-, E2b-]-E6/E7 until 13 days post tumor implantation, when tumors are expected to be −100 mm³.

### Example 5

### Induction of Immune Responses to HPV E6 and/or HPV E7 Agonist Epitope Variants

This example shows that the Ad5 [E1-, E2b-]-E6, Ad5 [E1-, E2b-]-E7, or Ad5 [E1-, E2b-]-E6/E7 products containing various agonist epitopes can be evaluated for the ability to induce immunotherapeutic responses in a similar fashion.

### Tumor Microenvironment Following Combination Immunotherapy

To analyze cell populations that contribute to delayed tumor growth and survival in Ad5 [E1-, E2b-]-E6, Ad5 [E1-, E2b-]-E7, or Ad5 [E1-, E2b-]-E6/E7 treated mice, tumor-infiltrating lymphocytes (TILs) are assessed by flow cytometry. Four groups of mice are implanted with 2×10⁵ TC-1 cells and began treatment 10 days later with two weekly immunizations of Ad5 [E1-, E2b-]-E6, Ad5 [E1-, E2b-]-E7, or Ad5 [E1-, E2b-]-E6/E7 plus anti-PD-1 antibody. On day 27 whole tumors are collected and processed as described in the materials and methods.

To further study if there exists synergistic/additive effect of anti-PD-1 antibody to Ad5 [E1-, E2b-]-E6, Ad5 [E1-, E2b-]-E7, or Ad5 [E1-, E2b-]-E6/E7 immunotherapy, the expression of PD-1, LAG-3, and CTLA-4 are examined on TILs.

### Example 6

### Clinical Trial of HPV E6 and/or HPV E7 Agonist Epitope Variant Vaccines

This example describes the use of Ad5 [E1-, E2b-]-E6, Ad5 [E1-, E2b-]-E7, or Ad5 [E1-, E2b-]-E6/E7 products containing various agonist epitopes for evaluation of safety and immunogenicity of related immunizations in subjects that are human papilloma virus type 16 (HPV-16) positive, in subjects with HPV-associated head and neck squamous cell carcinoma (HNSCC), and in subjects with HPV-associated cervical cancer.

Current interventions in HNSCC patients include therapy with cisplatin and radiation or cetuximab and radiation. However, many HNSCC patients that initially respond or do not respond ultimately relapse. The Ad5 [E1-, E2b-]-E6, Ad5 [E1-, E2b-]-E7, or Ad5 [E1-, E2b-]-E6/E7 vaccine is designed to induce anti-tumor T cell-mediated immune responses directed against the early 6 (E6) and early 7 (E7) genes of HPV. One of the important features of the Ad5 [E1-, E2b-]-E6, Ad5 [E1-, E2b-]-E7, or Ad5 [E1-, E2b-]-E6/E7 vaccine is that it can be combined with chemotherapy/radiation treatment.

The resulting recombinant replication-defective vector can be propagated in the newly engineered, proprietary human 293 based cell line (E.C7) that supplies the E1 and E2b gene functions in trans required for vector production.

Ad5 [E1-, E2b-]-E6, Ad5 [E1-, E2b-]-E7, or Ad5 [E1-, E2b-]-E6/E7 vaccine product is used to induce HPV E6 and/or HPV E7 specific cell-mediated immune responses in a safe and effective manner in subjects. An open-label, dose-escalation clinical study is conducted to evaluate the safety and immunogenicity of Ad5 [E1-, E2b-]-E6, Ad5 [E1-, E2b-]-E7 or Ad5 [E1-, E2b-]-E6/E7 vaccine injections. The dosage levels to be evaluated are 5×10¹⁰, 1×10¹¹ and 5×10¹¹ virus particles (VP) of Ad5 [E1-, E2b-]-E6/E7. Subjects are enrolled into successive increasing dosage levels involving three (3) cohorts of subjects that are monitored for dose-limiting toxicity (DLT). Each subject is given Ad5 [E1-, E2b-]-E6, Ad5 [E1-, E2b-]-E7, or Ad5 [E1-, E2b-]-E6/E7 vaccine by SQ injection every 3 weeks for 3 immunizations. Assessment of DLT for dose escalation is made after all subjects in a cohort have had a study visit at least 3 weeks after receiving their last dose of vaccine.

The subjects are animals, such as humans, non-human primates (e.g., rhesus or other types of macaques), mice, pigs, horses, donkeys, cows, sheep, rats, or fowls.

Induction of CMI Responses after Ad5 [E1-, E2b-]-E6, Ad5 [E1-, E2b-]-E7 or Ad5 [E1-, E2b-]-E6/E7 Vaccination as Assessed by Flow Cytometry

To assess CMI induction by flow cytometry following multiple homologous immunizations with Ad5 [E1-, E2b-]-E6, Ad5 [E1-, E2b-]-E7, or Ad5 [E1-, E2b-]-E6/E7 vaccine, groups of C57Bl/6 mice (n=5/group) are immunized three times SQ at 2-week intervals with 10¹⁰ VP of Ad5 [E1-, E2b-]-E6, Ad5 [E1-, E2b-]-E7, or Ad5 [E1-, E2b-]-E6/E7 vaccine. Two weeks following the last immunization, splenocytes are exposed to HPV E6 and/or HPV E7 peptides or irrelevant antigens and analyzed by flow cytometry for the number of IFN-γ and/or TNFα expressing T cells.

### Toxicology

An extensive pre-clinical toxicology study is conducted to assess the toxicity of Ad5 [E1-, E2b-]-E6, Ad5 [E1-, E2b-]-E7 or Ad5 [E1-, E2b-]-E6/E7 agonist epitope variant vaccine following SQ injections on in C57Bl/6 mice. Toxicity endpoints are assessed at various time points post-injection. The animals are administered with up to 3 SQ injections on days 1, 22, and 43, with either vehicle control or Ad5 [E1-, E2b-]-E6, Ad5 [E1-, E2b-]-E7, or Ad5 [E1-, E2b-]-E6/E7 vaccine at a dose consistent with that to be used in clinical trials accounting for difference in body mass. Evaluations consist of effects on body weights, body weight gain, food consumption pathology, blood hematology analyses, blood chemistry analyses, and test on coagulation time.

Treatment of Established HPV E6/E7-Expressing Tumors with Vaccine Alone

The effectiveness of treating established HPV E6 and/or HPV E7 expressing tumors in vivo with Ad5 [E1-, E2b-]-E6, Ad5 [E1-, E2b-]-E7, or Ad5 [E1-, E2b-]-E6/E7 vaccine is evaluated. C57Bl/6 mice are implanted SQ into the right subcostal with 10⁶ HPV E6 and/or HPV E7 expressing tumor cells on day 0. Tumors are expected to be palpable by days 4-6. On days 6, 13, and 20, mice are treated by SQ injections of 10¹⁰ VP of Ad5 [E1-, E2b-]-null (empty vector controls) or 10¹⁰ VP of Ad5 [E1-, E2b-]-E6, Ad5 [E1-, E2b-]-E7, or Ad5 [E1-, E2b-]-E6/E7 vaccine. All mice are monitored for tumor growth and tumor volumes calculated.

The results of this clinical study establish the safety and immunogenicity of using the new Ad5 [E1-, E2b-]-E6, Ad5 [E1-, E2b-]-E7, or Ad5 [E1-, E2b-]-E6/E7 agonist epitope variant vaccines.

### Example 7

### Clinical Trial of Ad5 [E1-, E2b-]-E6/E7 Vaccine in HPV-Positive Individuals

This example describes the evaluation of safety and immunogenicity of immunizations with the Ad5 [E1-, E2b-]-E6/E7 vaccine in subjects that are HPV-positive to eliminate or destroy HPV E6 and/or HPV E7 expressing cells.

The vaccine is designed to induce T cell-mediated immune responses directed against the early 6 (E6) and early 7 (E7) genes of HPV. The backbone of the vaccine is an adenovirus serotype 5 (Ad5) vector that has been modified by removal of the E1, E2b, and E3 genes and insertion of a modified fused non-oncogenic HPV E6/E7 gene. The resulting recombinant replication-defective vector can only be propagated in the newly engineered, proprietary human 293 based cell line (E.C7) that supplies the E1 and E2b gene functions in trans required for vector production.

The vaccine product is used to induce HPV E6 and/or HPV E7 specific cell-mediated immune responses in a safe and effective manner in subjects. An open-label, dose-escalation clinical study is conducted to evaluate the safety and immunogenicity of Ad5 [E1-, E2b-]-E6/E7 vaccine injections. Subjects are enrolled into successive increasing dosage levels involving three (3) cohorts of subjects that are monitored for dose-limiting toxicity (DLT). Each subject is given Ad5 [E1-, E2b-]-E6/E7 vaccine by subcutaneous injection. Assessment of DLT for dose escalation is made after all subjects in a cohort have had a study visit at least 3 weeks after receiving their last dose of vaccine. The Ad5 backbone expressing HPV E6/E7 is used for the immunization (vaccination) of subjects that are HPV positive.

A clinical study is also conducted to assess the efficacy of the Ad5 [E1-, E2b-]-E6/E7 vaccine in subjects that are HPV positive but do not have HPV-associated cancer to eliminate or destroy HPV E6 and/or HPV E7 expressing cells. Subjects are enrolled into a study where they are given the Ad5 [E1-, E2b-]-E6/E7 vaccine by subcutaneous injection. Subjects are monitored to evaluate temporal cellular and humoral responses to vaccination against the HPV E6 and E7 genes.

Subjects are vaccinated with the Ad5 [E1-, E2b-]-E6/E7 vaccine of the present disclosure in order to eliminate or destroy HPV E6- and/or HPV E7-expressing cells in HPV positive subjects. The subjects are animals, such as humans, non-human primates (e.g., rhesus or other types of macaques), mice, pigs, horses, donkeys, cows, sheep, rats, or fowls.

### Example 8

### Clinical Trial of Ad5 [E1-, E2b-]-E6/E7 Agonist Epitope Variant Vaccines in HPV-Positive Individuals

This example describes the use of Ad5 [E1-, E2b-]-E6, Ad5 [E1-, E2b-]-E7, or Ad5 [E1-, E2b-]-E6/E7 products containing various agonist epitopes for evaluation of safety and immunogenicity of related immunizations in subjects that are HPV-positive to eliminate or destroy HPV E6/E7 expressing cells.

The vaccine is designed to induce T cell-mediated immune responses directed against the early 6 (E6) and early 7 (E7) genes of HPV. The backbone of the vaccine is an adenovirus serotype 5 (Ad5) vector that has been modified by removal of the E1, E2b, and E3 genes, and insertion of a modified fused non-oncogenic HPV E6/E7 gene. The resulting recombinant replication-defective vector can only be propagated in the newly engineered, proprietary human 293 based cell line (E.C7) that supplies the E1 and E2b gene functions in trans required for vector production.

Ad5 [E1-, E2b-]-E6, Ad5 [E1-, E2b-]-E7, or Ad5 [E1-, E2b-]-E6/E7 vaccine product is used to induce HPV E6 and/or HPV E7 specific cell-mediated immune responses in a safe and effective manner in subjects that are HPV negative. An open-label, dose-escalation clinical study is conducted to evaluate the safety and immunogenicity of Ad5 [E1-, E2b-]-E6, Ad5 [E1-, E2b-]-E7, or Ad5 [E1-, E2b-]-E6/E7 vaccine injections. Subjects are enrolled into successive increasing dosage levels involving three (3) cohorts of subjects that are monitored for dose-limiting toxicity (DLT). Each subject is given Ad5 [E1-, E2b-]-E6, Ad5 [E1-, E2b-]-E7, or Ad5 [E1-, E2b-]-E6/E7 vaccine by SQ injection every 3 weeks for 3 immunizations. Assessment of DLT for dose escalation is made after all subjects in a cohort have had a study visit at least 3 weeks after receiving their last dose of vaccine.

A clinical study is also conducted to assess the efficacy of the Ad5 [E1-, E2b-]-E6, Ad5 [E1-, E2b-]-E7, or Ad5 [E1-, E2b-]-E6/E7 vaccines in subjects that are HPV-positive but do not have HPV-associated cancer to eliminate or destroy HPV E6 and/or HPV E7 expressing cells. Subjects are enrolled into a study where they are given the Ad5 [E1-, E2b-]-E6, Ad5 [E1-, E2b-]-E7, or Ad5 [E1-, E2b-]-E6/E7 vaccines by subcutaneous injection. Subjects are monitored to evaluate temporal cellular and humoral responses to vaccination against the HPV E6 and/or HPV E7 genes.

Subjects are vaccinated with the Ad5 [E1-, E2b-]-E6, Ad5 [E1-, E2b-]-E7, or Ad5 [E1-, E2b-]-E6/E7 vaccines of the present disclosure in order to eliminate or destroy HPV E6 and/or HPV E7 expressing cells in HPV-positive subject. Subjects are mammals, such as humans or mice.

The subjects are animals, such as humans, non-human primates (e.g., rhesus or other types of macaques), mice, pigs, horses, donkeys, cows, sheep, rats, or fowls.

### Example 9

### Phase I/Ib Study to Evaluate the Safety and Immunogenicity of Ad5 [E1-, E2b-]-HPV16-E6Δ/E7Δ in Healthy Individuals HPV-16 Positive by Oral Rinse or Pap Smear Samples

This example describes a Phase I/Ib trial evaluating the safety and immunogenicity of Ad5 [E1-, E2b-]-HPV16-E6Δ/E7Δ immunization in healthy individuals that are HPV-16 positive by oral rinse or pap smear samples. The study is conducted in two parts: the first part involves dose escalation using a 6 patient incremental design, and the second part involves the expansion of the maximum tolerated dose (MTD) or highest tested dose (HTD) (and MTD or HTD-1) to further evaluate safety, preliminary efficacy, and immunogenicity.

In the first part, 6 subjects are sequentially enrolled in each cohort. Subjects are assessed for dose-limiting toxicities (DLTs) at the following dosages: Cohort 1: 5×10⁹ virus particles (VP); Cohort 2: 5×10¹⁰ VP; and Cohort 3: 5×10¹¹ VP.

Dose expansion occurs when the MTD or HTD is determined. An additional 28 subjects are enrolled in the dose expansion component of the trial, for a total of 46 subjects.

Up to 46 subjects are enrolled in the study. In the dose escalation component, 6 subjects are sequentially enrolled starting at Cohort 1. In the dose expansion component (i.e., once the MTD or HTD is identified), an additional 28 subjects are enrolled for a total of 46 subjects in the MTD/HTD cohort to obtain further safety, preliminary efficacy, and immunogenicity data.

### Subject Inclusion Criteria

Subjects are selected for inclusion in the study based on one or more of the following criteria. Individuals are healthy and have an age >18, have been documented as HPV-16 positive as determined by oral rinses or pap smears, and/or have adequate hematologic function as measured by a white blood cell (WBC) count ≥3000/microliter, hemoglobin ≥9 g/dL, and platelets ≥75,000/microliter are eligible for inclusion in the study. Individuals with adequate renal and hepatic function as measured by a serum creatinine level <2.0 mg/dL, bilirubin <1.5 mg/dL (except for Gilbert's syndrome which will allow bilirubin ≤2.0 mg/dL), and ALT and AST levels ≤2.5 times the upper limit of normal and female individuals are either of non-child-bearing potential or use effective contraception are also eligible for inclusion in the study.

### Subject Exclusion Criteria

Subjects are excluded from the study based on one or more of the following criteria. Individuals who have an autoimmune disease, active hepatitis, HIV infection, or any serious intercurrent chronic or acute illness, pregnant women and nursing mothers, and/or individuals currently using any medications with known immunosuppressive effect including systemic intravenous or oral corticosteroid therapy are ineligible for the study. Individuals who are currently participating in a study using an investigational drug or device, have received any live-virus vaccine within 30 days prior to study entry, and/or have cervical dysplasia >CIN 1 or oropharyngeal lesions concerning for malignancy are also ineligible for the study.

### Study Design

This is a phase I/Ib clinical investigational study designed to test dosing, safety, and immunogenicity of the Ad5 [E1-, E2b-]-HPV16-E6^{Δ}/E7^{Δ} vaccine product in healthy individuals that are HPV-16 positive. The study involves up to three (3) cohorts of six (6) patients each in phase I that test escalating doses of the Ad5 [E1-, E2b-]-HPV16-E6^{Δ}/E7^{Δ} vaccine. In phase Ib, additional patients are tested, up to a total of 20 at the MTD, and 20 at MTD-1.

### Phase I

For Cohort 1, six patients receive Ad5 [E1-, E2b-]-HPV16-E6^{Δ}/E7^{Δ} at a dose of 5×10⁹ virus particles (VP) every 3 weeks for 3 immunizations. Enrollment of subjects is staggered such that the first immunization for each subject is separated by at least 1 week from the next subject, to allow for monitoring for adverse events in the prior subject. Assessment of dose-limiting toxicities (DLT) for dose escalation is made at least 2 weeks after the last patient in this cohort has received their last dose of vaccine. If there is ≤1/6 DLT, then patients begin enrolling into Cohort 2. If there are ≥2/6 DLTs in the first six subjects, the study is reevaluated, including the potential for lowering the initial dose further.

A DLT is defined as any of the following events. Subjects who exhibit a Grade 2 or higher allergic or immediate hypersensitivity reaction, a Grade 2 or higher autoimmune toxicity (with the exception of vitiligo and isolated laboratory abnormalities related to the thyroid not requiring medical intervention), and/or a Grade 2 or higher neurological toxicity are categorized as having experienced a DLT. Any subject who exhibits a Grade 3 or 4 major organ toxicity, a Grade 3 (ulceration, or necrosis) or higher injection site reaction, and/or a Grade 4 fever are also categorized as having experienced a DLT.

For Cohort 2, six patients receive Ad5 [E1-, E2b-]-HPV16-E6^{Δ}/E7^{Δ} at a dose of 5×10¹⁰ VP every 3 weeks for 3 immunizations. Enrollment of subjects is staggered such that the first immunization for each subject is separated by at least 1 week from the next subject, to allow for monitoring for adverse events in the prior subject. Assessment of DLT for dose escalation is made at least 2 weeks after the last patient in this cohort has received their last dose of vaccine. If there is ≤1/6 DLT, then patients begin enrolling into cohort 3. If ≥2/6 experience a DLT, then the MTD is defined as the next lowest dose (dose level #1) (5×10⁹ VP), and patients begin enrolling into Phase 1b at that dose level.

For Cohort 3, six patients receive Ad5 [E1-, E2b-]-HPV16-E6^{a}/E7Δ at a dose of 5×10¹¹ VP every 3 weeks for 3 immunizations. Enrollment of subjects is staggered such that the first immunization for each subject is separated by at least 1 week from the next subject, to allow for monitoring for adverse events in the prior subject. Assessment of DLT is made at least 2 weeks after the last patient in this cohort has received their last dose of vaccine. If there is ≤1/6 DLT, then the HTD is defined as 5×10¹¹ VP and patients begin enrolling into Phase Ib that dose level (dose level #3) and the next lower dose level (dose level #2). If ≥2/6 experience a DLT, then the MTD is defined as next lower dose (dose level #2) (5×10¹⁰ VP), and patients begin enrolling into Phase 1b at that dose level (dose level #2), and the next lower dose level (dose level #1).

Dose escalation is performed as shown in TABLE 4. No intra-patient dose escalations are permitted.

TABLE 4
Dose Levels

| Cohort | Ad5 [E1-, E2b-]-HPV16-E6^{Δ}/E7^{Δ} (VP) |
|---|---|
| 1 | 5 × 10⁹ |
| 2 | 5 × 10¹⁰ |
| 3 | 5 × 10¹¹ |

### Phase Ib

After an initial MTD or HTD is determined, subjects begin enrolling into two expansion cohorts, in the two highest tolerated dose cohorts. A total of 20 subjects are enrolled for each of 2 specific dose levels, these 20 include 6 from initial dose escalation in phase 1, plus a further 14 from phase 1b. Safety, immunogenicity, and anti-viral activity are assessed.

For Cohort 4, additional patients (N=14) receive Ad5 [E1-, E2b-]-HPV16-E6^{Δ}/E7^{Δ} at the MTD/HTD (−1) every 3 weeks for 3 immunizations. If there are ≥4 DLTs (out of 20 subjects at that specific dose level), this cohort stops enrollment, and the next lower dose begins enrolling, (if this cohort was dose level #1, the study is stopped and reevaluated).

For Cohort 5, additional patients (N=14) receive Ad5 [E1-, E2b-]-HPV16-E6^{Δ}/E7^{Δ} at the MTD/HTD every 3 weeks for 3 immunizations. If there are ≥4 DLTs (out of 20 subjects at the specific dose level), this cohort stops enrollment, and the next lower dose is now defined as the MTD.

Ad5 [E1-, E2b-]-HPV16-E6^{Δ}/E7^{Δ}

The investigational product is a non-replicating recombinant adenovirus serotype (Ad5) containing non-oncogenic early 6 (E6) and early 7 (E7) genes of HPV16 and is referred to as Ad5 [E1-, E2b-]-HPV16-E6^{Δ}/E7^{Δ}. The Ad5 [E1-, E2b-] vector is non-replicating and its genome does not integrate into the human genome. The study drug is described in TABLE 5.

TABLE 5
Investigational Product

| | |
|---|---|
| Product Name | Ad5 [E1-, E2b]-HPV16-E6^{Δ}/E7^{Δ} |
| Dosage Form | Supplied as a Frozen Liquid |
| Unit Dose | 5 × 10⁹ VP, 5 × 10¹⁰ VP, or 5 × 10¹¹ VP |
| Route of Administration | SQ injection |
| Physical Description | Ad5 [E1-, E2b-]-HPV16-E6Δ/E7Δ vaccine |
| | is supplied as a sterile, clear solution in a |
| | 2 mL single-dose vial. The vaccine is |
| | provided at a concentration of 5 × 10¹¹ VP |
| | per 1 mL), and contains ARM formulation |
| | buffer (20 mM TRIS, 25 mM NaCl, |
| | 2.5% glycerol, pH 8.0). Each vial contains |
| | approximately 0.7 mL of vaccine. The |
| | product should be stored at ≤−20° C. |
| Manufacturer | LONZA |

Ad5 [E1-, E2b-]-HPV16-E6^{Δ}/E7^{Δ} Dose Preparation and Administration

The dose of Ad5 [E1-, E2b-]-HPV16-E6^{Δ}/E7^{Δ} to be injected is 5×10⁹ VP (Cohort 1) per 1 mL, 5×10¹⁰ VP (Cohort 2) per 1 mL, or 5×10¹¹ VP (Cohort 3) per 1 mL. Prior to injection, the appropriate vial is from the freezer and allowed to thaw at controlled room temperature (20-25° C., 68-77° F.) for at least 20 minutes and not more than 30 minutes, after which it is kept at 2-8° C. (35-46° F.).

Each vial is sealed with a rubber stopper and has a white flip-off seal. The end user of the product flips the white plastic portion of the cap up/off with their thumb to expose the rubber stopper and then punctures the stopper with an injection needle to withdraw the liquid. The rubber stopper is secured to the vial with an aluminum-crimped seal.

The thawed vial is swirled and then, using aseptic technique, the pharmacist withdraws the appropriate volume from the appropriate vial using a 1 mL syringe.

The vaccine dose is injected as soon as possible using a 1 to ½ inch, 20 to 25-gauge needle. If the vaccine cannot be injected immediately, the syringe is returned to the pharmacy and properly disposed in accordance with institutional policy and procedure, and disposition must be recorded on the investigational product accountability record.

Storage of the vaccine in the vial at 2-8° C. (35-46° F.) does not exceed 8 hours. Once the vaccine has been thawed, it is not refrozen.

For dose preparation of the 5×10¹¹ virus particles, 1 mL of contents from the vial is withdrawn, injection site is prepared with alcohol, and administration to the subject by SC injection in the thigh is carried out without any further manipulation.

For dose preparation of the 5×10¹⁰ virus particles, from a 5.0-mL vial of 0.9% sterile saline, 0.50 mL of fluid is removed using a 1.0 mL tuberculin syringe, leaving 4.50 mL. Then, using another 1.0 mL tuberculin syringe, 0.50 mL is removed from the vial labeled Ad5 [E1-, E2b-]-HPV16-E6^{Δ}/E7^{Δ}, and delivered into the 4.5 mL of sterile saline remaining in the 5-mL sterile saline vial. The contents are mixed by inverting the 5 mL solution of diluted Ad5 [E1-, E2b-]-HPV16-E6^{Δ}/E7^{Δ}. 1 mL of the diluted Ad5 [E1-, E2b-]-HPV16-E6^{Δ}/E7^{Δ} is withdrawn, the injection site is prepared with alcohol, and administration to the subject by SQ injection in the thigh is carried out.

For dose preparation of the 5×10⁹ virus particles, from a 5.0-mL vial of 0.9% sterile saline, 0.05 mL of fluid is removed using a 0.50 mL tuberculin syringe, leaving 4.95 mL. Then, using another 0.50 mL tuberculin syringe, 0.05 mL is removed from the vial labeled Ad5 [E1-, E2b-]-HPV16-E6^{Δ}/E7^{Δ}, and delivered into the 4.95 mL of sterile saline remaining in the 5-mL sterile saline vial. The contents are mixed by inverting the 5 mL of diluted Ad5 [E1-, E2b-]-HPV16-E6^{Δ}/E7Δ. 1 mL of the diluted Ad5 [E1-, E2b-]-HPV16-E6^{Δ}/E7^{Δ} is withdrawn, the injection site is prepared with alcohol, and administration to the subject by SQ injection in the thigh is carried out.

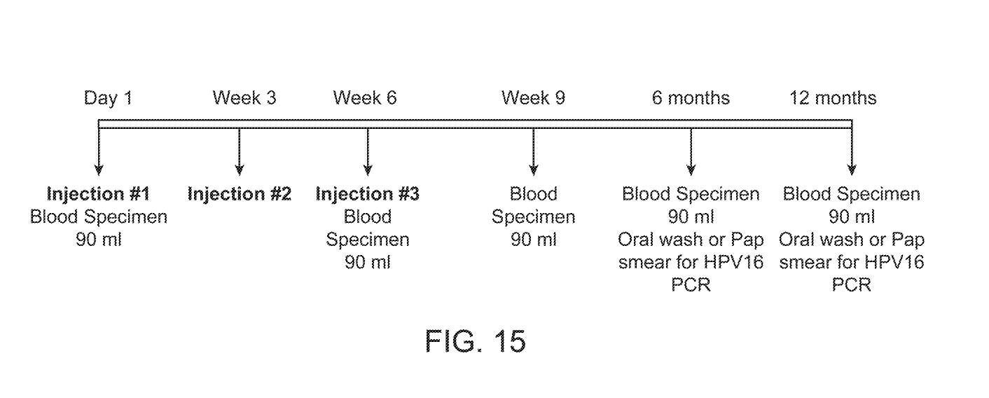

Ad5 [E1-, E2b-]-HPV16-E6^{Δ}/E7^{Δ} is administered on Day 1, Week 3, and Week 6 for a total of three injections (FIG. 15). All injections of the vaccine are given as a volume of 1 mL by SC injection in the thigh after preparation of the site with alcohol. Either thigh is used for the initial injection. Subsequent injections are given in the same thigh as the initial injection and are separated by at least 5 cm.

### Treatment Period Procedures and Evaluations

Ad5 [E1-, E2b-]-HPV16-E6^{Δ}/E7^{Δ} is administered on Day 1, Week 3 and Week 6 for a total of three injections. All study drug administration treatment occur within ±7 days of the planned visit date. Subjects are considered enrolled on Study Day 1 when the study drug is first administered.

Females of child-bearing potential undergo a urine pregnancy test prior to each injection. Subjects remain in the clinic for a minimum of 30 minutes after the first injection and 30 minutes after subsequent injections to allow for the evaluation of vital signs and for monitoring of injection site reactions. For the first injection, vital signs are assessed 30 minutes after the injection.

The subjects are provided patient diaries, a ruler, and a thermometer to monitor site reactions, temperature, and adverse events (Aes). The clinic staff contact subjects by telephone 72 hours following each injection to assess any constitutional symptoms.

### Exploratory Pharmacodynamic Assessments

Approximately 90 mL of the subject's peripheral blood is drawn at specific time points during the study. Blood is only drawn at month 6 or 12 if there is evidence of immune response at week 6 or 9 (FIG. 15). Subjects undergo a repeat Pap smear or oral rinse with HPV16 testing at 6 months and 12 months (+/−1 month) after the initial immunization.

### Sample Analysis

For the ELISpot Analysis, antigen specific CMI and cytolytic T lymphocyte (CTL) activity is assessed using an ELISpot assay. The CMI activity of T cells against HPV16-E6^{Δ}/E7^{Δ} is assessed by re-stimulating PBMCs with purified HPV16-E6^{Δ}/E7^{Δ} peptides and the numbers of IFN-γ secreting spot forming cells (SFC) determined. The CTL activity of cells against HPV16-E6^{Δ}/E7^{Δ} is assessed using a granzyme B ELISPOT assay that is an accepted test to measure functional CTLs. PBMCs are re-stimulated with purified HPV16-E6^{Δ}/E7^{Δ} peptides and the numbers of granzyme B secreting spot forming cells (SFC) determined. CMI responses are considered positive if ≥50 SFC are detected per 10⁶ cells after subtraction of the negative control and SFC are ≥2-fold higher than those observed in the negative control wells. Patient CMI responses in each cohort are determined at baseline, at 4-weeks after the 3^{rd} immunization, and at months 6 and 12 after the first immunization. Statistical analyses comparing immune responses (number of SFC) at each sampling point are performed employing the Student T tests and/or Mann-Whitney tests (PRISM, Graph Pad).

For the Flow Cytometry Analyses, to assess CD4+ and CD8+ T cell responses, PMBC samples from individual patients are assayed for IFN-γ and/or tumor necrosis factor alpha (TNF-α) expression using Flow Cytometry and intracellular cytokine staining methods. Briefly, 10⁶ PBMC cells/well are incubated 6 hours with 2.0 μg/ml HPV16-E6^{Δ}/E7^{Δ} peptide pools, 2.0 fig/ml SIV nef negative control peptide pool, or media alone. A protein transport inhibitor (GolgiStop) is added for the final 4 hours of the stimulation. After stimulation, cells are stained for CD4 and CD8, fixed, permeabilized, stained for IFN-γ and TNF-α, and then analyzed by flow cytometry. Statistical analyses comparing immune responses (percent IFN-γ and/or TNF-α expressing cells) at each sampling point are performed employing the Student T tests and/or Mann-Whitney tests (PRISM, Graph Pad).

Antibody Responses: Serum IgG antibody (Ab) responses to HPV16-E6^{Δ}/E7^{Δ} is measured employing a previously described quantitative ELISA technique using purified E6 and E7 proteins and Ad5 neutralizing antibody (NAb) is determined and reported as the inverse of the endpoint Ad5 NAb titer. Statistical analyses comparing immune responses at each sampling point (baseline, at each immunization, at 3-weeks after the 3^{rd} immunization, is performed employing the Student T tests and/or Mann-Whitney tests (PRISM, Graph Pad).

### Statistical Power Assumptions

For ELIspot CMI determinations, assuming a minimum activity of 50 (±10 SD) spot forming cells (SFC) from baseline PBMC samples (N=20) versus a minimum increase to 100 (±25 SD) SFC for PBMC samples (N=10) taken at time points during and after immunizations, the statistical power is >99% for a 95% confidence interval (two-tail test).

For flow cytometry studies, assuming a background level of approximately 0.5% (+0.5 SD) CD4+ or CD8+ IFN-γ and/or TNF-α expressing lymphocytes from baseline PBMC samples (N=10) versus a minimum increase to a level of 1.0% (±0.5 SD) CD4+ or CD8+ IFN-γ and/or TNF-α expressing lymphocytes for PBMC samples (N=10) taken at time points during and after immunizations, the statistical power is 88.5% for a 95% confidence interval (two-tail test).

For serum HPV16-E6^{Δ}/E7^{Δ} antibody determinations, assuming patient samples (N=10) have existing antibody levels of 10 nanogram IgG equivalents of Ab/ml (±5 SD) in baseline serum samples and these antibody levels increase to at least 15 nanogram IgG equivalents of Ab/ml (±5 SD) in serum samples taken at time points during and after immunizations, the statistical power is 88.5% for a 95% confidence interval (two-tail test).

For Ad5 neutralizing antibody (NAb) determinations, assuming patients (N=10) have pre-existing Ad5 immunity with inverse Ad5 NAb titer levels of 200 (±100 SD) and these levels increase to at least 400 (±100 SD) in serum samples (N=10) taken at time points during and after immunizations, the statistical power is >99% for a 95% confidence interval (two-tail test).

### Safety Analysis

DLTs are evaluated continuously in a cohort. An overall assessment of whether to escalate to the next dose level is made at least 3 weeks after the last subject in the previous cohort has received their first injection. A dose level is considered safe if <20% of subjects treated at a dose level experience a DLT (i.e., 0 of 3, ≤1 of 6, or ≤4 of 20 subjects). Safety is evaluated in 6 subjects at each dose level in the dose escalation component of the study. Safety continues to be monitored among additional subjects treated at the MTD or HTD in the dose expansion component of the study. A subject is considered evaluable for safety if treated with at least one injection. DLTs are observed through 9 weeks to accommodate the safety evaluation of multiple doses of Ad5 [E1-, E2b-]-HPV16-E6^{Δ}/E7^{Δ}.

Overall safety is assessed by descriptive analyses using tabulated frequencies of AEs by grade using CTCAE Version 4.0 within dose cohorts and for the overall study population in terms of treatment-emergent AEs, serious adverse events (SAEs), and clinically significant changes in safety laboratory tests, physical examinations, and vital signs.

### Exploratory Immune Response Analysis

The percentage of subjects with a positive immune response are evaluated by dose cohorts and overall. A positive immune response is defined by CMI reactivity in ex vivo stimulation assays, with flow cytometric readout (cytokine production or CD107 expression). Antigen-specific peptide challenge assays require a readout of >250 reactive T-cells/million cells above the background.

Immune response is assessed among the 20 subjects treated at the (MTD/HTD), and 20 subjects treated at the (MTD/HTD-1), (6 in dose escalation and 14 in dose expansion). The magnitude of response is described. A subject is considered evaluable for immune response if they receive at least three injections.

### Efficacy Analysis

The percentage of subjects that achieve a negative HPV-16 viral PCR test are determined and evaluated by dose cohort and overall. The 95% confidence interval of the response rate is evaluated.

### Example 10

### Phase I Study to Evaluate the Safety, Tolerability of Ad5 [E1-, E2b-]-HPV16-E6^{Δ}/E7^{Δ} in Individuals Having HPV-16 Positive Squamous Cell Carcinoma

This example describes the use of Ad5 [E1-, E2b-]-HPV16-E6^{Δ}/E7^{Δ}, an adenoviral vector encoding a modified/fused non-oncogenic HPV-E6/E7 gene, for evaluation of safety of Ad5 [E1-, E2b-]-HPV16-E6^{Δ}/E7^{Δ} when administered subcutaneously every 3 weeks for three injections in individuals that are HPV type 16 positive. Additionally, the pharmacodynamics (PDs) of Ad5 [E1-, E2b-]-HPV16-E6^{Δ}/E7^{Δ} is assessed by ELISpot analysis of cryopreserved PBMC samples for E6 and E7-specific CMI response, and efficacy of Ad5 [E1-, E2b-]-HPV16-E6^{Δ}/E7^{Δ} alone is determined using overall response rate (ORR), 6-month disease control rate (DCR), progression-free survival (PFS) rate, and overall survival (OS) rate.

This is a Phase I trial for individuals that have HPV-16 positive squamous cell carcinoma in one of the following sites: cervix, vagina, vulva, head/neck, anus, and penis. The study is conducted using a dose escalation 3+3 design to evaluate safety and tolerability. Three to 6 subjects are sequentially enrolled starting at dose cohort 1. Subjects are assessed for dose-limiting toxicities (DLTs): Cohort 1: 5×10¹⁰ virus particles (VP); Cohort 2: 5×10¹¹ VP; if needed, dose de-escalation cohort (cohort-1): 5×10⁹ VP.

Dose expansion in a Phase 1b study occurs when the MTD or HTD has been determined. Up to 12 subjects are enrolled in the study. Three to 6 subjects are sequentially enrolled starting at Cohort 1.

### Study Design

This is a Phase I trial in subjects with histologically or cytologically-confirmed HPV positive squamous cell carcinoma of the cervix, vagina, vulva, head/neck, anus, penis. The study involves using a standard modified Fibonacci cohort 3+3 design. Treatment starts at DL1 as outlined in TABLE 4. No intra-patient dose escalations are permitted.

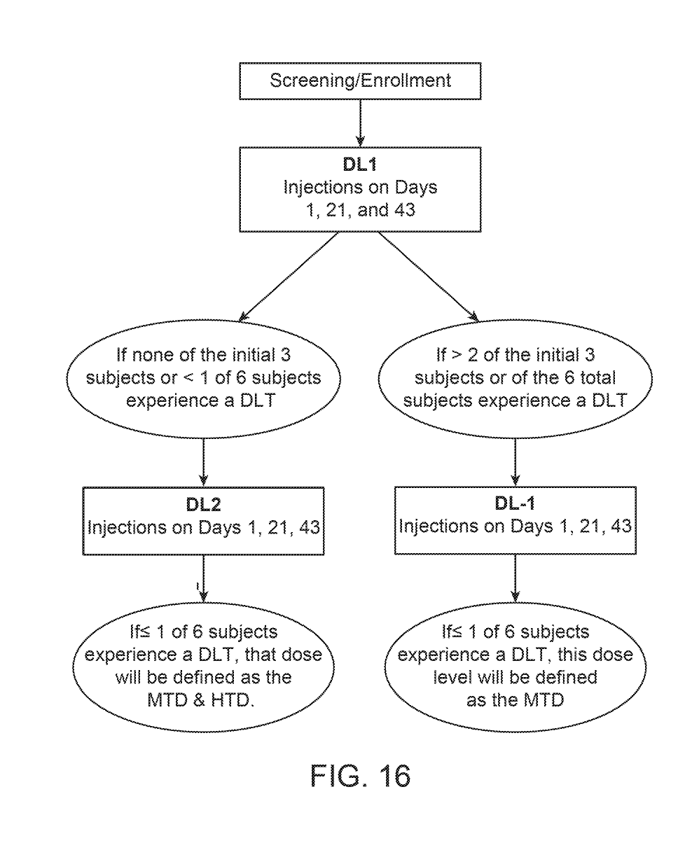

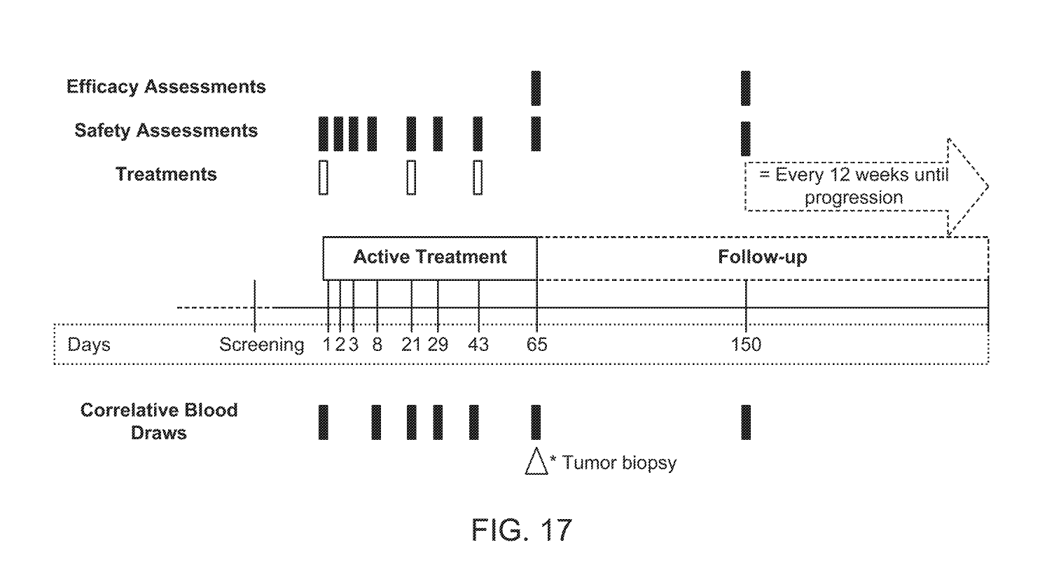

In this dose-escalation component, 3 to 6 subjects are sequentially enrolled starting at dose Cohort 1 (TABLE 6). During each cohort enrollment, a minimum of 7 days are required between enrollments. This allows for dose-limiting toxicity (DLT) monitoring in the prior subject before the next subject is treated. DLTs are monitored continuously. For a schematic of the study design as well as treatment and correlative biomarkers see FIG. 16 and FIG. 17, respectively.

TABLE 6
Dose Levels

| Dose Level | Dose | Number of |
|---|---|---|
| (DL) | (VP) | participants |
| DL −1 | 5 × 10⁹ | 3-6 |
| DL 1* | 5 × 10¹⁰ | 3-6 |
| DL 2 | 5 × 10¹¹ | 6 |

*starting dose

Events that occur within 4 weeks (28 days) following the last study treatment are evaluable for DLTs. TABLE 7 details which events are considered DLTs.

TABLE 7
DLTs

| Toxicity | DLT Definition |
|---|---|
| Non-Hematologic | Grade 3 non-hematologic adverse event attributed |
| | (definitely, probably or possibly) to study treatment |
| | ≥Grade 4 non-hematologic adverse event |
| | regardless of attribution, unless clearly unrelated to |
| | study treatment (i.e. due to disease progression) |
| Hematologic | ≥Grade 4 hematologic adverse event regardless of |
| | attribution, unless clearly unrelated to study |
| | treatment (i.e. due to disease progression) |

Ad5 [E1-, E2b-]-HPV16-E6^{Δ}/E7^{Δ} Vaccine

The investigational product is a non-replicating recombinant adenovirus serotype (Ad5) containing non-oncogenic early 6 (E6) and early 7 (E7) genes of HPV16 and is referred to as Ad5 [E1-, E2b-]-HPV16-E6^{Δ}/E7^{Δ}. The study drug has the designation Ad5 [E1-, E2b-]-HPV16-E6^{Δ}/E7^{Δ} and is described previously in TABLE 5. The Ad5 [E1-, E2b-] vectors is non-replicating and its genome does not integrative into the human genome.

Ad5 [E1-, E2b-]-HPV16-E6^{Δ}/E7^{Δ} Dose Preparation and Administration

The dose of Ad5 [E1-, E2b-]-HPV16-E6^{Δ}/E7^{Δ} to be injected is 5×10⁹ VP (for de-escalation Cohort-1) per mL, 5×10¹⁰ VP (Cohort 1) per mL, or 5×10¹¹ VP (Cohort 2) per 1 mL. Prior to injection, the appropriate vial is from the freezer and allowed to thaw at controlled room temperature (20-25° C., 68-77° F.) for at least 20 minutes and not more than 30 minutes, after which it is kept at 2-8° C. (35-46° F.).

Each vial is sealed with a rubber stopper and has a white flip-off seal. The end user of the product flips the white plastic portion of the cap up/off with their thumb to expose the rubber stopper and then punctures the stopper with an injection needle to withdraw the liquid. The rubber stopper is secured to the vial with an aluminum-crimped seal.

The thawed vial is swirled and then, using aseptic technique, the pharmacist withdraws the appropriate volume from the appropriate vial using a 1 mL syringe.

The vaccine dose is injected as soon as possible using a 1 to ½ inch, 20 to 25-gauge needle. If the vaccine cannot be injected immediately, the syringe is returned to the pharmacy and properly disposed in accordance with institutional policy and procedure, and disposition must be recorded on the investigational product accountability record.

Storage of the vaccine in the vial at 2-8° C. (35-46° F.) does not exceed 8 hours. Once the vaccine has been thawed, it is not refrozen.

For dose preparation of the 5×10¹¹ virus particles, 1 mL of contents from the vial is withdrawn, injection site is prepared with alcohol, and administration to the subject by SQ injection in the thigh is carried out without any further manipulation.

For dose preparation of the 5×10¹⁰ virus particles, from a 5.0-mL vial of 0.9% sterile saline, 0.50 mL of fluid is removed using a 1.0 mL tuberculin syringe, leaving 4.50 mL. Then, using another 1.0 mL tuberculin syringe, 0.50 mL is removed from the vial labeled Ad5 [E1-, E2b-]-HPV16-E6^{Δ}/E7, and delivered into the 4.5 mL of sterile saline remaining in the 5-mL sterile saline vial. The contents are mixed by inverting the 5 mL solution of diluted Ad5 [E1-, E2b-]-HPV16-E6^{Δ}/E7^{Δ}. 1 mL of the diluted Ad5 [E1-, E2b-]-HPV16-E6^{Δ}/E7^{Δ} is withdrawn, injection site is prepared with alcohol, and administration to the subject by SQ injection in the thigh is carried out.

For dose preparation of the 5×10⁹ virus particles, from a 5.0-mL vial of 0.9% sterile saline, 0.05 mL of fluid is removed using a 0.50 mL tuberculin syringe, leaving 4.95 mL. Then, using another 0.50 mL tuberculin syringe, 0.05 mL is removed from the vial labeled Ad5 [E1-, E2b-]-HPV16-E6^{Δ}/E7^{Δ}, and delivered into the 4.95 mL of sterile saline remaining in the 5-mL sterile saline vial. The contents are mixed by inverting the 5 mL of diluted Ad5 [E1-, E2b-]-HPV16-E6^{Δ}/E7^{Δ}. 1 mL of the diluted Ad5 [E1-, E2b-]-HPV16-E6^{Δ}/E7^{Δ} is withdrawn, the injection site is prepared with alcohol, and administration to the subject by SQinjection in the thigh is carried out.

All injections of the vaccine are given as a volume of 1 mL by SC injection in the thigh after preparation of the site with alcohol. Either thigh is used for the initial injection. Subsequent injections are given in the same thigh as the initial injection and are separated by at least 5 cm.

### Treatment Period Procedures and Evaluation

Ad5 [E1-, E2b-]-HPV16-E6^{Δ}/E7^{Δ} is administered at day 1, 21, and 43 for three injections. All study drug administration treatment should occur within ±2 days of the planned visit date except for day 1. Subjects are considered enrolled on day 1 when the study drug is administered.

Subjects must remain in the clinic for a minimum of 30 minutes after the first injection to allow for the evaluation of vital signs and for monitoring of injection site reactions. For the first injection, vital signs must be assessed 30 minutes after the injection.

The following procedures and evaluations are performed and documented in the subject's source records: directed physical examination, vital signs, and body weight; assessment of ECOG performance status, review of concomitant medications, AE assessment, clinical laboratory tests (chemistry: sodium, potassium chloride, bicarbonate, calcium, magnesium, phosphorus, glucose, BUN, serum creatinine, ALT, AST, alkaline phosphatase, lactate dehydrogenase (LDH), total protein, albumin, and total and direct bilirubin; hematology: CBC with differential and platelets with hemoglobin and hematocrit; urinalysis), collection of whole blood for exploratory immune analyses, and tumor imaging and assessment. Tumor imaging and assessment is performed at day 65 and then every 12 weeks (+7 days) thereafter, or earlier if clinically indicated. Objective response is confirmed at least 4 weeks (a minimum of 28 days) after the initial documented complete response (CR) or partial response (PR). Target and non-target lesions are documented and followed. RECIST Version 1.1 is followed for assessment of tumor response.

### Exploratory Pharmacodynamic Assessments

Approximately 90 mL of the subject's peripheral blood is drawn to evaluate the study drug's effect on the immune response at specific time points during the study and/or after a specified injection. Blood draws are done prior to each injection and approximately 3 weeks after the third injection (day 65) for a total of 4 timepoints. Six, 10-mL green top sodium heparin tubes for PBMC samples and two 8-mL serum-separating tubes for serum samples are drawn. Immune assessments are performed and include ELISpot assays, flow cytometry-based assays, and serum assays.

For analysis of PBMCs, pre- and post-therapy PBMCs, separated by Ficoll-Hypaque density gradient separation, are analyzed for antigen-specific immune responses using an intracellular cytokine staining assay. PBMCs are stimulated in vitro with overlapping 15-mer peptide pools encoding the tumor-associated antigen HER2. Control peptide pools involve the use of human leukocyte antigen peptide as a negative control and CEFT peptide mix as a positive control. CEFT is a mixture of peptides of CMV, Epstein-Barr virus, influenza, and tetanus toxin. Post-stimulation analyses of CD4 and CD8 T cells involve the production of IFN-γ, IL-2, tumor necrosis factor, and CD107a. If sufficient PBMCs are available, assays are also performed for the development of T cells to other tumor-associated antigens.

PBMCs are also evaluated for changes in standard immune cell types (CD4 and CD8 T cells, natural killer [NK] cells, regulatory T cells [Tregs], myeloid-derived suppressor cells [MDSCs], and dendritic cells) as well as 123 immune cell subsets. If sufficient PBMCs are available, PBMCs from selected subjects are analyzed for function of specific immune cell subsets, including CD4 and CD8 T cells, NK cells, Tregs, and MDSCs.

For the ELISpot analysis, antigen specific CMI and cytolytic T lymphocyte (CTL) activity are assessed using an ELISpot assays. The CMI activity of T cells against HPV16-E6^{Δ}/E7^{Δ} is assessed by re-stimulating PBMCs with purified HPV16-E6^{Δ}/E7^{Δ} peptides and the numbers of IFN-γ secreting spot forming cells (SFC) determined. The CTL activity of cells against HPV16-E6^{Δ}/E7^{Δ} is assessed using a granzyme B ELISPOT assay that is an accepted test to measure functional CTLs. PBMCs are re-stimulated with purified HPV16-E6^{Δ}/E7^{Δ} peptides and the numbers of granzyme B secreting spot forming cells (SFC) determined. Using previously described criteria (17,18), CMI responses are considered positive if ≥50 SFC are detected per 10⁶ cells after subtraction of the negative control and SFC are ≥2-fold higher than those observed in the negative control wells. Patient CMI responses in each cohort are determined at baseline, at 4-weeks after the 3rd immunization, and at months 6 and 12 after the first immunization. Statistical analyses comparing immune responses (number of SFC) at each sampling point are performed employing the Student T tests and/or Mann-Whitney tests (PRISM, Graph Pad).

For analysis of soluble factors, sera is analyzed pre- and post-therapy for the following soluble factors: soluble CD27, soluble CD40 ligand, and antibodies to HPV E6, antibodies to HPV E7, and antibodies to other tumor-associated antigens. Serum IgG antibody (Ab) responses to HPV16-E6 and/or HPV E7 are measured using a quantitative ELISA technique using purified E6 and E7 proteins and Ad5 neutralizing antibody (NAb) is determined and are reported as the inverse of the endpoint Ad5 NAb titer. Statistical analyses comparing immune responses at each sampling point (baseline, at each immunization, at 3-weeks after the 3rd immunization, are performed employing the Student T tests and/or Mann-Whitney tests (PRISM, Graph Pad).

### Exploratory Genomics and Proteomics Molecular Analysis

Exploratory genomics and proteomics molecular profiling are performed on FFPE tumor tissue and whole blood (subject matched normal comparator against the tumor tissue) by next-generation sequencing and mass spectrometry-based quantitative proteomics. Collection of tumor tissue and whole blood are requested for this study. Tumor tissues and whole blood are obtained at the baseline.

A single FFPE tumor tissue block is required for the extraction of tumor DNA, tumor RNA, and tumor protein. A whole blood sample is required for the extraction of subject normal DNA. Tumor tissue and whole blood are processed in CLIA-registered and CAP accredited/CLIA certified laboratories. TABLE 8 describes the collection schedule for molecular profiling.

TABLE 8
Schedule of Collection for Exploratory Molecular Profiling

| | |
|---|---|
| Exploratory Molecular Profiling | Baseline |

Whole blood (normal comparator against tumor)

| | |
|---|---|
| 1 PAXgene Blood DNA tube (2.5 mL)^{a} | ✓ |

Formalin-fixed, paraffin-embedded tumor tissue block/slides^{b}

| | |
|---|---|
| A minimum tissue surface area of 25 mm², 75 μm | ✓ |
| thick, with at least 30% malignant tissue | |

^{a}Whole blood to be collected at baseline only for genomic sequencing. Requires 2.5 mL of subject's whole blood in 1 PAXgene Blood DNA tube, provided in the Blood Specimen Kit.
^{b}FFPE tissue block/slides to be collected at baseline for genomic sequencing, RNA sequencing, and proteomic analysis. A single block meeting the minimum requirements for genomics and proteomics is required. FFPE tissue blocks to be collected per local pathology laboratory procedures.

### Inclusion Criteria

One or more of the following conditions must be met in order for subjects to be eligible for inclusion in the study. Individuals having histologically or cytologically-confirmed HPV 16 positive malignancy of one of the following types are: squamous cell carcinoma of the cervix, vagina, or vulva, head and neck, anus, or penis, individuals with a disease that is not treatable by curative-intent therapy (i.e., surgical resection, chemoradiation, etc.), and/or individuals with a progressive metastatic or recurrent disease treated with at least 1 prior regimen of therapy in the metastatic/recurrent setting, which must have included a platinum agent are eligible for inclusion in the study. Subjects who are eligible for the study must also be able to provide written informed consent for the trial and must be ≥18 years of age on day of signing informed consent. Subjects with measurable disease as determined by the Response Evaluation Criteria in Solid Tumors (RECIST) 1.1 are also eligible for inclusion in the study.

Further eligibility criteria include that the subject is willing to provide tissue from a recently obtained core or excisional biopsy of a tumor lesion (defined as a specimen obtained up to 30 days prior to enrollment), the subject is willing to undergo a repeat biopsy following treatment at day 65 (+7 days), and the subject is eligible if the subject has a performance status of 0 or 1 on the ECOG Performance Scale. Individuals eligible for inclusion in the study demonstrate adequate organ function as measured by white blood cells (WBCs) ≥2000/μL, neutrophils ≥1500/μL, platelets ≥100×10³/μL, hemoglobin ≥9.0 g/dL, creatinine serum ≥1.5×upper limit normal (ULN) or creatinine clearance (CrCl) ≥40 mL/minute (using Cockcroft/Gault formula), AST ≤3×ULN, and ALT ≤3×ULN, total bilirubin ≤1.5×ULN (except subjects with Gilbert Syndrome who can have total bilirubin <3.0 mg/dL).

If the subject is female and of childbearing potential, the individual should have a negative urine pregnancy within 24 hours prior to receiving the first dose of study medication in order to be eligible for inclusion in the study. If the urine test is positive or cannot be confirmed as negative, a serum pregnancy test will be required. Female subjects of childbearing potential should be willing to use two methods of birth control or be surgically sterile, or abstain from heterosexual activity for the course of the study through 30 days after the last dose of study medication in order to be eligible for inclusion. Subjects of childbearing potential are those who have not been surgically sterilized or have not been free from menses for >1 year. Finally, the subject is eligible if the subject is a male subject and agrees to use an adequate method of contraception starting with the first dose of study therapy through 30 days after the last dose of study therapy in order to be considered for inclusion in the study.

### Exclusion Criteria

The following cases are grounds for excluding subjects from the trial. Individuals receiving any other investigational agents, chemotherapy, immunotherapy, radiotherapy, or molecular targeted agents within four weeks of the start of the study treatment are not eligible for inclusion in the study. Subjects with a disease that is considered curable with local therapies are also ineligible. Further exclusion criteria include current participation in a study in which they are receiving study therapy and/or past participation in a study therapy or use of an investigational device within four weeks of the first dose of treatment.

Subjects with a diagnosis of immunodeficiency or are receiving systemic steroid therapy or any other form of immunosuppressive therapy within seven days prior to the first dose of trial treatment or having a known history of active TB (Bacillus Tuberculosis) are excluded from the clinical trial. Patients who have had a prior anti-cancer monoclonal antibody (mAb) within four weeks prior to study day 1 or who have not recovered (i.e., ≤Grade 1 or at baseline) from adverse events due to agents administered more than four weeks earlier are also considered ineligible for inclusion in this trial. Additionally, subjects with a known additional malignancy that is progressing or requires active treatment (exceptions include basal cell carcinoma of the skin or squamous cell carcinoma of the skin that has undergone potentially curative therapy or in situ cervical cancer), or a known active central nervous system (CNS) metastases and/or carcinomatous meningitis are excluded from the trial. Participants with CNS metastases treated with radiation are eligible, so long as they completed radiation ≥four weeks prior to enrollment and have no documented progression on imaging (CT of the head with IV contrast or MRI). These participants must be able to be stable off corticosteroids (≥10 mg or prednisone or equivalent for at least 2 weeks prior to enrollment).

Subjects with active autoimmune disease that has required systemic treatment in the past two years (i.e. with use of disease modifying agents, corticosteroids or immunosuppressive drugs) are excluded from the trial. However, replacement therapy (e.g., thyroxine, insulin, or physiologic corticosteroid replacement therapy for adrenal or pituitary insufficiency, etc.) is not considered a form of systemic treatment. Subjects with a known history of, or any evidence of active, non-infectious pneumonitis, an active infection requiring systemic therapy or a history or current evidence of any condition, therapy, or laboratory abnormality that might confound the results of the trial, interfere with the subject's participation for the full duration of the trial, or is not in the best interest of the subject to participate, in the opinion of the treating investigator are excluded from the trial. Subjects are excluded if they have a known psychiatric or substance abuse disorders that would interfere with cooperation with the requirements of the trial or if they are pregnant or breastfeeding, or expecting to conceive or father children within the projected duration of the trial, starting with the pre-screening or screening visit through 120 days after the last dose of trial treatment.

Finally subjects with a known history of Human Immunodeficiency Virus (HIV) (HIV 1/2 antibodies), known active Hepatitis B (e.g., HBsAg reactive) or Hepatitis C (e.g., HCV RNA [qualitative] is detected), or subjects who have received a live vaccine within 30 days of planned start of study therapy are ineligible for inclusion in this clinical trial. Seasonal influenza vaccines for injection are generally inactivated flu vaccines and are allowed, however intranasal influenza vaccines (e.g., Flu-Mist®) are live attenuated vaccines, and are not allowed.

### Statistical Considerations

This is a single-arm phase I study designed to determine the R2PD and adverse event profile of Ad5 [E1-, E2b-]-HPV16-E6^{Δ}/E7^{Δ} in patients with refractory advanced/metastatic HPV+ malignancies (squamous cell carcinoma of cervix, vulva, vagina, anal, penis, and head and neck) using a standard modified Fibonacci cohort 3+3 design as previously described. The R2PD is estimated overall and not be specific to disease type.

Sample size is based on the number of participants needed in each dose escalation cohort. Based on the DLs, a minimum of 9 participants and a maximum of 12 participants could be included.

### Safety Analysis

DLTs are evaluated continuously in a cohort. An overall assessment of whether to escalate to the next dose level is made at least 3 weeks after the last subject in the previous cohort has received their first injection. A dose level is considered safe if <33% of subjects treated at a dose level experience a DLT (i.e., 0 of 3, ≤1 of 6, ≤2 of 9, ≤3 of 12, ≤4 of 15, or ≤5 of 18 subjects). A DLT is defined above. Safety is evaluated in 3 or 6 subjects at each dose level in the dose escalation component of the study. A subject is considered evaluable for safety if treated with at least one injection. DLTs are observed through 9 weeks to accommodate the safety evaluation of multiple doses of the vaccine.

Overall safety is assessed by descriptive analyses using tabulated frequencies of AEs by grade using CTCAE Version 4.0 within dose cohorts and for the overall study population in terms of treatment-emergent AEs, SAEs, and clinically significant changes in safety laboratory tests, physical examinations, and vital signs.

### Efficacy Analysis

All subjects are followed for progression/survival every 3 months after day 65 until death. The percentage of subjects that achieve an objective confirmed complete or partial overall tumor response using RECIST Version 1.1 are evaluated by dose cohort and overall. The 95% confidence interval of the response rate is evaluated. Disease control (confirmed response or SD lasting for at least 6 months) is analyzed in a similar manner.

The duration of overall response is evaluated by dose cohort and overall. The duration of overall response is measured from the time measurement criteria are met for CR or PR (whichever is first recorded) until the first date that recurrent or PD is objectively documented (taking as reference for PD the smallest measurements recorded since the treatment started).

PFS is evaluated by dose cohort and overall using Kaplan-Meier methods. PFS is defined as the time from the date of first treatment to the date of disease progression or death (any cause) whichever occurs first. Subjects who do not have disease progression or have not died at the end of follow up are censored at the last known date the subject was progression free.

OS is evaluated by dose cohort and overall using Kaplan-Meier methods. OS is defined as the time from the date of first treatment to the date of death (any cause). Subjects who are alive at the end of follow up are censored at the last known date alive.

### Example 11

### Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously. Vaccines are administered a total of 3-times and each vaccination is separated by a 3 week interval. Thereafter, a booster injection is given every two months (bi-monthly). The subject is any animal, for example a mammal, such as a mouse, human, or non-human primate. Upon administration of the vaccine, the cellular and humoral responses are initiated against the HPV-expressing cancer and the cancer is eliminated.

### Example 12

### Combination Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7 and Co-Stimulatory Molecules

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3 week interval. Thereafter, bi-monthly booster injections are administered. The co-stimulatory molecule is B7-1, ICAM-1, or LFA-3. The subject is any animal, for example a mammal, such as a mouse, human, or non-human primate. Upon administration of the vaccine and co-stimulatory molecule, the cellular and humoral responses are initiated against the HPV-expressing cancer and the cancer is eliminated.

### Example 13

### Combination Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7 and Checkpoint Inhibitors

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously in combination with a checkpoint inhibitor. Vaccines are administered a total of 3 times and each vaccination is separated by a 3-week interval. Thereafter, bi-monthly booster injections are administered. The checkpoint inhibitor is an anti-PDL1 antibody, such as Avelumab. Avelumab is dosed and administered as per package insert labeling at 10 mg/kg. The subject is any animal, for example a mammal, such as a mouse, human, or non-human primate. Upon administration of the vaccine and the checkpoint inhibitor, the cellular and humoral responses are initiated against the HPV-expressing cancer and the cancer is eliminated.

### Example 14

### Combination Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7 and Engineered NK Cells

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered. Subjects are additionally administered engineered NK cells, specifically activated NK cells (aNK cells). aNK cells are infused on days −2, 12, 26, and 40 at a dose of 2×10⁹ cells per treatment. Subjects in need thereof have HPV-expressing cancer cells, such as HPV-associated or HPV-induced cancer. Subjects are any mammal, such as a human or a non-human primate.

### Example 15

### Combination Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7 and ALT-803

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered. Subjects are also administered a super-agonist/super-agonist complex, such as ALT-803, at a dose of 10 μg/kg SC on weeks 1, 2, 4, 5, 7, and 8, respectively. Subjects in need thereof have HPV-expressing cancer cells, such as HPV-associated or HPV-induced cancer. Subjects are any mammal, such as a human or a non-human animal.

### Example 16

### Combination Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7 and Low Dose Chemotherapy

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered.

Subjects are also administered low dose chemotherapy. The chemotherapy is cyclophosphamide. The chemotherapy is administered at a dose that is lower than the clinical standard of care dosing. For example, the chemotherapy is administered at 50 mg twice a day (BID) on days 1-5 and 8-12 every 2 weeks for a total of 8 weeks. Subjects in need thereof have HPV-expressing, such as HPV-associated or HPV-induced cancer. Subjects are any mammal, such as a human or a non-human animal.

### Example 17

Combination Treatment of HPV-Induced or HPV-Associated Cancer with Ad5 [E1-, E2b-]-HPV E6, Ad5 [E1-, E2b-]-HPV E7, and/or Ad5 [E1-, E2b-]-HPV E6/E7 and Low Dose Radiation

This example describes treatment of HPV-expressing cells in HPV-induced or HPV-associated cancer, in a subject in need thereof. Ad5 [E1-, E2b-] vectors encoding for E6, E7, and/or E6/E7 are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered.

Subjects are also administered low dose radiation. The low dose radiation is administered at a dose that is lower than the clinical standard of care dosing. Concurrent sterotactic body radiotherapy (SBRT) at 8 Gy is given on day 8, 22, 36, 50 (every 2 weeks for 4 doses). Radiation is administered to all feasible tumor sites using SBRT. Subjects in need thereof have HPV-expressing, such as HPV-associated or HPV-induced cancer. Subjects are any mammal, such as a human or a non-human animal.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| SEQ ID NO: | Sequence |
|---|---|
| SEQ ID NO: 1 | TCTCTCCNA |
| SEQ ID NO: 2 | CTCGAGGAAGCTTGCCGCCACCATGCACCAAAAGAGAACTGCA |
| | ATGTTTCAGGACCCACAGGAGCGACCCAGAAAGTTACCACAGT |
| | TATGCACAGAGGTGCAAACAACTATACATGATATAATATTAGA |
| | ATGTGTGTACTGCAAGCAACAGTTACTGCGACGTGAGGTATAT |
| | GACTTTGCTTTTCGGGATGGATGCATAGTATATAGAGATGGGA |
| | ATCCATATGCTGTATGTGATAAATGTTTAAAGTTTTATTCTAAA |
| | ATTAGTGAGTATAGACATTATTGTTATAGTTTGTATGGAACAAC |
| | ATTAGAACAGCAATACAACAAACCGTTGTGTGATTTGTTAATT |
| | AGGTGTATTAACTGTCAAAAGCCACTGTGTCCTGAAGAAAAGC |
| | AAAGACATCTGGACAAAAAGCAAAGATTCCATAATATAAGGG |
| | GTCGGTGGACCGGTCGATGTATGTCTTGTTGCAGATCATCAAG |
| | AACTCGTAGAGCAGCCGCGGCGTAATCATGCCTGGAGATACAC |
| | CTACATTGCATGAATATATGTTAGATTTGCAACCAGAGACAAC |
| | TGATCTCTACGGTTATGAGCAATTAAATGACAGCTCAGAGGAG |
| | GAGGATGAAATAGATGGTCCAGCTGGACAAGCAGCACCGGAC |
| | AGAGCCCATTACAATATTGTAACCTTTTGTTGCAAGTGTGACTC |
| | TACGCTTCGGAGGTGCGTACAAAGCACACACGTAGACATTCGT |
| | ACTTTGGAAGACCTGTTAATGGGCGTACTAGGAATTGTGTGCC |
| | CCATCTGTTCTCAGAAACCATGAGATATCGCGGCCGC |
| SEQ ID NO: 3 | CTCGAGGAAGCTTGCCGCCACCATGCACCAAAAGAGAACTGCA |
| | ATGTTTCAGGACCCACAGGAGCGACCCAGAAAGTTACCACAGT |
| | TATGCACAGAGCTGCAAACAACTATACATGATATAATATTAGA |
| | ATGTGTGTACTGCAAGCAACAGTTACTGCGACGTGAGGTATAT |
| | GACTTTGCTTTTCGGGATGGATGCATAGTATATAGAGATGGGA |
| | ATCCATATGCTGTATGTGATAAATGTTTAAAGTTTTATTCTAAA |
| | ATTAGTGAGTATAGACATTATTGTTATAGTTTGTATGGAACAAC |
| | ATTAGAACAGCTATACAACAAACCGTTGTGTGATGTGTTAATT |
| | AGGTGTATTAACTGTCAAAAGCCACTGTGTCCTGAAGAAAAGC |
| | AAAGACATCTGGACAAAAAGCAAAGATTCCATAATATAAGGG |
| | GTCGGTGGACCGGTCGATGTATGTCTTGTTGCAGATCATCAAG |
| | AACTCGTAGAGCAGCCGCGGCGTAATCATGCCTGGAGATACAC |
| | CTACATTGCATGAATATATGTTAGATTTGCAACCAGAGACAAC |
| | TGATCTCTACGGTTATGAGCAATTAAATGACAGCTCAGAGGAG |
| | GAGGATGAAATAGATGGTCCAGCTGGACAAGCAGCACCGGAC |
| | AGAGCCCATTACAATATTGTAACCTTTTGTTGCAAGTGTGACTC |
| | TACGCTTCGGAGGTGCGTACAAAGCACACACGTAGACATTCGT |
| | ACTTTGGAAGACCTGTTAATGGGCGTACTAGGAATTGTGTGCC |
| | CCATCTGTTCTCAGAAACCATGAGATATCGCGGCCGC |
| SEQ ID NO: 4 | CTCGAGGAAGCTTGCCGCCACCATGCACCAAAAGAGAACTGCA |
| | ATGTTTCAGGACCCACAGGAGCGACCCAGAAAGTTACCACAGT |
| | TATGCACAGAGGTGCAAACAACTATACATGATATAATATTAGA |
| | ATGTGTGTACTGCAAGCAACAGTTACTGCGACGTGAGGTATAT |
| | GACTTTGCTTTTCGGGATGGATGCATAGTATATAGAGATGGGA |
| | ATCCATATGCTGTATGTGATAAATGTTTAAAGTTTTATTCTAAA |
| | ATTAGTGAGTATAGACATTATTGTTATAGTTTGTATGGAACAAC |
| | ATTAGAACAGCTATACAACAAACCGTTGTGTGATGTGTTAATT |
| | AGGTGTATTAACTGTCAAAAGCCACTGTGTCCTGAAGAAAAGC |
| | AAAGACATCTGGACAAAAAGCAAAGATTCCATAATATAAGGG |
| | GTCGGTGGACCGGTCGATGTATGTCTTGTTGCAGATCATCAAG |
| | AACTCGTAGAGCAGCCGCGGCGTAATCATGCCTGGAGATACAC |
| | CTACATTGCATGAATATATGTTAGATTTGCAACCAGAGACAAC |
| | TGATCTCTACGGTTATGAGCAATTAAATGACAGCTCAGAGGAG |
| | GAGGATGAAATAGATGGTCCAGCTGGACAAGCAGCACCGGAC |
| | AGAGCCCATTACAATATTGTAACCTTTTGTTGCAAGTGTGACTC |
| | TACGCTTCGGAGGTGCGTACAAAGCACACACGTAGACATTCGT |
| | ACTTTGGAAGACCTGTTAATGGGCGTACTAGGAATTGTGTGCC |
| | CCATCTGTTCTCAGAAACCATGAGATATCGCGGCCGC |
| SEQ ID NO: 5 | ATGCACCAAAAGAGAACTGCAATGTTTCAGGACCCACAGGAGC |
| | GACCCAGAAAGTTACCACAGTTATGCACAGAGGTGCAAACAAC |
| | TATACATGATATAATATTAGAATGTGTGTACTGCAAGCAACAG |
| | TTACTGCGACGTGAGGTATATGACTTTGCTTTTCGGGATGGATG |
| | CATAGTATATAGAGATGGGAATCCATATGCTGTATGTGATAAA |
| | TGTTTAAAGTTTTATTCTAAAATTAGTGAGTATAGACATTATTG |
| | TTATAGTTTGTATGGAACAACATTAGAACAGCAATACAACAAA |
| | CCGTTGTGTGATTTGTTAATTAGGTGTATTAACTGTCAAAAGCC |
| | ACTGTGTCCTGAAGAAAAGCAAAGACATCTGGACAAAAAGCA |
| | AAGATTCCATAATATAAGGGGTCGGTGGACCGGTCGATGTATG |
| | TCTTGTTGCAGATCATCAAGAACTCGTAGAGCAGCCGCGGCGT |
| | GA |
| SEQ ID NO: 6 | ATGCACCAAAAGAGAACTGCAATGTTTCAGGACCCACAGGAGC |
| | GACCCAGAAAGTTACCACAGTTATGCACAGAGCTGCAAACAAC |
| | TATACATGATATAATATTAGAATGTGTGTACTGCAAGCAACAG |
| | TTACTGCGACGTGAGGTATATGACTTTGCTTTTCGGGATGGATG |
| | CATAGTATATAGAGATGGGAATCCATATGCTGTATGTGATAAA |
| | TGTTTAAAGTTTTATTCTAAAATTAGTGAGTATAGACATTATTG |
| | TTATAGTTTGTATGGAACAACATTAGAACAGCTATACAACAAA |
| | CCGTTGTGTGATGTGTTAATTAGGTGTATTAACTGTCAAAAGCC |
| | ACTGTGTCCTGAAGAAAAGCAAAGACATCTGGACAAAAAGCA |
| | AAGATTCCATAATATAAGGGGTCGGTGGACCGGTCGATGTATG |
| | TCTTGTTGCAGATCATCAAGAACTCGTAGAGCAGCCGCGGCGT |
| | GA |
| SEQ ID NO: 7 | ATGCACCAAAAGAGAACTGCAATGTTTCAGGACCCACAGGAGC |
| | GACCCAGAAAGTTACCACAGTTATGCACAGAGGTGCAAACAAC |
| | TATACATGATATAATATTAGAATGTGTGTACTGCAAGCAACAG |
| | TTACTGCGACGTGAGGTATATGACTTTGCTTTTCGGGATGGATG |
| | CATAGTATATAGAGATGGGAATCCATATGCTGTATGTGATAAA |
| | TGTTTAAAGTTTTATTCTAAAATTAGTGAGTATAGACATTATTG |
| | TTATAGTTTGTATGGAACAACATTAGAACAGCTATACAACAAA |
| | CCGTTGTGTGATGTGTTAATTAGGTGTATTAACTGTCAAAAGCC |
| | ACTGTGTCCTGAAGAAAAGCAAAGACATCTGGACAAAAAGCA |
| | AAGATTCCATAATATAAGGGGTCGGTGGACCGGTCGATGTATG |
| | TCTTGTTGCAGATCATCAAGAACTCGTAGAGCAGCCGCGGCGT |
| | GA |
| SEQ ID NO: 8 | MHQKRTAMFQDPQERPRKLPQLCTEVQTTIHDIILECVYCKQQLL |
| | RREVYDFAFRDGCIVYRDGNPYAVCDKCLKFYSKISEYRHYCYSL |
| | YGTTLEQQYNKPLCDLLIRCINCQKPLCPEEKQRHLDKKQRFHNIR |
| | GRWTGRCMSCCRSSRTRRAAAA |
| SEQ ID NO: 9 | MHQKRTAMFQDPQERPRKLPQLCTELQTTIHDIILECVYCKQQLL |
| | RREVYDFAFRDGCIVYRDGNPYAVCDKCLKFYSKISEYRHYCYSL |
| | YGTTLEQLYNKPLCDVLIRCINCQKPLCPEEKQRHLDKKQRFHNIR |
| | GRWTGRCMSCCRSSRTRRAAAA |
| SEQ ID NO: | MHQKRTAMFQDPQERPRKLPQLCTEVQTTIHDIILECVYCKQQLL |
| 10 | RREVYDFAFRDGCIVYRDGNPYAVCDKCLKFYSKISEYRHYCYSL |
| | YGTTLEQLYNKPLCDVLIRCINCQKPLCPEEKQRHLDKKQRFHNIR |
| | GRWTGRCMSCCRSSRTRRAAAA |
| SEQ ID NO: | ATGCCTGGAGATACACCTACATTGCATGAATATATGTTAGATTT |
| 11 | GCAACCAGAGACAACTGATCTCTACGGTTATGAGCAATTAAAT |
| | GACAGCTCAGAGGAGGAGGATGAAATAGATGGTCCAGCTGGA |
| | CAAGCAGCACCGGACAGAGCCCATTACAATATTGTAACCTTTT |
| | GTTGCAAGTGTGACTCTACGCTTCGGAGGTGCGTACAAAGCAC |
| | ACACGTAGACATTCGTACTTTGGAAGACCTGTTAATGGGCGTA |
| | CTAGGAATTGTGTGCCCCATCTGTTCTCAGAAACCATGA |
| SEQ ID NO: | MPGDTPTLHEYMLDLQPETTDLYGYEQLNDSSEEEDEIDGPAGQA |
| 12 | APDRAHYNIVTFCCKCDSTLRRCVQSTHVDIRTLEDLLMGVLGIV |
| | CPICSQKP |
| SEQ ID NO: | MHQKRTAMFQDPQERPRKLPQLCTELQTTIHDIILECVYCKQQLL |
| 13 | RREVYDFAFRDGCIVYRDGNPYAVCDKCLKFYSKISEYRHYCYSL |
| | YGTTLEQQYNKPLCDLLIRCINCQKPLCPEEKQRHLDKKQRFHNIR |
| | GRWTGRCMSCCRSSRTRRAAAA |
| SEQ ID NO: | MPGDTPTLHEYMLDLQPETTDLYGYEQLNDSSEEEDEIDGPAGQA |
| 14 | APDRAHYNIVTFCCKCDSTLRRCVQSTHVDIRTLEDLLMGTLGIVC |
| | PICSQKP |
| SEQ ID NO: | AAGCAGAGGCTCGTTTAGTGAACCGTCAGATGGTACCGTTTAA |
| 15 | ACTCGAGGTCGACGGTATCGATAAGCTTGATATCGAATTCGAG |
| | CTCGGTACCCCCGGTTAGTATAAAAGCAGACATTTTATGCACC |
| | AAAAGAGAACTGCAATGTTTCAGGACCCACAGGAGCGACCCA |
| | GAAAGTTACCACAGTTATGCACAGAGCTGCAAACAACTATACA |
| | TGATATAATATTAGAATGTGTGTACTGCAAGCAACAGTTACTG |
| | CGACGTGAGGTATATGACTTTGCTTTTCGGGATGGATGCATAGT |
| | ATATAGAGATGGGAATCCATATGCTGTATGTGATAAATGTTTA |
| | AAGTTTTATTCTAAAATTAGTGAGTATAGACATTATTGTTATAG |
| | TTTGTATGGAACAACATTAGAACAGCAATACAACAAACCGTTG |
| | TGTGATTTGTTAATTAGGTGTATTAACTGTCAAAAGCCACTGTG |
| | TCCTGAAGAAAAGCAAAGACATCTGGACAAAAAGCAAAGATT |
| | CCATAATATAAGGGGTCGGTGGACCGGTCGATGTATGTCTTGTT |
| | GCAGATCATCAAGAACTCGTAGAGCAGCCGCGGCGTAATCATG |
| | CCTGGAGATACACCTACATTGCATGAATATATGTTAGATTTGCA |
| | ACCAGAGACAACTGATCTCTACGGTTATGAGCAATTAAATGAC |
| | AGCTCAGAGGAGGAGGATGAAATAGATGGTCCAGCTGGACAA |
| | GCAGCACCGGACAGAGCCCATTACAATATTGTAACCTTTTGTTG |
| | CAAGTGTGACTCTACGCTTCGGAGGTGCGTACAAAGCACACAC |
| | GTAGACATTCGTACTTTGGAAGACCTGTTAATGGGCACACTAG |
| | GAATTGTGTGCCCCATCTGTTCTCAGAAACCATAATCTACCATG |
| | GCTGATCCTGCAGCATGCAAGCTGGGGATCCACTAGTTCTAGA |
| | GCGGCCGCCACAGCGGGGAGATCAGACATGATAGATACATTGA |
| | TGAGTTTGGACAAACCACAACTAGAATGCAGTGAAAAAATGCT |
| | TTATTGTGAAATTGTGATGCTATTGCTTATTTGTACATTATAGCT |
| | GCAATAAACAGTTACAACAACAATTGCATTCATTTATGTTCAG |
| | GTCAGGGGGAAGGTGTGGAGGTT |
| SEQ ID NO: | CATCATCAATAATATACCTTATTTTGGATTGAAGCCAATATGAT |
| 16 | AATGAGGGGGTGGAGTTTGTGACGTGGCGCGGGGCGTGGGAA |
| | CGGGGCGGGTGACGTAGTAGTGTGGCGGAAGTGTGATGTTGCA |
| | AGTGTGGCGGAACACATGTAAGCGACGGATGTGGCAAAAGTG |
| | ACGTTTTTGGTGTGCGCCGGTGTACACAGGAAGTGACAATTTTC |
| | GCGCGGTTTTAGGCGGATGTTGTAGTAAATTTGGGCGTAACCG |
| | AGTAAGATTTGGCCATTTTCGCGGGAAAACTGAATAAGAGGAA |
| | GTGAAATCTGAATAATTTTGTGTTACTCATAGCGCGTAATACTG |
| | TAATAGTAATCAATTACGGGGTCATTAGTTCATAGCCCATATAT |
| | GGAGTTCCGCGTTACATAACTTACGGTAAATGGCCCGCCTGGC |
| | TGACCGCCCAACGACCCCCGCCCATTGACGTCAATAATGACGT |
| | ATGTTCCCATAGTAACGCCAATAGGGACTTTCCATTGACGTCAA |
| | TGGGTGGAGTATTTACGGTAAACTGCCCACTTGGCAGTACATC |
| | AAGTGTATCATATGCCAAGTACGCCCCCTATTGACGTCAATGA |
| | CGGTAAATGGCCCGCCTGGCATTATGCCCAGTACATGACCTTAT |
| | GGGACTTTCCTACTTGGCAGTACATCTACGTATTAGTCATCGCT |
| | ATTACCATGGTGATGCGGTTTTGGCAGTACATCAATGGGCGTG |
| | GATAGCGGTTTGACTCACGGGGATTTCCAAGTCTCCACCCCATT |
| | GACGTCAATGGGAGTTTGTTTTGGCACCAAAATCAACGGGACT |
| | TTCCAAAATGTCGTAACAACTCCGCCCCATTGACGCAAATGGG |
| | CGGTAGGCGTGTACGGTGGGAGGTCTATATAAGCAGAGCTGGT |
| | TTAGTGAACCGTCAGATCCGCTAGAGATCTGGTACCGTCGACG |
| | CGGCCGCTCGAGCCTAAGCTTCTAGATGCATGCTCGAGCGGCC |
| | GCCAGTGTGATGGATATCTGCAGAATTCGCCCTTGCTCTCGAGG |
| | AAGCTTGCCGCCACCATGCACCAAAAGAGAACTGCAATGTTTC |
| | AGGACCCACAGGAGCGACCCAGAAAGTTACCACAGTTATGCAC |
| | AGAGGTGCAAACAACTATACATGATATAATATTAGAATGTGTG |
| | TACTGCAAGCAACAGTTACTGCGACGTGAGGTATATGACTTTG |
| | CTTTTCGGGATGGATGCATAGTATATAGAGATGGGAATCCATA |
| | TGCTGTATGTGATAAATGTTTAAAGTTTTATTCTAAAATTAGTG |
| | AGTATAGACATTATTGTTATAGTTTGTATGGAACAACATTAGAA |
| | CAGCTATACAACAAACCGTTGTGTGATGTGTTAATTAGGTGTAT |
| | TAACTGTCAAAAGCCACTGTGTCCTGAAGAAAAGCAAAGACAT |
| | CTGGACAAAAAGCAAAGATTCCATAATATAAGGGGTCGGTGGA |
| | CCGGTCGATGTATGTCTTGTTGCAGATCATCAAGAACTCGTAGA |
| | GCAGCCGCGGCGTAATCATGCCTGGAGATACACCTACATTGCA |
| | TGAATATATGTTAGATTTGCAACCAGAGACAACTGATCTCTAC |
| | GGTTATGAGCAATTAAATGACAGCTCAGAGGAGGAGGATGAA |
| | ATAGATGGTCCAGCTGGACAAGCAGCACCGGACAGAGCCCATT |
| | ACAATATTGTAACCTTTTGTTGCAAGTGTGACTCTACGCTTCGG |
| | AGGTGCGTACAAAGCACACACGTAGACATTCGTACTTTGGAAG |
| | ACCTGTTAATGGGCGTACTAGGAATTGTGTGCCCCATCTGTTCT |
| | CAGAAACCATGAGATATCGCGGCCGCCGATCCACCGGATCTAG |
| | ATAACTGATCATAATCAGCCATACCACATTTGTAGAGGTTTTAC |
| | TTGCTTTAAAAAACCTCCCACACCTCCCCCTGAACCTGAAACAT |
| | AAAATGAATGCAATTGTTGTTGTTAACTTGTTTATTGCAGCTTA |
| | TAATGGTTACAAATAAAGCAATAGCATCACAAATTTCACAAAT |
| | AAAGCATTTTTTTCACTGCATTCTAGTTGTGGTTTGTCCAAACT |
| | CATCAATGTATCTTAACGCGGATCTGGAAGGTGCTGAGGTACG |
| | ATGAGACCCGCACCAGGTGCAGACCCTGCGAGTGTGGCGGTAA |
| | ACATATTAGGAACCAGCCTGTGATGCTGGATGTGACCGAGGAG |
| | CTGAGGCCCGATCACTTGGTGCTGGCCTGCACCCGCGCTGAGTT |
| | TGGCTCTAGCGATGAAGATACAGATTGAGGTACTGAAATGTGT |
| | GGGCGTGGCTTAAGGGTGGGAAAGAATATATAAGGTGGGGGT |
| | CTTATGTAGTTTTGTATCTGTTTTGCAGCAGCCGCCGCCGCCAT |
| | GAGCACCAACTCGTTTGATGGAAGCATTGTGAGCTCATATTTG |
| | ACAACGCGCATGCCCCCATGGGCCGGGGTGCGTCAGAATGTGA |
| | TGGGCTCCAGCATTGATGGTCGCCCCGTCCTGCCCGCAAACTCT |
| | ACTACCTTGACCTACGAGACCGTGTCTGGAACGCCGTTGGAGA |
| | CTGCAGCCTCCGCCGCCGCTTCAGCCGCTGCAGCCACCGCCCG |
| | CGGGATTGTGACTGACTTTGCTTTCCTGAGCCCGCTTGCAAGCA |
| | GTGCAGCTTCCCGTTCATCCGCCCGCGATGACAAGTTGACGGCT |
| | CTTTTGGCACAATTGGATTCTTTGACCCGGGAACTTAATGTCGT |
| | TTCTCAGCAGCTGTTGGATCTGCGCCAGCAGGTTTCTGCCCTGA |
| | AGGCTTCCTCCCCTCCCAATGCGGTTTAAAACATAAATAAAAA |
| | ACCAGACTCTGTTTGGATTTGGATCAAGCAAGTGTCTTGCTGTC |
| | TTTATTTAGGGGTTTTGCGCGCGCGGTAGGCCCGGGACCAGCG |
| | GTCTCGGTCGTTGAGGGTCCTGTGTATTTTTTCCAGGACGTGGT |
| | AAAGGTGACTCTGGATGTTCAGATACATGGGCATAAGCCCGTC |
| | TCTGGGGTGGAGGTAGCACCACTGCAGAGCTTCATGCTGCGGG |
| | GTGGTGTTGTAGATGATCCAGTCGTAGCAGGAGCGCTGGGCGT |
| | GGTGCCTAAAAATGTCTTTCAGTAGCAAGCTGATTGCCAGGGG |
| | CAGGCCCTTGGTGTAAGTGTTTACAAAGCGGTTAAGCTGGGAT |
| | GGGTGCATACGTGGGGATATGAGATGCATCTTGGACTGTATTTT |
| | TAGGTTGGCTATGTTCCCAGCCATATCCCTCCGGGGATTCATGT |
| | TGTGCAGAACCACCAGCACAGTGTATCCGGTGCACTTGGGAAA |
| | TTTGTCATGTAGCTTAGAAGGAAATGCGTGGAAGAACTTGGAG |
| | ACGCCCTTGTGACCTCCAAGATTTTCCATGCATTCGTCCATAAT |
| | GATGGCAATGGGCCCACGGGCGGCGGCCTGGGCGAAGATATTT |
| | CTGGGATCACTAACGTCATAGTTGTGTTCCAGGATGAGATCGTC |
| | ATAGGCCATTTTTACAAAGCGCGGGCGGAGGGTGCCAGACTGC |
| | GGTATAATGGTTCCATCCGGCCCAGGGGCGTAGTTACCCTCAC |
| | AGATTTGCATTTCCCACGCTTTGAGTTCAGATGGGGGGATCATG |
| | TCTACCTGCGGGGCGATGAAGAAAACGGTTTCCGGGGTAGGGG |
| | AGATCAGCTGGGAAGAAAGCAGGTTCCTGAGCAGCTGCGACTT |
| | ACCGCAGCCGGTGGGCCCGTAAATCACACCTATTACCGGCTGC |
| | AACTGGTAGTTAAGAGAGCTGCAGCTGCCGTCATCCCTGAGCA |
| | GGGGGGCCACTTCGTTAAGCATGTCCCTGACTCGCATGTTTTCC |
| | CTGACCAAATCCGCCAGAAGGCGCTCGCCGCCCAGCGATAGCA |
| | GTTCTTGCAAGGAAGCAAAGTTTTTCAACGGTTTGAGACCGTCC |
| | GCCGTAGGCATGCTTTTGAGCGTTTGACCAAGCAGTTCCAGGC |
| | GGTCCCACAGCTCGGTCACCTGCTCTACGGCATCTCGATCCAGC |
| | ATATCTCCTCGTTTCGCGGGTTGGGGCGGCTTTCGCTGTACGGC |
| | AGTAGTCGGTGCTCGTCCAGACGGGCCAGGGTCATGTCTTTCC |
| | ACGGGCGCAGGGTCCTCGTCAGCGTAGTCTGGGTCACGGTGAA |
| | GGGGTGCGCTCCGGGCTGCGCGCTGGCCAGGGTGCGCTTGAGG |
| | CTGGTCCTGCTGGTGCTGAAGCGCTGCCGGTCTTCGCCCTGCGC |
| | GTCGGCCAGGTAGCATTTGACCATGGTGTCATAGTCCAGCCCCT |
| | CCGCGGCGTGGCCCTTGGCGCGCAGCTTGCCCTTGGAGGAGGC |
| | GCCGCACGAGGGGCAGTGCAGACTTTTGAGGGCGTAGAGCTTG |
| | GGCGCGAGAAATACCGATTCCGGGGAGTAGGCATCCGCGCCGC |
| | AGGCCCCGCAGACGGTCTCGCATTCCACGAGCCAGGTGAGCTC |
| | TGGCCGTTCGGGGTCAAAAACCAGGTTTCCCCCATGCTTTTTGA |
| | TGCGTTTCTTACCTCTGGTTTCCATGAGCCGGTGTCCACGCTCG |
| | GTGACGAAAAGGCTGTCCGTGTCCCCGTATACAGACTTGAGAG |
| | GCCTGTCCTCGAGCGGTGTTCCGCGGTCCTCCTCGTATAGAAAC |
| | TCGGACCACTCTGAGACAAAGGCTCGCGTCCAGGCCAGCACGA |
| | AGGAGGCTAAGTGGGAGGGGTAGCGGTCGTTGTCCACTAGGGG |
| | GTCCACTCGCTCCAGGGTGTGAAGACACATGTCGCCCTCTTCGG |
| | CATCAAGGAAGGTGATTGGTTTGTAGGTGTAGGCCACGTGACC |
| | GGGTGTTCCTGAAGGGGGGCTATAAAAGGGGGTGGGGGCGCG |
| | TTCGTCCTCACTCTCTTCCGCATCGCTGTCTGCGAGGGCCAGCT |
| | GTTGGGGTGAGTACTCCCTCTGAAAAGCGGGCATGACTTCTGC |
| | GCTAAGATTGTCAGTTTCCAAAAACGAGGAGGATTTGATATTC |
| | ACCTGGCCCGCGGTGATGCCTTTGAGGGTGGCCGCATCCATCT |
| | GGTCAGAAAAGACAATCTTTTTGTTGTCAAGCTTGGTGGCAAA |
| | CGACCCGTAGAGGGCGTTGGACAGCAACTTGGCGATGGAGCGC |
| | AGGGTTTGGTTTTTGTCGCGATCGGCGCGCTCCTTGGCCGCGAT |
| | GTTTAGCTGCACGTATTCGCGCGCAACGCACCGCCATTCGGGA |
| | AAGACGGTGGTGCGCTCGTCGGGCACCAGGTGCACGCGCCAAC |
| | CGCGGTTGTGCAGGGTGACAAGGTCAACGCTGGTGGCTACCTC |
| | TCCGCGTAGGCGCTCGTTGGTCCAGCAGAGGCGGCCGCCCTTG |
| | CGCGAGCAGAATGGCGGTAGGGGGTCTAGCTGCGTCTCGTCCG |
| | GGGGGTCTGCGTCCACGGTAAAGACCCCGGGCAGCAGGCGCGC |
| | GTCGAAGTAGTCTATCTTGCATCCTTGCAAGTCTAGCGCCTGCT |
| | GCCATGCGCGGGCGGCAAGCGCGCGCTCGTATGGGTTGAGTGG |
| | GGGACCCCATGGCATGGGGTGGGTGAGCGCGGAGGCGTACAT |
| | GCCGCAAATGTCGTAAACGTAGAGGGGCTCTCTGAGTATTCCA |
| | AGATATGTAGGGTAGCATCTTCCACCGCGGATGCTGGCGCGCA |
| | CGTAATCGTATAGTTCGTGCGAGGGAGCGAGGAGGTCGGGACC |
| | GAGGTTGCTACGGGCGGGCTGCTCTGCTCGGAAGACTATCTGC |
| | CTGAAGATGGCATGTGAGTTGGATGATATGGTTGGACGCTGGA |
| | AGACGTTGAAGCTGGCGTCTGTGAGACCTACCGCGTCACGCAC |
| | GAAGGAGGCGTAGGAGTCGCGCAGCTTGTTGACCAGCTCGGCG |
| | GTGACCTGCACGTCTAGGGCGCAGTAGTCCAGGGTTTCCTTGAT |
| | GATGTCATACTTATCCTGTCCCTTTTTTTTCCACAGCTCGCGGTT |
| | GAGGACAAACTCTTCGCGGTCTTTCCAGTACTCTTGGATCGGAA |
| | ACCCGTCGGCCTCCGAACGGTAAGAGCCTAGCATGTAGAACTG |
| | GTTGACGGCCTGGTAGGCGCAGCATCCCTTTTCTACGGGTAGC |
| | GCGTATGCCTGCGCGGCCTTCCGGCATGACCAGCATGAAGGGC |
| | ACGAGCTGCTTCCCAAAGGCCCCCATCCAAGTATAGGTCTCTA |
| | CATCGTAGGTGACAAAGAGACGCTCGGTGCGAGGATGCGAGCC |
| | GATCGGGAAGAACTGGATCTCCCGCCACCAATTGGAGGAGTGG |
| | CTATTGATGTGGTGAAAGTAGAAGTCCCTGCGACGGGCCGAAC |
| | ACTCGTGCTGGCTTTTGTAAAAACGTGCGCAGTACTGGCAGCG |
| | GTGCACGGGCTGTACATCCTGCACGAGGTTGACCTGACGACCG |
| | CGCACAAGGAAGCAGAGTGGGAATTTGAGCCCCTCGCCTGGCG |
| | GGTTTGGCTGGTGGTCTTCTACTTCGGCTGCTTGTCCTTGACCG |
| | TCTGGCTGCTCGAGGGGAGTTACGGTGGATCGGACCACCACGC |
| | CGCGCGAGCCCAAAGTCCAGATGTCCGCGCGCGGCGGTCGGAG |
| | CTTGATGACAACATCGCGCAGATGGGAGCTGTCCATGGTCTGG |
| | AGCTCCCGCGGCGTCAGGTCAGGCGGGAGCTCCTGCAGGTTTA |
| | CCTCGCATAGACGGGTCAGGGCGCGGGCTAGATCCAGGTGATA |
| | CCTAATTTCCAGGGGCTGGTTGGTGGCGGCGTCGATGGCTTGC |
| | AAGAGGCCGCATCCCCGCGGCGCGACTACGGTACCGCGCGGCG |
| | GGCGGTGGGCCGCGGGGGTGTCCTTGGATGATGCATCTAAAAG |
| | CGGTGACGCGGGCGAGCCCCCGGAGGTAGGGGGGGCTCCGGA |
| | CCCGCCGGGAGAGGGGGCAGGGGCACGTCGGCGCCGCGCGCG |
| | GGCAGGAGCTGGTGCTGCGCGCGTAGGTTGCTGGCGAACGCGA |
| | CGACGCGGCGGTTGATCTCCTGAATCTGGCGCCTCTGCGTGAA |
| | GACGACGGGCCCGGTGAGCTTGAACCTGAAAGAGAGTTCGACA |
| | GAATCAATTTCGGTGTCGTTGACGGCGGCCTGGCGCAAAATCT |
| | CCTGCACGTCTCCTGAGTTGTCTTGATAGGCGATCTCGGCCATG |
| | AACTGCTCGATCTCTTCCTCCTGGAGATCTCCGCGTCCGGCTCG |
| | CTCCACGGTGGCGGCGAGGTCGTTGGAAATGCGGGCCATGAGC |
| | TGCGAGAAGGCGTTGAGGCCTCCCTCGTTCCAGACGCGGCTGT |
| | AGACCACGCCCCCTTCGGCATCGCGGGCGCGCATGACCACCTG |
| | CGCGAGATTGAGCTCCACGTGCCGGGCGAAGACGGCGTAGTTT |
| | CGCAGGCGCTGAAAGAGGTAGTTGAGGGTGGTGGCGGTGTGTT |
| | CTGCCACGAAGAAGTACATAACCCAGCGTCGCAACGTGGATTC |
| | GTTGATAATTGTTGTGTAGGTACTCCGCCGCCGAGGGACCTGA |
| | GCGAGTCCGCATCGACCGGATCGGAAAACCTCTCGAGAAAGGC |
| | GTCTAACCAGTCACAGTCGCAAGGTAGGCTGAGCACCGTGGCG |
| | GGCGGCAGCGGGCGGCGGTCGGGGTTGTTTCTGGCGGAGGTGC |
| | TGCTGATGATGTAATTAAAGTAGGCGGTCTTGAGACGGCGGAT |
| | GGTCGACAGAAGCACCATGTCCTTGGGTCCGGCCTGCTGAATG |
| | CGCAGGCGGTCGGCCATGCCCCAGGCTTCGTTTTGACATCGGC |
| | GCAGGTCTTTGTAGTAGTCTTGCATGAGCCTTTCTACCGGCACT |
| | TCTTCTTCTCCTTCCTCTTGTCCTGCATCTCTTGCATCTATCGCT |
| | GCGGCGGCGGCGGAGTTTGGCCGTAGGTGGCGCCCTCTTCCTC |
| | CCATGCGTGTGACCCCGAAGCCCCTCATCGGCTGAAGCAGGGC |
| | TAGGTCGGCGACAACGCGCTCGGCTAATATGGCCTGCTGCACC |
| | TGCGTGAGGGTAGACTGGAAGTCATCCATGTCCACAAAGCGGT |
| | GGTATGCGCCCGTGTTGATGGTGTAAGTGCAGTTGGCCATAAC |
| | GGACCAGTTAACGGTCTGGTGACCCGGCTGCGAGAGCTCGGTG |
| | TACCTGAGACGCGAGTAAGCCCTCGAGTCAAATACGTAGTCGT |
| | TGCAAGTCCGCACCAGGTACTGGTATCCCACCAAAAAGTGCGG |
| | CGGCGGCTGGCGGTAGAGGGGCCAGCGTAGGGTGGCCGGGGC |
| | TCCGGGGGCGAGATCTTCCAACATAAGGCGATGATATCCGTAG |
| | ATGTACCTGGACATCCAGGTGATGCCGGCGGCGGTGGTGGAGG |
| | CGCGCGGAAAGTCGCGGACGCGGTTCCAGATGTTGCGCAGCGG |
| | CAAAAAGTGCTCCATGGTCGGGACGCTCTGGCCGGTCAGGCGC |
| | GCGCAATCGTTGACGCTCTAGCGTGCAAAAGGAGAGCCTGTAA |
| | GCGGGCACTCTTCCGTGGTCTGGTGGATAAATTCGCAAGGGTA |
| | TCATGGCGGACGACCGGGGTTCGAGCCCCGTATCCGGCCGTCC |
| | GCCGTGATCCATGCGGTTACCGCCCGCGTGTCGAACCCAGGTG |
| | TGCGACGTCAGACAACGGGGGAGTGCTCCTTTTGGCTTCCTTCC |
| | AGGCGCGGCGGCTGCTGCGCTAGCTTTTTTGGCCACTGGCCGC |
| | GCGCAGCGTAAGCGGTTAGGCTGGAAAGCGAAAGCATTAAGT |
| | GGCTCGCTCCCTGTAGCCGGAGGGTTATTTTCCAAGGGTTGAGT |
| | CGCGGGACCCCCGGTTCGAGTCTCGGACCGGCCGGACTGCGGC |
| | GAACGGGGGTTTGCCTCCCCGTCATGCAAGACCCCGCTTGCAA |
| | ATTCCTCCGGAAACAGGGACGAGCCCCTTTTTTGCTTTTCCCAG |
| | ATGCATCCGGTGCTGCGGCAGATGCGCCCCCCTCCTCAGCAGC |
| | GGCAAGAGCAAGAGCAGCGGCAGACATGCAGGGCACCCTCCC |
| | CTCCTCCTACCGCGTCAGGAGGGGCGACATCCGCGGTTGACGC |
| | GGCAGCAGATGGTGATTACGAACCCCCGCGGCGCCGGGCCCGG |
| | CACTACCTGGACTTGGAGGAGGGCGAGGGCCTGGCGCGGCTAG |
| | GAGCGCCCTCTCCTGAGCGGCACCCAAGGGTGCAGCTGAAGCG |
| | TGATACGCGTGAGGCGTACGTGCCGCGGCAGAACCTGTTTCGC |
| | GACCGCGAGGGAGAGGAGCCCGAGGAGATGCGGGATCGAAAG |
| | TTCCACGCAGGGCGCGAGCTGCGGCATGGCCTGAATCGCGAGC |
| | GGTTGCTGCGCGAGGAGGACTTTGAGCCCGACGCGCGAACCGG |
| | GATTAGTCCCGCGCGCGCACACGTGGCGGCCGCCGACCTGGTA |
| | ACCGCATACGAGCAGACGGTGAACCAGGAGATTAACTTTCAAA |
| | AAAGCTTTAACAACCACGTGCGTACGCTTGTGGCGCGCGAGGA |
| | GGTGGCTATAGGACTGATGCATCTGTGGGACTTTGTAAGCGCG |
| | CTGGAGCAAAACCCAAATAGCAAGCCGCTCATGGCGCAGCTGT |
| | TCCTTATAGTGCAGCACAGCAGGGACAACGAGGCATTCAGGGA |
| | TGCGCTGCTAAACATAGTAGAGCCCGAGGGCCGCTGGCTGCTC |
| | GATTTGATAAACATCCTGCAGAGCATAGTGGTGCAGGAGCGCA |
| | GCTTGAGCCTGGCTGACAAGGTGGCCGCCATCAACTATTCCAT |
| | GCTTAGCCTGGGCAAGTTTTACGCCCGCAAGATATACCATACC |
| | CCTTACGTTCCCATAGACAAGGAGGTAAAGATCGAGGGGTTCT |
| | ACATGCGCATGGCGCTGAAGGTGCTTACCTTGAGCGACGACCT |
| | GGGCGTTTATCGCAACGAGCGCATCCACAAGGCCGTGAGCGTG |
| | AGCCGGCGGCGCGAGCTCAGCGACCGCGAGCTGATGCACAGCC |
| | TGCAAAGGGCCCTGGCTGGCACGGGCAGCGGCGATAGAGAGG |
| | CCGAGTCCTACTTTGACGCGGGCGCTGACCTGCGCTGGGCCCC |
| | AAGCCGACGCGCCCTGGAGGCAGCTGGGGCCGGACCTGGGCTG |
| | GCGGTGGCACCCGCGCGCGCTGGCAACGTCGGCGGCGTGGAGG |
| | AATATGACGAGGACGATGAGTACGAGCCAGAGGACGGCGAGT |
| | ACTAAGCGGTGATGTTTCTGATCAGATGATGCAAGACGCAACG |
| | GACCCGGCGGTGCGGGCGGCGCTGCAGAGCCAGCCGTCCGGCC |
| | TTAACTCCACGGACGACTGGCGCCAGGTCATGGACCGCATCAT |
| | GTCGCTGACTGCGCGCAATCCTGACGCGTTCCGGCAGCAGCCG |
| | CAGGCCAACCGGCTCTCCGCAATTCTGGAAGCGGTGGTCCCGG |
| | CGCGCGCAAACCCCACGCACGAGAAGGTGCTGGCGATCGTAAA |
| | CGCGCTGGCCGAAAACAGGGCCATCCGGCCCGACGAGGCCGG |
| | CCTGGTCTACGACGCGCTGCTTCAGCGCGTGGCTCGTTACAACA |
| | GCGGCAACGTGCAGACCAACCTGGACCGGCTGGTGGGGGATGT |
| | GCGCGAGGCCGTGGCGCAGCGTGAGCGCGCGCAGCAGCAGGG |
| | CAACCTGGGCTCCATGGTTGCACTAAACGCCTTCCTGAGTACAC |
| | AGCCCGCCAACGTGCCGCGGGGACAGGAGGACTACACCAACTT |
| | TGTGAGCGCACTGCGGCTAATGGTGACTGAGACACCGCAAAGT |
| | GAGGTGTACCAGTCTGGGCCAGACTATTTTTTCCAGACCAGTA |
| | GACAAGGCCTGCAGACCGTAAACCTGAGCCAGGCTTTCAAAAA |
| | CTTGCAGGGGCTGTGGGGGGTGCGGGCTCCCACAGGCGACCGC |
| | GCGACCGTGTCTAGCTTGCTGACGCCCAACTCGCGCCTGTTGCT |
| | GCTGCTAATAGCGCCCTTCACGGACAGTGGCAGCGTGTCCCGG |
| | GACACATACCTAGGTCACTTGCTGACACTGTACCGCGAGGCCA |
| | TAGGTCAGGCGCATGTGGACGAGCATACTTTCCAGGAGATTAC |
| | AAGTGTCAGCCGCGCGCTGGGGCAGGAGGACACGGGCAGCCT |
| | GGAGGCAACCCTAAACTACCTGCTGACCAACCGGCGGCAGAAG |
| | ATCCCCTCGTTGCACAGTTTAAACAGCGAGGAGGAGCGCATTT |
| | TGCGCTACGTGCAGCAGAGCGTGAGCCTTAACCTGATGCGCGA |
| | CGGGGTAACGCCCAGCGTGGCGCTGGACATGACCGCGCGCAAC |
| | ATGGAACCGGGCATGTATGCCTCAAACCGGCCGTTTATCAACC |
| | GCCTAATGGACTACTTGCATCGCGCGGCCGCCGTGAACCCCGA |
| | GTATTTCACCAATGCCATCTTGAACCCGCACTGGCTACCGCCCC |
| | CTGGTTTCTACACCGGGGGATTCGAGGTGCCCGAGGGTAACGA |
| | TGGATTCCTCTGGGACGACATAGACGACAGCGTGTTTTCCCCGC |
| | AACCGCAGACCCTGCTAGAGTTGCAACAGCGCGAGCAGGCAG |
| | AGGCGGCGCTGCGAAAGGAAAGCTTCCGCAGGCCAAGCAGCTT |
| | GTCCGATCTAGGCGCTGCGGCCCCGCGGTCAGATGCTAGTAGC |
| | CCATTTCCAAGCTTGATAGGGTCTCTTACCAGCACTCGCACCAC |
| | CCGCCCGCGCCTGCTGGGCGAGGAGGAGTACCTAAACAACTCG |
| | CTGCTGCAGCCGCAGCGCGAAAAAAACCTGCCTCCGGCATTTC |
| | CCAACAACGGGATAGAGAGCCTAGTGGACAAGATGAGTAGAT |
| | GGAAGACGTACGCGCAGGAGCACAGGGACGTGCCAGGCCCGC |
| | GCCCGCCCACCCGTCGTCAAAGGCACGACCGTCAGCGGGGTCT |
| | GGTGTGGGAGGACGATGACTCGGCAGACGACAGCAGCGTCCTG |
| | GATTTGGGAGGGAGTGGCAACCCGTTTGCGCACCTTCGCCCCA |
| | GGCTGGGGAGAATGTTTTAAAAAAAAAAAAGCATGATGCAAA |
| | ATAAAAAACTCACCAAGGCCATGGCACCGAGCGTTGGTTTTCT |
| | TGTATTCCCCTTAGTATGCGGCGCGCGGCGATGTATGAGGAAG |
| | GTCCTCCTCCCTCCTACGAGAGTGTGGTGAGCGCGGCGCCAGT |
| | GGCGGCGGCGCTGGGTTCTCCCTTCGATGCTCCCCTGGACCCGC |
| | CGTTTGTGCCTCCGCGGTACCTGCGGCCTACCGGGGGGAGAAA |
| | CAGCATCCGTTACTCTGAGTTGGCACCCCTATTCGACACCACCC |
| | GTGTGTACCTGGTGGACAACAAGTCAACGGATGTGGCATCCCT |
| | GAACTACCAGAACGACCACAGCAACTTTCTGACCACGGTCATT |
| | CAAAACAATGACTACAGCCCGGGGGAGGCAAGCACACAGACC |
| | ATCAATCTTGACGACCGGTCGCACTGGGGCGGCGACCTGAAAA |
| | CCATCCTGCATACCAACATGCCAAATGTGAACGAGTTCATGTTT |
| | ACCAATAAGTTTAAGGCGCGGGTGATGGTGTCGCGCTTGCCTA |
| | CTAAGGACAATCAGGTGGAGCTGAAATACGAGTGGGTGGAGTT |
| | CACGCTGCCCGAGGGCAACTACTCCGAGACCATGACCATAGAC |
| | CTTATGAACAACGCGATCGTGGAGCACTACTTGAAAGTGGGCA |
| | GACAGAACGGGGTTCTGGAAAGCGACATCGGGGTAAAGTTTGA |
| | CACCCGCAACTTCAGACTGGGGTTTGACCCCGTCACTGGTCTTG |
| | TCATGCCTGGGGTATATACAAACGAAGCCTTCCATCCAGACAT |
| | CATTTTGCTGCCAGGATGCGGGGTGGACTTCACCCACAGCCGC |
| | CTGAGCAACTTGTTGGGCATCCGCAAGCGGCAACCCTTCCAGG |
| | AGGGCTTTAGGATCACCTACGATGATCTGGAGGGTGGTAACAT |
| | TCCCGCACTGTTGGATGTGGACGCCTACCAGGCGAGCTTGAAA |
| | GATGACACCGAACAGGGCGGGGGTGGCGCAGGCGGCAGCAAC |
| | AGCAGTGGCAGCGGCGCGGAAGAGAACTCCAACGCGGCAGCC |
| | GCGGCAATGCAGCCGGTGGAGGACATGAACGATCATGCCATTC |
| | GCGGCGACACCTTTGCCACACGGGCTGAGGAGAAGCGCGCTGA |
| | GGCCGAAGCAGCGGCCGAAGCTGCCGCCCCCGCTGCGCAACCC |
| | GAGGTCGAGAAGCCTCAGAAGAAACCGGTGATCAAACCCCTG |
| | ACAGAGGACAGCAAGAAACGCAGTTACAACCTAATAAGCAAT |
| | GACAGCACCTTCACCCAGTACCGCAGCTGGTACCTTGCATACA |
| | ACTACGGCGACCCTCAGACCGGAATCCGCTCATGGACCCTGCT |
| | TTGCACTCCTGACGTAACCTGCGGCTCGGAGCAGGTCTACTGGT |
| | CGTTGCCAGACATGATGCAAGACCCCGTGACCTTCCGCTCCAC |
| | GCGCCAGATCAGCAACTTTCCGGTGGTGGGCGCCGAGCTGTTG |
| | CCCGTGCACTCCAAGAGCTTCTACAACGACCAGGCCGTCTACT |
| | CCCAACTCATCCGCCAGTTTACCTCTCTGACCCACGTGTTCAAT |
| | CGCTTTCCCGAGAACCAGATTTTGGCGCGCCCGCCAGCCCCCA |
| | CCATCACCACCGTCAGTGAAAACGTTCCTGCTCTCACAGATCAC |
| | GGGACGCTACCGCTGCGCAACAGCATCGGAGGAGTCCAGCGA |
| | GTGACCATTACTGACGCCAGACGCCGCACCTGCCCCTACGTTTA |
| | CAAGGCCCTGGGCATAGTCTCGCCGCGCGTCCTATCGAGCCGC |
| | ACTTTTTGAGCAAGCATGTCCATCCTTATATCGCCCAGCAATAA |
| | CACAGGCTGGGGCCTGCGCTTCCCAAGCAAGATGTTTGGCGGG |
| | GCCAAGAAGCGCTCCGACCAACACCCAGTGCGCGTGCGCGGGC |
| | ACTACCGCGCGCCCTGGGGCGCGCACAAACGCGGCCGCACTGG |
| | GCGCACCACCGTCGATGACGCCATCGACGCGGTGGTGGAGGAG |
| | GCGCGCAACTACACGCCCACGCCGCCACCAGTGTCCACAGTGG |
| | ACGCGGCCATTCAGACCGTGGTGCGCGGAGCCCGGCGCTATGC |
| | TAAAATGAAGAGACGGCGGAGGCGCGTAGCACGTCGCCACCG |
| | CCGCCGACCCGGCACTGCCGCCCAACGCGCGGCGGCGGCCCTG |
| | CTTAACCGCGCACGTCGCACCGGCCGACGGGCGGCCATGCGGG |
| | CCGCTCGAAGGCTGGCCGCGGGTATTGTCACTGTGCCCCCCAG |
| | GTCCAGGCGACGAGCGGCCGCCGCAGCAGCCGCGGCCATTAGT |
| | GCTATGACTCAGGGTCGCAGGGGCAACGTGTATTGGGTGCGCG |
| | ACTCGGTTAGCGGCCTGCGCGTGCCCGTGCGCACCCGCCCCCC |
| | GCGCAACTAGATTGCAAGAAAAAACTACTTAGACTCGTACTGT |
| | TGTATGTATCCAGCGGCGGCGGCGCGCAACGAAGCTATGTCCA |
| | AGCGCAAAATCAAAGAAGAGATGCTCCAGGTCATCGCGCCGG |
| | AGATCTATGGCCCCCCGAAGAAGGAAGAGCAGGATTACAAGC |
| | CCCGAAAGCTAAAGCGGGTCAAAAAGAAAAAGAAAGATGATG |
| | ATGATGAACTTGACGACGAGGTGGAACTGCTGCACGCTACCGC |
| | GCCCAGGCGACGGGTACAGTGGAAAGGTCGACGCGTAAAACG |
| | TGTTTTGCGACCCGGCACCACCGTAGTCTTTACGCCCGGTGAGC |
| | GCTCCACCCGCACCTACAAGCGCGTGTATGATGAGGTGTACGG |
| | CGACGAGGACCTGCTTGAGCAGGCCAACGAGCGCCTCGGGGA |
| | GTTTGCCTACGGAAAGCGGCATAAGGACATGCTGGCGTTGCCG |
| | CTGGACGAGGGCAACCCAACACCTAGCCTAAAGCCCGTAACAC |
| | TGCAGCAGGTGCTGCCCGCGCTTGCACCGTCCGAAGAAAAGCG |
| | CGGCCTAAAGCGCGAGTCTGGTGACTTGGCACCCACCGTGCAG |
| | CTGATGGTACCCAAGCGCCAGCGACTGGAAGATGTCTTGGAAA |
| | AAATGACCGTGGAACCTGGGCTGGAGCCCGAGGTCCGCGTGCG |
| | GCCAATCAAGCAGGTGGCGCCGGGACTGGGCGTGCAGACCGTG |
| | GACGTTCAGATACCCACTACCAGTAGCACCAGTATTGCCACCG |
| | CCACAGAGGGCATGGAGACACAAACGTCCCCGGTTGCCTCAGC |
| | GGTGGCGGATGCCGCGGTGCAGGCGGTCGCTGCGGCCGCGTCC |
| | AAGACCTCTACGGAGGTGCAAACGGACCCGTGGATGTTTCGCG |
| | TTTCAGCCCCCCGGCGCCCGCGCCGTTCGAGGAAGTACGGCGC |
| | CGCCAGCGCGCTACTGCCCGAATATGCCCTACATCCTTCCATTG |
| | CGCCTACCCCCGGCTATCGTGGCTACACCTACCGCCCCAGAAG |
| | ACGAGCAACTACCCGACGCCGAACCACCACTGGAACCCGCCGC |
| | CGCCGTCGCCGTCGCCAGCCCGTGCTGGCCCCGATTTCCGTGCG |
| | CAGGGTGGCTCGCGAAGGAGGCAGGACCCTGGTGCTGCCAACA |
| | GCGCGCTACCACCCCAGCATCGTTTAAAAGCCGGTCTTTGTGGT |
| | TCTTGCAGATATGGCCCTCACCTGCCGCCTCCGTTTCCCGGTGC |
| | CGGGATTCCGAGGAAGAATGCACCGTAGGAGGGGCATGGCCG |
| | GCCACGGCCTGACGGGCGGCATGCGTCGTGCGCACCACCGGCG |
| | GCGGCGCGCGTCGCACCGTCGCATGCGCGGCGGTATCCTGCCC |
| | CTCCTTATTCCACTGATCGCCGCGGCGATTGGCGCCGTGCCCGG |
| | AATTGCATCCGTGGCCTTGCAGGCGCAGAGACACTGATTAAAA |
| | ACAAGTTGCATGTGGAAAAATCAAAATAAAAAGTCTGGACTCT |
| | CACGCTCGCTTGGTCCTGTAACTATTTTGTAGAATGGAAGACAT |
| | CAACTTTGCGTCTCTGGCCCCGCGACACGGCTCGCGCCCGTTCA |
| | TGGGAAACTGGCAAGATATCGGCACCAGCAATATGAGCGGTGG |
| | CGCCTTCAGCTGGGGCTCGCTGTGGAGCGGCATTAAAAATTTC |
| | GGTTCCACCGTTAAGAACTATGGCAGCAAGGCCTGGAACAGCA |
| | GCACAGGCCAGATGCTGAGGGATAAGTTGAAAGAGCAAAATTT |
| | CCAACAAAAGGTGGTAGATGGCCTGGCCTCTGGCATTAGCGGG |
| | GTGGTGGACCTGGCCAACCAGGCAGTGCAAAATAAGATTAACA |
| | GTAAGCTTGATCCCCGCCCTCCCGTAGAGGAGCCTCCACCGGC |
| | CGTGGAGACAGTGTCTCCAGAGGGGCGTGGCGAAAAGCGTCCG |
| | CGCCCCGACAGGGAAGAAACTCTGGTGACGCAAATAGACGAG |
| | CCTCCCTCGTACGAGGAGGCACTAAAGCAAGGCCTGCCCACCA |
| | CCCGTCCCATCGCGCCCATGGCTACCGGAGTGCTGGGCCAGCA |
| | CACACCCGTAACGCTGGACCTGCCTCCCCCCGCCGACACCCAG |
| | CAGAAACCTGTGCTGCCAGGCCCGACCGCCGTTGTTGTAACCC |
| | GTCCTAGCCGCGCGTCCCTGCGCCGCGCCGCCAGCGGTCCGCG |
| | ATCGTTGCGGCCCGTAGCCAGTGGCAACTGGCAAAGCACACTG |
| | AACAGCATCGTGGGTCTGGGGGTGCAATCCCTGAAGCGCCGAC |
| | GATGCTTCTGATAGCTAACGTGTCGTATGTGTGTCATGTATGCG |
| | TCCATGTCGCCGCCAGAGGAGCTGCTGAGCCGCCGCGCGCCCG |
| | CTTTCCAAGATGGCTACCCCTTCGATGATGCCGCAGTGGTCTTA |
| | CATGCACATCTCGGGCCAGGACGCCTCGGAGTACCTGAGCCCC |
| | GGGCTGGTGCAGTTTGCCCGCGCCACCGAGACGTACTTCAGCC |
| | TGAATAACAAGTTTAGAAACCCCACGGTGGCGCCTACGCACGA |
| | CGTGACCACAGACCGGTCCCAGCGTTTGACGCTGCGGTTCATC |
| | CCTGTGGACCGTGAGGATACTGCGTACTCGTACAAGGCGCGGT |
| | TCACCCTAGCTGTGGGTGATAACCGTGTGCTGGACATGGCTTCC |
| | ACGTACTTTGACATCCGCGGCGTGCTGGACAGGGGCCCTACTTT |
| | TAAGCCCTACTCTGGCACTGCCTACAACGCCCTGGCTCCCAAG |
| | GGTGCCCCAAATCCTTGCGAATGGGATGAAGCTGCTACTGCTC |
| | TTGAAATAAACCTAGAAGAAGAGGACGATGACAACGAAGACG |
| | AAGTAGACGAGCAAGCTGAGCAGCAAAAAACTCACGTATTTGG |
| | GCAGGCGCCTTATTCTGGTATAAATATTACAAAGGAGGGTATT |
| | CAAATAGGTGTCGAAGGTCAAACACCTAAATATGCCGATAAAA |
| | CATTTCAACCTGAACCTCAAATAGGAGAATCTCAGTGGTACGA |
| | AACAGAAATTAATCATGCAGCTGGGAGAGTCCTAAAAAAGACT |
| | ACCCCAATGAAACCATGTTACGGTTCATATGCAAAACCCACAA |
| | ATGAAAATGGAGGGCAAGGCATTCTTGTAAAGCAACAAAATG |
| | GAAAGCTAGAAAGTCAAGTGGAAATGCAATTTTTCTCAACTAC |
| | TGAGGCAGCCGCAGGCAATGGTGATAACTTGACTCCTAAAGTG |
| | GTATTGTACAGTGAAGATGTAGATATAGAAACCCCAGACACTC |
| | ATATTTCTTACATGCCCACTATTAAGGAAGGTAACTCACGAGA |
| | ACTAATGGGCCAACAATCTATGCCCAACAGGCCTAATTACATT |
| | GCTTTTAGGGACAATTTTATTGGTCTAATGTATTACAACAGCAC |
| | GGGTAATATGGGTGTTCTGGCGGGCCAAGCATCGCAGTTGAAT |
| | GCTGTTGTAGATTTGCAAGACAGAAACACAGAGCTTTCATACC |
| | AGCTTTTGCTTGATTCCATTGGTGATAGAACCAGGTACTTTTCT |
| | ATGTGGAATCAGGCTGTTGACAGCTATGATCCAGATGTTAGAA |
| | TTATTGAAAATCATGGAACTGAAGATGAACTTCCAAATTACTG |
| | CTTTCCACTGGGAGGTGTGATTAATACAGAGACTCTTACCAAG |
| | GTAAAACCTAAAACAGGTCAGGAAAATGGATGGGAAAAAGAT |
| | GCTACAGAATTTTCAGATAAAAATGAAATAAGAGTTGGAAATA |
| | ATTTTGCCATGGAAATCAATCTAAATGCCAACCTGTGGAGAAA |
| | TTTCCTGTACTCCAACATAGCGCTGTATTTGCCCGACAAGCTAA |
| | AGTACAGTCCTTCCAACGTAAAAATTTCTGATAACCCAAACAC |
| | CTACGACTACATGAACAAGCGAGTGGTGGCTCCCGGGCTAGTG |
| | GACTGCTACATTAACCTTGGAGCACGCTGGTCCCTTGACTATAT |
| | GGACAACGTCAACCCATTTAACCACCACCGCAATGCTGGCCTG |
| | CGCTACCGCTCAATGTTGCTGGGCAATGGTCGCTATGTGCCCTT |
| | CCACATCCAGGTGCCTCAGAAGTTCTTTGCCATTAAAAACCTCC |
| | TTCTCCTGCCGGGCTCATACACCTACGAGTGGAACTTCAGGAA |
| | GGATGTTAACATGGTTCTGCAGAGCTCCCTAGGAAATGACCTA |
| | AGGGTTGACGGAGCCAGCATTAAGTTTGATAGCATTTGCCTTTA |
| | CGCCACCTTCTTCCCCATGGCCCACAACACCGCCTCCACGCTTG |
| | AGGCCATGCTTAGAAACGACACCAACGACCAGTCCTTTAACGA |
| | CTATCTCTCCGCCGCCAACATGCTCTACCCTATACCCGCCAACG |
| | CTACCAACGTGCCCATATCCATCCCCTCCCGCAACTGGGCGGCT |
| | TTCCGCGGCTGGGCCTTCACGCGCCTTAAGACTAAGGAAACCC |
| | CATCACTGGGCTCGGGCTACGACCCTTATTACACCTACTCTGGC |
| | TCTATACCCTACCTAGATGGAACCTTTTACCTCAACCACACCTT |
| | TAAGAAGGTGGCCATTACCTTTGACTCTTCTGTCAGCTGGCCTG |
| | GCAATGACCGCCTGCTTACCCCCAACGAGTTTGAAATTAAGCG |
| | CTCAGTTGACGGGGAGGGTTACAACGTTGCCCAGTGTAACATG |
| | ACCAAAGACTGGTTCCTGGTACAAATGCTAGCTAACTATAACA |
| | TTGGCTACCAGGGCTTCTATATCCCAGAGAGCTACAAGGACCG |
| | CATGTACTCCTTCTTTAGAAACTTCCAGCCCATGAGCCGTCAGG |
| | TGGTGGATGATACTAAATACAAGGACTACCAACAGGTGGGCAT |
| | CCTACACCAACACAACAACTCTGGATTTGTTGGCTACCTTGCCC |
| | CCACCATGCGCGAAGGACAGGCCTACCCTGCTAACTTCCCCTA |
| | TCCGCTTATAGGCAAGACCGCAGTTGACAGCATTACCCAGAAA |
| | AAGTTTCTTTGCGATCGCACCCTTTGGCGCATCCCATTCTCCAG |
| | TAACTTTATGTCCATGGGCGCACTCACAGACCTGGGCCAAAAC |
| | CTTCTCTACGCCAACTCCGCCCACGCGCTAGACATGACTTTTGA |
| | GGTGGATCCCATGGACGAGCCCACCCTTCTTTATGTTTTGTTTG |
| | AAGTCTTTGACGTGGTCCGTGTGCACCAGCCGCACCGCGGCGT |
| | CATCGAAACCGTGTACCTGCGCACGCCCTTCTCGGCCGGCAAC |
| | GCCACAACATAAAGAAGCAAGCAACATCAACAACAGCTGCCG |
| | CCATGGGCTCCAGTGAGCAGGAACTGAAAGCCATTGTCAAAGA |
| | TCTTGGTTGTGGGCCATATTTTTTGGGCACCTATGACAAGCGCT |
| | TTCCAGGCTTTGTTTCTCCACACAAGCTCGCCTGCGCCATAGTC |
| | AATACGGCCGGTCGCGAGACTGGGGGCGTACACTGGATGGCCT |
| | TTGCCTGGAACCCGCACTCAAAAACATGCTACCTCTTTGAGCCC |
| | TTTGGCTTTTCTGACCAGCGACTCAAGCAGGTTTACCAGTTTGA |
| | GTACGAGTCACTCCTGCGCCGTAGCGCCATTGCTTCTTCCCCCG |
| | ACCGCTGTATAACGCTGGAAAAGTCCACCCAAAGCGTACAGGG |
| | GCCCAACTCGGCCGCCTGTGGACTATTCTGCTGCATGTTTCTCC |
| | ACGCCTTTGCCAACTGGCCCCAAACTCCCATGGATCACAACCC |
| | CACCATGAACCTTATTACCGGGGTACCCAACTCCATGCTCAAC |
| | AGTCCCCAGGTACAGCCCACCCTGCGTCGCAACCAGGAACAGC |
| | TCTACAGCTTCCTGGAGCGCCACTCGCCCTACTTCCGCAGCCAC |
| | AGTGCGCAGATTAGGAGCGCCACTTCTTTTTGTCACTTGAAAAA |
| | CATGTAAAAATAATGTACTAGAGACACTTTCAATAAAGGCAAA |
| | TGCTTTTATTTGTACACTCTCGGGTGATTATTTACCCCCACCCTT |
| | GCCGTCTGCGCCGTTTAAAAATCAAAGGGGTTCTGCCGCGCAT |
| | CGCTATGCGCCACTGGCAGGGACACGTTGCGATACTGGTGTTT |
| | AGTGCTCCACTTAAACTCAGGCACAACCATCCGCGGCAGCTCG |
| | GTGAAGTTTTCACTCCACAGGCTGCGCACCATCACCAACGCGTT |
| | TAGCAGGTCGGGCGCCGATATCTTGAAGTCGCAGTTGGGGCCT |
| | CCGCCCTGCGCGCGCGAGTTGCGATACACAGGGTTGCAGCACT |
| | GGAACACTATCAGCGCCGGGTGGTGCACGCTGGCCAGCACGCT |
| | CTTGTCGGAGATCAGATCCGCGTCCAGGTCCTCCGCGTTGCTCA |
| | GGGCGAACGGAGTCAACTTTGGTAGCTGCCTTCCCAAAAAGGG |
| | CGCGTGCCCAGGCTTTGAGTTGCACTCGCACCGTAGTGGCATC |
| | AAAAGGTGACCGTGCCCGGTCTGGGCGTTAGGATACAGCGCCT |
| | GCATAAAAGCCTTGATCTGCTTAAAAGCCACCTGAGCCTTTGC |
| | GCCTTCAGAGAAGAACATGCCGCAAGACTTGCCGGAAAACTGA |
| | TTGGCCGGACAGGCCGCGTCGTGCACGCAGCACCTTGCGTCGG |
| | TGTTGGAGATCTGCACCACATTTCGGCCCCACCGGTTCTTCACG |
| | ATCTTGGCCTTGCTAGACTGCTCCTTCAGCGCGCGCTGCCCGTT |
| | TTCGCTCGTCACATCCATTTCAATCACGTGCTCCTTATTTATCAT |
| | AATGCTTCCGTGTAGACACTTAAGCTCGCCTTCGATCTCAGCGC |
| | AGCGGTGCAGCCACAACGCGCAGCCCGTGGGCTCGTGATGCTT |
| | GTAGGTCACCTCTGCAAACGACTGCAGGTACGCCTGCAGGAAT |
| | CGCCCCATCATCGTCACAAAGGTCTTGTTGCTGGTGAAGGTCA |
| | GCTGCAACCCGCGGTGCTCCTCGTTCAGCCAGGTCTTGCATACG |
| | GCCGCCAGAGCTTCCACTTGGTCAGGCAGTAGTTTGAAGTTCG |
| | CCTTTAGATCGTTATCCACGTGGTACTTGTCCATCAGCGCGCGC |
| | GCAGCCTCCATGCCCTTCTCCCACGCAGACACGATCGGCACAC |
| | TCAGCGGGTTCATCACCGTAATTTCACTTTCCGCTTCGCTGGGC |
| | TCTTCCTCTTCCTCTTGCGTCCGCATACCACGCGCCACTGGGTC |
| | GTCTTCATTCAGCCGCCGCACTGTGCGCTTACCTCCTTTGCCAT |
| | GCTTGATTAGCACCGGTGGGTTGCTGAAACCCACCATTTGTAGC |
| | GCCACATCTTCTCTTTCTTCCTCGCTGTCCACGATTACCTCTGGT |
| | GATGGCGGGCGCTCGGGCTTGGGAGAAGGGCGCTTCTTTTTCTT |
| | CTTGGGCGCAATGGCCAAATCCGCCGCCGAGGTCGATGGCCGC |
| | GGGCTGGGTGTGCGCGGCACCAGCGCGTCTTGTGATGAGTCTT |
| | CCTCGTCCTCGGACTCGATACGCCGCCTCATCCGCTTTTTTGGG |
| | GGCGCCCGGGGAGGCGGCGGCGACGGGGACGGGGACGACACG |
| | TCCTCCATGGTTGGGGGACGTCGCGCCGCACCGCGTCCGCGCT |
| | CGGGGGTGGTTTCGCGCTGCTCCTCTTCCCGACTGGCCATTTCC |
| | TTCTCCTATAGGCAGAAAAAGATCATGGAGTCAGTCGAGAAGA |
| | AGGACAGCCTAACCGCCCCCTCTGAGTTCGCCACCACCGCCTC |
| | CACCGATGCCGCCAACGCGCCTACCACCTTCCCCGTCGAGGCA |
| | CCCCCGCTTGAGGAGGAGGAAGTGATTATCGAGCAGGACCCAG |
| | GTTTTGTAAGCGAAGACGACGAGGACCGCTCAGTACCAACAGA |
| | GGATAAAAAGCAAGACCAGGACAACGCAGAGGCAAACGAGGA |
| | ACAAGTCGGGCGGGGGGACGAAAGGCATGGCGACTACCTAGA |
| | TGTGGGAGACGACGTGCTGTTGAAGCATCTGCAGCGCCAGTGC |
| | GCCATTATCTGCGACGCGTTGCAAGAGCGCAGCGATGTGCCCC |
| | TCGCCATAGCGGATGTCAGCCTTGCCTACGAACGCCACCTATTC |
| | TCACCGCGCGTACCCCCCAAACGCCAAGAAAACGGCACATGCG |
| | AGCCCAACCCGCGCCTCAACTTCTACCCCGTATTTGCCGTGCCA |
| | GAGGTGCTTGCCACCTATCACATCTTTTTCCAAAACTGCAAGAT |
| | ACCCCTATCCTGCCGTGCCAACCGCAGCCGAGCGGACAAGCAG |
| | CTGGCCTTGCGGCAGGGCGCTGTCATACCTGATATCGCCTCGCT |
| | CAACGAAGTGCCAAAAATCTTTGAGGGTCTTGGACGCGACGAG |
| | AAGCGCGCGGCAAACGCTCTGCAACAGGAAAACAGCGAAAAT |
| | GAAAGTCACTCTGGAGTGTTGGTGGAACTCGAGGGTGACAACG |
| | CGCGCCTAGCCGTACTAAAACGCAGCATCGAGGTCACCCACTT |
| | TGCCTACCCGGCACTTAACCTACCCCCCAAGGTCATGAGCACA |
| | GTCATGAGTGAGCTGATCGTGCGCCGTGCGCAGCCCCTGGAGA |
| | GGGATGCAAATTTGCAAGAACAAACAGAGGAGGGCCTACCCG |
| | CAGTTGGCGACGAGCAGCTAGCGCGCTGGCTTCAAACGCGCGA |
| | GCCTGCCGACTTGGAGGAGCGACGCAAACTAATGATGGCCGCA |
| | GTGCTCGTTACCGTGGAGCTTGAGTGCATGCAGCGGTTCTTTGC |
| | TGACCCGGAGATGCAGCGCAAGCTAGAGGAAACATTGCACTAC |
| | ACCTTTCGACAGGGCTACGTACGCCAGGCCTGCAAGATCTCCA |
| | ACGTGGAGCTCTGCAACCTGGTCTCCTACCTTGGAATTTTGCAC |
| | GAAAACCGCCTTGGGCAAAACGTGCTTCATTCCACGCTCAAGG |
| | GCGAGGCGCGCCGCGACTACGTCCGCGACTGCGTTTACTTATTT |
| | CTATGCTACACCTGGCAGACGGCCATGGGCGTTTGGCAGCAGT |
| | GCTTGGAGGAGTGCAACCTCAAGGAGCTGCAGAAACTGCTAAA |
| | GCAAAACTTGAAGGACCTATGGACGGCCTTCAACGAGCGCTCC |
| | GTGGCCGCGCACCTGGCGGACATCATTTTCCCCGAACGCCTGCT |
| | TAAAACCCTGCAACAGGGTCTGCCAGACTTCACCAGTCAAAGC |
| | ATGTTGCAGAACTTTAGGAACTTTATCCTAGAGCGCTCAGGAA |
| | TCTTGCCCGCCACCTGCTGTGCACTTCCTAGCGACTTTGTGCCC |
| | ATTAAGTACCGCGAATGCCCTCCGCCGCTTTGGGGCCACTGCTA |
| | CCTTCTGCAGCTAGCCAACTACCTTGCCTACCACTCTGACATAA |
| | TGGAAGACGTGAGCGGTGACGGTCTACTGGAGTGTCACTGTCG |
| | CTGCAACCTATGCACCCCGCACCGCTCCCTGGTTTGCAATTCGC |
| | AGCTGCTTAACGAAAGTCAAATTATCGGTACCTTTGAGCTGCA |
| | GGGTCCCTCGCCTGACGAAAAGTCCGCGGCTCCGGGGTTGAAA |
| | CTCACTCCGGGGCTGTGGACGTCGGCTTACCTTCGCAAATTTGT |
| | ACCTGAGGACTACCACGCCCACGAGATTAGGTTCTACGAAGAC |
| | CAATCCCGCCCGCCTAATGCGGAGCTTACCGCCTGCGTCATTAC |
| | CCAGGGCCACATTCTTGGCCAATTGCAAGCCATCAACAAAGCC |
| | CGCCAAGAGTTTCTGCTACGAAAGGGACGGGGGGTTTACTTGG |
| | ACCCCCAGTCCGGCGAGGAGCTCAACCCAATCCCCCCGCCGCC |
| | GCAGCCCTATCAGCAGCAGCCGCGGGCCCTTGCTTCCCAGGAT |
| | GGCACCCAAAAAGAAGCTGCAGCTGCCGCCGCCACCCACGGAC |
| | GAGGAGGAATACTGGGACAGTCAGGCAGAGGAGGTTTTGGAC |
| | GAGGAGGAGGAGGACATGATGGAAGACTGGGAGAGCCTAGAC |
| | GAGGAAGCTTCCGAGGTCGAAGAGGTGTCAGACGAAACACCG |
| | TCACCCTCGGTCGCATTCCCCTCGCCGGCGCCCCAGAAATCGGC |
| | AACCGGTTCCAGCATGGCTACAACCTCCGCTCCTCAGGCGCCG |
| | CCGGCACTGCCCGTTCGCCGACCCAACCGTAGATGGGACACCA |
| | CTGGAACCAGGGCCGGTAAGTCCAAGCAGCCGCCGCCGTTAGC |
| | CCAAGAGCAACAACAGCGCCAAGGCTACCGCTCATGGCGCGG |
| | GCACAAGAACGCCATAGTTGCTTGCTTGCAAGACTGTGGGGGC |
| | AACATCTCCTTCGCCCGCCGCTTTCTTCTCTACCATCACGGCGT |
| | GGCCTTCCCCCGTAACATCCTGCATTACTACCGTCATCTCTACA |
| | GCCCATACTGCACCGGCGGCAGCGGCAGCAACAGCAGCGGCC |
| | ACACAGAAGCAAAGGCGACCGGATAGCAAGACTCTGACAAAG |
| | CCCAAGAAATCCACAGCGGCGGCAGCAGCAGGAGGAGGAGCG |
| | CTGCGTCTGGCGCCCAACGAACCCGTATCGACCCGCGAGCTTA |
| | GAAACAGGATTTTTCCCACTCTGTATGCTATATTTCAACAGAGC |
| | AGGGGCCAAGAACAAGAGCTGAAAATAAAAAACAGGTCTCTG |
| | CGATCCCTCACCCGCAGCTGCCTGTATCACAAAAGCGAAGATC |
| | AGCTTCGGCGCACGCTGGAAGACGCGGAGGCTCTCTTCAGTAA |
| | ATACTGCGCGCTGACTCTTAAGGACTAGTTTCGCGCCCTTTCTC |
| | AAATTTAAGCGCGAAAACTACGTCATCTCCAGCGGCCACACCC |
| | GGCGCCAGCACCTGTTGTCAGCGCCATTATGAGCAAGGAAATT |
| | CCCACGCCCTACATGTGGAGTTACCAGCCACAAATGGGACTTG |
| | CGGCTGGAGCTGCCCAAGACTACTCAACCCGAATAAACTACAT |
| | GAGCGCGGGACCCCACATGATATCCCGGGTCAACGGAATACGC |
| | GCCCACCGAAACCGAATTCTCCTGGAACAGGCGGCTATTACCA |
| | CCACACCTCGTAATAACCTTAATCCCCGTAGTTGGCCCGCTGCC |
| | CTGGTGTACCAGGAAAGTCCCGCTCCCACCACTGTGGTACTTCC |
| | CAGAGACGCCCAGGCCGAAGTTCAGATGACTAACTCAGGGGCG |
| | CAGCTTGCGGGCGGCTTTCGTCACAGGGTGCGGTCGCCCGGGC |
| | AGGGTATAACTCACCTGACAATCAGAGGGCGAGGTATTCAGCT |
| | CAACGACGAGTCGGTGAGCTCCTCGCTTGGTCTCCGTCCGGAC |
| | GGGACATTTCAGATCGGCGGCGCCGGCCGCTCTTCATTCACGC |
| | CTCGTCAGGCAATCCTAACTCTGCAGACCTCGTCCTCTGAGCCG |
| | CGCTCTGGAGGCATTGGAACTCTGCAATTTATTGAGGAGTTTGT |
| | GCCATCGGTCTACTTTAACCCCTTCTCGGGACCTCCCGGCCACT |
| | ATCCGGATCAATTTATTCCTAACTTTGACGCGGTAAAGGACTCG |
| | GCGGACGGCTACGACTGAATGTTAAGTGGAGAGGCAGAGCAA |
| | CTGCGCCTGAAACACCTGGTCCACTGTCGCCGCCACAAGTGCTT |
| | TGCCCGCGACTCCGGTGAGTTTTGCTACTTTGAATTGCCCGAGG |
| | ATCATATCGAGGGCCCGGCGCACGGCGTCCGGCTTACCGCCCA |
| | GGGAGAGCTTGCCCGTAGCCTGATTCGGGAGTTTACCCAGCGC |
| | CCCCTGCTAGTTGAGCGGGACAGGGGACCCTGTGTTCTCACTGT |
| | GATTTGCAACTGTCCTAACCCTGGATTACATCAAGATCCTCTAG |
| | TTAATGTCAGGTCGCCTAAGTCGATTAACTAGAGTACCCGGGG |
| | ATCTTATTCCCTTTAACTAATAAAAAAAAATAATAAAGCATCA |
| | CTTACTTAAAATCAGTTAGCAAATTTCTGTCCAGTTTATTCAGC |
| | AGCACCTCCTTGCCCTCCTCCCAGCTCTGGTATTGCAGCTTCCT |
| | CCTGGCTGCAAACTTTCTCCACAATCTAAATGGAATGTCAGTTT |
| | CCTCCTGTTCCTGTCCATCCGCACCCACTATCTTCATGTTGTTGC |
| | AGATGAAGCGCGCAAGACCGTCTGAAGATACCTTCAACCCCGT |
| | GTATCCATATGACACGGAAACCGGTCCTCCAACTGTGCCTTTTC |
| | TTACTCCTCCCTTTGTATCCCCCAATGGGTTTCAAGAGAGTCCC |
| | CCTGGGGTACTCTCTTTGCGCCTATCCGAACCTCTAGTTACCTC |
| | CAATGGCATGCTTGCGCTCAAAATGGGCAACGGCCTCTCTCTG |
| | GACGAGGCCGGCAACCTTACCTCCCAAAATGTAACCACTGTGA |
| | GCCCACCTCTCAAAAAAACCAAGTCAAACATAAACCTGGAAAT |
| | ATCTGCACCCCTCACAGTTACCTCAGAAGCCCTAACTGTGGCTG |
| | CCGCCGCACCTCTAATGGTCGCGGGCAACACACTCACCATGCA |
| | ATCACAGGCCCCGCTAACCGTGCACGACTCCAAACTTAGCATT |
| | GCCACCCAAGGACCCCTCACAGTGTCAGAAGGAAAGCTAGCCC |
| | TGCAAACATCAGGCCCCCTCACCACCACCGATAGCAGTACCCT |
| | TACTATCACTGCCTCACCCCCTCTAACTACTGCCACTGGTAGCT |
| | TGGGCATTGACTTGAAAGAGCCCATTTATACACAAAATGGAAA |
| | ACTAGGACTAAAGTACGGGGCTCCTTTGCATGTAACAGACGAC |
| | CTAAACACTTTGACCGTAGCAACTGGTCCAGGTGTGACTATTA |
| | ATAATACTTCCTTGCAAACTAAAGTTACTGGAGCCTTGGGTTTT |
| | GATTCACAAGGCAATATGCAACTTAATGTAGCAGGAGGACTAA |
| | GGATTGATTCTCAAAACAGACGCCTTATACTTGATGTTAGTTAT |
| | CCGTTTGATGCTCAAAACCAACTAAATCTAAGACTAGGACAGG |
| | GCCCTCTTTTTATAAACTCAGCCCACAACTTGGATATTAACTAC |
| | AACAAAGGCCTTTACTTGTTTACAGCTTCAAACAATTCCAAAA |
| | AGCTTGAGGTTAACCTAAGCACTGCCAAGGGGTTGATGTTTGA |
| | CGCTACAGCCATAGCCATTAATGCAGGAGATGGGCTTGAATTT |
| | GGTTCACCTAATGCACCAAACACAAATCCCCTCAAAACAAAAA |
| | TTGGCCATGGCCTAGAATTTGATTCAAACAAGGCTATGGTTCCT |
| | AAACTAGGAACTGGCCTTAGTTTTGACAGCACAGGTGCCATTA |
| | CAGTAGGAAACAAAAATAATGATAAGCTAACTTTGTGGACCAC |
| | ACCAGCTCCATCTCCTAACTGTAGACTAAATGCAGAGAAAGAT |
| | GCTAAACTCACTTTGGTCTTAACAAAATGTGGCAGTCAAATACT |
| | TGCTACAGTTTCAGTTTTGGCTGTTAAAGGCAGTTTGGCTCCAA |
| | TATCTGGAACAGTTCAAAGTGCTCATCTTATTATAAGATTTGAC |
| | GAAAATGGAGTGCTACTAAACAATTCCTTCCTGGACCCAGAAT |
| | ATTGGAACTTTAGAAATGGAGATCTTACTGAAGGCACAGCCTA |
| | TACAAACGCTGTTGGATTTATGCCTAACCTATCAGCTTATCCAA |
| | AATCTCACGGTAAAACTGCCAAAAGTAACATTGTCAGTCAAGT |
| | TTACTTAAACGGAGACAAAACTAAACCTGTAACACTAACCATT |
| | ACACTAAACGGTACACAGGAAACAGGAGACACAACTCCAAGT |
| | GCATACTCTATGTCATTTTCATGGGACTGGTCTGGCCACAACTA |
| | CATTAATGAAATATTTGCCACATCCTCTTACACTTTTTCATACA |
| | TTGCCCAAGAATAAAGAATCGTTTGTGTTATGTTTCAACGTGTT |
| | TATTTTTCAATTGCAGAAAATTTCAAGTCATTTTTCATTCAGTA |
| | GTATAGCCCCACCACCACATAGCTTATACAGATCACCGTACCTT |
| | AATCAAACTCACAGAACCCTAGTATTCAACCTGCCACCTCCCTC |
| | CCAACACACAGAGTACACAGTCCTTTCTCCCCGGCTGGCCTTAA |
| | AAAGCATCATATCATGGGTAACAGACATATTCTTAGGTGTTAT |
| | ATTCCACACGGTTTCCTGTCGAGCCAAACGCTCATCAGTGATAT |
| | TAATAAACTCCCCGGGCAGCTCACTTAAGTTCATGTCGCTGTCC |
| | AGCTGCTGAGCCACAGGCTGCTGTCCAACTTGCGGTTGCTTAAC |
| | GGGCGGCGAAGGAGAAGTCCACGCCTACATGGGGGTAGAGTC |
| | ATAATCGTGCATCAGGATAGGGCGGTGGTGCTGCAGCAGCGCG |
| | CGAATAAACTGCTGCCGCCGCCGCTCCGTCCTGCAGGAATACA |
| | ACATGGCAGTGGTCTCCTCAGCGATGATTCGCACCGCCCGCAG |
| | CATAAGGCGCCTTGTCCTCCGGGCACAGCAGCGCACCCTGATC |
| | TCACTTAAATCAGCACAGTAACTGCAGCACAGCACCACAATAT |
| | TGTTCAAAATCCCACAGTGCAAGGCGCTGTATCCAAAGCTCAT |
| | GGCGGGGACCACAGAACCCACGTGGCCATCATACCACAAGCGC |
| | AGGTAGATTAAGTGGCGACCCCTCATAAACACGCTGGACATAA |
| | ACATTACCTCTTTTGGCATGTTGTAATTCACCACCTCCCGGTAC |
| | CATATAAACCTCTGATTAAACATGGCGCCATCCACCACCATCCT |
| | AAACCAGCTGGCCAAAACCTGCCCGCCGGCTATACACTGCAGG |
| | GAACCGGGACTGGAACAATGACAGTGGAGAGCCCAGGACTCG |
| | TAACCATGGATCATCATGCTCGTCATGATATCAATGTTGGCACA |
| | ACACAGGCACACGTGCATACACTTCCTCAGGATTACAAGCTCC |
| | TCCCGCGTTAGAACCATATCCCAGGGAACAACCCATTCCTGAA |
| | TCAGCGTAAATCCCACACTGCAGGGAAGACCTCGCACGTAACT |
| | CACGTTGTGCATTGTCAAAGTGTTACATTCGGGCAGCAGCGGA |
| | TGATCCTCCAGTATGGTAGCGCGGGTTTCTGTCTCAAAAGGAG |
| | GTAGACGATCCCTACTGTACGGAGTGCGCCGAGACAACCGAGA |
| | TCGTGTTGGTCGTAGTGTCATGCCAAATGGAACGCCGGACGTA |
| | GTCATATTTCCTGAAGCAAAACCAGGTGCGGGCGTGACAAACA |
| | GATCTGCGTCTCCGGTCTCGCCGCTTAGATCGCTCTGTGTAGTA |
| | GTTGTAGTATATCCACTCTCTCAAAGCATCCAGGCGCCCCCTGG |
| | CTTCGGGTTCTATGTAAACTCCTTCATGCGCCGCTGCCCTGATA |
| | ACATCCACCACCGCAGAATAAGCCACACCCAGCCAACCTACAC |
| | ATTCGTTCTGCGAGTCACACACGGGAGGAGCGGGAAGAGCTGG |
| | AAGAACCATGTTTTTTTTTTTATTCCAAAAGATTATCCAAAACC |
| | TCAAAATGAAGATCTATTAAGTGAACGCGCTCCCCTCCGGTGG |
| | CGTGGTCAAACTCTACAGCCAAAGAACAGATAATGGCATTTGT |
| | AAGATGTTGCACAATGGCTTCCAAAAGGCAAACGGCCCTCACG |
| | TCCAAGTGGACGTAAAGGCTAAACCCTTCAGGGTGAATCTCCT |
| | CTATAAACATTCCAGCACCTTCAACCATGCCCAAATAATTCTCA |
| | TCTCGCCACCTTCTCAATATATCTCTAAGCAAATCCCGAATATT |
| | AAGTCCGGCCATTGTAAAAATCTGCTCCAGAGCGCCCTCCACC |
| | TTCAGCCTCAAGCAGCGAATCATGATTGCAAAAATTCAGGTTC |
| | CTCACAGACCTGTATAAGATTCAAAAGCGGAACATTAACAAAA |
| | ATACCGCGATCCCGTAGGTCCCTTCGCAGGGCCAGCTGAACAT |
| | AATCGTGCAGGTCTGCACGGACCAGCGCGGCCACTTCCCCGCC |
| | AGGAACCATGACAAAAGAACCCACACTGATTATGACACGCATA |
| | CTCGGAGCTATGCTAACCAGCGTAGCCCCGATGTAAGCTTGTT |
| | GCATGGGCGGCGATATAAAATGCAAGGTGCTGCTCAAAAAATC |
| | AGGCAAAGCCTCGCGCAAAAAAGAAAGCACATCGTAGTCATG |
| | CTCATGCAGATAAAGGCAGGTAAGCTCCGGAACCACCACAGAA |
| | AAAGACACCATTTTTCTCTCAAACATGTCTGCGGGTTTCTGCAT |
| | AAACACAAAATAAAATAACAAAAAAACATTTAAACATTAGAA |
| | GCCTGTCTTACAACAGGAAAAACAACCCTTATAAGCATAAGAC |
| | GGACTACGGCCATGCCGGCGTGACCGTAAAAAAACTGGTCACC |
| | GTGATTAAAAAGCACCACCGACAGCTCCTCGGTCATGTCCGGA |
| | GTCATAATGTAAGACTCGGTAAACACATCAGGTTGATTCACAT |
| | CGGTCAGTGCTAAAAAGCGACCGAAATAGCCCGGGGGAATAC |
| | ATACCCGCAGGCGTAGAGACAACATTACAGCCCCCATAGGAGG |
| | TATAACAAAATTAATAGGAGAGAAAAACACATAAACACCTGA |
| | AAAACCCTCCTGCCTAGGCAAAATAGCACCCTCCCGCTCCAGA |
| | ACAACATACAGCGCTTCCACAGCGGCAGCCATAACAGTCAGCC |
| | TTACCAGTAAAAAAGAAAACCTATTAAAAAAACACCACTCGAC |
| | ACGGCACCAGCTCAATCAGTCACAGTGTAAAAAAGGGCCAAGT |
| | GCAGAGCGAGTATATATAGGACTAAAAAATGACGTAACGGTTA |
| | AAGTCCACAAAAAACACCCAGAAAACCGCACGCGAACCTACG |
| | CCCAGAAACGAAAGCCAAAAAACCCACAACTTCCTCAAATCGT |
| | CACTTCCGTTTTCCCACGTTACGTCACTTCCCATTTTAAGAAAA |
| | CTACAATTCCCAACACATACAAGTTACTCCGCCCTAAAACCTAC |
| | GTCACCCGCCCCGTTCCCACGCCCCGCGCCACGTCACAAACTCC |
| | ACCCCCTCATTATCATATTGGCTTCAATCCAAAATAAGGTATAT |
| | TATTGATGAT |
| SEQ ID NO: | CATCATCAATAATATACCTTATTTTGGATTGAAGCCAATATGAT |
| 17 | AATGAGGGGGTGGAGTTTGTGACGTGGCGCGGGGCGTGGGAA |
| | CGGGGCGGGTGACGTAGTAGTGTGGCGGAAGTGTGATGTTGCA |
| | AGTGTGGCGGAACACATGTAAGCGACGGATGTGGCAAAAGTG |
| | ACGTTTTTGGTGTGCGCCGGTGTACACAGGAAGTGACAATTTTC |
| | GCGCGGTTTTAGGCGGATGTTGTAGTAAATTTGGGCGTAACCG |
| | AGTAAGATTTGGCCATTTTCGCGGGAAAACTGAATAAGAGGAA |
| | GTGAAATCTGAATAATTTTGTGTTACTCATAGCGCGTAATACTG |
| | TAATAGTAATCAATTACGGGGTCATTAGTTCATAGCCCATATAT |
| | GGAGTTCCGCGTTACATAACTTACGGTAAATGGCCCGCCTGGC |
| | TGACCGCCCAACGACCCCCGCCCATTGACGTCAATAATGACGT |
| | ATGTTCCCATAGTAACGCCAATAGGGACTTTCCATTGACGTCAA |
| | TGGGTGGAGTATTTACGGTAAACTGCCCACTTGGCAGTACATC |
| | AAGTGTATCATATGCCAAGTACGCCCCCTATTGACGTCAATGA |
| | CGGTAAATGGCCCGCCTGGCATTATGCCCAGTACATGACCTTAT |
| | GGGACTTTCCTACTTGGCAGTACATCTACGTATTAGTCATCGCT |
| | ATTACCATGGTGATGCGGTTTTGGCAGTACATCAATGGGCGTG |
| | GATAGCGGTTTGACTCACGGGGATTTCCAAGTCTCCACCCCATT |
| | GACGTCAATGGGAGTTTGTTTTGGCACCAAAATCAACGGGACT |
| | TTCCAAAATGTCGTAACAACTCCGCCCCATTGACGCAAATGGG |
| | CGGTAGGCGTGTACGGTGGGAGGTCTATATAAGCAGAGCTGGT |
| | TTAGTGAACCGTCAGATCCGCTAGAGATCTGGTACCGTCGACG |
| | CGGCCGCTCGAGCCTAAGCTTCTAGATGCATGCTCGAGCGGCC |
| | GCCAGTGTGATGGATATCTGCAGAATTCGCCCTTGCTCGATCCA |
| | CCGGATCTAGATAACTGATCATAATCAGCCATACCACATTTGTA |
| | GAGGTTTTACTTGCTTTAAAAAACCTCCCACACCTCCCCCTGAA |
| | CCTGAAACATAAAATGAATGCAATTGTTGTTGTTAACTTGTTTA |
| | TTGCAGCTTATAATGGTTACAAATAAAGCAATAGCATCACAAA |
| | TTTCACAAATAAAGCATTTTTTTCACTGCATTCTAGTTGTGGTTT |
| | GTCCAAACTCATCAATGTATCTTAACGCGGATCTGGAAGGTGC |
| | TGAGGTACGATGAGACCCGCACCAGGTGCAGACCCTGCGAGTG |
| | TGGCGGTAAACATATTAGGAACCAGCCTGTGATGCTGGATGTG |
| | ACCGAGGAGCTGAGGCCCGATCACTTGGTGCTGGCCTGCACCC |
| | GCGCTGAGTTTGGCTCTAGCGATGAAGATACAGATTGAGGTAC |
| | TGAAATGTGTGGGCGTGGCTTAAGGGTGGGAAAGAATATATAA |
| | GGTGGGGGTCTTATGTAGTTTTGTATCTGTTTTGCAGCAGCCGC |
| | CGCCGCCATGAGCACCAACTCGTTTGATGGAAGCATTGTGAGC |
| | TCATATTTGACAACGCGCATGCCCCCATGGGCCGGGGTGCGTC |
| | AGAATGTGATGGGCTCCAGCATTGATGGTCGCCCCGTCCTGCC |
| | CGCAAACTCTACTACCTTGACCTACGAGACCGTGTCTGGAACG |
| | CCGTTGGAGACTGCAGCCTCCGCCGCCGCTTCAGCCGCTGCAG |
| | CCACCGCCCGCGGGATTGTGACTGACTTTGCTTTCCTGAGCCCG |
| | CTTGCAAGCAGTGCAGCTTCCCGTTCATCCGCCCGCGATGACA |
| | AGTTGACGGCTCTTTTGGCACAATTGGATTCTTTGACCCGGGAA |
| | CTTAATGTCGTTTCTCAGCAGCTGTTGGATCTGCGCCAGCAGGT |
| | TTCTGCCCTGAAGGCTTCCTCCCCTCCCAATGCGGTTTAAAACA |
| | TAAATAAAAAACCAGACTCTGTTTGGATTTGGATCAAGCAAGT |
| | GTCTTGCTGTCTTTATTTAGGGGTTTTGCGCGCGCGGTAGGCCC |
| | GGGACCAGCGGTCTCGGTCGTTGAGGGTCCTGTGTATTTTTTCC |
| | AGGACGTGGTAAAGGTGACTCTGGATGTTCAGATACATGGGCA |
| | TAAGCCCGTCTCTGGGGTGGAGGTAGCACCACTGCAGAGCTTC |
| | ATGCTGCGGGGTGGTGTTGTAGATGATCCAGTCGTAGCAGGAG |
| | CGCTGGGCGTGGTGCCTAAAAATGTCTTTCAGTAGCAAGCTGA |
| | TTGCCAGGGGCAGGCCCTTGGTGTAAGTGTTTACAAAGCGGTT |
| | AAGCTGGGATGGGTGCATACGTGGGGATATGAGATGCATCTTG |
| | GACTGTATTTTTAGGTTGGCTATGTTCCCAGCCATATCCCTCCG |
| | GGGATTCATGTTGTGCAGAACCACCAGCACAGTGTATCCGGTG |
| | CACTTGGGAAATTTGTCATGTAGCTTAGAAGGAAATGCGTGGA |
| | AGAACTTGGAGACGCCCTTGTGACCTCCAAGATTTTCCATGCAT |
| | TCGTCCATAATGATGGCAATGGGCCCACGGGCGGCGGCCTGGG |
| | CGAAGATATTTCTGGGATCACTAACGTCATAGTTGTGTTCCAGG |
| | ATGAGATCGTCATAGGCCATTTTTACAAAGCGCGGGCGGAGGG |
| | TGCCAGACTGCGGTATAATGGTTCCATCCGGCCCAGGGGCGTA |
| | GTTACCCTCACAGATTTGCATTTCCCACGCTTTGAGTTCAGATG |
| | GGGGGATCATGTCTACCTGCGGGGCGATGAAGAAAACGGTTTC |
| | CGGGGTAGGGGAGATCAGCTGGGAAGAAAGCAGGTTCCTGAG |
| | CAGCTGCGACTTACCGCAGCCGGTGGGCCCGTAAATCACACCT |
| | ATTACCGGCTGCAACTGGTAGTTAAGAGAGCTGCAGCTGCCGT |
| | CATCCCTGAGCAGGGGGGCCACTTCGTTAAGCATGTCCCTGAC |
| | TCGCATGTTTTCCCTGACCAAATCCGCCAGAAGGCGCTCGCCGC |
| | CCAGCGATAGCAGTTCTTGCAAGGAAGCAAAGTTTTTCAACGG |
| | TTTGAGACCGTCCGCCGTAGGCATGCTTTTGAGCGTTTGACCAA |
| | GCAGTTCCAGGCGGTCCCACAGCTCGGTCACCTGCTCTACGGC |
| | ATCTCGATCCAGCATATCTCCTCGTTTCGCGGGTTGGGGCGGCT |
| | TTCGCTGTACGGCAGTAGTCGGTGCTCGTCCAGACGGGCCAGG |
| | GTCATGTCTTTCCACGGGCGCAGGGTCCTCGTCAGCGTAGTCTG |
| | GGTCACGGTGAAGGGGTGCGCTCCGGGCTGCGCGCTGGCCAGG |
| | GTGCGCTTGAGGCTGGTCCTGCTGGTGCTGAAGCGCTGCCGGT |
| | CTTCGCCCTGCGCGTCGGCCAGGTAGCATTTGACCATGGTGTCA |
| | TAGTCCAGCCCCTCCGCGGCGTGGCCCTTGGCGCGCAGCTTGCC |
| | CTTGGAGGAGGCGCCGCACGAGGGGCAGTGCAGACTTTTGAGG |
| | GCGTAGAGCTTGGGCGCGAGAAATACCGATTCCGGGGAGTAGG |
| | CATCCGCGCCGCAGGCCCCGCAGACGGTCTCGCATTCCACGAG |
| | CCAGGTGAGCTCTGGCCGTTCGGGGTCAAAAACCAGGTTTCCC |
| | CCATGCTTTTTGATGCGTTTCTTACCTCTGGTTTCCATGAGCCGG |
| | TGTCCACGCTCGGTGACGAAAAGGCTGTCCGTGTCCCCGTATA |
| | CAGACTTGAGAGGCCTGTCCTCGAGCGGTGTTCCGCGGTCCTCC |
| | TCGTATAGAAACTCGGACCACTCTGAGACAAAGGCTCGCGTCC |
| | AGGCCAGCACGAAGGAGGCTAAGTGGGAGGGGTAGCGGTCGT |
| | TGTCCACTAGGGGGTCCACTCGCTCCAGGGTGTGAAGACACAT |
| | GTCGCCCTCTTCGGCATCAAGGAAGGTGATTGGTTTGTAGGTGT |
| | AGGCCACGTGACCGGGTGTTCCTGAAGGGGGGCTATAAAAGGG |
| | GGTGGGGGCGCGTTCGTCCTCACTCTCTTCCGCATCGCTGTCTG |
| | CGAGGGCCAGCTGTTGGGGTGAGTACTCCCTCTGAAAAGCGGG |
| | CATGACTTCTGCGCTAAGATTGTCAGTTTCCAAAAACGAGGAG |
| | GATTTGATATTCACCTGGCCCGCGGTGATGCCTTTGAGGGTGGC |
| | CGCATCCATCTGGTCAGAAAAGACAATCTTTTTGTTGTCAAGCT |
| | TGGTGGCAAACGACCCGTAGAGGGCGTTGGACAGCAACTTGGC |
| | GATGGAGCGCAGGGTTTGGTTTTTGTCGCGATCGGCGCGCTCCT |
| | TGGCCGCGATGTTTAGCTGCACGTATTCGCGCGCAACGCACCG |
| | CCATTCGGGAAAGACGGTGGTGCGCTCGTCGGGCACCAGGTGC |
| | ACGCGCCAACCGCGGTTGTGCAGGGTGACAAGGTCAACGCTGG |
| | TGGCTACCTCTCCGCGTAGGCGCTCGTTGGTCCAGCAGAGGCG |
| | GCCGCCCTTGCGCGAGCAGAATGGCGGTAGGGGGTCTAGCTGC |
| | GTCTCGTCCGGGGGGTCTGCGTCCACGGTAAAGACCCCGGGCA |
| | GCAGGCGCGCGTCGAAGTAGTCTATCTTGCATCCTTGCAAGTCT |
| | AGCGCCTGCTGCCATGCGCGGGCGGCAAGCGCGCGCTCGTATG |
| | GGTTGAGTGGGGGACCCCATGGCATGGGGTGGGTGAGCGCGG |
| | AGGCGTACATGCCGCAAATGTCGTAAACGTAGAGGGGCTCTCT |
| | GAGTATTCCAAGATATGTAGGGTAGCATCTTCCACCGCGGATG |
| | CTGGCGCGCACGTAATCGTATAGTTCGTGCGAGGGAGCGAGGA |
| | GGTCGGGACCGAGGTTGCTACGGGCGGGCTGCTCTGCTCGGAA |
| | GACTATCTGCCTGAAGATGGCATGTGAGTTGGATGATATGGTT |
| | GGACGCTGGAAGACGTTGAAGCTGGCGTCTGTGAGACCTACCG |
| | CGTCACGCACGAAGGAGGCGTAGGAGTCGCGCAGCTTGTTGAC |
| | CAGCTCGGCGGTGACCTGCACGTCTAGGGCGCAGTAGTCCAGG |
| | GTTTCCTTGATGATGTCATACTTATCCTGTCCCTTTTTTTTCCAC |
| | AGCTCGCGGTTGAGGACAAACTCTTCGCGGTCTTTCCAGTACTC |
| | TTGGATCGGAAACCCGTCGGCCTCCGAACGGTAAGAGCCTAGC |
| | ATGTAGAACTGGTTGACGGCCTGGTAGGCGCAGCATCCCTTTTC |
| | TACGGGTAGCGCGTATGCCTGCGCGGCCTTCCGGCATGACCAG |
| | CATGAAGGGCACGAGCTGCTTCCCAAAGGCCCCCATCCAAGTA |
| | TAGGTCTCTACATCGTAGGTGACAAAGAGACGCTCGGTGCGAG |
| | GATGCGAGCCGATCGGGAAGAACTGGATCTCCCGCCACCAATT |
| | GGAGGAGTGGCTATTGATGTGGTGAAAGTAGAAGTCCCTGCGA |
| | CGGGCCGAACACTCGTGCTGGCTTTTGTAAAAACGTGCGCAGT |
| | ACTGGCAGCGGTGCACGGGCTGTACATCCTGCACGAGGTTGAC |
| | CTGACGACCGCGCACAAGGAAGCAGAGTGGGAATTTGAGCCCC |
| | TCGCCTGGCGGGTTTGGCTGGTGGTCTTCTACTTCGGCTGCTTG |
| | TCCTTGACCGTCTGGCTGCTCGAGGGGAGTTACGGTGGATCGG |
| | ACCACCACGCCGCGCGAGCCCAAAGTCCAGATGTCCGCGCGCG |
| | GCGGTCGGAGCTTGATGACAACATCGCGCAGATGGGAGCTGTC |
| | CATGGTCTGGAGCTCCCGCGGCGTCAGGTCAGGCGGGAGCTCC |
| | TGCAGGTTTACCTCGCATAGACGGGTCAGGGCGCGGGCTAGAT |
| | CCAGGTGATACCTAATTTCCAGGGGCTGGTTGGTGGCGGCGTC |
| | GATGGCTTGCAAGAGGCCGCATCCCCGCGGCGCGACTACGGTA |
| | CCGCGCGGCGGGCGGTGGGCCGCGGGGGTGTCCTTGGATGATG |
| | CATCTAAAAGCGGTGACGCGGGCGAGCCCCCGGAGGTAGGGG |
| | GGGCTCCGGACCCGCCGGGAGAGGGGGCAGGGGCACGTCGGC |
| | GCCGCGCGCGGGCAGGAGCTGGTGCTGCGCGCGTAGGTTGCTG |
| | GCGAACGCGACGACGCGGCGGTTGATCTCCTGAATCTGGCGCC |
| | TCTGCGTGAAGACGACGGGCCCGGTGAGCTTGAACCTGAAAGA |
| | GAGTTCGACAGAATCAATTTCGGTGTCGTTGACGGCGGCCTGG |
| | CGCAAAATCTCCTGCACGTCTCCTGAGTTGTCTTGATAGGCGAT |
| | CTCGGCCATGAACTGCTCGATCTCTTCCTCCTGGAGATCTCCGC |
| | GTCCGGCTCGCTCCACGGTGGCGGCGAGGTCGTTGGAAATGCG |
| | GGCCATGAGCTGCGAGAAGGCGTTGAGGCCTCCCTCGTTCCAG |
| | ACGCGGCTGTAGACCACGCCCCCTTCGGCATCGCGGGCGCGCA |
| | TGACCACCTGCGCGAGATTGAGCTCCACGTGCCGGGCGAAGAC |
| | GGCGTAGTTTCGCAGGCGCTGAAAGAGGTAGTTGAGGGTGGTG |
| | GCGGTGTGTTCTGCCACGAAGAAGTACATAACCCAGCGTCGCA |
| | ACGTGGATTCGTTGATAATTGTTGTGTAGGTACTCCGCCGCCGA |
| | GGGACCTGAGCGAGTCCGCATCGACCGGATCGGAAAACCTCTC |
| | GAGAAAGGCGTCTAACCAGTCACAGTCGCAAGGTAGGCTGAGC |
| | ACCGTGGCGGGCGGCAGCGGGCGGCGGTCGGGGTTGTTTCTGG |
| | CGGAGGTGCTGCTGATGATGTAATTAAAGTAGGCGGTCTTGAG |
| | ACGGCGGATGGTCGACAGAAGCACCATGTCCTTGGGTCCGGCC |
| | TGCTGAATGCGCAGGCGGTCGGCCATGCCCCAGGCTTCGTTTTG |
| | ACATCGGCGCAGGTCTTTGTAGTAGTCTTGCATGAGCCTTTCTA |
| | CCGGCACTTCTTCTTCTCCTTCCTCTTGTCCTGCATCTCTTGCAT |
| | CTATCGCTGCGGCGGCGGCGGAGTTTGGCCGTAGGTGGCGCCC |
| | TCTTCCTCCCATGCGTGTGACCCCGAAGCCCCTCATCGGCTGAA |
| | GCAGGGCTAGGTCGGCGACAACGCGCTCGGCTAATATGGCCTG |
| | CTGCACCTGCGTGAGGGTAGACTGGAAGTCATCCATGTCCACA |
| | AAGCGGTGGTATGCGCCCGTGTTGATGGTGTAAGTGCAGTTGG |
| | CCATAACGGACCAGTTAACGGTCTGGTGACCCGGCTGCGAGAG |
| | CTCGGTGTACCTGAGACGCGAGTAAGCCCTCGAGTCAAATACG |
| | TAGTCGTTGCAAGTCCGCACCAGGTACTGGTATCCCACCAAAA |
| | AGTGCGGCGGCGGCTGGCGGTAGAGGGGCCAGCGTAGGGTGG |
| | CCGGGGCTCCGGGGGCGAGATCTTCCAACATAAGGCGATGATA |
| | TCCGTAGATGTACCTGGACATCCAGGTGATGCCGGCGGCGGTG |
| | GTGGAGGCGCGCGGAAAGTCGCGGACGCGGTTCCAGATGTTGC |
| | GCAGCGGCAAAAAGTGCTCCATGGTCGGGACGCTCTGGCCGGT |
| | CAGGCGCGCGCAATCGTTGACGCTCTAGCGTGCAAAAGGAGAG |
| | CCTGTAAGCGGGCACTCTTCCGTGGTCTGGTGGATAAATTCGCA |
| | AGGGTATCATGGCGGACGACCGGGGTTCGAGCCCCGTATCCGG |
| | CCGTCCGCCGTGATCCATGCGGTTACCGCCCGCGTGTCGAACCC |
| | AGGTGTGCGACGTCAGACAACGGGGGAGTGCTCCTTTTGGCTT |
| | CCTTCCAGGCGCGGCGGCTGCTGCGCTAGCTTTTTTGGCCACTG |
| | GCCGCGCGCAGCGTAAGCGGTTAGGCTGGAAAGCGAAAGCATT |
| | AAGTGGCTCGCTCCCTGTAGCCGGAGGGTTATTTTCCAAGGGTT |
| | GAGTCGCGGGACCCCCGGTTCGAGTCTCGGACCGGCCGGACTG |
| | CGGCGAACGGGGGTTTGCCTCCCCGTCATGCAAGACCCCGCTT |
| | GCAAATTCCTCCGGAAACAGGGACGAGCCCCTTTTTTGCTTTTC |
| | CCAGATGCATCCGGTGCTGCGGCAGATGCGCCCCCCTCCTCAG |
| | CAGCGGCAAGAGCAAGAGCAGCGGCAGACATGCAGGGCACCC |
| | TCCCCTCCTCCTACCGCGTCAGGAGGGGCGACATCCGCGGTTG |
| | ACGCGGCAGCAGATGGTGATTACGAACCCCCGCGGCGCCGGGC |
| | CCGGCACTACCTGGACTTGGAGGAGGGCGAGGGCCTGGCGCGG |
| | CTAGGAGCGCCCTCTCCTGAGCGGCACCCAAGGGTGCAGCTGA |
| | AGCGTGATACGCGTGAGGCGTACGTGCCGCGGCAGAACCTGTT |
| | TCGCGACCGCGAGGGAGAGGAGCCCGAGGAGATGCGGGATCG |
| | AAAGTTCCACGCAGGGCGCGAGCTGCGGCATGGCCTGAATCGC |
| | GAGCGGTTGCTGCGCGAGGAGGACTTTGAGCCCGACGCGCGAA |
| | CCGGGATTAGTCCCGCGCGCGCACACGTGGCGGCCGCCGACCT |
| | GGTAACCGCATACGAGCAGACGGTGAACCAGGAGATTAACTTT |
| | CAAAAAAGCTTTAACAACCACGTGCGTACGCTTGTGGCGCGCG |
| | AGGAGGTGGCTATAGGACTGATGCATCTGTGGGACTTTGTAAG |
| | CGCGCTGGAGCAAAACCCAAATAGCAAGCCGCTCATGGCGCAG |
| | CTGTTCCTTATAGTGCAGCACAGCAGGGACAACGAGGCATTCA |
| | GGGATGCGCTGCTAAACATAGTAGAGCCCGAGGGCCGCTGGCT |
| | GCTCGATTTGATAAACATCCTGCAGAGCATAGTGGTGCAGGAG |
| | CGCAGCTTGAGCCTGGCTGACAAGGTGGCCGCCATCAACTATT |
| | CCATGCTTAGCCTGGGCAAGTTTTACGCCCGCAAGATATACCAT |
| | ACCCCTTACGTTCCCATAGACAAGGAGGTAAAGATCGAGGGGT |
| | TCTACATGCGCATGGCGCTGAAGGTGCTTACCTTGAGCGACGA |
| | CCTGGGCGTTTATCGCAACGAGCGCATCCACAAGGCCGTGAGC |
| | GTGAGCCGGCGGCGCGAGCTCAGCGACCGCGAGCTGATGCACA |
| | GCCTGCAAAGGGCCCTGGCTGGCACGGGCAGCGGCGATAGAG |
| | AGGCCGAGTCCTACTTTGACGCGGGCGCTGACCTGCGCTGGGC |
| | CCCAAGCCGACGCGCCCTGGAGGCAGCTGGGGCCGGACCTGGG |
| | CTGGCGGTGGCACCCGCGCGCGCTGGCAACGTCGGCGGCGTGG |
| | AGGAATATGACGAGGACGATGAGTACGAGCCAGAGGACGGCG |
| | AGTACTAAGCGGTGATGTTTCTGATCAGATGATGCAAGACGCA |
| | ACGGACCCGGCGGTGCGGGCGGCGCTGCAGAGCCAGCCGTCCG |
| | GCCTTAACTCCACGGACGACTGGCGCCAGGTCATGGACCGCAT |
| | CATGTCGCTGACTGCGCGCAATCCTGACGCGTTCCGGCAGCAG |
| | CCGCAGGCCAACCGGCTCTCCGCAATTCTGGAAGCGGTGGTCC |
| | CGGCGCGCGCAAACCCCACGCACGAGAAGGTGCTGGCGATCGT |
| | AAACGCGCTGGCCGAAAACAGGGCCATCCGGCCCGACGAGGC |
| | CGGCCTGGTCTACGACGCGCTGCTTCAGCGCGTGGCTCGTTACA |
| | ACAGCGGCAACGTGCAGACCAACCTGGACCGGCTGGTGGGGG |
| | ATGTGCGCGAGGCCGTGGCGCAGCGTGAGCGCGCGCAGCAGC |
| | AGGGCAACCTGGGCTCCATGGTTGCACTAAACGCCTTCCTGAG |
| | TACACAGCCCGCCAACGTGCCGCGGGGACAGGAGGACTACACC |
| | AACTTTGTGAGCGCACTGCGGCTAATGGTGACTGAGACACCGC |
| | AAAGTGAGGTGTACCAGTCTGGGCCAGACTATTTTTTCCAGAC |
| | CAGTAGACAAGGCCTGCAGACCGTAAACCTGAGCCAGGCTTTC |
| | AAAAACTTGCAGGGGCTGTGGGGGGTGCGGGCTCCCACAGGCG |
| | ACCGCGCGACCGTGTCTAGCTTGCTGACGCCCAACTCGCGCCT |
| | GTTGCTGCTGCTAATAGCGCCCTTCACGGACAGTGGCAGCGTG |
| | TCCCGGGACACATACCTAGGTCACTTGCTGACACTGTACCGCG |
| | AGGCCATAGGTCAGGCGCATGTGGACGAGCATACTTTCCAGGA |
| | GATTACAAGTGTCAGCCGCGCGCTGGGGCAGGAGGACACGGG |
| | CAGCCTGGAGGCAACCCTAAACTACCTGCTGACCAACCGGCGG |
| | CAGAAGATCCCCTCGTTGCACAGTTTAAACAGCGAGGAGGAGC |
| | GCATTTTGCGCTACGTGCAGCAGAGCGTGAGCCTTAACCTGAT |
| | GCGCGACGGGGTAACGCCCAGCGTGGCGCTGGACATGACCGCG |
| | CGCAACATGGAACCGGGCATGTATGCCTCAAACCGGCCGTTTA |
| | TCAACCGCCTAATGGACTACTTGCATCGCGCGGCCGCCGTGAA |
| | CCCCGAGTATTTCACCAATGCCATCTTGAACCCGCACTGGCTAC |
| | CGCCCCCTGGTTTCTACACCGGGGGATTCGAGGTGCCCGAGGG |
| | TAACGATGGATTCCTCTGGGACGACATAGACGACAGCGTGTTT |
| | TCCCCGCAACCGCAGACCCTGCTAGAGTTGCAACAGCGCGAGC |
| | AGGCAGAGGCGGCGCTGCGAAAGGAAAGCTTCCGCAGGCCAA |
| | GCAGCTTGTCCGATCTAGGCGCTGCGGCCCCGCGGTCAGATGC |
| | TAGTAGCCCATTTCCAAGCTTGATAGGGTCTCTTACCAGCACTC |
| | GCACCACCCGCCCGCGCCTGCTGGGCGAGGAGGAGTACCTAAA |
| | CAACTCGCTGCTGCAGCCGCAGCGCGAAAAAAACCTGCCTCCG |
| | GCATTTCCCAACAACGGGATAGAGAGCCTAGTGGACAAGATGA |
| | GTAGATGGAAGACGTACGCGCAGGAGCACAGGGACGTGCCAG |
| | GCCCGCGCCCGCCCACCCGTCGTCAAAGGCACGACCGTCAGCG |
| | GGGTCTGGTGTGGGAGGACGATGACTCGGCAGACGACAGCAG |
| | CGTCCTGGATTTGGGAGGGAGTGGCAACCCGTTTGCGCACCTT |
| | CGCCCCAGGCTGGGGAGAATGTTTTAAAAAAAAAAAAGCATG |
| | ATGCAAAATAAAAAACTCACCAAGGCCATGGCACCGAGCGTTG |
| | GTTTTCTTGTATTCCCCTTAGTATGCGGCGCGCGGCGATGTATG |
| | AGGAAGGTCCTCCTCCCTCCTACGAGAGTGTGGTGAGCGCGGC |
| | GCCAGTGGCGGCGGCGCTGGGTTCTCCCTTCGATGCTCCCCTGG |
| | ACCCGCCGTTTGTGCCTCCGCGGTACCTGCGGCCTACCGGGGG |
| | GAGAAACAGCATCCGTTACTCTGAGTTGGCACCCCTATTCGAC |
| | ACCACCCGTGTGTACCTGGTGGACAACAAGTCAACGGATGTGG |
| | CATCCCTGAACTACCAGAACGACCACAGCAACTTTCTGACCAC |
| | GGTCATTCAAAACAATGACTACAGCCCGGGGGAGGCAAGCAC |
| | ACAGACCATCAATCTTGACGACCGGTCGCACTGGGGCGGCGAC |
| | CTGAAAACCATCCTGCATACCAACATGCCAAATGTGAACGAGT |
| | TCATGTTTACCAATAAGTTTAAGGCGCGGGTGATGGTGTCGCG |
| | CTTGCCTACTAAGGACAATCAGGTGGAGCTGAAATACGAGTGG |
| | GTGGAGTTCACGCTGCCCGAGGGCAACTACTCCGAGACCATGA |
| | CCATAGACCTTATGAACAACGCGATCGTGGAGCACTACTTGAA |
| | AGTGGGCAGACAGAACGGGGTTCTGGAAAGCGACATCGGGGT |
| | AAAGTTTGACACCCGCAACTTCAGACTGGGGTTTGACCCCGTC |
| | ACTGGTCTTGTCATGCCTGGGGTATATACAAACGAAGCCTTCCA |
| | TCCAGACATCATTTTGCTGCCAGGATGCGGGGTGGACTTCACCC |
| | ACAGCCGCCTGAGCAACTTGTTGGGCATCCGCAAGCGGCAACC |
| | CTTCCAGGAGGGCTTTAGGATCACCTACGATGATCTGGAGGGT |
| | GGTAACATTCCCGCACTGTTGGATGTGGACGCCTACCAGGCGA |
| | GCTTGAAAGATGACACCGAACAGGGCGGGGGTGGCGCAGGCG |
| | GCAGCAACAGCAGTGGCAGCGGCGCGGAAGAGAACTCCAACG |
| | CGGCAGCCGCGGCAATGCAGCCGGTGGAGGACATGAACGATC |
| | ATGCCATTCGCGGCGACACCTTTGCCACACGGGCTGAGGAGAA |
| | GCGCGCTGAGGCCGAAGCAGCGGCCGAAGCTGCCGCCCCCGCT |
| | GCGCAACCCGAGGTCGAGAAGCCTCAGAAGAAACCGGTGATC |
| | AAACCCCTGACAGAGGACAGCAAGAAACGCAGTTACAACCTA |
| | ATAAGCAATGACAGCACCTTCACCCAGTACCGCAGCTGGTACC |
| | TTGCATACAACTACGGCGACCCTCAGACCGGAATCCGCTCATG |
| | GACCCTGCTTTGCACTCCTGACGTAACCTGCGGCTCGGAGCAG |
| | GTCTACTGGTCGTTGCCAGACATGATGCAAGACCCCGTGACCTT |
| | CCGCTCCACGCGCCAGATCAGCAACTTTCCGGTGGTGGGCGCC |
| | GAGCTGTTGCCCGTGCACTCCAAGAGCTTCTACAACGACCAGG |
| | CCGTCTACTCCCAACTCATCCGCCAGTTTACCTCTCTGACCCAC |
| | GTGTTCAATCGCTTTCCCGAGAACCAGATTTTGGCGCGCCCGCC |
| | AGCCCCCACCATCACCACCGTCAGTGAAAACGTTCCTGCTCTCA |
| | CAGATCACGGGACGCTACCGCTGCGCAACAGCATCGGAGGAGT |
| | CCAGCGAGTGACCATTACTGACGCCAGACGCCGCACCTGCCCC |
| | TACGTTTACAAGGCCCTGGGCATAGTCTCGCCGCGCGTCCTATC |
| | GAGCCGCACTTTTTGAGCAAGCATGTCCATCCTTATATCGCCCA |
| | GCAATAACACAGGCTGGGGCCTGCGCTTCCCAAGCAAGATGTT |
| | TGGCGGGGCCAAGAAGCGCTCCGACCAACACCCAGTGCGCGTG |
| | CGCGGGCACTACCGCGCGCCCTGGGGCGCGCACAAACGCGGCC |
| | GCACTGGGCGCACCACCGTCGATGACGCCATCGACGCGGTGGT |
| | GGAGGAGGCGCGCAACTACACGCCCACGCCGCCACCAGTGTCC |
| | ACAGTGGACGCGGCCATTCAGACCGTGGTGCGCGGAGCCCGGC |
| | GCTATGCTAAAATGAAGAGACGGCGGAGGCGCGTAGCACGTC |
| | GCCACCGCCGCCGACCCGGCACTGCCGCCCAACGCGCGGCGGC |
| | GGCCCTGCTTAACCGCGCACGTCGCACCGGCCGACGGGCGGCC |
| | ATGCGGGCCGCTCGAAGGCTGGCCGCGGGTATTGTCACTGTGC |
| | CCCCCAGGTCCAGGCGACGAGCGGCCGCCGCAGCAGCCGCGGC |
| | CATTAGTGCTATGACTCAGGGTCGCAGGGGCAACGTGTATTGG |
| | GTGCGCGACTCGGTTAGCGGCCTGCGCGTGCCCGTGCGCACCC |
| | GCCCCCCGCGCAACTAGATTGCAAGAAAAAACTACTTAGACTC |
| | GTACTGTTGTATGTATCCAGCGGCGGCGGCGCGCAACGAAGCT |
| | ATGTCCAAGCGCAAAATCAAAGAAGAGATGCTCCAGGTCATCG |
| | CGCCGGAGATCTATGGCCCCCCGAAGAAGGAAGAGCAGGATT |
| | ACAAGCCCCGAAAGCTAAAGCGGGTCAAAAAGAAAAAGAAAG |
| | ATGATGATGATGAACTTGACGACGAGGTGGAACTGCTGCACGC |
| | TACCGCGCCCAGGCGACGGGTACAGTGGAAAGGTCGACGCGTA |
| | AAACGTGTTTTGCGACCCGGCACCACCGTAGTCTTTACGCCCGG |
| | TGAGCGCTCCACCCGCACCTACAAGCGCGTGTATGATGAGGTG |
| | TACGGCGACGAGGACCTGCTTGAGCAGGCCAACGAGCGCCTCG |
| | GGGAGTTTGCCTACGGAAAGCGGCATAAGGACATGCTGGCGTT |
| | GCCGCTGGACGAGGGCAACCCAACACCTAGCCTAAAGCCCGTA |
| | ACACTGCAGCAGGTGCTGCCCGCGCTTGCACCGTCCGAAGAAA |
| | AGCGCGGCCTAAAGCGCGAGTCTGGTGACTTGGCACCCACCGT |
| | GCAGCTGATGGTACCCAAGCGCCAGCGACTGGAAGATGTCTTG |
| | GAAAAAATGACCGTGGAACCTGGGCTGGAGCCCGAGGTCCGC |
| | GTGCGGCCAATCAAGCAGGTGGCGCCGGGACTGGGCGTGCAG |
| | ACCGTGGACGTTCAGATACCCACTACCAGTAGCACCAGTATTG |
| | CCACCGCCACAGAGGGCATGGAGACACAAACGTCCCCGGTTGC |
| | CTCAGCGGTGGCGGATGCCGCGGTGCAGGCGGTCGCTGCGGCC |
| | GCGTCCAAGACCTCTACGGAGGTGCAAACGGACCCGTGGATGT |
| | TTCGCGTTTCAGCCCCCCGGCGCCCGCGCCGTTCGAGGAAGTA |
| | CGGCGCCGCCAGCGCGCTACTGCCCGAATATGCCCTACATCCTT |
| | CCATTGCGCCTACCCCCGGCTATCGTGGCTACACCTACCGCCCC |
| | AGAAGACGAGCAACTACCCGACGCCGAACCACCACTGGAACC |
| | CGCCGCCGCCGTCGCCGTCGCCAGCCCGTGCTGGCCCCGATTTC |
| | CGTGCGCAGGGTGGCTCGCGAAGGAGGCAGGACCCTGGTGCTG |
| | CCAACAGCGCGCTACCACCCCAGCATCGTTTAAAAGCCGGTCT |
| | TTGTGGTTCTTGCAGATATGGCCCTCACCTGCCGCCTCCGTTTC |
| | CCGGTGCCGGGATTCCGAGGAAGAATGCACCGTAGGAGGGGC |
| | ATGGCCGGCCACGGCCTGACGGGCGGCATGCGTCGTGCGCACC |
| | ACCGGCGGCGGCGCGCGTCGCACCGTCGCATGCGCGGCGGTAT |
| | CCTGCCCCTCCTTATTCCACTGATCGCCGCGGCGATTGGCGCCG |
| | TGCCCGGAATTGCATCCGTGGCCTTGCAGGCGCAGAGACACTG |
| | ATTAAAAACAAGTTGCATGTGGAAAAATCAAAATAAAAAGTCT |
| | GGACTCTCACGCTCGCTTGGTCCTGTAACTATTTTGTAGAATGG |
| | AAGACATCAACTTTGCGTCTCTGGCCCCGCGACACGGCTCGCG |
| | CCCGTTCATGGGAAACTGGCAAGATATCGGCACCAGCAATATG |
| | AGCGGTGGCGCCTTCAGCTGGGGCTCGCTGTGGAGCGGCATTA |
| | AAAATTTCGGTTCCACCGTTAAGAACTATGGCAGCAAGGCCTG |
| | GAACAGCAGCACAGGCCAGATGCTGAGGGATAAGTTGAAAGA |
| | GCAAAATTTCCAACAAAAGGTGGTAGATGGCCTGGCCTCTGGC |
| | ATTAGCGGGGTGGTGGACCTGGCCAACCAGGCAGTGCAAAATA |
| | AGATTAACAGTAAGCTTGATCCCCGCCCTCCCGTAGAGGAGCC |
| | TCCACCGGCCGTGGAGACAGTGTCTCCAGAGGGGCGTGGCGAA |
| | AAGCGTCCGCGCCCCGACAGGGAAGAAACTCTGGTGACGCAA |
| | ATAGACGAGCCTCCCTCGTACGAGGAGGCACTAAAGCAAGGCC |
| | TGCCCACCACCCGTCCCATCGCGCCCATGGCTACCGGAGTGCT |
| | GGGCCAGCACACACCCGTAACGCTGGACCTGCCTCCCCCCGCC |
| | GACACCCAGCAGAAACCTGTGCTGCCAGGCCCGACCGCCGTTG |
| | TTGTAACCCGTCCTAGCCGCGCGTCCCTGCGCCGCGCCGCCAGC |
| | GGTCCGCGATCGTTGCGGCCCGTAGCCAGTGGCAACTGGCAAA |
| | GCACACTGAACAGCATCGTGGGTCTGGGGGTGCAATCCCTGAA |
| | GCGCCGACGATGCTTCTGATAGCTAACGTGTCGTATGTGTGTCA |
| | TGTATGCGTCCATGTCGCCGCCAGAGGAGCTGCTGAGCCGCCG |
| | CGCGCCCGCTTTCCAAGATGGCTACCCCTTCGATGATGCCGCAG |
| | TGGTCTTACATGCACATCTCGGGCCAGGACGCCTCGGAGTACC |
| | TGAGCCCCGGGCTGGTGCAGTTTGCCCGCGCCACCGAGACGTA |
| | CTTCAGCCTGAATAACAAGTTTAGAAACCCCACGGTGGCGCCT |
| | ACGCACGACGTGACCACAGACCGGTCCCAGCGTTTGACGCTGC |
| | GGTTCATCCCTGTGGACCGTGAGGATACTGCGTACTCGTACAA |
| | GGCGCGGTTCACCCTAGCTGTGGGTGATAACCGTGTGCTGGAC |
| | ATGGCTTCCACGTACTTTGACATCCGCGGCGTGCTGGACAGGG |
| | GCCCTACTTTTAAGCCCTACTCTGGCACTGCCTACAACGCCCTG |
| | GCTCCCAAGGGTGCCCCAAATCCTTGCGAATGGGATGAAGCTG |
| | CTACTGCTCTTGAAATAAACCTAGAAGAAGAGGACGATGACAA |
| | CGAAGACGAAGTAGACGAGCAAGCTGAGCAGCAAAAAACTCA |
| | CGTATTTGGGCAGGCGCCTTATTCTGGTATAAATATTACAAAGG |
| | AGGGTATTCAAATAGGTGTCGAAGGTCAAACACCTAAATATGC |
| | CGATAAAACATTTCAACCTGAACCTCAAATAGGAGAATCTCAG |
| | TGGTACGAAACAGAAATTAATCATGCAGCTGGGAGAGTCCTAA |
| | AAAAGACTACCCCAATGAAACCATGTTACGGTTCATATGCAAA |
| | ACCCACAAATGAAAATGGAGGGCAAGGCATTCTTGTAAAGCAA |
| | CAAAATGGAAAGCTAGAAAGTCAAGTGGAAATGCAATTTTTCT |
| | CAACTACTGAGGCAGCCGCAGGCAATGGTGATAACTTGACTCC |
| | TAAAGTGGTATTGTACAGTGAAGATGTAGATATAGAAACCCCA |
| | GACACTCATATTTCTTACATGCCCACTATTAAGGAAGGTAACTC |
| | ACGAGAACTAATGGGCCAACAATCTATGCCCAACAGGCCTAAT |
| | TACATTGCTTTTAGGGACAATTTTATTGGTCTAATGTATTACAA |
| | CAGCACGGGTAATATGGGTGTTCTGGCGGGCCAAGCATCGCAG |
| | TTGAATGCTGTTGTAGATTTGCAAGACAGAAACACAGAGCTTT |
| | CATACCAGCTTTTGCTTGATTCCATTGGTGATAGAACCAGGTAC |
| | TTTTCTATGTGGAATCAGGCTGTTGACAGCTATGATCCAGATGT |
| | TAGAATTATTGAAAATCATGGAACTGAAGATGAACTTCCAAAT |
| | TACTGCTTTCCACTGGGAGGTGTGATTAATACAGAGACTCTTAC |
| | CAAGGTAAAACCTAAAACAGGTCAGGAAAATGGATGGGAAAA |
| | AGATGCTACAGAATTTTCAGATAAAAATGAAATAAGAGTTGGA |
| | AATAATTTTGCCATGGAAATCAATCTAAATGCCAACCTGTGGA |
| | GAAATTTCCTGTACTCCAACATAGCGCTGTATTTGCCCGACAAG |
| | CTAAAGTACAGTCCTTCCAACGTAAAAATTTCTGATAACCCAA |
| | ACACCTACGACTACATGAACAAGCGAGTGGTGGCTCCCGGGCT |
| | AGTGGACTGCTACATTAACCTTGGAGCACGCTGGTCCCTTGACT |
| | ATATGGACAACGTCAACCCATTTAACCACCACCGCAATGCTGG |
| | CCTGCGCTACCGCTCAATGTTGCTGGGCAATGGTCGCTATGTGC |
| | CCTTCCACATCCAGGTGCCTCAGAAGTTCTTTGCCATTAAAAAC |
| | CTCCTTCTCCTGCCGGGCTCATACACCTACGAGTGGAACTTCAG |
| | GAAGGATGTTAACATGGTTCTGCAGAGCTCCCTAGGAAATGAC |
| | CTAAGGGTTGACGGAGCCAGCATTAAGTTTGATAGCATTTGCC |
| | TTTACGCCACCTTCTTCCCCATGGCCCACAACACCGCCTCCACG |
| | CTTGAGGCCATGCTTAGAAACGACACCAACGACCAGTCCTTTA |
| | ACGACTATCTCTCCGCCGCCAACATGCTCTACCCTATACCCGCC |
| | AACGCTACCAACGTGCCCATATCCATCCCCTCCCGCAACTGGG |
| | CGGCTTTCCGCGGCTGGGCCTTCACGCGCCTTAAGACTAAGGA |
| | AACCCCATCACTGGGCTCGGGCTACGACCCTTATTACACCTACT |
| | CTGGCTCTATACCCTACCTAGATGGAACCTTTTACCTCAACCAC |
| | ACCTTTAAGAAGGTGGCCATTACCTTTGACTCTTCTGTCAGCTG |
| | GCCTGGCAATGACCGCCTGCTTACCCCCAACGAGTTTGAAATT |
| | AAGCGCTCAGTTGACGGGGAGGGTTACAACGTTGCCCAGTGTA |
| | ACATGACCAAAGACTGGTTCCTGGTACAAATGCTAGCTAACTA |
| | TAACATTGGCTACCAGGGCTTCTATATCCCAGAGAGCTACAAG |
| | GACCGCATGTACTCCTTCTTTAGAAACTTCCAGCCCATGAGCCG |
| | TCAGGTGGTGGATGATACTAAATACAAGGACTACCAACAGGTG |
| | GGCATCCTACACCAACACAACAACTCTGGATTTGTTGGCTACCT |
| | TGCCCCCACCATGCGCGAAGGACAGGCCTACCCTGCTAACTTC |
| | CCCTATCCGCTTATAGGCAAGACCGCAGTTGACAGCATTACCC |
| | AGAAAAAGTTTCTTTGCGATCGCACCCTTTGGCGCATCCCATTC |
| | TCCAGTAACTTTATGTCCATGGGCGCACTCACAGACCTGGGCC |
| | AAAACCTTCTCTACGCCAACTCCGCCCACGCGCTAGACATGAC |
| | TTTTGAGGTGGATCCCATGGACGAGCCCACCCTTCTTTATGTTT |
| | TGTTTGAAGTCTTTGACGTGGTCCGTGTGCACCAGCCGCACCGC |
| | GGCGTCATCGAAACCGTGTACCTGCGCACGCCCTTCTCGGCCG |
| | GCAACGCCACAACATAAAGAAGCAAGCAACATCAACAACAGC |
| | TGCCGCCATGGGCTCCAGTGAGCAGGAACTGAAAGCCATTGTC |
| | AAAGATCTTGGTTGTGGGCCATATTTTTTGGGCACCTATGACAA |
| | GCGCTTTCCAGGCTTTGTTTCTCCACACAAGCTCGCCTGCGCCA |
| | TAGTCAATACGGCCGGTCGCGAGACTGGGGGCGTACACTGGAT |
| | GGCCTTTGCCTGGAACCCGCACTCAAAAACATGCTACCTCTTTG |
| | AGCCCTTTGGCTTTTCTGACCAGCGACTCAAGCAGGTTTACCAG |
| | TTTGAGTACGAGTCACTCCTGCGCCGTAGCGCCATTGCTTCTTC |
| | CCCCGACCGCTGTATAACGCTGGAAAAGTCCACCCAAAGCGTA |
| | CAGGGGCCCAACTCGGCCGCCTGTGGACTATTCTGCTGCATGTT |
| | TCTCCACGCCTTTGCCAACTGGCCCCAAACTCCCATGGATCACA |
| | ACCCCACCATGAACCTTATTACCGGGGTACCCAACTCCATGCTC |
| | AACAGTCCCCAGGTACAGCCCACCCTGCGTCGCAACCAGGAAC |
| | AGCTCTACAGCTTCCTGGAGCGCCACTCGCCCTACTTCCGCAGC |
| | CACAGTGCGCAGATTAGGAGCGCCACTTCTTTTTGTCACTTGAA |
| | AAACATGTAAAAATAATGTACTAGAGACACTTTCAATAAAGGC |
| | AAATGCTTTTATTTGTACACTCTCGGGTGATTATTTACCCCCAC |
| | CCTTGCCGTCTGCGCCGTTTAAAAATCAAAGGGGTTCTGCCGCG |
| | CATCGCTATGCGCCACTGGCAGGGACACGTTGCGATACTGGTG |
| | TTTAGTGCTCCACTTAAACTCAGGCACAACCATCCGCGGCAGCT |
| | CGGTGAAGTTTTCACTCCACAGGCTGCGCACCATCACCAACGC |
| | GTTTAGCAGGTCGGGCGCCGATATCTTGAAGTCGCAGTTGGGG |
| | CCTCCGCCCTGCGCGCGCGAGTTGCGATACACAGGGTTGCAGC |
| | ACTGGAACACTATCAGCGCCGGGTGGTGCACGCTGGCCAGCAC |
| | GCTCTTGTCGGAGATCAGATCCGCGTCCAGGTCCTCCGCGTTGC |
| | TCAGGGCGAACGGAGTCAACTTTGGTAGCTGCCTTCCCAAAAA |
| | GGGCGCGTGCCCAGGCTTTGAGTTGCACTCGCACCGTAGTGGC |
| | ATCAAAAGGTGACCGTGCCCGGTCTGGGCGTTAGGATACAGCG |
| | CCTGCATAAAAGCCTTGATCTGCTTAAAAGCCACCTGAGCCTTT |
| | GCGCCTTCAGAGAAGAACATGCCGCAAGACTTGCCGGAAAACT |
| | GATTGGCCGGACAGGCCGCGTCGTGCACGCAGCACCTTGCGTC |
| | GGTGTTGGAGATCTGCACCACATTTCGGCCCCACCGGTTCTTCA |
| | CGATCTTGGCCTTGCTAGACTGCTCCTTCAGCGCGCGCTGCCCG |
| | TTTTCGCTCGTCACATCCATTTCAATCACGTGCTCCTTATTTATC |
| | ATAATGCTTCCGTGTAGACACTTAAGCTCGCCTTCGATCTCAGC |
| | GCAGCGGTGCAGCCACAACGCGCAGCCCGTGGGCTCGTGATGC |
| | TTGTAGGTCACCTCTGCAAACGACTGCAGGTACGCCTGCAGGA |
| | ATCGCCCCATCATCGTCACAAAGGTCTTGTTGCTGGTGAAGGTC |
| | AGCTGCAACCCGCGGTGCTCCTCGTTCAGCCAGGTCTTGCATAC |
| | GGCCGCCAGAGCTTCCACTTGGTCAGGCAGTAGTTTGAAGTTC |
| | GCCTTTAGATCGTTATCCACGTGGTACTTGTCCATCAGCGCGCG |
| | CGCAGCCTCCATGCCCTTCTCCCACGCAGACACGATCGGCACA |
| | CTCAGCGGGTTCATCACCGTAATTTCACTTTCCGCTTCGCTGGG |
| | CTCTTCCTCTTCCTCTTGCGTCCGCATACCACGCGCCACTGGGT |
| | CGTCTTCATTCAGCCGCCGCACTGTGCGCTTACCTCCTTTGCCA |
| | TGCTTGATTAGCACCGGTGGGTTGCTGAAACCCACCATTTGTAG |
| | CGCCACATCTTCTCTTTCTTCCTCGCTGTCCACGATTACCTCTGG |
| | TGATGGCGGGCGCTCGGGCTTGGGAGAAGGGCGCTTCTTTTTCT |
| | TCTTGGGCGCAATGGCCAAATCCGCCGCCGAGGTCGATGGCCG |
| | CGGGCTGGGTGTGCGCGGCACCAGCGCGTCTTGTGATGAGTCT |
| | TCCTCGTCCTCGGACTCGATACGCCGCCTCATCCGCTTTTTTGG |
| | GGGCGCCCGGGGAGGCGGCGGCGACGGGGACGGGGACGACAC |
| | GTCCTCCATGGTTGGGGGACGTCGCGCCGCACCGCGTCCGCGC |
| | TCGGGGGTGGTTTCGCGCTGCTCCTCTTCCCGACTGGCCATTTC |
| | CTTCTCCTATAGGCAGAAAAAGATCATGGAGTCAGTCGAGAAG |
| | AAGGACAGCCTAACCGCCCCCTCTGAGTTCGCCACCACCGCCT |
| | CCACCGATGCCGCCAACGCGCCTACCACCTTCCCCGTCGAGGC |
| | ACCCCCGCTTGAGGAGGAGGAAGTGATTATCGAGCAGGACCCA |
| | GGTTTTGTAAGCGAAGACGACGAGGACCGCTCAGTACCAACAG |
| | AGGATAAAAAGCAAGACCAGGACAACGCAGAGGCAAACGAGG |
| | AACAAGTCGGGCGGGGGGACGAAAGGCATGGCGACTACCTAG |
| | ATGTGGGAGACGACGTGCTGTTGAAGCATCTGCAGCGCCAGTG |
| | CGCCATTATCTGCGACGCGTTGCAAGAGCGCAGCGATGTGCCC |
| | CTCGCCATAGCGGATGTCAGCCTTGCCTACGAACGCCACCTATT |
| | CTCACCGCGCGTACCCCCCAAACGCCAAGAAAACGGCACATGC |
| | GAGCCCAACCCGCGCCTCAACTTCTACCCCGTATTTGCCGTGCC |
| | AGAGGTGCTTGCCACCTATCACATCTTTTTCCAAAACTGCAAGA |
| | TACCCCTATCCTGCCGTGCCAACCGCAGCCGAGCGGACAAGCA |
| | GCTGGCCTTGCGGCAGGGCGCTGTCATACCTGATATCGCCTCGC |
| | TCAACGAAGTGCCAAAAATCTTTGAGGGTCTTGGACGCGACGA |
| | GAAGCGCGCGGCAAACGCTCTGCAACAGGAAAACAGCGAAAA |
| | TGAAAGTCACTCTGGAGTGTTGGTGGAACTCGAGGGTGACAAC |
| | GCGCGCCTAGCCGTACTAAAACGCAGCATCGAGGTCACCCACT |
| | TTGCCTACCCGGCACTTAACCTACCCCCCAAGGTCATGAGCAC |
| | AGTCATGAGTGAGCTGATCGTGCGCCGTGCGCAGCCCCTGGAG |
| | AGGGATGCAAATTTGCAAGAACAAACAGAGGAGGGCCTACCC |
| | GCAGTTGGCGACGAGCAGCTAGCGCGCTGGCTTCAAACGCGCG |
| | AGCCTGCCGACTTGGAGGAGCGACGCAAACTAATGATGGCCGC |
| | AGTGCTCGTTACCGTGGAGCTTGAGTGCATGCAGCGGTTCTTTG |
| | CTGACCCGGAGATGCAGCGCAAGCTAGAGGAAACATTGCACTA |
| | CACCTTTCGACAGGGCTACGTACGCCAGGCCTGCAAGATCTCC |
| | AACGTGGAGCTCTGCAACCTGGTCTCCTACCTTGGAATTTTGCA |
| | CGAAAACCGCCTTGGGCAAAACGTGCTTCATTCCACGCTCAAG |
| | GGCGAGGCGCGCCGCGACTACGTCCGCGACTGCGTTTACTTAT |
| | TTCTATGCTACACCTGGCAGACGGCCATGGGCGTTTGGCAGCA |
| | GTGCTTGGAGGAGTGCAACCTCAAGGAGCTGCAGAAACTGCTA |
| | AAGCAAAACTTGAAGGACCTATGGACGGCCTTCAACGAGCGCT |
| | CCGTGGCCGCGCACCTGGCGGACATCATTTTCCCCGAACGCCT |
| | GCTTAAAACCCTGCAACAGGGTCTGCCAGACTTCACCAGTCAA |
| | AGCATGTTGCAGAACTTTAGGAACTTTATCCTAGAGCGCTCAG |
| | GAATCTTGCCCGCCACCTGCTGTGCACTTCCTAGCGACTTTGTG |
| | CCCATTAAGTACCGCGAATGCCCTCCGCCGCTTTGGGGCCACTG |
| | CTACCTTCTGCAGCTAGCCAACTACCTTGCCTACCACTCTGACA |
| | TAATGGAAGACGTGAGCGGTGACGGTCTACTGGAGTGTCACTG |
| | TCGCTGCAACCTATGCACCCCGCACCGCTCCCTGGTTTGCAATT |
| | CGCAGCTGCTTAACGAAAGTCAAATTATCGGTACCTTTGAGCT |
| | GCAGGGTCCCTCGCCTGACGAAAAGTCCGCGGCTCCGGGGTTG |
| | AAACTCACTCCGGGGCTGTGGACGTCGGCTTACCTTCGCAAATT |
| | TGTACCTGAGGACTACCACGCCCACGAGATTAGGTTCTACGAA |
| | GACCAATCCCGCCCGCCTAATGCGGAGCTTACCGCCTGCGTCA |
| | TTACCCAGGGCCACATTCTTGGCCAATTGCAAGCCATCAACAA |
| | AGCCCGCCAAGAGTTTCTGCTACGAAAGGGACGGGGGGTTTAC |
| | TTGGACCCCCAGTCCGGCGAGGAGCTCAACCCAATCCCCCCGC |
| | CGCCGCAGCCCTATCAGCAGCAGCCGCGGGCCCTTGCTTCCCA |
| | GGATGGCACCCAAAAAGAAGCTGCAGCTGCCGCCGCCACCCAC |
| | GGACGAGGAGGAATACTGGGACAGTCAGGCAGAGGAGGTTTT |
| | GGACGAGGAGGAGGAGGACATGATGGAAGACTGGGAGAGCCT |
| | AGACGAGGAAGCTTCCGAGGTCGAAGAGGTGTCAGACGAAAC |
| | ACCGTCACCCTCGGTCGCATTCCCCTCGCCGGCGCCCCAGAAAT |
| | CGGCAACCGGTTCCAGCATGGCTACAACCTCCGCTCCTCAGGC |
| | GCCGCCGGCACTGCCCGTTCGCCGACCCAACCGTAGATGGGAC |
| | ACCACTGGAACCAGGGCCGGTAAGTCCAAGCAGCCGCCGCCGT |
| | TAGCCCAAGAGCAACAACAGCGCCAAGGCTACCGCTCATGGCG |
| | CGGGCACAAGAACGCCATAGTTGCTTGCTTGCAAGACTGTGGG |
| | GGCAACATCTCCTTCGCCCGCCGCTTTCTTCTCTACCATCACGG |
| | CGTGGCCTTCCCCCGTAACATCCTGCATTACTACCGTCATCTCT |
| | ACAGCCCATACTGCACCGGCGGCAGCGGCAGCAACAGCAGCG |
| | GCCACACAGAAGCAAAGGCGACCGGATAGCAAGACTCTGACA |
| | AAGCCCAAGAAATCCACAGCGGCGGCAGCAGCAGGAGGAGGA |
| | GCGCTGCGTCTGGCGCCCAACGAACCCGTATCGACCCGCGAGC |
| | TTAGAAACAGGATTTTTCCCACTCTGTATGCTATATTTCAACAG |
| | AGCAGGGGCCAAGAACAAGAGCTGAAAATAAAAAACAGGTCT |
| | CTGCGATCCCTCACCCGCAGCTGCCTGTATCACAAAAGCGAAG |
| | ATCAGCTTCGGCGCACGCTGGAAGACGCGGAGGCTCTCTTCAG |
| | TAAATACTGCGCGCTGACTCTTAAGGACTAGTTTCGCGCCCTTT |
| | CTCAAATTTAAGCGCGAAAACTACGTCATCTCCAGCGGCCACA |
| | CCCGGCGCCAGCACCTGTTGTCAGCGCCATTATGAGCAAGGAA |
| | ATTCCCACGCCCTACATGTGGAGTTACCAGCCACAAATGGGAC |
| | TTGCGGCTGGAGCTGCCCAAGACTACTCAACCCGAATAAACTA |
| | CATGAGCGCGGGACCCCACATGATATCCCGGGTCAACGGAATA |
| | CGCGCCCACCGAAACCGAATTCTCCTGGAACAGGCGGCTATTA |
| | CCACCACACCTCGTAATAACCTTAATCCCCGTAGTTGGCCCGCT |
| | GCCCTGGTGTACCAGGAAAGTCCCGCTCCCACCACTGTGGTAC |
| | TTCCCAGAGACGCCCAGGCCGAAGTTCAGATGACTAACTCAGG |
| | GGCGCAGCTTGCGGGCGGCTTTCGTCACAGGGTGCGGTCGCCC |
| | GGGCAGGGTATAACTCACCTGACAATCAGAGGGCGAGGTATTC |
| | AGCTCAACGACGAGTCGGTGAGCTCCTCGCTTGGTCTCCGTCCG |
| | GACGGGACATTTCAGATCGGCGGCGCCGGCCGCTCTTCATTCA |
| | CGCCTCGTCAGGCAATCCTAACTCTGCAGACCTCGTCCTCTGAG |
| | CCGCGCTCTGGAGGCATTGGAACTCTGCAATTTATTGAGGAGTT |
| | TGTGCCATCGGTCTACTTTAACCCCTTCTCGGGACCTCCCGGCC |
| | ACTATCCGGATCAATTTATTCCTAACTTTGACGCGGTAAAGGAC |
| | TCGGCGGACGGCTACGACTGAATGTTAAGTGGAGAGGCAGAGC |
| | AACTGCGCCTGAAACACCTGGTCCACTGTCGCCGCCACAAGTG |
| | CTTTGCCCGCGACTCCGGTGAGTTTTGCTACTTTGAATTGCCCG |
| | AGGATCATATCGAGGGCCCGGCGCACGGCGTCCGGCTTACCGC |
| | CCAGGGAGAGCTTGCCCGTAGCCTGATTCGGGAGTTTACCCAG |
| | CGCCCCCTGCTAGTTGAGCGGGACAGGGGACCCTGTGTTCTCA |
| | CTGTGATTTGCAACTGTCCTAACCCTGGATTACATCAAGATCCT |
| | CTAGTTAATGTCAGGTCGCCTAAGTCGATTAACTAGAGTACCC |
| | GGGGATCTTATTCCCTTTAACTAATAAAAAAAAATAATAAAGC |
| | ATCACTTACTTAAAATCAGTTAGCAAATTTCTGTCCAGTTTATT |
| | CAGCAGCACCTCCTTGCCCTCCTCCCAGCTCTGGTATTGCAGCT |
| | TCCTCCTGGCTGCAAACTTTCTCCACAATCTAAATGGAATGTCA |
| | GTTTCCTCCTGTTCCTGTCCATCCGCACCCACTATCTTCATGTTG |
| | TTGCAGATGAAGCGCGCAAGACCGTCTGAAGATACCTTCAACC |
| | CCGTGTATCCATATGACACGGAAACCGGTCCTCCAACTGTGCCT |
| | TTTCTTACTCCTCCCTTTGTATCCCCCAATGGGTTTCAAGAGAG |
| | TCCCCCTGGGGTACTCTCTTTGCGCCTATCCGAACCTCTAGTTA |
| | CCTCCAATGGCATGCTTGCGCTCAAAATGGGCAACGGCCTCTCT |
| | CTGGACGAGGCCGGCAACCTTACCTCCCAAAATGTAACCACTG |
| | TGAGCCCACCTCTCAAAAAAACCAAGTCAAACATAAACCTGGA |
| | AATATCTGCACCCCTCACAGTTACCTCAGAAGCCCTAACTGTGG |
| | CTGCCGCCGCACCTCTAATGGTCGCGGGCAACACACTCACCAT |
| | GCAATCACAGGCCCCGCTAACCGTGCACGACTCCAAACTTAGC |
| | ATTGCCACCCAAGGACCCCTCACAGTGTCAGAAGGAAAGCTAG |
| | CCCTGCAAACATCAGGCCCCCTCACCACCACCGATAGCAGTAC |
| | CCTTACTATCACTGCCTCACCCCCTCTAACTACTGCCACTGGTA |
| | GCTTGGGCATTGACTTGAAAGAGCCCATTTATACACAAAATGG |
| | AAAACTAGGACTAAAGTACGGGGCTCCTTTGCATGTAACAGAC |
| | GACCTAAACACTTTGACCGTAGCAACTGGTCCAGGTGTGACTA |
| | TTAATAATACTTCCTTGCAAACTAAAGTTACTGGAGCCTTGGGT |
| | TTTGATTCACAAGGCAATATGCAACTTAATGTAGCAGGAGGAC |
| | TAAGGATTGATTCTCAAAACAGACGCCTTATACTTGATGTTAGT |
| | TATCCGTTTGATGCTCAAAACCAACTAAATCTAAGACTAGGAC |
| | AGGGCCCTCTTTTTATAAACTCAGCCCACAACTTGGATATTAAC |
| | TACAACAAAGGCCTTTACTTGTTTACAGCTTCAAACAATTCCAA |
| | AAAGCTTGAGGTTAACCTAAGCACTGCCAAGGGGTTGATGTTT |
| | GACGCTACAGCCATAGCCATTAATGCAGGAGATGGGCTTGAAT |
| | TTGGTTCACCTAATGCACCAAACACAAATCCCCTCAAAACAAA |
| | AATTGGCCATGGCCTAGAATTTGATTCAAACAAGGCTATGGTT |
| | CCTAAACTAGGAACTGGCCTTAGTTTTGACAGCACAGGTGCCA |
| | TTACAGTAGGAAACAAAAATAATGATAAGCTAACTTTGTGGAC |
| | CACACCAGCTCCATCTCCTAACTGTAGACTAAATGCAGAGAAA |
| | GATGCTAAACTCACTTTGGTCTTAACAAAATGTGGCAGTCAAA |
| | TACTTGCTACAGTTTCAGTTTTGGCTGTTAAAGGCAGTTTGGCT |
| | CCAATATCTGGAACAGTTCAAAGTGCTCATCTTATTATAAGATT |
| | TGACGAAAATGGAGTGCTACTAAACAATTCCTTCCTGGACCCA |
| | GAATATTGGAACTTTAGAAATGGAGATCTTACTGAAGGCACAG |
| | CCTATACAAACGCTGTTGGATTTATGCCTAACCTATCAGCTTAT |
| | CCAAAATCTCACGGTAAAACTGCCAAAAGTAACATTGTCAGTC |
| | AAGTTTACTTAAACGGAGACAAAACTAAACCTGTAACACTAAC |
| | CATTACACTAAACGGTACACAGGAAACAGGAGACACAACTCCA |
| | AGTGCATACTCTATGTCATTTTCATGGGACTGGTCTGGCCACAA |
| | CTACATTAATGAAATATTTGCCACATCCTCTTACACTTTTTCAT |
| | ACATTGCCCAAGAATAAAGAATCGTTTGTGTTATGTTTCAACGT |
| | GTTTATTTTTCAATTGCAGAAAATTTCAAGTCATTTTTCATTCAG |
| | TAGTATAGCCCCACCACCACATAGCTTATACAGATCACCGTAC |
| | CTTAATCAAACTCACAGAACCCTAGTATTCAACCTGCCACCTCC |
| | CTCCCAACACACAGAGTACACAGTCCTTTCTCCCCGGCTGGCCT |
| | TAAAAAGCATCATATCATGGGTAACAGACATATTCTTAGGTGT |
| | TATATTCCACACGGTTTCCTGTCGAGCCAAACGCTCATCAGTGA |
| | TATTAATAAACTCCCCGGGCAGCTCACTTAAGTTCATGTCGCTG |
| | TCCAGCTGCTGAGCCACAGGCTGCTGTCCAACTTGCGGTTGCTT |
| | AACGGGCGGCGAAGGAGAAGTCCACGCCTACATGGGGGTAGA |
| | GTCATAATCGTGCATCAGGATAGGGCGGTGGTGCTGCAGCAGC |
| | GCGCGAATAAACTGCTGCCGCCGCCGCTCCGTCCTGCAGGAAT |
| | ACAACATGGCAGTGGTCTCCTCAGCGATGATTCGCACCGCCCG |
| | CAGCATAAGGCGCCTTGTCCTCCGGGCACAGCAGCGCACCCTG |
| | ATCTCACTTAAATCAGCACAGTAACTGCAGCACAGCACCACAA |
| | TATTGTTCAAAATCCCACAGTGCAAGGCGCTGTATCCAAAGCT |
| | CATGGCGGGGACCACAGAACCCACGTGGCCATCATACCACAAG |
| | CGCAGGTAGATTAAGTGGCGACCCCTCATAAACACGCTGGACA |
| | TAAACATTACCTCTTTTGGCATGTTGTAATTCACCACCTCCCGG |
| | TACCATATAAACCTCTGATTAAACATGGCGCCATCCACCACCAT |
| | CCTAAACCAGCTGGCCAAAACCTGCCCGCCGGCTATACACTGC |
| | AGGGAACCGGGACTGGAACAATGACAGTGGAGAGCCCAGGAC |
| | TCGTAACCATGGATCATCATGCTCGTCATGATATCAATGTTGGC |
| | ACAACACAGGCACACGTGCATACACTTCCTCAGGATTACAAGC |
| | TCCTCCCGCGTTAGAACCATATCCCAGGGAACAACCCATTCCTG |
| | AATCAGCGTAAATCCCACACTGCAGGGAAGACCTCGCACGTAA |
| | CTCACGTTGTGCATTGTCAAAGTGTTACATTCGGGCAGCAGCG |
| | GATGATCCTCCAGTATGGTAGCGCGGGTTTCTGTCTCAAAAGG |
| | AGGTAGACGATCCCTACTGTACGGAGTGCGCCGAGACAACCGA |
| | GATCGTGTTGGTCGTAGTGTCATGCCAAATGGAACGCCGGACG |
| | TAGTCATATTTCCTGAAGCAAAACCAGGTGCGGGCGTGACAAA |
| | CAGATCTGCGTCTCCGGTCTCGCCGCTTAGATCGCTCTGTGTAG |
| | TAGTTGTAGTATATCCACTCTCTCAAAGCATCCAGGCGCCCCCT |
| | GGCTTCGGGTTCTATGTAAACTCCTTCATGCGCCGCTGCCCTGA |
| | TAACATCCACCACCGCAGAATAAGCCACACCCAGCCAACCTAC |
| | ACATTCGTTCTGCGAGTCACACACGGGAGGAGCGGGAAGAGCT |
| | GGAAGAACCATGTTTTTTTTTTTATTCCAAAAGATTATCCAAAA |
| | CCTCAAAATGAAGATCTATTAAGTGAACGCGCTCCCCTCCGGT |
| | GGCGTGGTCAAACTCTACAGCCAAAGAACAGATAATGGCATTT |
| | GTAAGATGTTGCACAATGGCTTCCAAAAGGCAAACGGCCCTCA |
| | CGTCCAAGTGGACGTAAAGGCTAAACCCTTCAGGGTGAATCTC |
| | CTCTATAAACATTCCAGCACCTTCAACCATGCCCAAATAATTCT |
| | CATCTCGCCACCTTCTCAATATATCTCTAAGCAAATCCCGAATA |
| | TTAAGTCCGGCCATTGTAAAAATCTGCTCCAGAGCGCCCTCCAC |
| | CTTCAGCCTCAAGCAGCGAATCATGATTGCAAAAATTCAGGTT |
| | CCTCACAGACCTGTATAAGATTCAAAAGCGGAACATTAACAAA |
| | AATACCGCGATCCCGTAGGTCCCTTCGCAGGGCCAGCTGAACA |
| | TAATCGTGCAGGTCTGCACGGACCAGCGCGGCCACTTCCCCGC |
| | CAGGAACCATGACAAAAGAACCCACACTGATTATGACACGCAT |
| | ACTCGGAGCTATGCTAACCAGCGTAGCCCCGATGTAAGCTTGT |
| | TGCATGGGCGGCGATATAAAATGCAAGGTGCTGCTCAAAAAAT |
| | CAGGCAAAGCCTCGCGCAAAAAAGAAAGCACATCGTAGTCAT |
| | GCTCATGCAGATAAAGGCAGGTAAGCTCCGGAACCACCACAGA |
| | AAAAGACACCATTTTTCTCTCAAACATGTCTGCGGGTTTCTGCA |
| | TAAACACAAAATAAAATAACAAAAAAACATTTAAACATTAGA |
| | AGCCTGTCTTACAACAGGAAAAACAACCCTTATAAGCATAAGA |
| | CGGACTACGGCCATGCCGGCGTGACCGTAAAAAAACTGGTCAC |
| | CGTGATTAAAAAGCACCACCGACAGCTCCTCGGTCATGTCCGG |
| | AGTCATAATGTAAGACTCGGTAAACACATCAGGTTGATTCACA |
| | TCGGTCAGTGCTAAAAAGCGACCGAAATAGCCCGGGGGAATAC |
| | ATACCCGCAGGCGTAGAGACAACATTACAGCCCCCATAGGAGG |
| | TATAACAAAATTAATAGGAGAGAAAAACACATAAACACCTGA |
| | AAAACCCTCCTGCCTAGGCAAAATAGCACCCTCCCGCTCCAGA |
| | ACAACATACAGCGCTTCCACAGCGGCAGCCATAACAGTCAGCC |
| | TTACCAGTAAAAAAGAAAACCTATTAAAAAAACACCACTCGAC |
| | ACGGCACCAGCTCAATCAGTCACAGTGTAAAAAAGGGCCAAGT |
| | GCAGAGCGAGTATATATAGGACTAAAAAATGACGTAACGGTTA |
| | AAGTCCACAAAAAACACCCAGAAAACCGCACGCGAACCTACG |
| | CCCAGAAACGAAAGCCAAAAAACCCACAACTTCCTCAAATCGT |
| | CACTTCCGTTTTCCCACGTTACGTCACTTCCCATTTTAAGAAAA |
| | CTACAATTCCCAACACATACAAGTTACTCCGCCCTAAAACCTAC |
| | GTCACCCGCCCCGTTCCCACGCCCCGCGCCACGTCACAAACTCC |
| | ACCCCCTCATTATCATATTGGCTTCAATCCAAAATAAGGTATAT |
| | TATTGATGAT |
| SEQ ID NO: | CTCGAGGAAGCTTGCCGCCACCATGCACCAAAAGAGAACTGCA |
| 18 | ATGTTTCAGGACCCACAGGAGCGACCCAGAAAGTTACCACAGT |
| | TATGCACAGAGGTGCAAACAACTATACATGATATAATATTAGA |
| | ATGTGTGTACTGCAAGCAACAGTTACTGCGACGTGAGGTATAT |
| | GACTTTGCTTTTCGGGATGGATGCATAGTATATAGAGATGGGA |
| | ATCCATATGCTGTATGTGATAAATGTTTAAAGTTTTATTCTAAA |
| | ATTAGTGAGTATAGACATTATTGTTATAGTTTGTATGGAACAAC |
| | ATTAGAACAGCAATACAACAAACCGTTGTGTGATTTGTTAATT |
| | AGGTGTATTAACTGTCAAAAGCCACTGTGTCCTGAAGAAAAGC |
| | AAAGACATCTGGACAAAAAGCAAAGATTCCATAATATAAGGG |
| | GTCGGTGGACCGGTCGATGTATGTCTTGTTGCAGATCATCAAG |
| | AACTCGTAGAGCAGCCGCGGCGTGAGATATCGCGGCCGC |
| SEQ ID NO: | CTCGAGGAAGCTTGCCGCCACCATGCACCAAAAGAGAACTGCA |
| 19 | ATGTTTCAGGACCCACAGGAGCGACCCAGAAAGTTACCACAGT |
| | TATGCACAGAGCTGCAAACAACTATACATGATATAATATTAGA |
| | ATGTGTGTACTGCAAGCAACAGTTACTGCGACGTGAGGTATAT |
| | GACTTTGCTTTTCGGGATGGATGCATAGTATATAGAGATGGGA |
| | ATCCATATGCTGTATGTGATAAATGTTTAAAGTTTTATTCTAAA |
| | ATTAGTGAGTATAGACATTATTGTTATAGTTTGTATGGAACAAC |
| | ATTAGAACAGCTATACAACAAACCGTTGTGTGATGTGTTAATT |
| | AGGTGTATTAACTGTCAAAAGCCACTGTGTCCTGAAGAAAAGC |
| | AAAGACATCTGGACAAAAAGCAAAGATTCCATAATATAAGGG |
| | GTCGGTGGACCGGTCGATGTATGTCTTGTTGCAGATCATCAAG |
| | AACTCGTAGAGCAGCCGCGGCGTGAGATATCGCGGCCGC |
| SEQ ID NO: | CTCGAGGAAGCTTGCCGCCACCATGCACCAAAAGAGAACTGCA |
| 20 | ATGTTTCAGGACCCACAGGAGCGACCCAGAAAGTTACCACAGT |
| | TATGCACAGAGGTGCAAACAACTATACATGATATAATATTAGA |
| | ATGTGTGTACTGCAAGCAACAGTTACTGCGACGTGAGGTATAT |
| | GACTTTGCTTTTCGGGATGGATGCATAGTATATAGAGATGGGA |
| | ATCCATATGCTGTATGTGATAAATGTTTAAAGTTTTATTCTAAA |
| | ATTAGTGAGTATAGACATTATTGTTATAGTTTGTATGGAACAAC |
| | ATTAGAACAGCTATACAACAAACCGTTGTGTGATGTGTTAATT |
| | AGGTGTATTAACTGTCAAAAGCCACTGTGTCCTGAAGAAAAGC |
| | AAAGACATCTGGACAAAAAGCAAAGATTCCATAATATAAGGG |
| | GTCGGTGGACCGGTCGATGTATGTCTTGTTGCAGATCATCAAG |
| | AACTCGTAGAGCAGCCGCGGCGTGAGATATCGCGGCCGC |
| SEQ ID NO: | CTCGAGGAAGCTTGCCGCCACCATGCCTGGAGATACACCTACA |
| 21 | TTGCATGAATATATGTTAGATTTGCAACCAGAGACAACTGATCT |
| | CTACGGTTATGAGCAATTAAATGACAGCTCAGAGGAGGAGGAT |
| | GAAATAGATGGTCCAGCTGGACAAGCAGCACCGGACAGAGCC |
| | CATTACAATATTGTAACCTTTTGTTGCAAGTGTGACTCTACGCT |
| | TCGGAGGTGCGTACAAAGCACACACGTAGACATTCGTACTTTG |
| | GAAGACCTGTTAATGGGCGTACTAGGAATTGTGTGCCCCATCT |
| | GTTCTCAGAAACCATGAGATATCGCGGCCGC |
| SEQ ID NO: | ATGGAGTCTCCCTCGGCCCCTCCCCACAGATGGTGCATCCCCTG |
| 22 | GCAGAGGCTCCTGCTCACAGCCTCACTTCTAACCTTCTGGAACC |
| | CGCCCACCACTGCCAAGCTCACTATTGAATCCACGCCGTTCAAT |
| | GTCGCAGAGGGGAAGGAGGTGCTTCTACTTGTCCACAATCTGC |
| | CCCAGCATCTTTTTGGCTACAGCTGGTACAAAGGTGAAAGAGT |
| | GGATGGCAACCGTCAAATTATAGGATATGTAATAGGAACTCAA |
| | CAAGCTACCCCAGGGCCCGCATACAGTGGTCGAGAGATAATAT |
| | ACCCCAATGCATCCCTGCTGATCCAGAACATCATCCAGAATGA |
| | CACAGGATTCTACACCCTACACGTCATAAAGTCAGATCTTGTG |
| | AATGAAGAAGCAACTGGCCAGTTCCGGGTATACCCGGAGCTGC |
| | CCAAGCCCTCCATCTCCAGCAACAACTCCAAACCCGTGGAGGA |
| | CAAGGATGCTGTGGCCTTCACCTGTGAACCTGAGACTCAGGAC |
| | GCAACCTACCTGTGGTGGGTAAACAATCAGAGCCTCCCGGTCA |
| | GTCCCAGGCTGCAGCTGTCCAATGGCAACAGGACCCTCACTCT |
| | ATTCAATGTCACAAGAAATGACACAGCAAGCTACAAATGTGAA |
| | ACCCAGAACCCAGTGAGTGCCAGGCGCAGTGATTCAGTCATCC |
| | TGAATGTCCTCTATGGCCCGGATGCCCCCACCATTTCCCCTCTA |
| | AACACATCTTACAGATCAGGGGAAAATCTGAACCTCTCCTGCC |
| | ACGCAGCCTCTAACCCACCTGCACAGTACTCTTGGTTTGTCAAT |
| | GGGACTTTCCAGCAATCCACCCAAGAGCTCTTTATCCCCAACAT |
| | CACTGTGAATAATAGTGGATCCTATACGTGCCAAGCCCATAAC |
| | TCAGACACTGGCCTCAATAGGACCACAGTCACGACGATCACAG |
| | TCTATGCAGAGCCACCCAAACCCTTCATCACCAGCAACAACTC |
| | CAACCCCGTGGAGGATGAGGATGCTGTAGCCTTAACCTGTGAA |
| | CCTGAGATTCAGAACACAACCTACCTGTGGTGGGTAAATAATC |
| | AGAGCCTCCCGGTCAGTCCCAGGCTGCAGCTGTCCAATGACAA |
| | CAGGACCCTCACTCTACTCAGTGTCACAAGGAATGATGTAGGA |
| | CCCTATGAGTGTGGAATCCAGAACGAATTAAGTGTTGACCACA |
| | GCGACCCAGTCATCCTGAATGTCCTCTATGGCCCAGACGACCC |
| | CACCATTTCCCCCTCATACACCTATTACCGTCCAGGGGTGAACC |
| | TCAGCCTCTCCTGCCATGCAGCCTCTAACCCACCTGCACAGTAT |
| | TCTTGGCTGATTGATGGGAACATCCAGCAACACACACAAGAGC |
| | TCTTTATCTCCAACATCACTGAGAAGAACAGCGGACTCTATACC |
| | TGCCAGGCCAATAACTCAGCCAGTGGCCACAGCAGGACTACAG |
| | TCAAGACAATCACAGTCTCTGCGGAGCTGCCCAAGCCCTCCAT |
| | CTCCAGCAACAACTCCAAACCCGTGGAGGACAAGGATGCTGTG |
| | GCCTTCACCTGTGAACCTGAGGCTCAGAACACAACCTACCTGT |
| | GGTGGGTAAATGGTCAGAGCCTCCCAGTCAGTCCCAGGCTGCA |
| | GCTGTCCAATGGCAACAGGACCCTCACTCTATTCAATGTCACA |
| | AGAAATGACGCAAGAGCCTATGTATGTGGAATCCAGAACTCAG |
| | TGAGTGCAAACCGCAGTGACCCAGTCACCCTGGATGTCCTCTA |
| | TGGGCCGGACACCCCCATCATTTCCCCCCCAGACTCGTCTTACC |
| | TTTCGGGAGCGGACCTCAACCTCTCCTGCCACTCGGCCTCTAAC |
| | CCATCCCCGCAGTATTCTTGGCGTATCAATGGGATACCGCAGC |
| | AACACACACAAGTTCTCTTTATCGCCAAAATCACGCCAAATAA |
| | TAACGGGACCTATGCCTGTTTTGTCTCTAACTTGGCTACTGGCC |
| | GCAATAATTCCATAGTCAAGAGCATCACAGTCTCTGCATCTGG |
| | AACTTCTCCTGGTCTCTCAGCTGGGGCCACTGTCGGCATCATGA |
| | TTGGAGTGCTGGTTGGGGTTGCTCTGATATAG |
| SEQ ID NO: | YLSGANLNL |
| 23 | |
| SEQ ID NO: | YLSGADLNL |
| 24 | |
| SEQ ID NO: | CATCATCAATAATATACCTTATTTTGGATTGAAGCCAATATGAT |
| 25 | AATGAGGGGGTGGAGTTTGTGACGTGGCGCGGGGCGTGGGAA |
| | CGGGGCGGGTGACGTAGTAGTGTGGCGGAAGTGTGATGTTGCA |
| | AGTGTGGCGGAACACATGTAAGCGACGGATGTGGCAAAAGTG |
| | ACGTTTTTGGTGTGCGCCGGTGTACACAGGAAGTGACAATTTTC |
| | GCGCGGTTTTAGGCGGATGTTGTAGTAAATTTGGGCGTAACCG |
| | AGTAAGATTTGGCCATTTTCGCGGGAAAACTGAATAAGAGGAA |
| | GTGAAATCTGAATAATTTTGTGTTACTCATAGCGCGTAATACTG |
| | TAATAGTAATCAATTACGGGGTCATTAGTTCATAGCCCATATAT |
| | GGAGTTCCGCGTTACATAACTTACGGTAAATGGCCCGCCTGGC |
| | TGACCGCCCAACGACCCCCGCCCATTGACGTCAATAATGACGT |
| | ATGTTCCCATAGTAACGCCAATAGGGACTTTCCATTGACGTCAA |
| | TGGGTGGAGTATTTACGGTAAACTGCCCACTTGGCAGTACATC |
| | AAGTGTATCATATGCCAAGTACGCCCCCTATTGACGTCAATGA |
| | CGGTAAATGGCCCGCCTGGCATTATGCCCAGTACATGACCTTAT |
| | GGGACTTTCCTACTTGGCAGTACATCTACGTATTAGTCATCGCT |
| | ATTACCATGGTGATGCGGTTTTGGCAGTACATCAATGGGCGTG |
| | GATAGCGGTTTGACTCACGGGGATTTCCAAGTCTCCACCCCATT |
| | GACGTCAATGGGAGTTTGTTTTGGCACCAAAATCAACGGGACT |
| | TTCCAAAATGTCGTAACAACTCCGCCCCATTGACGCAAATGGG |
| | CGGTAGGCGTGTACGGTGGGAGGTCTATATAAGCAGAGCTGGT |
| | TTAGTGAACCGTCAGATCCGCTAGAGATCTGGTACCGTCGACG |
| | CGGCCGCTCGAGCCTAAGCTTGGTACCGAGCTCGGATCCACTA |
| | GTAACGGCCGCCAGTGTGCTGGAATTCGGCTTAAAGGTACCCA |
| | GAGCAGACAGCCGCCACCATGGAGTCTCCCTCGGCCCCTCCCC |
| | ACAGATGGTGCATCCCCTGGCAGAGGCTCCTGCTCACAGCCTC |
| | ACTTCTAACCTTCTGGAACCCGCCCACCACTGCCAAGCTCACTA |
| | TTGAATCCACGCCGTTCAATGTCGCAGAGGGGAAGGAGGTGCT |
| | TCTACTTGTCCACAATCTGCCCCAGCATCTTTTTGGCTACAGCT |
| | GGTACAAAGGTGAAAGAGTGGATGGCAACCGTCAAATTATAG |
| | GATATGTAATAGGAACTCAACAAGCTACCCCAGGGCCCGCATA |
| | CAGTGGTCGAGAGATAATATACCCCAATGCATCCCTGCTGATC |
| | CAGAACATCATCCAGAATGACACAGGATTCTACACCCTACACG |
| | TCATAAAGTCAGATCTTGTGAATGAAGAAGCAACTGGCCAGTT |
| | CCGGGTATACCCGGAGCTGCCCAAGCCCTCCATCTCCAGCAAC |
| | AACTCCAAACCCGTGGAGGACAAGGATGCTGTGGCCTTCACCT |
| | GTGAACCTGAGACTCAGGACGCAACCTACCTGTGGTGGGTAAA |
| | CAATCAGAGCCTCCCGGTCAGTCCCAGGCTGCAGCTGTCCAAT |
| | GGCAACAGGACCCTCACTCTATTCAATGTCACAAGAAATGACA |
| | CAGCAAGCTACAAATGTGAAACCCAGAACCCAGTGAGTGCCAG |
| | GCGCAGTGATTCAGTCATCCTGAATGTCCTCTATGGCCCGGATG |
| | CCCCCACCATTTCCCCTCTAAACACATCTTACAGATCAGGGGAA |
| | AATCTGAACCTCTCCTGCCACGCAGCCTCTAACCCACCTGCACA |
| | GTACTCTTGGTTTGTCAATGGGACTTTCCAGCAATCCACCCAAG |
| | AGCTCTTTATCCCCAACATCACTGTGAATAATAGTGGATCCTAT |
| | ACGTGCCAAGCCCATAACTCAGACACTGGCCTCAATAGGACCA |
| | CAGTCACGACGATCACAGTCTATGCAGAGCCACCCAAACCCTT |
| | CATCACCAGCAACAACTCCAACCCCGTGGAGGATGAGGATGCT |
| | GTAGCCTTAACCTGTGAACCTGAGATTCAGAACACAACCTACC |
| | TGTGGTGGGTAAATAATCAGAGCCTCCCGGTCAGTCCCAGGCT |
| | GCAGCTGTCCAATGACAACAGGACCCTCACTCTACTCAGTGTC |
| | ACAAGGAATGATGTAGGACCCTATGAGTGTGGAATCCAGAACG |
| | AATTAAGTGTTGACCACAGCGACCCAGTCATCCTGAATGTCCTC |
| | TATGGCCCAGACGACCCCACCATTTCCCCCTCATACACCTATTA |
| | CCGTCCAGGGGTGAACCTCAGCCTCTCCTGCCATGCAGCCTCTA |
| | ACCCACCTGCACAGTATTCTTGGCTGATTGATGGGAACATCCA |
| | GCAACACACACAAGAGCTCTTTATCTCCAACATCACTGAGAAG |
| | AACAGCGGACTCTATACCTGCCAGGCCAATAACTCAGCCAGTG |
| | GCCACAGCAGGACTACAGTCAAGACAATCACAGTCTCTGCGGA |
| | GCTGCCCAAGCCCTCCATCTCCAGCAACAACTCCAAACCCGTG |
| | GAGGACAAGGATGCTGTGGCCTTCACCTGTGAACCTGAGGCTC |
| | AGAACACAACCTACCTGTGGTGGGTAAATGGTCAGAGCCTCCC |
| | AGTCAGTCCCAGGCTGCAGCTGTCCAATGGCAACAGGACCCTC |
| | ACTCTATTCAATGTCACAAGAAATGACGCAAGAGCCTATGTAT |
| | GTGGAATCCAGAACTCAGTGAGTGCAAACCGCAGTGACCCAGT |
| | CACCCTGGATGTCCTCTATGGGCCGGACACCCCCATCATTTCCC |
| | CCCCAGACTCGTCTTACCTTTCGGGAGCGGACCTCAACCTCTCC |
| | TGCCACTCGGCCTCTAACCCATCCCCGCAGTATTCTTGGCGTAT |
| | CAATGGGATACCGCAGCAACACACACAAGTTCTCTTTATCGCC |
| | AAAATCACGCCAAATAATAACGGGACCTATGCCTGTTTTGTCTC |
| | TAACTTGGCTACTGGCCGCAATAATTCCATAGTCAAGAGCATC |
| | ACAGTCTCTGCATCTGGAACTTCTCCTGGTCTCTCAGCTGGGGC |
| | CACTGTCGGCATCATGATTGGAGTGCTGGTTGGGGTTGCTCTGA |
| | TATAGCAGCCCTGGTGTAGTTTCTTCATTTCAGGAAGACTGACA |
| | GTTGTTTTGCTTCTTCCTTAAAGCATTTGCAACAGCTACAGTCT |
| | AAAATTGCTTCTTTACCAAGGATATTTACAGAAAAGACTCTGA |
| | CCAGAGATCGAGACCATCCTCTAGATAAGATATCCGATCCACC |
| | GGATCTAGATAACTGATCATAATCAGCCATACCACATTTGTAG |
| | AGGTTTTACTTGCTTTAAAAAACCTCCCACACCTCCCCCTGAAC |
| | CTGAAACATAAAATGAATGCAATTGTTGTTGTTAACTTGTTTAT |
| | TGCAGCTTATAATGGTTACAAATAAAGCAATAGCATCACAAAT |
| | TTCACAAATAAAGCATTTTTTTCACTGCATTCTAGTTGTGGTTT |
| | GTCCAAACTCATCAATGTATCTTAACGCGGATCTGGGCGTGGTT |
| | AAGGGTGGGAAAGAATATATAAGGTGGGGGTCTTATGTAGTTT |
| | TGTATCTGTTTTGCAGCAGCCGCCGCCGCCATGAGCACCAACTC |
| | GTTTGATGGAAGCATTGTGAGCTCATATTTGACAACGCGCATG |
| | CCCCCATGGGCCGGGGTGCGTCAGAATGTGATGGGCTCCAGCA |
| | TTGATGGTCGCCCCGTCCTGCCCGCAAACTCTACTACCTTGACC |
| | TACGAGACCGTGTCTGGAACGCCGTTGGAGACTGCAGCCTCCG |
| | CCGCCGCTTCAGCCGCTGCAGCCACCGCCCGCGGGATTGTGAC |
| | TGACTTTGCTTTCCTGAGCCCGCTTGCAAGCAGTGCAGCTTCCC |
| | GTTCATCCGCCCGCGATGACAAGTTGACGGCTCTTTTGGCACAA |
| | TTGGATTCTTTGACCCGGGAACTTAATGTCGTTTCTCAGCAGCT |
| | GTTGGATCTGCGCCAGCAGGTTTCTGCCCTGAAGGCTTCCTCCC |
| | CTCCCAATGCGGTTTAAAACATAAATAAAAAACCAGACTCTGT |
| | TTGGATTTGGATCAAGCAAGTGTCTTGCTGTCTTTATTTAGGGG |
| | TTTTGCGCGCGCGGTAGGCCCGGGACCAGCGGTCTCGGTCGTT |
| | GAGGGTCCTGTGTATTTTTTCCAGGACGTGGTAAAGGTGACTCT |
| | GGATGTTCAGATACATGGGCATAAGCCCGTCTCTGGGGTGGAG |
| | GTAGCACCACTGCAGAGCTTCATGCTGCGGGGTGGTGTTGTAG |
| | ATGATCCAGTCGTAGCAGGAGCGCTGGGCGTGGTGCCTAAAAA |
| | TGTCTTTCAGTAGCAAGCTGATTGCCAGGGGCAGGCCCTTGGT |
| | GTAAGTGTTTACAAAGCGGTTAAGCTGGGATGGGTGCATACGT |
| | GGGGATATGAGATGCATCTTGGACTGTATTTTTAGGTTGGCTAT |
| | GTTCCCAGCCATATCCCTCCGGGGATTCATGTTGTGCAGAACCA |
| | CCAGCACAGTGTATCCGGTGCACTTGGGAAATTTGTCATGTAG |
| | CTTAGAAGGAAATGCGTGGAAGAACTTGGAGACGCCCTTGTGA |
| | CCTCCAAGATTTTCCATGCATTCGTCCATAATGATGGCAATGGG |
| | CCCACGGGCGGCGGCCTGGGCGAAGATATTTCTGGGATCACTA |
| | ACGTCATAGTTGTGTTCCAGGATGAGATCGTCATAGGCCATTTT |
| | TACAAAGCGCGGGCGGAGGGTGCCAGACTGCGGTATAATGGTT |
| | CCATCCGGCCCAGGGGCGTAGTTACCCTCACAGATTTGCATTTC |
| | CCACGCTTTGAGTTCAGATGGGGGGATCATGTCTACCTGCGGG |
| | GCGATGAAGAAAACGGTTTCCGGGGTAGGGGAGATCAGCTGG |
| | GAAGAAAGCAGGTTCCTGAGCAGCTGCGACTTACCGCAGCCGG |
| | TGGGCCCGTAAATCACACCTATTACCGGCTGCAACTGGTAGTT |
| | AAGAGAGCTGCAGCTGCCGTCATCCCTGAGCAGGGGGGCCACT |
| | TCGTTAAGCATGTCCCTGACTCGCATGTTTTCCCTGACCAAATC |
| | CGCCAGAAGGCGCTCGCCGCCCAGCGATAGCAGTTCTTGCAAG |
| | GAAGCAAAGTTTTTCAACGGTTTGAGACCGTCCGCCGTAGGCA |
| | TGCTTTTGAGCGTTTGACCAAGCAGTTCCAGGCGGTCCCACAGC |
| | TCGGTCACCTGCTCTACGGCATCTCGATCCAGCATATCTCCTCG |
| | TTTCGCGGGTTGGGGCGGCTTTCGCTGTACGGCAGTAGTCGGTG |
| | CTCGTCCAGACGGGCCAGGGTCATGTCTTTCCACGGGCGCAGG |
| | GTCCTCGTCAGCGTAGTCTGGGTCACGGTGAAGGGGTGCGCTC |
| | CGGGCTGCGCGCTGGCCAGGGTGCGCTTGAGGCTGGTCCTGCT |
| | GGTGCTGAAGCGCTGCCGGTCTTCGCCCTGCGCGTCGGCCAGG |
| | TAGCATTTGACCATGGTGTCATAGTCCAGCCCCTCCGCGGCGTG |
| | GCCCTTGGCGCGCAGCTTGCCCTTGGAGGAGGCGCCGCACGAG |
| | GGGCAGTGCAGACTTTTGAGGGCGTAGAGCTTGGGCGCGAGAA |
| | ATACCGATTCCGGGGAGTAGGCATCCGCGCCGCAGGCCCCGCA |
| | GACGGTCTCGCATTCCACGAGCCAGGTGAGCTCTGGCCGTTCG |
| | GGGTCAAAAACCAGGTTTCCCCCATGCTTTTTGATGCGTTTCTT |
| | ACCTCTGGTTTCCATGAGCCGGTGTCCACGCTCGGTGACGAAA |
| | AGGCTGTCCGTGTCCCCGTATACAGACTTGAGAGGCCTGTCCTC |
| | GAGCGGTGTTCCGCGGTCCTCCTCGTATAGAAACTCGGACCAC |
| | TCTGAGACAAAGGCTCGCGTCCAGGCCAGCACGAAGGAGGCTA |
| | AGTGGGAGGGGTAGCGGTCGTTGTCCACTAGGGGGTCCACTCG |
| | CTCCAGGGTGTGAAGACACATGTCGCCCTCTTCGGCATCAAGG |
| | AAGGTGATTGGTTTGTAGGTGTAGGCCACGTGACCGGGTGTTC |
| | CTGAAGGGGGGCTATAAAAGGGGGTGGGGGCGCGTTCGTCCTC |
| | ACTCTCTTCCGCATCGCTGTCTGCGAGGGCCAGCTGTTGGGGTG |
| | AGTACTCCCTCTGAAAAGCGGGCATGACTTCTGCGCTAAGATT |
| | GTCAGTTTCCAAAAACGAGGAGGATTTGATATTCACCTGGCCC |
| | GCGGTGATGCCTTTGAGGGTGGCCGCATCCATCTGGTCAGAAA |
| | AGACAATCTTTTTGTTGTCAAGCTTGGTGGCAAACGACCCGTAG |
| | AGGGCGTTGGACAGCAACTTGGCGATGGAGCGCAGGGTTTGGT |
| | TTTTGTCGCGATCGGCGCGCTCCTTGGCCGCGATGTTTAGCTGC |
| | ACGTATTCGCGCGCAACGCACCGCCATTCGGGAAAGACGGTGG |
| | TGCGCTCGTCGGGCACCAGGTGCACGCGCCAACCGCGGTTGTG |
| | CAGGGTGACAAGGTCAACGCTGGTGGCTACCTCTCCGCGTAGG |
| | CGCTCGTTGGTCCAGCAGAGGCGGCCGCCCTTGCGCGAGCAGA |
| | ATGGCGGTAGGGGGTCTAGCTGCGTCTCGTCCGGGGGGTCTGC |
| | GTCCACGGTAAAGACCCCGGGCAGCAGGCGCGCGTCGAAGTA |
| | GTCTATCTTGCATCCTTGCAAGTCTAGCGCCTGCTGCCATGCGC |
| | GGGCGGCAAGCGCGCGCTCGTATGGGTTGAGTGGGGGACCCCA |
| | TGGCATGGGGTGGGTGAGCGCGGAGGCGTACATGCCGCAAATG |
| | TCGTAAACGTAGAGGGGCTCTCTGAGTATTCCAAGATATGTAG |
| | GGTAGCATCTTCCACCGCGGATGCTGGCGCGCACGTAATCGTA |
| | TAGTTCGTGCGAGGGAGCGAGGAGGTCGGGACCGAGGTTGCTA |
| | CGGGCGGGCTGCTCTGCTCGGAAGACTATCTGCCTGAAGATGG |
| | CATGTGAGTTGGATGATATGGTTGGACGCTGGAAGACGTTGAA |
| | GCTGGCGTCTGTGAGACCTACCGCGTCACGCACGAAGGAGGCG |
| | TAGGAGTCGCGCAGCTTGTTGACCAGCTCGGCGGTGACCTGCA |
| | CGTCTAGGGCGCAGTAGTCCAGGGTTTCCTTGATGATGTCATAC |
| | TTATCCTGTCCCTTTTTTTTCCACAGCTCGCGGTTGAGGACAAA |
| | CTCTTCGCGGTCTTTCCAGTACTCTTGGATCGGAAACCCGTCGG |
| | CCTCCGAACGGTAAGAGCCTAGCATGTAGAACTGGTTGACGGC |
| | CTGGTAGGCGCAGCATCCCTTTTCTACGGGTAGCGCGTATGCCT |
| | GCGCGGCCTTCCGGCATGACCAGCATGAAGGGCACGAGCTGCT |
| | TCCCAAAGGCCCCCATCCAAGTATAGGTCTCTACATCGTAGGT |
| | GACAAAGAGACGCTCGGTGCGAGGATGCGAGCCGATCGGGAA |
| | GAACTGGATCTCCCGCCACCAATTGGAGGAGTGGCTATTGATG |
| | TGGTGAAAGTAGAAGTCCCTGCGACGGGCCGAACACTCGTGCT |
| | GGCTTTTGTAAAAACGTGCGCAGTACTGGCAGCGGTGCACGGG |
| | CTGTACATCCTGCACGAGGTTGACCTGACGACCGCGCACAAGG |
| | AAGCAGAGTGGGAATTTGAGCCCCTCGCCTGGCGGGTTTGGCT |
| | GGTGGTCTTCTACTTCGGCTGCTTGTCCTTGACCGTCTGGCTGC |
| | TCGAGGGGAGTTACGGTGGATCGGACCACCACGCCGCGCGAGC |
| | CCAAAGTCCAGATGTCCGCGCGCGGCGGTCGGAGCTTGATGAC |
| | AACATCGCGCAGATGGGAGCTGTCCATGGTCTGGAGCTCCCGC |
| | GGCGTCAGGTCAGGCGGGAGCTCCTGCAGGTTTACCTCGCATA |
| | GACGGGTCAGGGCGCGGGCTAGATCCAGGTGATACCTAATTTC |
| | CAGGGGCTGGTTGGTGGCGGCGTCGATGGCTTGCAAGAGGCCG |
| | CATCCCCGCGGCGCGACTACGGTACCGCGCGGCGGGCGGTGGG |
| | CCGCGGGGGTGTCCTTGGATGATGCATCTAAAAGCGGTGACGC |
| | GGGCGAGCCCCCGGAGGTAGGGGGGGCTCCGGACCCGCCGGG |
| | AGAGGGGGCAGGGGCACGTCGGCGCCGCGCGCGGGCAGGAGC |
| | TGGTGCTGCGCGCGTAGGTTGCTGGCGAACGCGACGACGCGGC |
| | GGTTGATCTCCTGAATCTGGCGCCTCTGCGTGAAGACGACGGG |
| | CCCGGTGAGCTTGAACCTGAAAGAGAGTTCGACAGAATCAATT |
| | TCGGTGTCGTTGACGGCGGCCTGGCGCAAAATCTCCTGCACGT |
| | CTCCTGAGTTGTCTTGATAGGCGATCTCGGCCATGAACTGCTCG |
| | ATCTCTTCCTCCTGGAGATCTCCGCGTCCGGCTCGCTCCACGGT |
| | GGCGGCGAGGTCGTTGGAAATGCGGGCCATGAGCTGCGAGAA |
| | GGCGTTGAGGCCTCCCTCGTTCCAGACGCGGCTGTAGACCACG |
| | CCCCCTTCGGCATCGCGGGCGCGCATGACCACCTGCGCGAGAT |
| | TGAGCTCCACGTGCCGGGCGAAGACGGCGTAGTTTCGCAGGCG |
| | CTGAAAGAGGTAGTTGAGGGTGGTGGCGGTGTGTTCTGCCACG |
| | AAGAAGTACATAACCCAGCGTCGCAACGTGGATTCGTTGATAA |
| | TTGTTGTGTAGGTACTCCGCCGCCGAGGGACCTGAGCGAGTCC |
| | GCATCGACCGGATCGGAAAACCTCTCGAGAAAGGCGTCTAACC |
| | AGTCACAGTCGCAAGGTAGGCTGAGCACCGTGGCGGGCGGCA |
| | GCGGGCGGCGGTCGGGGTTGTTTCTGGCGGAGGTGCTGCTGAT |
| | GATGTAATTAAAGTAGGCGGTCTTGAGACGGCGGATGGTCGAC |
| | AGAAGCACCATGTCCTTGGGTCCGGCCTGCTGAATGCGCAGGC |
| | GGTCGGCCATGCCCCAGGCTTCGTTTTGACATCGGCGCAGGTCT |
| | TTGTAGTAGTCTTGCATGAGCCTTTCTACCGGCACTTCTTCTTCT |
| | CCTTCCTCTTGTCCTGCATCTCTTGCATCTATCGCTGCGGCGGC |
| | GGCGGAGTTTGGCCGTAGGTGGCGCCCTCTTCCTCCCATGCGTG |
| | TGACCCCGAAGCCCCTCATCGGCTGAAGCAGGGCTAGGTCGGC |
| | GACAACGCGCTCGGCTAATATGGCCTGCTGCACCTGCGTGAGG |
| | GTAGACTGGAAGTCATCCATGTCCACAAAGCGGTGGTATGCGC |
| | CCGTGTTGATGGTGTAAGTGCAGTTGGCCATAACGGACCAGTT |
| | AACGGTCTGGTGACCCGGCTGCGAGAGCTCGGTGTACCTGAGA |
| | CGCGAGTAAGCCCTCGAGTCAAATACGTAGTCGTTGCAAGTCC |
| | GCACCAGGTACTGGTATCCCACCAAAAAGTGCGGCGGCGGCTG |
| | GCGGTAGAGGGGCCAGCGTAGGGTGGCCGGGGCTCCGGGGGC |
| | GAGATCTTCCAACATAAGGCGATGATATCCGTAGATGTACCTG |
| | GACATCCAGGTGATGCCGGCGGCGGTGGTGGAGGCGCGCGGA |
| | AAGTCGCGGACGCGGTTCCAGATGTTGCGCAGCGGCAAAAAGT |
| | GCTCCATGGTCGGGACGCTCTGGCCGGTCAGGCGCGCGCAATC |
| | GTTGACGCTCTAGCGTGCAAAAGGAGAGCCTGTAAGCGGGCAC |
| | TCTTCCGTGGTCTGGTGGATAAATTCGCAAGGGTATCATGGCG |
| | GACGACCGGGGTTCGAGCCCCGTATCCGGCCGTCCGCCGTGAT |
| | CCATGCGGTTACCGCCCGCGTGTCGAACCCAGGTGTGCGACGT |
| | CAGACAACGGGGGAGTGCTCCTTTTGGCTTCCTTCCAGGCGCG |
| | GCGGCTGCTGCGCTAGCTTTTTTGGCCACTGGCCGCGCGCAGCG |
| | TAAGCGGTTAGGCTGGAAAGCGAAAGCATTAAGTGGCTCGCTC |
| | CCTGTAGCCGGAGGGTTATTTTCCAAGGGTTGAGTCGCGGGAC |
| | CCCCGGTTCGAGTCTCGGACCGGCCGGACTGCGGCGAACGGGG |
| | GTTTGCCTCCCCGTCATGCAAGACCCCGCTTGCAAATTCCTCCG |
| | GAAACAGGGACGAGCCCCTTTTTTGCTTTTCCCAGATGCATCCG |
| | GTGCTGCGGCAGATGCGCCCCCCTCCTCAGCAGCGGCAAGAGC |
| | AAGAGCAGCGGCAGACATGCAGGGCACCCTCCCCTCCTCCTAC |
| | CGCGTCAGGAGGGGCGACATCCGCGGTTGACGCGGCAGCAGAT |
| | GGTGATTACGAACCCCCGCGGCGCCGGGCCCGGCACTACCTGG |
| | ACTTGGAGGAGGGCGAGGGCCTGGCGCGGCTAGGAGCGCCCTC |
| | TCCTGAGCGGCACCCAAGGGTGCAGCTGAAGCGTGATACGCGT |
| | GAGGCGTACGTGCCGCGGCAGAACCTGTTTCGCGACCGCGAGG |
| | GAGAGGAGCCCGAGGAGATGCGGGATCGAAAGTTCCACGCAG |
| | GGCGCGAGCTGCGGCATGGCCTGAATCGCGAGCGGTTGCTGCG |
| | CGAGGAGGACTTTGAGCCCGACGCGCGAACCGGGATTAGTCCC |
| | GCGCGCGCACACGTGGCGGCCGCCGACCTGGTAACCGCATACG |
| | AGCAGACGGTGAACCAGGAGATTAACTTTCAAAAAAGCTTTAA |
| | CAACCACGTGCGTACGCTTGTGGCGCGCGAGGAGGTGGCTATA |
| | GGACTGATGCATCTGTGGGACTTTGTAAGCGCGCTGGAGCAAA |
| | ACCCAAATAGCAAGCCGCTCATGGCGCAGCTGTTCCTTATAGT |
| | GCAGCACAGCAGGGACAACGAGGCATTCAGGGATGCGCTGCT |
| | AAACATAGTAGAGCCCGAGGGCCGCTGGCTGCTCGATTTGATA |
| | AACATCCTGCAGAGCATAGTGGTGCAGGAGCGCAGCTTGAGCC |
| | TGGCTGACAAGGTGGCCGCCATCAACTATTCCATGCTTAGCCTG |
| | GGCAAGTTTTACGCCCGCAAGATATACCATACCCCTTACGTTCC |
| | CATAGACAAGGAGGTAAAGATCGAGGGGTTCTACATGCGCATG |
| | GCGCTGAAGGTGCTTACCTTGAGCGACGACCTGGGCGTTTATC |
| | GCAACGAGCGCATCCACAAGGCCGTGAGCGTGAGCCGGCGGC |
| | GCGAGCTCAGCGACCGCGAGCTGATGCACAGCCTGCAAAGGGC |
| | CCTGGCTGGCACGGGCAGCGGCGATAGAGAGGCCGAGTCCTAC |
| | TTTGACGCGGGCGCTGACCTGCGCTGGGCCCCAAGCCGACGCG |
| | CCCTGGAGGCAGCTGGGGCCGGACCTGGGCTGGCGGTGGCACC |
| | CGCGCGCGCTGGCAACGTCGGCGGCGTGGAGGAATATGACGA |
| | GGACGATGAGTACGAGCCAGAGGACGGCGAGTACTAAGCGGT |
| | GATGTTTCTGATCAGATGATGCAAGACGCAACGGACCCGGCGG |
| | TGCGGGCGGCGCTGCAGAGCCAGCCGTCCGGCCTTAACTCCAC |
| | GGACGACTGGCGCCAGGTCATGGACCGCATCATGTCGCTGACT |
| | GCGCGCAATCCTGACGCGTTCCGGCAGCAGCCGCAGGCCAACC |
| | GGCTCTCCGCAATTCTGGAAGCGGTGGTCCCGGCGCGCGCAAA |
| | CCCCACGCACGAGAAGGTGCTGGCGATCGTAAACGCGCTGGCC |
| | GAAAACAGGGCCATCCGGCCCGACGAGGCCGGCCTGGTCTACG |
| | ACGCGCTGCTTCAGCGCGTGGCTCGTTACAACAGCGGCAACGT |
| | GCAGACCAACCTGGACCGGCTGGTGGGGGATGTGCGCGAGGCC |
| | GTGGCGCAGCGTGAGCGCGCGCAGCAGCAGGGCAACCTGGGC |
| | TCCATGGTTGCACTAAACGCCTTCCTGAGTACACAGCCCGCCA |
| | ACGTGCCGCGGGGACAGGAGGACTACACCAACTTTGTGAGCGC |
| | ACTGCGGCTAATGGTGACTGAGACACCGCAAAGTGAGGTGTAC |
| | CAGTCTGGGCCAGACTATTTTTTCCAGACCAGTAGACAAGGCC |
| | TGCAGACCGTAAACCTGAGCCAGGCTTTCAAAAACTTGCAGGG |
| | GCTGTGGGGGGTGCGGGCTCCCACAGGCGACCGCGCGACCGTG |
| | TCTAGCTTGCTGACGCCCAACTCGCGCCTGTTGCTGCTGCTAAT |
| | AGCGCCCTTCACGGACAGTGGCAGCGTGTCCCGGGACACATAC |
| | CTAGGTCACTTGCTGACACTGTACCGCGAGGCCATAGGTCAGG |
| | CGCATGTGGACGAGCATACTTTCCAGGAGATTACAAGTGTCAG |
| | CCGCGCGCTGGGGCAGGAGGACACGGGCAGCCTGGAGGCAAC |
| | CCTAAACTACCTGCTGACCAACCGGCGGCAGAAGATCCCCTCG |
| | TTGCACAGTTTAAACAGCGAGGAGGAGCGCATTTTGCGCTACG |
| | TGCAGCAGAGCGTGAGCCTTAACCTGATGCGCGACGGGGTAAC |
| | GCCCAGCGTGGCGCTGGACATGACCGCGCGCAACATGGAACCG |
| | GGCATGTATGCCTCAAACCGGCCGTTTATCAACCGCCTAATGG |
| | ACTACTTGCATCGCGCGGCCGCCGTGAACCCCGAGTATTTCACC |
| | AATGCCATCTTGAACCCGCACTGGCTACCGCCCCCTGGTTTCTA |
| | CACCGGGGGATTCGAGGTGCCCGAGGGTAACGATGGATTCCTC |
| | TGGGACGACATAGACGACAGCGTGTTTTCCCCGCAACCGCAGA |
| | CCCTGCTAGAGTTGCAACAGCGCGAGCAGGCAGAGGCGGCGCT |
| | GCGAAAGGAAAGCTTCCGCAGGCCAAGCAGCTTGTCCGATCTA |
| | GGCGCTGCGGCCCCGCGGTCAGATGCTAGTAGCCCATTTCCAA |
| | GCTTGATAGGGTCTCTTACCAGCACTCGCACCACCCGCCCGCGC |
| | CTGCTGGGCGAGGAGGAGTACCTAAACAACTCGCTGCTGCAGC |
| | CGCAGCGCGAAAAAAACCTGCCTCCGGCATTTCCCAACAACGG |
| | GATAGAGAGCCTAGTGGACAAGATGAGTAGATGGAAGACGTA |
| | CGCGCAGGAGCACAGGGACGTGCCAGGCCCGCGCCCGCCCACC |
| | CGTCGTCAAAGGCACGACCGTCAGCGGGGTCTGGTGTGGGAGG |
| | ACGATGACTCGGCAGACGACAGCAGCGTCCTGGATTTGGGAGG |
| | GAGTGGCAACCCGTTTGCGCACCTTCGCCCCAGGCTGGGGAGA |
| | ATGTTTTAAAAAAAAAAAAGCATGATGCAAAATAAAAAACTCA |
| | CCAAGGCCATGGCACCGAGCGTTGGTTTTCTTGTATTCCCCTTA |
| | GTATGCGGCGCGCGGCGATGTATGAGGAAGGTCCTCCTCCCTC |
| | CTACGAGAGTGTGGTGAGCGCGGCGCCAGTGGCGGCGGCGCTG |
| | GGTTCTCCCTTCGATGCTCCCCTGGACCCGCCGTTTGTGCCTCC |
| | GCGGTACCTGCGGCCTACCGGGGGGAGAAACAGCATCCGTTAC |
| | TCTGAGTTGGCACCCCTATTCGACACCACCCGTGTGTACCTGGT |
| | GGACAACAAGTCAACGGATGTGGCATCCCTGAACTACCAGAAC |
| | GACCACAGCAACTTTCTGACCACGGTCATTCAAAACAATGACT |
| | ACAGCCCGGGGGAGGCAAGCACACAGACCATCAATCTTGACG |
| | ACCGGTCGCACTGGGGCGGCGACCTGAAAACCATCCTGCATAC |
| | CAACATGCCAAATGTGAACGAGTTCATGTTTACCAATAAGTTT |
| | AAGGCGCGGGTGATGGTGTCGCGCTTGCCTACTAAGGACAATC |
| | AGGTGGAGCTGAAATACGAGTGGGTGGAGTTCACGCTGCCCGA |
| | GGGCAACTACTCCGAGACCATGACCATAGACCTTATGAACAAC |
| | GCGATCGTGGAGCACTACTTGAAAGTGGGCAGACAGAACGGG |
| | GTTCTGGAAAGCGACATCGGGGTAAAGTTTGACACCCGCAACT |
| | TCAGACTGGGGTTTGACCCCGTCACTGGTCTTGTCATGCCTGGG |
| | GTATATACAAACGAAGCCTTCCATCCAGACATCATTTTGCTGCC |
| | AGGATGCGGGGTGGACTTCACCCACAGCCGCCTGAGCAACTTG |
| | TTGGGCATCCGCAAGCGGCAACCCTTCCAGGAGGGCTTTAGGA |
| | TCACCTACGATGATCTGGAGGGTGGTAACATTCCCGCACTGTTG |
| | GATGTGGACGCCTACCAGGCGAGCTTGAAAGATGACACCGAAC |
| | AGGGCGGGGGTGGCGCAGGCGGCAGCAACAGCAGTGGCAGCG |
| | GCGCGGAAGAGAACTCCAACGCGGCAGCCGCGGCAATGCAGC |
| | CGGTGGAGGACATGAACGATCATGCCATTCGCGGCGACACCTT |
| | TGCCACACGGGCTGAGGAGAAGCGCGCTGAGGCCGAAGCAGC |
| | GGCCGAAGCTGCCGCCCCCGCTGCGCAACCCGAGGTCGAGAAG |
| | CCTCAGAAGAAACCGGTGATCAAACCCCTGACAGAGGACAGC |
| | AAGAAACGCAGTTACAACCTAATAAGCAATGACAGCACCTTCA |
| | CCCAGTACCGCAGCTGGTACCTTGCATACAACTACGGCGACCC |
| | TCAGACCGGAATCCGCTCATGGACCCTGCTTTGCACTCCTGACG |
| | TAACCTGCGGCTCGGAGCAGGTCTACTGGTCGTTGCCAGACAT |
| | GATGCAAGACCCCGTGACCTTCCGCTCCACGCGCCAGATCAGC |
| | AACTTTCCGGTGGTGGGCGCCGAGCTGTTGCCCGTGCACTCCA |
| | AGAGCTTCTACAACGACCAGGCCGTCTACTCCCAACTCATCCG |
| | CCAGTTTACCTCTCTGACCCACGTGTTCAATCGCTTTCCCGAGA |
| | ACCAGATTTTGGCGCGCCCGCCAGCCCCCACCATCACCACCGT |
| | CAGTGAAAACGTTCCTGCTCTCACAGATCACGGGACGCTACCG |
| | CTGCGCAACAGCATCGGAGGAGTCCAGCGAGTGACCATTACTG |
| | ACGCCAGACGCCGCACCTGCCCCTACGTTTACAAGGCCCTGGG |
| | CATAGTCTCGCCGCGCGTCCTATCGAGCCGCACTTTTTGAGCAA |
| | GCATGTCCATCCTTATATCGCCCAGCAATAACACAGGCTGGGG |
| | CCTGCGCTTCCCAAGCAAGATGTTTGGCGGGGCCAAGAAGCGC |
| | TCCGACCAACACCCAGTGCGCGTGCGCGGGCACTACCGCGCGC |
| | CCTGGGGCGCGCACAAACGCGGCCGCACTGGGCGCACCACCGT |
| | CGATGACGCCATCGACGCGGTGGTGGAGGAGGCGCGCAACTAC |
| | ACGCCCACGCCGCCACCAGTGTCCACAGTGGACGCGGCCATTC |
| | AGACCGTGGTGCGCGGAGCCCGGCGCTATGCTAAAATGAAGAG |
| | ACGGCGGAGGCGCGTAGCACGTCGCCACCGCCGCCGACCCGGC |
| | ACTGCCGCCCAACGCGCGGCGGCGGCCCTGCTTAACCGCGCAC |
| | GTCGCACCGGCCGACGGGCGGCCATGCGGGCCGCTCGAAGGCT |
| | GGCCGCGGGTATTGTCACTGTGCCCCCCAGGTCCAGGCGACGA |
| | GCGGCCGCCGCAGCAGCCGCGGCCATTAGTGCTATGACTCAGG |
| | GTCGCAGGGGCAACGTGTATTGGGTGCGCGACTCGGTTAGCGG |
| | CCTGCGCGTGCCCGTGCGCACCCGCCCCCCGCGCAACTAGATT |
| | GCAAGAAAAAACTACTTAGACTCGTACTGTTGTATGTATCCAG |
| | CGGCGGCGGCGCGCAACGAAGCTATGTCCAAGCGCAAAATCA |
| | AAGAAGAGATGCTCCAGGTCATCGCGCCGGAGATCTATGGCCC |
| | CCCGAAGAAGGAAGAGCAGGATTACAAGCCCCGAAAGCTAAA |
| | GCGGGTCAAAAAGAAAAAGAAAGATGATGATGATGAACTTGA |
| | CGACGAGGTGGAACTGCTGCACGCTACCGCGCCCAGGCGACGG |
| | GTACAGTGGAAAGGTCGACGCGTAAAACGTGTTTTGCGACCCG |
| | GCACCACCGTAGTCTTTACGCCCGGTGAGCGCTCCACCCGCAC |
| | CTACAAGCGCGTGTATGATGAGGTGTACGGCGACGAGGACCTG |
| | CTTGAGCAGGCCAACGAGCGCCTCGGGGAGTTTGCCTACGGAA |
| | AGCGGCATAAGGACATGCTGGCGTTGCCGCTGGACGAGGGCAA |
| | CCCAACACCTAGCCTAAAGCCCGTAACACTGCAGCAGGTGCTG |
| | CCCGCGCTTGCACCGTCCGAAGAAAAGCGCGGCCTAAAGCGCG |
| | AGTCTGGTGACTTGGCACCCACCGTGCAGCTGATGGTACCCAA |
| | GCGCCAGCGACTGGAAGATGTCTTGGAAAAAATGACCGTGGAA |
| | CCTGGGCTGGAGCCCGAGGTCCGCGTGCGGCCAATCAAGCAGG |
| | TGGCGCCGGGACTGGGCGTGCAGACCGTGGACGTTCAGATACC |
| | CACTACCAGTAGCACCAGTATTGCCACCGCCACAGAGGGCATG |
| | GAGACACAAACGTCCCCGGTTGCCTCAGCGGTGGCGGATGCCG |
| | CGGTGCAGGCGGTCGCTGCGGCCGCGTCCAAGACCTCTACGGA |
| | GGTGCAAACGGACCCGTGGATGTTTCGCGTTTCAGCCCCCCGG |
| | CGCCCGCGCCGTTCGAGGAAGTACGGCGCCGCCAGCGCGCTAC |
| | TGCCCGAATATGCCCTACATCCTTCCATTGCGCCTACCCCCGGC |
| | TATCGTGGCTACACCTACCGCCCCAGAAGACGAGCAACTACCC |
| | GACGCCGAACCACCACTGGAACCCGCCGCCGCCGTCGCCGTCG |
| | CCAGCCCGTGCTGGCCCCGATTTCCGTGCGCAGGGTGGCTCGC |
| | GAAGGAGGCAGGACCCTGGTGCTGCCAACAGCGCGCTACCACC |
| | CCAGCATCGTTTAAAAGCCGGTCTTTGTGGTTCTTGCAGATATG |
| | GCCCTCACCTGCCGCCTCCGTTTCCCGGTGCCGGGATTCCGAGG |
| | AAGAATGCACCGTAGGAGGGGCATGGCCGGCCACGGCCTGAC |
| | GGGCGGCATGCGTCGTGCGCACCACCGGCGGCGGCGCGCGTCG |
| | CACCGTCGCATGCGCGGCGGTATCCTGCCCCTCCTTATTCCACT |
| | GATCGCCGCGGCGATTGGCGCCGTGCCCGGAATTGCATCCGTG |
| | GCCTTGCAGGCGCAGAGACACTGATTAAAAACAAGTTGCATGT |
| | GGAAAAATCAAAATAAAAAGTCTGGACTCTCACGCTCGCTTGG |
| | TCCTGTAACTATTTTGTAGAATGGAAGACATCAACTTTGCGTCT |
| | CTGGCCCCGCGACACGGCTCGCGCCCGTTCATGGGAAACTGGC |
| | AAGATATCGGCACCAGCAATATGAGCGGTGGCGCCTTCAGCTG |
| | GGGCTCGCTGTGGAGCGGCATTAAAAATTTCGGTTCCACCGTT |
| | AAGAACTATGGCAGCAAGGCCTGGAACAGCAGCACAGGCCAG |
| | ATGCTGAGGGATAAGTTGAAAGAGCAAAATTTCCAACAAAAG |
| | GTGGTAGATGGCCTGGCCTCTGGCATTAGCGGGGTGGTGGACC |
| | TGGCCAACCAGGCAGTGCAAAATAAGATTAACAGTAAGCTTGA |
| | TCCCCGCCCTCCCGTAGAGGAGCCTCCACCGGCCGTGGAGACA |
| | GTGTCTCCAGAGGGGCGTGGCGAAAAGCGTCCGCGCCCCGACA |
| | GGGAAGAAACTCTGGTGACGCAAATAGACGAGCCTCCCTCGTA |
| | CGAGGAGGCACTAAAGCAAGGCCTGCCCACCACCCGTCCCATC |
| | GCGCCCATGGCTACCGGAGTGCTGGGCCAGCACACACCCGTAA |
| | CGCTGGACCTGCCTCCCCCCGCCGACACCCAGCAGAAACCTGT |
| | GCTGCCAGGCCCGACCGCCGTTGTTGTAACCCGTCCTAGCCGC |
| | GCGTCCCTGCGCCGCGCCGCCAGCGGTCCGCGATCGTTGCGGC |
| | CCGTAGCCAGTGGCAACTGGCAAAGCACACTGAACAGCATCGT |
| | GGGTCTGGGGGTGCAATCCCTGAAGCGCCGACGATGCTTCTGA |
| | TAGCTAACGTGTCGTATGTGTGTCATGTATGCGTCCATGTCGCC |
| | GCCAGAGGAGCTGCTGAGCCGCCGCGCGCCCGCTTTCCAAGAT |
| | GGCTACCCCTTCGATGATGCCGCAGTGGTCTTACATGCACATCT |
| | CGGGCCAGGACGCCTCGGAGTACCTGAGCCCCGGGCTGGTGCA |
| | GTTTGCCCGCGCCACCGAGACGTACTTCAGCCTGAATAACAAG |
| | TTTAGAAACCCCACGGTGGCGCCTACGCACGACGTGACCACAG |
| | ACCGGTCCCAGCGTTTGACGCTGCGGTTCATCCCTGTGGACCGT |
| | GAGGATACTGCGTACTCGTACAAGGCGCGGTTCACCCTAGCTG |
| | TGGGTGATAACCGTGTGCTGGACATGGCTTCCACGTACTTTGAC |
| | ATCCGCGGCGTGCTGGACAGGGGCCCTACTTTTAAGCCCTACTC |
| | TGGCACTGCCTACAACGCCCTGGCTCCCAAGGGTGCCCCAAAT |
| | CCTTGCGAATGGGATGAAGCTGCTACTGCTCTTGAAATAAACC |
| | TAGAAGAAGAGGACGATGACAACGAAGACGAAGTAGACGAGC |
| | AAGCTGAGCAGCAAAAAACTCACGTATTTGGGCAGGCGCCTTA |
| | TTCTGGTATAAATATTACAAAGGAGGGTATTCAAATAGGTGTC |
| | GAAGGTCAAACACCTAAATATGCCGATAAAACATTTCAACCTG |
| | AACCTCAAATAGGAGAATCTCAGTGGTACGAAACAGAAATTAA |
| | TCATGCAGCTGGGAGAGTCCTAAAAAAGACTACCCCAATGAAA |
| | CCATGTTACGGTTCATATGCAAAACCCACAAATGAAAATGGAG |
| | GGCAAGGCATTCTTGTAAAGCAACAAAATGGAAAGCTAGAAA |
| | GTCAAGTGGAAATGCAATTTTTCTCAACTACTGAGGCAGCCGC |
| | AGGCAATGGTGATAACTTGACTCCTAAAGTGGTATTGTACAGT |
| | GAAGATGTAGATATAGAAACCCCAGACACTCATATTTCTTACA |
| | TGCCCACTATTAAGGAAGGTAACTCACGAGAACTAATGGGCCA |
| | ACAATCTATGCCCAACAGGCCTAATTACATTGCTTTTAGGGACA |
| | ATTTTATTGGTCTAATGTATTACAACAGCACGGGTAATATGGGT |
| | GTTCTGGCGGGCCAAGCATCGCAGTTGAATGCTGTTGTAGATTT |
| | GCAAGACAGAAACACAGAGCTTTCATACCAGCTTTTGCTTGAT |
| | TCCATTGGTGATAGAACCAGGTACTTTTCTATGTGGAATCAGGC |
| | TGTTGACAGCTATGATCCAGATGTTAGAATTATTGAAAATCATG |
| | GAACTGAAGATGAACTTCCAAATTACTGCTTTCCACTGGGAGG |
| | TGTGATTAATACAGAGACTCTTACCAAGGTAAAACCTAAAACA |
| | GGTCAGGAAAATGGATGGGAAAAAGATGCTACAGAATTTTCAG |
| | ATAAAAATGAAATAAGAGTTGGAAATAATTTTGCCATGGAAAT |
| | CAATCTAAATGCCAACCTGTGGAGAAATTTCCTGTACTCCAAC |
| | ATAGCGCTGTATTTGCCCGACAAGCTAAAGTACAGTCCTTCCA |
| | ACGTAAAAATTTCTGATAACCCAAACACCTACGACTACATGAA |
| | CAAGCGAGTGGTGGCTCCCGGGCTAGTGGACTGCTACATTAAC |
| | CTTGGAGCACGCTGGTCCCTTGACTATATGGACAACGTCAACC |
| | CATTTAACCACCACCGCAATGCTGGCCTGCGCTACCGCTCAATG |
| | TTGCTGGGCAATGGTCGCTATGTGCCCTTCCACATCCAGGTGCC |
| | TCAGAAGTTCTTTGCCATTAAAAACCTCCTTCTCCTGCCGGGCT |
| | CATACACCTACGAGTGGAACTTCAGGAAGGATGTTAACATGGT |
| | TCTGCAGAGCTCCCTAGGAAATGACCTAAGGGTTGACGGAGCC |
| | AGCATTAAGTTTGATAGCATTTGCCTTTACGCCACCTTCTTCCC |
| | CATGGCCCACAACACCGCCTCCACGCTTGAGGCCATGCTTAGA |
| | AACGACACCAACGACCAGTCCTTTAACGACTATCTCTCCGCCG |
| | CCAACATGCTCTACCCTATACCCGCCAACGCTACCAACGTGCCC |
| | ATATCCATCCCCTCCCGCAACTGGGCGGCTTTCCGCGGCTGGGC |
| | CTTCACGCGCCTTAAGACTAAGGAAACCCCATCACTGGGCTCG |
| | GGCTACGACCCTTATTACACCTACTCTGGCTCTATACCCTACCT |
| | AGATGGAACCTTTTACCTCAACCACACCTTTAAGAAGGTGGCC |
| | ATTACCTTTGACTCTTCTGTCAGCTGGCCTGGCAATGACCGCCT |
| | GCTTACCCCCAACGAGTTTGAAATTAAGCGCTCAGTTGACGGG |
| | GAGGGTTACAACGTTGCCCAGTGTAACATGACCAAAGACTGGT |
| | TCCTGGTACAAATGCTAGCTAACTATAACATTGGCTACCAGGG |
| | CTTCTATATCCCAGAGAGCTACAAGGACCGCATGTACTCCTTCT |
| | TTAGAAACTTCCAGCCCATGAGCCGTCAGGTGGTGGATGATAC |
| | TAAATACAAGGACTACCAACAGGTGGGCATCCTACACCAACAC |
| | AACAACTCTGGATTTGTTGGCTACCTTGCCCCCACCATGCGCGA |
| | AGGACAGGCCTACCCTGCTAACTTCCCCTATCCGCTTATAGGCA |
| | AGACCGCAGTTGACAGCATTACCCAGAAAAAGTTTCTTTGCGA |
| | TCGCACCCTTTGGCGCATCCCATTCTCCAGTAACTTTATGTCCA |
| | TGGGCGCACTCACAGACCTGGGCCAAAACCTTCTCTACGCCAA |
| | CTCCGCCCACGCGCTAGACATGACTTTTGAGGTGGATCCCATG |
| | GACGAGCCCACCCTTCTTTATGTTTTGTTTGAAGTCTTTGACGT |
| | GGTCCGTGTGCACCAGCCGCACCGCGGCGTCATCGAAACCGTG |
| | TACCTGCGCACGCCCTTCTCGGCCGGCAACGCCACAACATAAA |
| | GAAGCAAGCAACATCAACAACAGCTGCCGCCATGGGCTCCAGT |
| | GAGCAGGAACTGAAAGCCATTGTCAAAGATCTTGGTTGTGGGC |
| | CATATTTTTTGGGCACCTATGACAAGCGCTTTCCAGGCTTTGTT |
| | TCTCCACACAAGCTCGCCTGCGCCATAGTCAATACGGCCGGTC |
| | GCGAGACTGGGGGCGTACACTGGATGGCCTTTGCCTGGAACCC |
| | GCACTCAAAAACATGCTACCTCTTTGAGCCCTTTGGCTTTTCTG |
| | ACCAGCGACTCAAGCAGGTTTACCAGTTTGAGTACGAGTCACT |
| | CCTGCGCCGTAGCGCCATTGCTTCTTCCCCCGACCGCTGTATAA |
| | CGCTGGAAAAGTCCACCCAAAGCGTACAGGGGCCCAACTCGGC |
| | CGCCTGTGGACTATTCTGCTGCATGTTTCTCCACGCCTTTGCCA |
| | ACTGGCCCCAAACTCCCATGGATCACAACCCCACCATGAACCT |
| | TATTACCGGGGTACCCAACTCCATGCTCAACAGTCCCCAGGTA |
| | CAGCCCACCCTGCGTCGCAACCAGGAACAGCTCTACAGCTTCC |
| | TGGAGCGCCACTCGCCCTACTTCCGCAGCCACAGTGCGCAGAT |
| | TAGGAGCGCCACTTCTTTTTGTCACTTGAAAAACATGTAAAAAT |
| | AATGTACTAGAGACACTTTCAATAAAGGCAAATGCTTTTATTTG |
| | TACACTCTCGGGTGATTATTTACCCCCACCCTTGCCGTCTGCGC |
| | CGTTTAAAAATCAAAGGGGTTCTGCCGCGCATCGCTATGCGCC |
| | ACTGGCAGGGACACGTTGCGATACTGGTGTTTAGTGCTCCACTT |
| | AAACTCAGGCACAACCATCCGCGGCAGCTCGGTGAAGTTTTCA |
| | CTCCACAGGCTGCGCACCATCACCAACGCGTTTAGCAGGTCGG |
| | GCGCCGATATCTTGAAGTCGCAGTTGGGGCCTCCGCCCTGCGC |
| | GCGCGAGTTGCGATACACAGGGTTGCAGCACTGGAACACTATC |
| | AGCGCCGGGTGGTGCACGCTGGCCAGCACGCTCTTGTCGGAGA |
| | TCAGATCCGCGTCCAGGTCCTCCGCGTTGCTCAGGGCGAACGG |
| | AGTCAACTTTGGTAGCTGCCTTCCCAAAAAGGGCGCGTGCCCA |
| | GGCTTTGAGTTGCACTCGCACCGTAGTGGCATCAAAAGGTGAC |
| | CGTGCCCGGTCTGGGCGTTAGGATACAGCGCCTGCATAAAAGC |
| | CTTGATCTGCTTAAAAGCCACCTGAGCCTTTGCGCCTTCAGAGA |
| | AGAACATGCCGCAAGACTTGCCGGAAAACTGATTGGCCGGACA |
| | GGCCGCGTCGTGCACGCAGCACCTTGCGTCGGTGTTGGAGATC |
| | TGCACCACATTTCGGCCCCACCGGTTCTTCACGATCTTGGCCTT |
| | GCTAGACTGCTCCTTCAGCGCGCGCTGCCCGTTTTCGCTCGTCA |
| | CATCCATTTCAATCACGTGCTCCTTATTTATCATAATGCTTCCGT |
| | GTAGACACTTAAGCTCGCCTTCGATCTCAGCGCAGCGGTGCAG |
| | CCACAACGCGCAGCCCGTGGGCTCGTGATGCTTGTAGGTCACC |
| | TCTGCAAACGACTGCAGGTACGCCTGCAGGAATCGCCCCATCA |
| | TCGTCACAAAGGTCTTGTTGCTGGTGAAGGTCAGCTGCAACCC |
| | GCGGTGCTCCTCGTTCAGCCAGGTCTTGCATACGGCCGCCAGA |
| | GCTTCCACTTGGTCAGGCAGTAGTTTGAAGTTCGCCTTTAGATC |
| | GTTATCCACGTGGTACTTGTCCATCAGCGCGCGCGCAGCCTCCA |
| | TGCCCTTCTCCCACGCAGACACGATCGGCACACTCAGCGGGTT |
| | CATCACCGTAATTTCACTTTCCGCTTCGCTGGGCTCTTCCTCTTC |
| | CTCTTGCGTCCGCATACCACGCGCCACTGGGTCGTCTTCATTCA |
| | GCCGCCGCACTGTGCGCTTACCTCCTTTGCCATGCTTGATTAGC |
| | ACCGGTGGGTTGCTGAAACCCACCATTTGTAGCGCCACATCTTC |
| | TCTTTCTTCCTCGCTGTCCACGATTACCTCTGGTGATGGCGGGC |
| | GCTCGGGCTTGGGAGAAGGGCGCTTCTTTTTCTTCTTGGGCGCA |
| | ATGGCCAAATCCGCCGCCGAGGTCGATGGCCGCGGGCTGGGTG |
| | TGCGCGGCACCAGCGCGTCTTGTGATGAGTCTTCCTCGTCCTCG |
| | GACTCGATACGCCGCCTCATCCGCTTTTTTGGGGGCGCCCGGGG |
| | AGGCGGCGGCGACGGGGACGGGGACGACACGTCCTCCATGGTT |
| | GGGGGACGTCGCGCCGCACCGCGTCCGCGCTCGGGGGTGGTTT |
| | CGCGCTGCTCCTCTTCCCGACTGGCCATTTCCTTCTCCTATAGG |
| | CAGAAAAAGATCATGGAGTCAGTCGAGAAGAAGGACAGCCTA |
| | ACCGCCCCCTCTGAGTTCGCCACCACCGCCTCCACCGATGCCGC |
| | CAACGCGCCTACCACCTTCCCCGTCGAGGCACCCCCGCTTGAG |
| | GAGGAGGAAGTGATTATCGAGCAGGACCCAGGTTTTGTAAGCG |
| | AAGACGACGAGGACCGCTCAGTACCAACAGAGGATAAAAAGC |
| | AAGACCAGGACAACGCAGAGGCAAACGAGGAACAAGTCGGGC |
| | GGGGGGACGAAAGGCATGGCGACTACCTAGATGTGGGAGACG |
| | ACGTGCTGTTGAAGCATCTGCAGCGCCAGTGCGCCATTATCTGC |
| | GACGCGTTGCAAGAGCGCAGCGATGTGCCCCTCGCCATAGCGG |
| | ATGTCAGCCTTGCCTACGAACGCCACCTATTCTCACCGCGCGTA |
| | CCCCCCAAACGCCAAGAAAACGGCACATGCGAGCCCAACCCGC |
| | GCCTCAACTTCTACCCCGTATTTGCCGTGCCAGAGGTGCTTGCC |
| | ACCTATCACATCTTTTTCCAAAACTGCAAGATACCCCTATCCTG |
| | CCGTGCCAACCGCAGCCGAGCGGACAAGCAGCTGGCCTTGCGG |
| | CAGGGCGCTGTCATACCTGATATCGCCTCGCTCAACGAAGTGC |
| | CAAAAATCTTTGAGGGTCTTGGACGCGACGAGAAGCGCGCGGC |
| | AAACGCTCTGCAACAGGAAAACAGCGAAAATGAAAGTCACTCT |
| | GGAGTGTTGGTGGAACTCGAGGGTGACAACGCGCGCCTAGCCG |
| | TACTAAAACGCAGCATCGAGGTCACCCACTTTGCCTACCCGGC |
| | ACTTAACCTACCCCCCAAGGTCATGAGCACAGTCATGAGTGAG |
| | CTGATCGTGCGCCGTGCGCAGCCCCTGGAGAGGGATGCAAATT |
| | TGCAAGAACAAACAGAGGAGGGCCTACCCGCAGTTGGCGACG |
| | AGCAGCTAGCGCGCTGGCTTCAAACGCGCGAGCCTGCCGACTT |
| | GGAGGAGCGACGCAAACTAATGATGGCCGCAGTGCTCGTTACC |
| | GTGGAGCTTGAGTGCATGCAGCGGTTCTTTGCTGACCCGGAGA |
| | TGCAGCGCAAGCTAGAGGAAACATTGCACTACACCTTTCGACA |
| | GGGCTACGTACGCCAGGCCTGCAAGATCTCCAACGTGGAGCTC |
| | TGCAACCTGGTCTCCTACCTTGGAATTTTGCACGAAAACCGCCT |
| | TGGGCAAAACGTGCTTCATTCCACGCTCAAGGGCGAGGCGCGC |
| | CGCGACTACGTCCGCGACTGCGTTTACTTATTTCTATGCTACAC |
| | CTGGCAGACGGCCATGGGCGTTTGGCAGCAGTGCTTGGAGGAG |
| | TGCAACCTCAAGGAGCTGCAGAAACTGCTAAAGCAAAACTTGA |
| | AGGACCTATGGACGGCCTTCAACGAGCGCTCCGTGGCCGCGCA |
| | CCTGGCGGACATCATTTTCCCCGAACGCCTGCTTAAAACCCTGC |
| | AACAGGGTCTGCCAGACTTCACCAGTCAAAGCATGTTGCAGAA |
| | CTTTAGGAACTTTATCCTAGAGCGCTCAGGAATCTTGCCCGCCA |
| | CCTGCTGTGCACTTCCTAGCGACTTTGTGCCCATTAAGTACCGC |
| | GAATGCCCTCCGCCGCTTTGGGGCCACTGCTACCTTCTGCAGCT |
| | AGCCAACTACCTTGCCTACCACTCTGACATAATGGAAGACGTG |
| | AGCGGTGACGGTCTACTGGAGTGTCACTGTCGCTGCAACCTAT |
| | GCACCCCGCACCGCTCCCTGGTTTGCAATTCGCAGCTGCTTAAC |
| | GAAAGTCAAATTATCGGTACCTTTGAGCTGCAGGGTCCCTCGC |
| | CTGACGAAAAGTCCGCGGCTCCGGGGTTGAAACTCACTCCGGG |
| | GCTGTGGACGTCGGCTTACCTTCGCAAATTTGTACCTGAGGACT |
| | ACCACGCCCACGAGATTAGGTTCTACGAAGACCAATCCCGCCC |
| | GCCTAATGCGGAGCTTACCGCCTGCGTCATTACCCAGGGCCAC |
| | ATTCTTGGCCAATTGCAAGCCATCAACAAAGCCCGCCAAGAGT |
| | TTCTGCTACGAAAGGGACGGGGGGTTTACTTGGACCCCCAGTC |
| | CGGCGAGGAGCTCAACCCAATCCCCCCGCCGCCGCAGCCCTAT |
| | CAGCAGCAGCCGCGGGCCCTTGCTTCCCAGGATGGCACCCAAA |
| | AAGAAGCTGCAGCTGCCGCCGCCACCCACGGACGAGGAGGAA |
| | TACTGGGACAGTCAGGCAGAGGAGGTTTTGGACGAGGAGGAG |
| | GAGGACATGATGGAAGACTGGGAGAGCCTAGACGAGGAAGCT |
| | TCCGAGGTCGAAGAGGTGTCAGACGAAACACCGTCACCCTCGG |
| | TCGCATTCCCCTCGCCGGCGCCCCAGAAATCGGCAACCGGTTC |
| | CAGCATGGCTACAACCTCCGCTCCTCAGGCGCCGCCGGCACTG |
| | CCCGTTCGCCGACCCAACCGTAGATGGGACACCACTGGAACCA |
| | GGGCCGGTAAGTCCAAGCAGCCGCCGCCGTTAGCCCAAGAGCA |
| | ACAACAGCGCCAAGGCTACCGCTCATGGCGCGGGCACAAGAA |
| | CGCCATAGTTGCTTGCTTGCAAGACTGTGGGGGCAACATCTCCT |
| | TCGCCCGCCGCTTTCTTCTCTACCATCACGGCGTGGCCTTCCCC |
| | CGTAACATCCTGCATTACTACCGTCATCTCTACAGCCCATACTG |
| | CACCGGCGGCAGCGGCAGCAACAGCAGCGGCCACACAGAAGC |
| | AAAGGCGACCGGATAGCAAGACTCTGACAAAGCCCAAGAAAT |
| | CCACAGCGGCGGCAGCAGCAGGAGGAGGAGCGCTGCGTCTGG |
| | CGCCCAACGAACCCGTATCGACCCGCGAGCTTAGAAACAGGAT |
| | TTTTCCCACTCTGTATGCTATATTTCAACAGAGCAGGGGCCAAG |
| | AACAAGAGCTGAAAATAAAAAACAGGTCTCTGCGATCCCTCAC |
| | CCGCAGCTGCCTGTATCACAAAAGCGAAGATCAGCTTCGGCGC |
| | ACGCTGGAAGACGCGGAGGCTCTCTTCAGTAAATACTGCGCGC |
| | TGACTCTTAAGGACTAGTTTCGCGCCCTTTCTCAAATTTAAGCG |
| | CGAAAACTACGTCATCTCCAGCGGCCACACCCGGCGCCAGCAC |
| | CTGTTGTCAGCGCCATTATGAGCAAGGAAATTCCCACGCCCTA |
| | CATGTGGAGTTACCAGCCACAAATGGGACTTGCGGCTGGAGCT |
| | GCCCAAGACTACTCAACCCGAATAAACTACATGAGCGCGGGAC |
| | CCCACATGATATCCCGGGTCAACGGAATACGCGCCCACCGAAA |
| | CCGAATTCTCCTGGAACAGGCGGCTATTACCACCACACCTCGT |
| | AATAACCTTAATCCCCGTAGTTGGCCCGCTGCCCTGGTGTACCA |
| | GGAAAGTCCCGCTCCCACCACTGTGGTACTTCCCAGAGACGCC |
| | CAGGCCGAAGTTCAGATGACTAACTCAGGGGCGCAGCTTGCGG |
| | GCGGCTTTCGTCACAGGGTGCGGTCGCCCGGGCAGGGTATAAC |
| | TCACCTGACAATCAGAGGGCGAGGTATTCAGCTCAACGACGAG |
| | TCGGTGAGCTCCTCGCTTGGTCTCCGTCCGGACGGGACATTTCA |
| | GATCGGCGGCGCCGGCCGCTCTTCATTCACGCCTCGTCAGGCA |
| | ATCCTAACTCTGCAGACCTCGTCCTCTGAGCCGCGCTCTGGAGG |
| | CATTGGAACTCTGCAATTTATTGAGGAGTTTGTGCCATCGGTCT |
| | ACTTTAACCCCTTCTCGGGACCTCCCGGCCACTATCCGGATCAA |
| | TTTATTCCTAACTTTGACGCGGTAAAGGACTCGGCGGACGGCT |
| | ACGACTGAATGTTAAGTGGAGAGGCAGAGCAACTGCGCCTGAA |
| | ACACCTGGTCCACTGTCGCCGCCACAAGTGCTTTGCCCGCGACT |
| | CCGGTGAGTTTTGCTACTTTGAATTGCCCGAGGATCATATCGAG |
| | GGCCCGGCGCACGGCGTCCGGCTTACCGCCCAGGGAGAGCTTG |
| | CCCGTAGCCTGATTCGGGAGTTTACCCAGCGCCCCCTGCTAGTT |
| | GAGCGGGACAGGGGACCCTGTGTTCTCACTGTGATTTGCAACT |
| | GTCCTAACCCTGGATTACATCAAGATCCTCTAGTTAATGTCAGG |
| | TCGCCTAAGTCGATTAACTAGAGTACCCGGGGATCTTATTCCCT |
| | TTAACTAATAAAAAAAAATAATAAAGCATCACTTACTTAAAAT |
| | CAGTTAGCAAATTTCTGTCCAGTTTATTCAGCAGCACCTCCTTG |
| | CCCTCCTCCCAGCTCTGGTATTGCAGCTTCCTCCTGGCTGCAAA |
| | CTTTCTCCACAATCTAAATGGAATGTCAGTTTCCTCCTGTTCCT |
| | GTCCATCCGCACCCACTATCTTCATGTTGTTGCAGATGAAGCGC |
| | GCAAGACCGTCTGAAGATACCTTCAACCCCGTGTATCCATATG |
| | ACACGGAAACCGGTCCTCCAACTGTGCCTTTTCTTACTCCTCCC |
| | TTTGTATCCCCCAATGGGTTTCAAGAGAGTCCCCCTGGGGTACT |
| | CTCTTTGCGCCTATCCGAACCTCTAGTTACCTCCAATGGCATGC |
| | TTGCGCTCAAAATGGGCAACGGCCTCTCTCTGGACGAGGCCGG |
| | CAACCTTACCTCCCAAAATGTAACCACTGTGAGCCCACCTCTCA |
| | AAAAAACCAAGTCAAACATAAACCTGGAAATATCTGCACCCCT |
| | CACAGTTACCTCAGAAGCCCTAACTGTGGCTGCCGCCGCACCT |
| | CTAATGGTCGCGGGCAACACACTCACCATGCAATCACAGGCCC |
| | CGCTAACCGTGCACGACTCCAAACTTAGCATTGCCACCCAAGG |
| | ACCCCTCACAGTGTCAGAAGGAAAGCTAGCCCTGCAAACATCA |
| | GGCCCCCTCACCACCACCGATAGCAGTACCCTTACTATCACTGC |
| | CTCACCCCCTCTAACTACTGCCACTGGTAGCTTGGGCATTGACT |
| | TGAAAGAGCCCATTTATACACAAAATGGAAAACTAGGACTAAA |
| | GTACGGGGCTCCTTTGCATGTAACAGACGACCTAAACACTTTG |
| | ACCGTAGCAACTGGTCCAGGTGTGACTATTAATAATACTTCCTT |
| | GCAAACTAAAGTTACTGGAGCCTTGGGTTTTGATTCACAAGGC |
| | AATATGCAACTTAATGTAGCAGGAGGACTAAGGATTGATTCTC |
| | AAAACAGACGCCTTATACTTGATGTTAGTTATCCGTTTGATGCT |
| | CAAAACCAACTAAATCTAAGACTAGGACAGGGCCCTCTTTTTA |
| | TAAACTCAGCCCACAACTTGGATATTAACTACAACAAAGGCCT |
| | TTACTTGTTTACAGCTTCAAACAATTCCAAAAAGCTTGAGGTTA |
| | ACCTAAGCACTGCCAAGGGGTTGATGTTTGACGCTACAGCCAT |
| | AGCCATTAATGCAGGAGATGGGCTTGAATTTGGTTCACCTAAT |
| | GCACCAAACACAAATCCCCTCAAAACAAAAATTGGCCATGGCC |
| | TAGAATTTGATTCAAACAAGGCTATGGTTCCTAAACTAGGAAC |
| | TGGCCTTAGTTTTGACAGCACAGGTGCCATTACAGTAGGAAAC |
| | AAAAATAATGATAAGCTAACTTTGTGGACCACACCAGCTCCAT |
| | CTCCTAACTGTAGACTAAATGCAGAGAAAGATGCTAAACTCAC |
| | TTTGGTCTTAACAAAATGTGGCAGTCAAATACTTGCTACAGTTT |
| | CAGTTTTGGCTGTTAAAGGCAGTTTGGCTCCAATATCTGGAACA |
| | GTTCAAAGTGCTCATCTTATTATAAGATTTGACGAAAATGGAGT |
| | GCTACTAAACAATTCCTTCCTGGACCCAGAATATTGGAACTTTA |
| | GAAATGGAGATCTTACTGAAGGCACAGCCTATACAAACGCTGT |
| | TGGATTTATGCCTAACCTATCAGCTTATCCAAAATCTCACGGTA |
| | AAACTGCCAAAAGTAACATTGTCAGTCAAGTTTACTTAAACGG |
| | AGACAAAACTAAACCTGTAACACTAACCATTACACTAAACGGT |
| | ACACAGGAAACAGGAGACACAACTCCAAGTGCATACTCTATGT |
| | CATTTTCATGGGACTGGTCTGGCCACAACTACATTAATGAAATA |
| | TTTGCCACATCCTCTTACACTTTTTCATACATTGCCCAAGAATA |
| | AAGAATCGTTTGTGTTATGTTTCAACGTGTTTATTTTTCAATTGC |
| | AGAAAATTTCAAGTCATTTTTCATTCAGTAGTATAGCCCCACCA |
| | CCACATAGCTTATACAGATCACCGTACCTTAATCAAACTCACA |
| | GAACCCTAGTATTCAACCTGCCACCTCCCTCCCAACACACAGA |
| | GTACACAGTCCTTTCTCCCCGGCTGGCCTTAAAAAGCATCATAT |
| | CATGGGTAACAGACATATTCTTAGGTGTTATATTCCACACGGTT |
| | TCCTGTCGAGCCAAACGCTCATCAGTGATATTAATAAACTCCCC |
| | GGGCAGCTCACTTAAGTTCATGTCGCTGTCCAGCTGCTGAGCCA |
| | CAGGCTGCTGTCCAACTTGCGGTTGCTTAACGGGCGGCGAAGG |
| | AGAAGTCCACGCCTACATGGGGGTAGAGTCATAATCGTGCATC |
| | AGGATAGGGCGGTGGTGCTGCAGCAGCGCGCGAATAAACTGCT |
| | GCCGCCGCCGCTCCGTCCTGCAGGAATACAACATGGCAGTGGT |
| | CTCCTCAGCGATGATTCGCACCGCCCGCAGCATAAGGCGCCTT |
| | GTCCTCCGGGCACAGCAGCGCACCCTGATCTCACTTAAATCAG |
| | CACAGTAACTGCAGCACAGCACCACAATATTGTTCAAAATCCC |
| | ACAGTGCAAGGCGCTGTATCCAAAGCTCATGGCGGGGACCACA |
| | GAACCCACGTGGCCATCATACCACAAGCGCAGGTAGATTAAGT |
| | GGCGACCCCTCATAAACACGCTGGACATAAACATTACCTCTTTT |
| | GGCATGTTGTAATTCACCACCTCCCGGTACCATATAAACCTCTG |
| | ATTAAACATGGCGCCATCCACCACCATCCTAAACCAGCTGGCC |
| | AAAACCTGCCCGCCGGCTATACACTGCAGGGAACCGGGACTGG |
| | AACAATGACAGTGGAGAGCCCAGGACTCGTAACCATGGATCAT |
| | CATGCTCGTCATGATATCAATGTTGGCACAACACAGGCACACG |
| | TGCATACACTTCCTCAGGATTACAAGCTCCTCCCGCGTTAGAAC |
| | CATATCCCAGGGAACAACCCATTCCTGAATCAGCGTAAATCCC |
| | ACACTGCAGGGAAGACCTCGCACGTAACTCACGTTGTGCATTG |
| | TCAAAGTGTTACATTCGGGCAGCAGCGGATGATCCTCCAGTAT |
| | GGTAGCGCGGGTTTCTGTCTCAAAAGGAGGTAGACGATCCCTA |
| | CTGTACGGAGTGCGCCGAGACAACCGAGATCGTGTTGGTCGTA |
| | GTGTCATGCCAAATGGAACGCCGGACGTAGTCATATTTCCTGA |
| | AGCAAAACCAGGTGCGGGCGTGACAAACAGATCTGCGTCTCCG |
| | GTCTCGCCGCTTAGATCGCTCTGTGTAGTAGTTGTAGTATATCC |
| | ACTCTCTCAAAGCATCCAGGCGCCCCCTGGCTTCGGGTTCTATG |
| | TAAACTCCTTCATGCGCCGCTGCCCTGATAACATCCACCACCGC |
| | AGAATAAGCCACACCCAGCCAACCTACACATTCGTTCTGCGAG |
| | TCACACACGGGAGGAGCGGGAAGAGCTGGAAGAACCATGTTTT |
| | TTTTTTTATTCCAAAAGATTATCCAAAACCTCAAAATGAAGATC |
| | TATTAAGTGAACGCGCTCCCCTCCGGTGGCGTGGTCAAACTCTA |
| | CAGCCAAAGAACAGATAATGGCATTTGTAAGATGTTGCACAAT |
| | GGCTTCCAAAAGGCAAACGGCCCTCACGTCCAAGTGGACGTAA |
| | AGGCTAAACCCTTCAGGGTGAATCTCCTCTATAAACATTCCAGC |
| | ACCTTCAACCATGCCCAAATAATTCTCATCTCGCCACCTTCTCA |
| | ATATATCTCTAAGCAAATCCCGAATATTAAGTCCGGCCATTGTA |
| | AAAATCTGCTCCAGAGCGCCCTCCACCTTCAGCCTCAAGCAGC |
| | GAATCATGATTGCAAAAATTCAGGTTCCTCACAGACCTGTATA |
| | AGATTCAAAAGCGGAACATTAACAAAAATACCGCGATCCCGTA |
| | GGTCCCTTCGCAGGGCCAGCTGAACATAATCGTGCAGGTCTGC |
| | ACGGACCAGCGCGGCCACTTCCCCGCCAGGAACCATGACAAAA |
| | GAACCCACACTGATTATGACACGCATACTCGGAGCTATGCTAA |
| | CCAGCGTAGCCCCGATGTAAGCTTGTTGCATGGGCGGCGATAT |
| | AAAATGCAAGGTGCTGCTCAAAAAATCAGGCAAAGCCTCGCGC |
| | AAAAAAGAAAGCACATCGTAGTCATGCTCATGCAGATAAAGGC |
| | AGGTAAGCTCCGGAACCACCACAGAAAAAGACACCATTTTTCT |
| | CTCAAACATGTCTGCGGGTTTCTGCATAAACACAAAATAAAAT |
| | AACAAAAAAACATTTAAACATTAGAAGCCTGTCTTACAACAGG |
| | AAAAACAACCCTTATAAGCATAAGACGGACTACGGCCATGCCG |
| | GCGTGACCGTAAAAAAACTGGTCACCGTGATTAAAAAGCACCA |
| | CCGACAGCTCCTCGGTCATGTCCGGAGTCATAATGTAAGACTC |
| | GGTAAACACATCAGGTTGATTCACATCGGTCAGTGCTAAAAAG |
| | CGACCGAAATAGCCCGGGGGAATACATACCCGCAGGCGTAGA |
| | GACAACATTACAGCCCCCATAGGAGGTATAACAAAATTAATAG |
| | GAGAGAAAAACACATAAACACCTGAAAAACCCTCCTGCCTAGG |
| | CAAAATAGCACCCTCCCGCTCCAGAACAACATACAGCGCTTCC |
| | ACAGCGGCAGCCATAACAGTCAGCCTTACCAGTAAAAAAGAA |
| | AACCTATTAAAAAAACACCACTCGACACGGCACCAGCTCAATC |
| | AGTCACAGTGTAAAAAAGGGCCAAGTGCAGAGCGAGTATATAT |
| | AGGACTAAAAAATGACGTAACGGTTAAAGTCCACAAAAAACA |
| | CCCAGAAAACCGCACGCGAACCTACGCCCAGAAACGAAAGCC |
| | AAAAAACCCACAACTTCCTCAAATCGTCACTTCCGTTTTCCCAC |
| | GTTACGTCACTTCCCATTTTAAGAAAACTACAATTCCCAACACA |
| | TACAAGTTACTCCGCCCTAAAACCTACGTCACCCGCCCCGTTCC |
| | CACGCCCCGCGCCACGTCACAAACTCCACCCCCTCATTATCATA |
| | TTGGCTTCAATCCAAAATAAGGTATATTATTGATGAT |
| SEQ ID NO: | ATGACACCGGGCACCCAGTCTCCTTTCTTCCTGCTGCTGCTCCT |
| 26 | CACAGTGCTTACAGTTGTTACGGGTTCTGGTCATGCAAGCTCTA |
| | CCCCAGGTGGAGAAAAGGAGACTTCGGCTACCCAGAGAAGTTC |
| | AGTGCCCAGCTCTACTGAGAAGAATGCTGTGAGTATGACCAGC |
| | AGCGTACTCTCCAGCCACAGCCCCGGTTCAGGCTCCTCCACCAC |
| | TCAGGGACAGGATGTCACTCTGGCCCCGGCCACGGAACCAGCT |
| | TCAGGTTCAGCTGCCCTTTGGGGACAGGATGTCACCTCGGTCCC |
| | AGTCACCAGGCCAGCCCTGGGCTCCACCACCCCGCCAGCCCAC |
| | GATGTCACCTCAGCCCCGGACAACAAGCCAGCCCCGGGCTCCA |
| | CCGCCCCCCCAGCCCACGGTGTCACCTCGTATCTTGACACCAGG |
| | CCGGCCCCGGTTTATCTTGCCCCCCCAGCCCATGGTGTCACCTC |
| | GGCCCCGGACAACAGGCCCGCCTTGGGCTCCACCGCCCCTCCA |
| | GTCCACAATGTCACCTCGGCCTCAGGCTCTGCATCAGGCTCAGC |
| | TTCTACTCTGGTGCACAACGGCACCTCTGCCAGGGCTACCACA |
| | ACCCCAGCCAGCAAGAGCACTCCATTCTCAATTCCCAGCCACC |
| | ACTCTGATACTCCTACCACCCTTGCCAGCCATAGCACCAAGACT |
| | GATGCCAGTAGCACTCACCATAGCACGGTACCTCCTCTCACCTC |
| | CTCCAATCACAGCACTTCTCCCCAGTTGTCTACTGGGGTCTCTT |
| | TCTTTTTCCTGTCTTTTCACATTTCAAACCTCCAGTTTAATTCCT |
| | CTCTGGAAGATCCCAGCACCGACTACTACCAAGAGCTGCAGAG |
| | AGACATTTCTGAAATGTTTTTGCAGATTTATAAACAAGGGGGTT |
| | TTCTGGGCCTCTCCAATATTAAGTTCAGGCCAGGATCTGTGGTG |
| | GTACAATTGACTCTGGCCTTCCGAGAAGGTACCATCAATGTCC |
| | ACGACGTGGAGACACAGTTCAATCAGTATAAAACGGAAGCAG |
| | CCTCTCGATATAACCTGACGATCTCAGACGTCAGCGTGAGTGA |
| | TGTGCCATTTCCTTTCTCTGCCCAGTCTGGGGCTGGGGTGCCAG |
| | GCTGGGGCATCGCGCTGCTGGTGCTGGTCTGTGTTCTGGTTTAT |
| | CTGGCCATTGTCTATCTCATTGCCTTGGCTGTCGCTCAGGTTCG |
| | CCGAAAGAACTACGGGCAGCTGGACATCTTTCCAGCCCGGGAT |
| | AAATACCATCCTATGAGCGAGTACGCTCTTTACCACACCCATG |
| | GGCGCTATGTGCCCCCTAGCAGTCTTTTCCGTAGCCCCTATGAG |
| | AAGGTTTCTGCAGGTAATGGTGGCAGCTATCTCTCTTACACAAA |
| | CCCAGCAGTGGCAGCCGCTTCTGCCAACTTGTAG |
| SEQ ID NO: | MTPGTQSPFFLLLLLTVLTVVTGSGHASSTPGGEKETSATQRSSVP |
| 27 | SSTEKNAVSMTSSVLSSHSPGSGSSTTQGQDVTLAPATEPASGSAA |
| | LWGQDVTSVPVTRPALGSTTPPAHDVTSAPDNKPAPGSTAPPAHG |
| | VTSYLDTRPAPVYLAPPAHGVTSAPDNRPALGSTAPPVHNVTSAS |
| | GSASGSASTLVHNGTSARATTTPASKSTPFSIPSHHSDTPTTLASHS |
| | TKTDASSTHHSTVPPLTSSNHSTSPQLSTGVSFFFLSFHISNLQFNSS |
| | LEDPSTDYYQELQRDISEMFLQIYKQGGFLGLSNIKFRPGSVVVQL |
| | TLAFREGTTNVHDVETQFNQYKTEAASRYNLTISDVSVSDVPFPFS |
| | AQSGAGVPGWGIALLVLVCVLVYLAIVYLIALAVAQVRRKNYGQ |
| | LDIFPARDKYHPMSEYALYHTHGRYVPPSSLFRSPYEKVSAGNGG |
| | SYLSYTNPAVAAASANL |
| SEQ ID NO: | ATGAGCTCCCCTGGCACCGAGAGCGCGGGAAAGAGCCTGCAGT |
| 28 | ACCGAGTGGACCACCTGCTGAGCGCCGTGGAGAATGAGCTGCA |
| | GGCGGGCAGCGAGAAGGGCGACCCCACAGAGCGCGAACTGCG |
| | CGTGGGCCTGGAGGAGAGCGAGCTGTGGCTGCGCTTCAAGGAG |
| | CTCACCAATGAGATGATCGTGACCAAGAACGGCAGGAGGATGT |
| | TTCCGGTGCTGAAGGTGAACGTGTCTGGCCTGGACCCCAACGC |
| | CATGTACTCCTTCCTGCTGGACTTCGTGGCGGCGGACAACCACC |
| | GCTGGAAGTACGTGAACGGGGAATGGGTGCCGGGGGGCAAGC |
| | CGGAGCCGCAGGCGCCCAGCTGCGTCTACATCCACCCCGACTC |
| | GCCCAACTTCGGGGCCCACTGGATGAAGGCTCCCGTCTCCTTCA |
| | GCAAAGTCAAGCTCACCAACAAGCTCAACGGAGGGGGCCAGA |
| | TCATGCTGAACTCCTTGCATAAGTATGAGCCTCGAATCCACATA |
| | GTGAGAGTTGGGGGTCCACAGCGCATGATCACCAGCCACTGCT |
| | TCCCTGAGACCCAGTTCATAGCGGTGACTGCTAGAAGTGATCA |
| | CAAAGAGATGATGGAGGAACCCGGAGACAGCCAGCAACCTGG |
| | GTACTCCCAATGGGGGTGGCTTCTTCCTGGAACCAGCACCGTGT |
| | GTCCACCTGCAAATCCTCATCCTCAGTTTGGAGGTGCCCTCTCC |
| | CTCCCCTCCACGCACAGCTGTGACAGGTACCCAACCCTGAGGA |
| | GCCACCGGTCCTCACCCTACCCCAGCCCCTATGCTCATCGGAAC |
| | AATTCTCCAACCTATTCTGACAACTCACCTGCATGTTTATCCAT |
| | GCTGCAATCCCATGACAATTGGTCCAGCCTTGGAATGCCTGCCC |
| | ATCCCAGCATGCTCCCCGTGAGCCACAATGCCAGCCCACCTAC |
| | CAGCTCCAGTCAGTACCCCAGCCTGTGGTCTGTGAGCAACGGC |
| | GCCGTCACCCCGGGCTCCCAGGCAGCAGCCGTGTCCAACGGGC |
| | TGGGGGCCCAGTTCTTCCGGGGCTCCCCCGCGCACTACACACC |
| | CCTCACCCATCCGGTCTCGGCGCCCTCTTCCTCGGGATCCCCAC |
| | TGTACGAAGGGGCGGCCGCGGCCACAGACATCGTGGACAGCC |
| | AGTACGACGCCGCAGCCCAAGGCCGCCTCATAGCCTCATGGAC |
| | ACCTGTGTCGCCACCTTCCATGTGA |

1. A composition comprising a replication-defective virus vector comprising a nucleic acid sequence comprising one or more of:
a) a nucleic acid sequence encoding an amino acid sequence at least 80%, at least 85%, at least 90%, at least 92%, at least 95%, at least 97%, or at least 99% identical to SEQ ID NO: 8, SEQ ID NO: 9, or SEQ ID NO: 10;
b) a nucleic acid sequence encoding an amino acid sequence at least 80%, at least 85%, at least 90%, at least 92%, at least 95%, at least 97%, or at least 99% identical to SEQ ID NO: 12;
c) a nucleic acid sequence having a region at least 80%, at least 85%, at least 90%, at least 92%, at least 95%, at least 97%, or at least 99% identical to SEQ ID NO: 2, SEQ ID NO: 3, or SEQ ID NO: 4;
d) a nucleic acid sequence having a region at least 80%, at least 85%, at least 90%, at least 92%, at least 95%, at least 97%, or at least 99% identical to SEQ ID NO: 5, SEQ ID NO: 18, SEQ ID NO: 6, SEQ ID NO: 19, or SEQ ID NO: 7, SEQ ID NO: 20; and
e) a nucleic acid sequence having a region at least 80%, at least 85%, at least 90%, at least 92%, at least 95%, at least 97%, or at least 99% identical to SEQ ID NO: 11, or SEQ ID NO: 21.

2.-16. (canceled)

17. The composition of claim 1, wherein the vector is an adenovirus vector.

18. The composition of claim 17, wherein the vector comprises a deletion in an E1 region, an E2b region, an E3 region, an E4 region, or a combination thereof.

19. (canceled)

20. (canceled)

21. The composition of claim 1, wherein the composition or the vector further comprises a nucleic acid sequences encoding a costimulatory molecule

22. The composition of claim 21, wherein the costimulatory molecule comprises B7, ICAM-1, LFA-3, or a combination thereof.

23. (canceled)

24. The composition of claim 1, wherein the composition further comprises a plurality of nucleic acid sequences encoding a plurality of costimulatory molecules positioned in the same replication-defective virus vector.

25. The composition of claim 1, wherein the composition further comprises a plurality of nucleic acid sequences encoding a plurality of costimulatory molecules positioned in separate replication-defective virus vectors.

26. The composition of claim 1, wherein the composition comprises at least 5×10¹¹ replication-defective virus vectors.

27. The composition of claim 1, wherein the composition comprises a nucleotide sequence encoding a fusion protein comprising HPV E6 and HPV E7.

28. The composition of claim 1, wherein the composition comprises:
a first replication defective adenovirus vector comprising: a deletion in the E2b region, and a nucleic acid sequence encoding HPV E6; and
a second replication defective adenovirus vector comprising: a deletion in the E2b region, and a nucleic acid sequence encoding HPV E7.

29. The composition of claim 1, wherein the replication-defective virus vector further comprises a nucleic acid sequence encoding a selectable marker.

30. (canceled)

31. (canceled)

32. The composition of claim 1, wherein the antigen is a non-oncogenic HPV antigen.

33. The composition of claim 1, wherein the antigen binds to HLA-A2, HLA-A3, HLA-A24, or a combination thereof.

34. (canceled)

35. (canceled)

36. (canceled)

37. The composition of claim 1, wherein the replication-defective virus further comprises a nucleic acid sequence encoding one or more additional target antigens or immunological epitopes thereof.

38. (canceled)

39. The composition of claim 37, wherein the one or more additional target antigens is CEA, folate receptor alpha, WT1, HPV E6, HPV E7, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, PSCA, PSMA, PAP, Tyrosinase, TRP-1, TRP-2, ART-4, CAMEL, Cyp-B, Her2/neu, BRCA1, BRACHYURY, BRACHYURY (TIVS7-2, polymorphism), BRACHYURY (IVS7 T/C polymorphism), T BRACHYURY, T, hTERT, hTRT, iCE, MUC1, MUC1 (VNTR polymorphism), MUC1c, MUC1n, MUC2, PRAME, P15, RU1, RU2, SART-1, SART-3, WT1, AFP, (3-catenin/m, Caspase-8/m, CDK-4/m, Her2/neu, Her3, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist, or a combination thereof.

40. (canceled)

41. (canceled)

42. (canceled)

43. (canceled)

44. The composition of claim 1, wherein the composition comprises from at least 1×10⁹ virus particles to at least 5×10¹² virus particles.

45. (canceled)

46. (canceled)

47. The composition of claim 1, wherein the replication-defective virus vector further comprises a nucleic acid sequence encoding an immunological fusion partner.

48. A pharmaceutical composition comprising the composition according to claim 1 and a pharmaceutically acceptable carrier.

49. A host cell comprising the composition according to claim 1.

50. A method of preparing a tumor vaccine, comprising preparing a composition according to claim 1.

51. A method of enhancing an HPV-specific immune response in a subject in need thereof, the method comprising administering a therapeutically effective amount of the composition of claim 1 to the subject.

52. A method of preventing or treating a HPV-induced cancer in a subject in need thereof, the method comprising administering a therapeutically effective amount of the composition of claim 1 to the subject.

53.-90. (canceled)

91. A method of reducing HPV-expressing cells in a subject in need thereof, the method comprising administering an effective amount of a composition comprising a replication-defective virus vector comprising a nucleic acid sequence encoding a modified HPV E6, a modified HPV E7 antigen, or a combination thereof.

92. The method of claim 91, wherein the nucleic acid sequence encodes a modified HPV E6 and a modified HPV E7.

93. The method of claim 91, wherein the replication-defective virus vector comprises:
a) a nucleic acid sequence encoding an amino acid sequence at least 80%, at least 85%, at least 90%, at least 92%, at least 95%, at least 97%, or at least 99% identical to SEQ ID NO: 8, SEQ ID NO: 9, or SEQ ID NO: 10;
b) a nucleic acid sequence encoding an amino acid sequence at least 80%, at least 85%, at least 90%, at least 92%, at least 95%, at least 97%, or at least 99% identical to SEQ ID NO: 12;
c) a nucleic acid sequence having a region at least 80%, at least 85%, at least 90%, at least 92%, at least 95%, at least 97%, or at least 99% identical to SEQ ID NO: 2, SEQ ID NO: 3, or SEQ ID NO: 4;
d) a nucleic acid sequence having a region at least 80%, at least 85%, at least 90%, at least 92%, at least 95%, at least 97%, or at least 99% identical to SEQ ID NO: 5, SEQ ID NO: 18, SEQ ID NO: 6, SEQ ID NO: 19, SEQ ID NO: 7, SEQ ID NO: 20;
e) a nucleic acid sequence having a region at least 80%, at least 85%, at least 90%, at least 92%, at least 95%, at least 97%, or at least 99% identical to SEQ ID NO: 11 or SEQ ID NO: 21;
f) a nucleic acid sequence encoding an amino acid sequence at least 80%, at least 85%, at least 90%, at least 92%, at least 95%, at least 97%, or at least 99% identical to SEQ ID NO: 13;
g) a nucleic acid sequence encoding an amino acid sequence at least 80%, at least 85%, at least 90%, at least 92%, at least 95%, at least 97%, or at least 99% identical to SEQ ID NO: 14; or
h) a nucleic acid sequence comprising a region at least 80%, at least 85%, at least 90%, at least 92%, at least 95%, at least 97%, or at least 99% identical to SEQ ID NO: 15.

94. The method of claim 91, wherein the administering eliminates HPV E6 or HPV E7-expressing cells in the subject.

95.-132. (canceled)


SEQUENCE LISTING (120 sequences)

SEQ ID NO: 1 - DNA, 9 bp, Gallus gallus
  modified_base (8)..(8): a, c, t, g, unknown or other
tctctccna

SEQ ID NO: 2 - DNA, 812 bp, Artificial Sequence
  Description of Artificial Sequence Synthetic polynucleotide
ctcgaggaagcttgccgccaccatgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtaatcatgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcgtactaggaattgtgtgccccatctgttctcagaaaccatgagatatcgcggccgc

SEQ ID NO: 3 - DNA, 812 bp, Artificial Sequence
  Description of Artificial Sequence Synthetic polynucleotide
ctcgaggaagcttgccgccaccatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtaatcatgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcgtactaggaattgtgtgccccatctgttctcagaaaccatgagatatcgcggccgc

SEQ ID NO: 4 - DNA, 812 bp, Artificial Sequence
  Description of Artificial Sequence Synthetic polynucleotide
ctcgaggaagcttgccgccaccatgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtaatcatgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcgtactaggaattgtgtgccccatctgttctcagaaaccatgagatatcgcggccgc

SEQ ID NO: 5 - DNA, 477 bp, Artificial Sequence
  Description of Artificial Sequence Synthetic polynucleotide
atgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtga

SEQ ID NO: 6 - DNA, 477 bp, Artificial Sequence
  Description of Artificial Sequence Synthetic polynucleotide
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtga

SEQ ID NO: 7 - DNA, 477 bp, Artificial Sequence
  Description of Artificial Sequence Synthetic polynucleotide
atgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtga

SEQ ID NO: 8 - protein, 158 aa, Artificial Sequence
  Description of Artificial Sequence Synthetic polypeptide
MHQKRTAMFQDPQERPRKLPQLCTEVQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAA

SEQ ID NO: 9 - protein, 158 aa, Artificial Sequence
  Description of Artificial Sequence Synthetic polypeptide
MHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQLYNKPLCDVLIRCINCQKPLCPEEKQRHLDKKQRFHNIRGRWTGRCMSCCRSSRTRRAAAA

SEQ ID NO: 10 - protein, 158 aa, Artificial Sequence
  Description of Artificial Sequence Synthetic polypeptide
MHQKRTAMFQDPQERPRKLPQLCTEVQTTIHDIILECVYCKQQLLRREVYDFAFRDGCIVYRDGNPYAVCDKCLKFYSKISEYRHYCYSLYGTTLEQLYNKPLCDVLIRCINCQKPLCPEEKQRHLDKKQRFHNIRGRWTGRCMSCCRSSRTRRAAAA

SEQ ID NO: 11 - DNA, 297 bp, Artificial Sequence
  Description of Artificial Sequence Synthetic polynucleotide
atgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcgtactaggaattgtgtgccccatctgttctcagaaaccatga

SEQ ID NO: 12 - protein, 98 aa, Artificial Sequence
  Description of Artificial Sequence Synthetic polypeptide
MPGDTPTLHEYMLDLQPETTDLYGYEQLNDSSEEEDEIDGPAGQAAPDRAHYNIVTFCCKCDSTLRRCVQSTHVDIRTLEDLLMGVLGIVCPICSQKP

SEQ ID NO: 13 - protein, 158 aa, Artificial Sequence
  Description of Artificial Sequence Synthetic polypeptide
MHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAA

SEQ ID NO: 14 - protein, 98 aa, Artificial Sequence
  Description of Artificial Sequence Synthetic polypeptide
MPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKP

SEQ ID NO: 15 - DNA, 1147 bp, Artificial Sequence
  Description of Artificial Sequence Synthetic polynucleotide
aagcagaggctcgtttagtgaaccgtcagatggtaccgtttaaactcgaggtcgacggtatcgataagcttgatatcgaattcgagctcggtacccccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtaatcatgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcacactaggaattgtgtgccccatctgttctcagaaaccataatctaccatggctgatcctgcagcatgcaagctggggatccactagttctagagcggccgccacagcggggagatcagacatgatagatacattgatgagtttggacaaaccacaactagaatgcagtgaaaaaatgctttattgtgaaattgtgatgctattgcttatttgtacattatagctgcaataaacagttacaacaacaattgcattcatttatgttcaggtcagggggaaggtgtggaggtt

SEQ ID NO: 16 - DNA, 31026 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagatgcatgctcgagcggccgccagtgtgatggatatctgcagaattcgcccttgctctcgaggaagcttgccgccaccatgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtaatcatgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcgtactaggaattgtgtgccccatctgttctcagaaaccatgagatatcgcggccgccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 17 - DNA, 30214 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagatgcatgctcgagcggccgccagtgtgatggatatctgcagaattcgcccttgctcgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 18 - DNA, 513 bp, Artificial Sequence
  Description of Artificial Sequence Synthetic polynucleotide
ctcgaggaagcttgccgccaccatgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtgagatatcgcggccgc

SEQ ID NO: 19 - DNA, 513 bp, Artificial Sequence
  Description of Artificial Sequence Synthetic polynucleotide
ctcgaggaagcttgccgccaccatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtgagatatcgcggccgc

SEQ ID NO: 20 - DNA, 513 bp, Artificial Sequence
  Description of Artificial Sequence Synthetic polynucleotide
ctcgaggaagcttgccgccaccatgcaccaaaagagaactgcaatgtttcaggacccacaggagcgacccagaaagttaccacagttatgcacagaggtgcaaacaactatacatgatataatattagaatgtgtgtactgcaagcaacagttactgcgacgtgaggtatatgactttgcttttcgggatggatgcatagtatatagagatgggaatccatatgctgtatgtgataaatgtttaaagttttattctaaaattagtgagtatagacattattgttatagtttgtatggaacaacattagaacagctatacaacaaaccgttgtgtgatgtgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaactcgtagagcagccgcggcgtgagatatcgcggccgc

SEQ ID NO: 21 - DNA, 333 bp, Artificial Sequence
  Description of Artificial Sequence Synthetic polynucleotide
ctcgaggaagcttgccgccaccatgcctggagatacacctacattgcatgaatatatgttagatttgcaaccagagacaactgatctctacggttatgagcaattaaatgacagctcagaggaggaggatgaaatagatggtccagctggacaagcagcaccggacagagcccattacaatattgtaaccttttgttgcaagtgtgactctacgcttcggaggtgcgtacaaagcacacacgtagacattcgtactttggaagacctgttaatgggcgtactaggaattgtgtgccccatctgttctcagaaaccatgagatatcgcggccgc

SEQ ID NO: 22 - DNA, 2109 bp, Homo sapiens
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 23 - protein, 9 aa, Homo sapiens
YLSGANLNL

SEQ ID NO: 24 - protein, 9 aa, Homo sapiens
YLSGADLNL

SEQ ID NO: 25 - DNA, 32315 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccgagctcggatccactagtaacggccgccagtgtgctggaattcggcttaaaggtacccagagcagacagccgccaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 26 - DNA, 1428 bp, Homo sapiens
atgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtag

SEQ ID NO: 27 - protein, 475 aa, Artificial Sequence
  Description of Artificial Sequence Synthetic polypeptide
MTPGTQSPFFLLLLLTVLTVVTGSGHASSTPGGEKETSATQRSSVPSSTEKNAVSMTSSVLSSHSPGSGSSTTQGQDVTLAPATEPASGSAALWGQDVTSVPVTRPALGSTTPPAHDVTSAPDNKPAPGSTAPPAHGVTSYLDTRPAPVYLAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVYLAIVYLIALAVAQVRRKNYGQLDIFPARDKYHPMSEYALYHTHGRYVPPSSLFRSPYEKVSAGNGGSYLSYTNPAVAAASANL

SEQ ID NO: 28 - DNA, 1233 bp, Homo sapiens
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 29 - protein, 132 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 30 - protein, 230 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFDDDDKDPPDPHQPDMTKGYCPGGRWGFGDLAVCDGEKYPDGSFWHQWMQTWFTGPQFYFDCVSGGEPLPGPPPPGGCGGAIPSEQPNAP

SEQ ID NO: 31 - protein, 578 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYSGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 32 - protein, 220 aa, Artificial Sequence [sequence omitted]

SEQ ID NO: 33 - protein, 729 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 34 - protein, 30 aa, Artificial Sequence
  Description of Artificial Sequence Synthetic polypeptide
TAASDNFQLSQGGQGFAIPIGQAMAIAGQI

SEQ ID NO: 35 - protein, 128 aa, Artificial Sequence
  Description of Artificial Sequence Synthetic polypeptide
TAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 36 - protein, 128 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAE

SEQ ID NO: 37 - protein, 355 aa, Mycobacterium tuberculosis
MSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 38 - protein, 364 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPAFFTDVNQMYDVLLNKSGATGVFTDFPDTGVEFLKGIK

SEQ ID NO: 39 - protein, 313 aa, Streptococcus pneumonae
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPEFRMSQMA

SEQ ID NO: 40 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 41 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 42 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 43 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 44 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 45 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 47 - protein, 152 aa, Homo sapiens
MSRLPVLLLLQLLVRPGLQAPMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 48 - protein, 153 aa, Homo sapiens [sequence omitted]

SEQ ID NO: 49 - protein, 134 aa, Homo sapiens
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 50 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 51 - protein, 140 aa, Homo sapiens
MVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 52 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 53 - protein, 145 aa, Homo sapiens [sequence omitted]

SEQ ID NO: 54 - protein, 136 aa, Homo sapiens [sequence omitted]

SEQ ID NO: 55 - protein, 656 aa, Homo sapiens
MEGDGSDPEPPDAGEDSKSENGENAPIYCICRKPDINCFMIGCDNCNEWFHGDCIRITEKMAKAIREWYCRECREKDPKLEIRYRHKKSRERDGNERDSSEPRDEGGGRKRPVPDPNLQRRAGSGTGVGAMLARGSASPHKSSPQPLVATPSQHHQQQQQQIKRSARMCGECEACRRTEDCGHCDFCRDMKKFGGPNKIRQKCRLRQCQLRARESYKYFPSSLSPVTPSESLPRPRRPLPTQQQPQPSQKLGRIREDEGAVASSTVKEPPEATATPEPLSDEDLPLDPDLYQDFCAGAFDDNGLPWMSDTEESPFLDPALRKRAVKVKHVKRREKKSEKKKEERYKRHRQKQKHKDKWKHPERADAKDPASLPQCLGPGCVRPAQPSSKYCSDDCGMKLAANRIYEILPQRIQQWQQSPCIAEEHGKKLLERIRREQQSARTRLQEMERRFHELEAIILRAKQQAVREDEESNEGDSDDTDLQIFCVSCGHPINPRVALRHMERCYAKYESQTSFGSMYPTRIEGATRLFCDVYNPQSKTYCKRLQVLCPEHSRDPKVPADEVCGCPLVRDVFELTGDFCRLPKRQCNRHYCWEKLRRAEVDLERVRVWYKLDELFEQERNVRTAMTNRAGLLALMLHQTIQHDPLTTDLRSSADR

SEQ ID NO: 56 - protein, 124 aa, Vibrio cholerae
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 57 - protein, 258 aa, Vibrio cholerae
MVKIIFVFFIFLSSFSYANDDKLYRADSRPPDEIKQSGGLMPRGQNEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 58 - protein, 124 aa, Vibrio cholerae
MIKLKFGVFFTVLLSSAYAHGTPQNITDLCAEYHNTQIHTLNDKILSYTESLAGNREMAIITFKNGATFQVEVPGSQHIDSQKKAIERMKDTLRIAYLTEAKVEKLCVWNNKTPHAIAAISMAN

SEQ ID NO: 59 - protein, 18 aa, Homo sapiens
DPNAPKRPPSAFFLFCSE

SEQ ID NO: 60 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 61 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 62 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 63 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 64 - protein, 7 aa, Artificial Sequence
  Description of Artificial Sequence Synthetic peptide
QEINSSY

SEQ ID NO: 65 - protein, 7 aa, Artificial Sequence
  Description of Artificial Sequence Synthetic peptide
SHPRLSA

SEQ ID NO: 66 - protein, 7 aa, Artificial Sequence
  Description of Artificial Sequence Synthetic peptide
SMPNPMV

SEQ ID NO: 67 - protein, 7 aa, Artificial Sequence
  Description of Artificial Sequence Synthetic peptide
GLQQVLL

SEQ ID NO: 68 - protein, 7 aa, Artificial Sequence
  Description of Artificial Sequence Synthetic peptide
HELSVLL

SEQ ID NO: 69 - protein, 7 aa, Artificial Sequence
  Description of Artificial Sequence Synthetic peptide
YAPQRLP

SEQ ID NO: 70 - protein, 7 aa, Artificial Sequence
  Description of Artificial Sequence Synthetic peptide
TPRTLPT

SEQ ID NO: 71 - protein, 7 aa, Artificial Sequence
  Description of Artificial Sequence Synthetic peptide
APVHSSI

SEQ ID NO: 72 - protein, 7 aa, Artificial Sequence
  Description of Artificial Sequence Synthetic peptide
APPHALS

SEQ ID NO: 73 - protein, 7 aa, Artificial Sequence
  Description of Artificial Sequence Synthetic peptide
TFSNRFI

SEQ ID NO: 74 - protein, 7 aa, Artificial Sequence
  Description of Artificial Sequence Synthetic peptide
VVPTPPY

SEQ ID NO: 75 - protein, 7 aa, Artificial Sequence
  Description of Artificial Sequence Synthetic peptide
ELAPDSP

SEQ ID NO: 76 - protein, 69 aa, Shigella dysenteria 1
TPDCVTGKVEYTKYNDDDTFTVKVGDKELFTNRWNLQSLLLSAQITGMTVTIKQNACHNGGGFSEVIFR

SEQ ID NO: 77 - protein, 560 aa, Corynephage omega
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 78 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 79 - protein, 297 aa, Artificial Sequence
  Description of Artificial Sequence Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 81 - protein, 1331 aa, Homo sapiens [sequence omitted]

SEQ ID NO: 82 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 83 - protein, 476 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 84 - protein, 234 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 85 - protein, 11 aa, Artificial Sequence
  Description of Artificial Sequence Synthetic peptide
MAVPMQLSCSR

SEQ ID NO: 86 - protein, 4 aa, Artificial Sequence
  Description of Artificial Sequence Synthetic peptide
RSTG

SEQ ID NO: 87 - protein, 2 aa, Artificial Sequence
  Description of Artificial Sequence Synthetic peptide
TR

SEQ ID NO: 88 - protein, 3 aa, Artificial Sequence
  Description of Artificial Sequence Synthetic peptide
RSQ

SEQ ID NO: 89 - protein, 5 aa, Artificial Sequence
  Description of Artificial Sequence Synthetic peptide
RSAGE

SEQ ID NO: 90 - protein, 2 aa, Artificial Sequence
  Description of Artificial Sequence Synthetic peptide
RS

SEQ ID NO: 91 - protein, 2 aa, Artificial Sequence
  Description of Artificial Sequence Synthetic peptide
GG

SEQ ID NO: 92 - protein, 9 aa, Artificial Sequence
  Description of Artificial Sequence Synthetic peptide
GSGGSGGSG

SEQ ID NO: 93 - protein, 11 aa, Artificial Sequence
  Description of Artificial Sequence Synthetic peptide
GGSGGSGGSGG

SEQ ID NO: 94 - protein, 14 aa, Artificial Sequence
  Description of Artificial Sequence Synthetic peptide
GGSGGSGGSGGSGG

SEQ ID NO: 95 - protein, 17 aa, Artificial Sequence
  Description of Artificial Sequence Synthetic peptide
GGSGGSGGSGGSGGSGG

SEQ ID NO: 96 - protein, 20 aa, Artificial Sequence
  Description of Artificial Sequence Synthetic peptide
GGSGGSGGSGGSGGSGGSGG

SEQ ID NO: 97 - protein, 23 aa, Artificial Sequence
  Description of Artificial Sequence Synthetic peptide
GGSGGSGGSGGSGGSGGSGGSGG

SEQ ID NO: 98 - protein, 16 aa, Artificial Sequence
  Description of Artificial Sequence Synthetic peptide
GGSGGSGGSGGSGGSG

SEQ ID NO: 99 - protein, 16 aa, Artificial Sequence
  Description of Artificial Sequence Synthetic peptide
GSGGSGGSGGSGGSGG

SEQ ID NO: 100 - protein, 9 aa, Artificial Sequence
  Description of Artificial Sequence Synthetic peptide
EQVWGMAVR

SEQ ID NO: 101 - protein, 15 aa, Artificial Sequence
  Description of Artificial Sequence Synthetic peptide
CQGPEQVWGMAVREL

SEQ ID NO: 102 - protein, 9 aa, Artificial Sequence
  Description of Artificial Sequence Synthetic peptide
GETVTMPCP

SEQ ID NO: 103 - protein, 15 aa, Artificial Sequence
  Description of Artificial Sequence Synthetic peptide
NVGETVTMPCPKVFS

SEQ ID NO: 104 - protein, 9 aa, Artificial Sequence
  Description of Artificial Sequence Synthetic peptide
GLGAQCSEA

SEQ ID NO: 105 - protein, 15 aa, Artificial Sequence
  Description of Artificial Sequence Synthetic peptide
NNGLGAQCSEAVTLN

SEQ ID NO: 106 - protein, 9 aa, Artificial Sequence
  Description of Artificial Sequence Synthetic peptide
RKLTTELTI

SEQ ID NO: 107 - protein, 15 aa, Artificial Sequence
  Description of Artificial Sequence Synthetic peptide
LGPERRKLTTELTII

SEQ ID NO: 108 - protein, 9 aa, Artificial Sequence
  Description of Artificial Sequence Synthetic peptide
PERRKLTTE

SEQ ID NO: 109 - protein, 9 aa, Artificial Sequence
  Description of Artificial Sequence Synthetic peptide
MDWVWMDTT

SEQ ID NO: 110 - protein, 15 aa, Artificial Sequence
  Description of Artificial Sequence Synthetic peptide
AVMDWVWMDTTLSLS

SEQ ID NO: 111 - protein, 9 aa, Artificial Sequence
  Description of Artificial Sequence Synthetic peptide
VWMDTTLSL

SEQ ID NO: 112 - protein, 9 aa, Artificial Sequence
  Description of Artificial Sequence Synthetic peptide
GKTLNPSQT

SEQ ID NO: 113 - protein, 15 aa, Artificial Sequence
  Description of Artificial Sequence Synthetic peptide
SWFREGKTLNPSQTS

SEQ ID NO: 114 - protein, 9 aa, Artificial Sequence
  Description of Artificial Sequence Synthetic peptide
REGKTLNPS

SEQ ID NO: 115 - protein, 9 aa, Artificial Sequence
  Description of Artificial Sequence Synthetic peptide
VRNATSYRC

SEQ ID NO: 116 - protein, 15 aa, Artificial Sequence
  Description of Artificial Sequence Synthetic peptide
LPNVTVRNATSYRCG

SEQ ID NO: 117 - protein, 9 aa, Artificial Sequence
  Description of Artificial Sequence Synthetic peptide
NVTVRNATS

SEQ ID NO: 118 - protein, 9 aa, Artificial Sequence
  Description of Artificial Sequence Synthetic peptide
FAMAQIPSL

SEQ ID NO: 119 - protein, 15 aa, Artificial Sequence
  Description of Artificial Sequence Synthetic peptide
PFAMAQIPSLSLRAV

SEQ ID NO: 120 - protein, 9 aa, Artificial Sequence
  Description of Artificial Sequence Synthetic peptide
AQIPSLSLR